SEQUENCE LISTING (321 sequences)

SEQ ID NO: 1 - protein, 196 aa, Artificial Sequence
  Synthetic Construct
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSK

SEQ ID NO: 2 - protein, 588 aa, Artificial Sequence
  Synthetic Construct
AGGGCCACTCCAGAGAATTACCTTTTCCAGGGACGGCAGGAATGCTACGCGTTTAATGGGACACAGCGCTTCCTGGAGAGATACATCTACAACCGGGAGGAGTTCGCGCGCTTCGACAGCGACGTGGGGGAGTTCCGGGCGGTGACGGAGCTGGGGCGGCCTGCTGCGGAGTACTGGAACAGCCAGAAGGACATCCTGGAGGAGAAGCGGGCAGTGCCGGACAGGATGTGCAGACACAACTACGAGCTGGGCGGGCCCATGACCCTGCAGCGCCGAGTCCAGCCTAGGGTGAATGTTTCCCCCTCCAAGAAGGGGCCCTTGCAGCACCACAACCTGCTTGTCTGCCACGTGACGGATTTCTACCCAGGCAGCATTCAAGTCCGATGGTTCCTGAATGGACAGGAGGAAACAGCTGGGGTCGTGTCCACCAACCTGATCCGTAATGGAGACTGGACCTTCCAGATCCTGGTGATGCTGGAAATGACCCCCCAGCAGGGAGATGTCTACACCTGCCAAGTGGAGCACACCAGCCTGGATAGTCCTGTCACCGTGGAGTGGAAGGCACAGTCTGATTCTGCCCGGAGTAAG

SEQ ID NO: 3 - protein, 196 aa, Artificial Sequence
  Synthetic Construct
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNWLVCHVTDFYPGSIQVRWFLNGQEETAGVMSTNLIRNGDWTFQILVMLEMTPQQGDVYTCQVEHTSLDSPVTVEWKAQSDSARSK

SEQ ID NO: 4 - protein, 265 aa, Artificial Sequence
  Synthetic Construct
MMRPIVLVLLFATSALARATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNWLVCHVTDFYPGSIQVRWFLNGQEETAGVMSTNLIRNGDWTFQILVMLEMTPQQGDVYTCQVEHTSLDSPVTVEWKAQSDSARSKGGGGSLEIEAAFLERENTALETRVAELRQRVQRLRNRVSQYRTRYGPLGGGK

SEQ ID NO: 5 - protein, 258 aa, Artificial Sequence
  Synthetic Construct
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 6 - protein, 192 aa, Artificial Sequence
  Synthetic Construct
IKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTET

SEQ ID NO: 7 - protein, 576 aa, Artificial Sequence
  Synthetic Construct
ATCAAGGCCGACCACGTGTCCACATACGCCGCCTTCGTGCAGACCCACAGACCCACCGGCGAGTTCATGTTCGAGTTCGACGAGGACGAGATGTTCTACGTGGACCTGGACAAGAAAGAAACCGTGTGGCACCTGGAAGAGTTCGGCCAGGCCTTCAGCTTTGAGGCCCAGGGCGGACTGGCCAATATCGCCATCCTGAACAACAACCTGAACACCCTGATCCAGCGGAGCAACCACACCCAGGCCACCAACGATCCCCCCGAAGTGACCGTGTTCCCCAAAGAACCCGTGGAACTGGGCCAGCCCAATACCCTGATCTGCCACATCGACAAGTTCTTCCCCCCCGTGCTGAACGTGACCTGGCTGTGCAATGGCGAGCTCGTGACAGAGGGCGTGGCCGAGTCTCTGTTCCTGCCCAGAACCGACTACAGCTTCCACAAGTTCCACTACCTGACCTTCGTGCCCAGCGCCGAGGACTTCTACGACTGCAGAGTGGAACACTGGGGCCTGGACCAGCCCCTGCTGAAACATTGGGAAGCCCAGGAACCCATCCAGATGCCCGAGACAACCGAGACA

SEQ ID NO: 8 - protein, 269 aa, Artificial Sequence
  Synthetic Construct
MMRPIVLVLLFATSALAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETGGGGSLEIRAAFLRQRNTALRTEVAELEQEVQRLENEVSQYETRYGPLGGGKGSHHHHHH

SEQ ID NO: 9 - protein, 17 aa, Artificial Sequence
  Synthetic Construct
MMRPIVLVLLFATSALA

SEQ ID NO: 10 - protein, 458 aa, Artificial Sequence
  Synthetic Construct
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 11 - protein, 198 aa, Artificial Sequence
  Synthetic Construct
RDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 12 - protein, 594 aa, Artificial Sequence
  Synthetic Construct
AGAGACTCTCCCGAGGATTTCGTGTACCAGTTTAAGGGCCTGTGCTACTTCACCAACGGGACGGAGCGCGTGCGGGGTGTGACCAGACACATCTATAACCGAGAGGAGTACGTGCGCTTCGACAGCGACGTGGGGGTGTACCGGGCAGTGACGCCGCAGGGGCGGCCTGTTGCCGAGTACTGGAACAGCCAGAAGGAAGTCCTGGAGGGGGCCCGGGCGTCGGTGGACAGGGTGTGCAGACACAACTACGAGGTGGCGTACCGCGGGATCCTGCAGAGGAGAGTGGAGCCCACAGTGACCATCTCCCCATCCAGGACAGAGGCCCTCAACCACCACAACCTGCTGATCTGCTCGGTGACAGATTTCTATCCAAGCCAGATCAAAGTCCGGTGGTTTCGGAATGATCAGGAGGAGACAGCCGGCGTTGTGTCCACCCCCCTCATTAGGAACGGTGACTGGACCTTCCAGATCCTGGTGATGCTGGAAATGACTCCCCAGCGTGGAGATGTCTACACCTGCCACGTGGAGCACCCCAGCCTCCAGAGCCCCATCACCGTGGAGTGGCGGGCTCAGTCTGAATCTGCCCAGAGCAAG

SEQ ID NO: 13 - protein, 198 aa, Artificial Sequence
  Synthetic Construct
RDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALQHHNWLVCHVTDFYPSQIKVRWFRNDQEETAGVMSTNLIRNGDWTFQILVMLEMTPQRGDVYTCHVEHPSLQSPITVEWRAQSESAQSK

SEQ ID NO: 14 - protein, 267 aa, Artificial Sequence
  Synthetic Construct
MMRPIVLVLLFATSALARDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALQHHNWLVCHVTDFYPSQIKVRWFRNDQEETAGVMSTNLIRNGDWTFQILVMLEMTPQRGDVYTCHVEHPSLQSPITVEWRAQSESAQSKGGGGSLEIEAAFLERENTALETRVAELRQRVQRLRNRVSQYRTRYGPLGGGK

SEQ ID NO: 15 - protein, 261 aa, Artificial Sequence [sequence omitted]

SEQ ID NO: 16 - protein, 195 aa, Artificial Sequence
  Synthetic Construct
EDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 17 - protein, 585 aa, Artificial Sequence
  Synthetic Construct
GAGGACATCGTGGCCGATCACGTGGCAAGCTGCGGCGTGAACCTGTACCAGTTCTACGGCCCCTCTGGCCAGTACACCCATGAATTTGATGGAGATGAGGAGTTCTACGTGGACCTGGAGAGGAAGGAGACTGCCTGGCGGTGGCCTGAGTTCAGCAAATTTGGAGGTTTTGACCCGCAGGGTGCACTGAGAAACATGGCTGTGGCAAAACACAACTTGAACATCATGATTAAACGCTACAACTCTACCGCTGCTACCAATGAGGTTCCTGAGGTCACAGTGTTTTCCAAGTCTCCCGTGACACTGGGTCAGCCCAACACCCTCATTTGTCTTGTGGACAACATCTTTCCTCCTGTGGTCAACATCACATGGCTGAGCAATGGGCAGTCAGTCACAGAAGGTGTTTCTGAGACCAGCTTCCTCTCCAAGAGTGATCATTCCTTCTTCAAGATCAGTTACCTCACCTTCCTCCCTTCTGCTGATGAGATTTATGACTGCAAGGTGGAGCACTGGGGCCTGGACCAGCCTCTTCTGAAACACTGGGAGCCTGAGATTCCAGCCCCTATGTCAGAGCTCACAGAGACT

SEQ ID NO: 18 - protein, 272 aa, Artificial Sequence
  Synthetic Construct
MMRPIVLVLLFATSALAEDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETGGGGSLEIRAAFLRQRNTALRTEVAELEQEVQRLENEVSQYETRYGPLGGGKGSHHHHHH

SEQ ID NO: 19 - protein, 198 aa, Artificial Sequence
  Synthetic Construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 20 - protein, 594 aa, Artificial Sequence
  Synthetic Construct
GGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACCTCCTGGTCTGCTCTGTGAGTGGTTTCTATCCAGGCAGCATTGAAGTCAGGTGGTTCCGGAACGGCCAGGAAGAGAAGGCTGGGGTGGTGTCCACAGGCCTGATCCAGAATGGAGATTGGACCTTCCAGACCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAG

SEQ ID NO: 21 - protein, 198 aa, Artificial Sequence
  Synthetic Construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSK

SEQ ID NO: 22 - protein, 267 aa, Artificial Sequence
  Synthetic Construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSKGGGGSLEIEAAFLERENTALETRVAELRQRVQRLRNRVSQYRTRYGPLGGGK

SEQ ID NO: 23 - protein, 266 aa, Artificial Sequence
  Synthetic Construct
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 24 - protein, 192 aa, Artificial Sequence
  Synthetic Construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEN

SEQ ID NO: 25 - protein, 576 aa, Artificial Sequence
  Synthetic Construct
ATCAAAGAAGAACATGTGATCATCCAGGCCGAGTTCTATCTGAATCCTGACCAATCAGGCGAGTTTATGTTTGACTTTGATGGTGATGAGATTTTCCATGTGGATATGGCAAAGAAGGAGACGGTCTGGCGGCTTGAAGAATTTGGACGATTTGCCAGCTTTGAGGCTCAAGGTGCATTGGCCAACATAGCTGTGGACAAAGCCAACCTGGAAATCATGACAAAGCGCTCCAACTATACTCCGATCACCAATGTACCTCCAGAGGTAACTGTGCTCACAAACAGCCCTGTGGAACTGAGAGAGCCCAACGTCCTCATCTGTTTCATAGACAAGTTCACCCCACCAGTGGTCAATGTCACGTGGCTTCGAAATGGAAAACCTGTCACCACAGGAGTGTCAGAGACAGTCTTCCTGCCCAGGGAAGACCACCTTTTCCGCAAGTTCCACTATCTCCCCTTCCTGCCCTCAACTGAGGACGTTTACGACTGCAGGGTGGAGCACTGGGGCTTGGATGAGCCTCTTCTCAAGCACTGGGAGTTTGATGCTCCAAGCCCTCTCCCAGAGACTACAGAGAAC

SEQ ID NO: 26 - protein, 269 aa, Artificial Sequence
  Synthetic Construct
MMRPIVLVLLFATSALAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENGGGGSLEIRAAFLRQRNTALRTEVAELEQEVQRLENEVSQYETRYGPLGGGKGSHHHHHH

SEQ ID NO: 27 - protein, 295 aa, Artificial Sequence
  Synthetic Construct
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 28 - protein, 5654 aa, Artificial Sequence
  Synthetic Construct
MSVGRRKLALLWALALALACTRHTGHAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTDQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGAQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENEKYAQHWCSQLTDADGPFGRCHAAVKPGTYYSNCMFDTCNCERSEDCLCAALSSYVHACAAKGVQLGGWRDGVCTKPMTTCPKSMTYHYHVSTCQPTCRSLSEGDITCSVGFIPVDGCICPKGTFLDDTGKCVQASNCPCYHRGSMIPNGESVHDSGAICTCTHGKLSCIGGQAPAPVCAAPMVFFDCRNATPGDTGAGCQKSCHTLDMTCYSPQCVPGCVCPDGLVADGEGGCITAEDCPCVHNEASYRAGQTIRVGCNTCTCDSRMWRCTDDPCLATCAVYGDGHYLTFDGQSYSFNGDCEYTLVQNHCGGKDSTQDSFRVVTENVPCGTTGTTCSKAIKIFLGGFELKLSHGKVEVIGTDESQEVPYTIRQMGIYLVVDTDIGLVLLWDKKTSIFINLSPEFKGRVCGLCGNFDDIAVNDFATRSRSVVGDVLEFGNSWKLSPSCPDALAPKDPCTANPFRKSWAQKQCSILHGPTFAACHAHVEPARYYEACVNDACACDSGGDCECFCTAVAAYAQACHEVGLCVSWRTPSICPLFCDYYNPEGQCEWHYQPCGVPCLRTCRNPRGDCLRDVRGLEGCYPKCPPEAPIFDEDKMQCVATCPTPPLPPRCHVHGKSYRPGAVVPSDKNCQSCLCTERGVECTYKAEACVCTYNGQRFHPGDVIYHTTDGTGGCISARCGANGTIERRVYPCSPTTPVPPTTFSFSTPPLVVSSTHTPSNGPSSAHTGPPSSAWPTTAGTSPRTRLPTASASLPPVCGEKCLWSPWMDVSRPGRGTDSGDFDTLENLRAHGYRVCESPRSVECRAEDAPGVPLRALGQRVQCSPDVGLTCRNREQASGLCYNYQIRVQCCTPLPCSTSSSPAQTTPPTTSKTTETRASGSSAPSSTPGTVSLSTARTTPAPGTATSVKKTFSTPSPPPVPATSTSSMSTTAPGTSVVSSKPTPTEPSTSSCLQELCTWTEWIDGSYPAPGINGGDFDTFQNLRDEGYTFCESPRSVQCRAESFPNTPLADLGQDVICSHTEGLICLNKNQLPPICYNYEIRIQCCETVNVCRDITRLPKTVATTRPTPHPTGAQTQTTFTTHMPSASTEQPTATSRGGPTATSVTQGTHTTLVTRNCHPRCTWTKWFDVDFPSPGPHGGDKETYNNIIRSGEKICRRPEEITRLQCRAKSHPEVSIEHLGQVVQCSREEGLVCRNQDQQGPFKMCLNYEVRVLCCETPRGCHMTSTPGSTSSSPAQTTPSTTSKTTETQASGSSAPSSTPGTVSLSTARTTPAPGTATSVKKTFSTPSPPPVPATSTSSMSTTAPGTSVVSSKPTPTEPSTSSCLQELCTWTEWIDGSYPAPGINGGDFDTFQNLRDEGYTFCESPRSVQCRAESFPNTPLADLGQDVICSHTEGLICLNKNQLPPICYNYEIRIQCCETVNVCRDITRPPKTVATTRPTPHPTGAQTQTTFTTHMPSASTEQPTATSRGGPTATSVTQGTHTTPVTRNCHPRCTWTTWFDVDFPSPGPHGGDKETYNNIIRSGEKICRRPEEITRLQCRAKSHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTYAHTTSTTSAPTARTTSAPTTRTTSASPASTTSGPGNTPSPVPTTSTISAPTTSITSAPTTSTTSAPTSSTTSGPGTTPSPVPTTSITSAPTTSTTSAPTTSTTSARTSSTTSATTTSRISGPETTPSPVPTTSTTSATTTSTTSAPTTSTTSAPTSSTTSSPQTSTTSAPTTSTTSGPGTTPSPVPTTSTTSAPTTRTTSAPKSSTTSAATTSTTSGPETTPRPVPTTSTTSSPTTSTTSAPTTSTTSASTTSTTSGAGTTPSPVPTTSTTSAPTTSTTSAPISSTTSATTTSTTSGPGTTPSPVPTTSTTSAPTTSTTSGPGTTPSAVPTTSITSAPTTSTNSAPISSTTSATTTSRISGPETTPSPVPTASTTSASTTSTTSGPGTTPSPVPTTSTISVPTTSTTSASTTSTTSASTTSTTSGPGTTPSPVPTTSTTSAPTTSTTSAPTTSTISAPTTSTTSATTTSTTSAPTPRRTSAPTTSTISASTTSTTSATTTSTTSATTTSTISAPTTSTTLSPTTSTTSTTITSTTSAPISSTTSTPQTSTTSAPTTSTTSGPGTTSSPVPTTSTTSAPTTSTTSAPTTRTTSVPTSSTTSTATTSTTSGPGTTPSPVPTTSTTSAPTTRTTSAPTTSTTSAPTTSTTSAPTSSTTSATTTSTISVPTTSTTSVPGTTPSPVPTTSTISVPTTSTTSASTTSTTSGPGTTPSPVPTTSTTSAPTTSTTSAPTTSTISAPTTSTPSAPTTSTTLAPTTSTTSAPTTSTTSTPTSSTTSSPQTSTTSASTTSITSGPGTTPSPVPTTSTTSAPTTSTTSAATTSTISAPTTSTTSAPTTSTTSASTASKTSGLGTTPSPIPTTSTTSPPTTSTTSASTASKTSGPGTTPSPVPTTSTIFAPRTSTTSASTTSTTPGPGTTPSPVPTTSTASVSKTSTSHVSISKTTHSQPVTRDCHLRCTWTKWFDIDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSSTTSGSGTTPSPVTTTSTASVSKTSTSHVSVSKTTHSQPVTRDCHPRCTWTKWFDVDFPSPGPHGGDKETYNNIIRSGEKICRRPEEITRLQCRAKSHPEVSIEHLGQVVQCSREEGLVCRNQDQQGPFKMCLNYEVRVLCCETPKGCPVTSTSVTAPSTPSGRATSPTQSTSSWQKSRTTTLVTSSITSTTQTSTTSAPTTSTTPASIPSTTSAPTTSTTSAPTTSTTSAPTTSTTSTPQTTTSSAPTSSTTSAPTTSTISAPTTSTISAPTTSTTSAPTASTTSAPTSTSSAPTTNTTSAPTTSTTSAPITSTISAPTTSTTSTPQTSTISSPTTSTTSTPQTSTTSSPTTSTTSAPTTSTTSAPTTSTTSTPQTSISSAPTSSTTSAPTASTISAPTTSTTSFHTTSTTSPPTSSTSSTPQTSKTSAATSSTTSGSGTTPSPVPTTSTASVSKTSTSHVSVSKTTHSQPVTRDCHPRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTIPASTPSTTSAPTTSTTSAPTTSTTSAPTHRTTSGPTTSTTLAPTTSTTSAPTTSTNSAPTTSTISASTTSTISAPTTSTISSPTSSTTSTPQTSKTSAATSSTTSGSGTTPSPVPTTSTTSASTTSTTSAPTTSTTSGPGTTPSPVPSTSTTSAATTSTTSAPTTRTTSAPTSSMTSGPGTTPSPVPTTSTTSAPTTSTTSGPGTTPSPVPTTSTTSAPITSTTSGPGSTPSPVPTTSTTSAPTTSTTSASTASTTSGPGTTPSPVPTTSTTSAPTTRTTSASTASTTSGPGSTPSPVPTTSTTSAPTTRTTPASTASTTSGPGTTPSPVPTTSTTSASTTSTISLPTTSTTSAPITSMTSGPGTTPSPVPTTSTTSAPTTSTTSASTASTTSGPGTTPSPVPTTSTTSAPTTSTTSASTASTTSGPGTSLSPVPTTSTTSAPTTSTTSGPGTTPSPVPTTSTTSAPTTSTTSGPGTTPSPVPTTSTTPVSKTSTSHLSVSKTTHSQPVTSDCHPLCAWTKWFDVDFPSPGPHGGDKETYNNIIRSGEKICRRPEEITRLQCRAESHPEVNIEHLGQVVQCSREEGLVCRNQDQQGPFKMCLNYEVRVLCCETPRGCPVTSVTPYGTSPTNALYPSLSTSMVSASVASTSVASSSVASSSVAYSTQTCFCNVADRLYPAGSTIYRHRDLAGHCYYALCSQDCQVVRGVDSDCPSTTLPPAPATSPSISTSEPVTELGCPNAVPPRKKGETWATPNCSEATCEGNNVISLRPRTCPRVEKPTCANGYPAVKVADQDGCCHHYQCQCVCSGWGDPHYITFDGTYYTFLDNCTYVLVQQIVPVYGHFRVLVDNYFCGAEDGLSCPRSIILEYHQDRVVLTRKPVHGVMTNEIIFNNKVVSPGFRKNGIVVSRIGVKMYATIPELGVQVMFSGLIFSVEVPFSKFANNTEGQCGTCTNDRKDECRTPRGTVVASCSEMSGLWNVSIPDQPACHRPHPTPTTVGPTTVGSTTVGPTTVGSTTVGPTTPPAPCLPSPICQLILSKVFEPCHTVIPPLLFYEGCVFDRCHMTDLDVVCSSLELYAALCASHDICIDWRGRTGHMCPFTCPADKVYQPCGPSNPSYCYGNDSASLGALPEAGPITEGCFCPEGMTLFSTSAQVCVPTGCPRCLGPHGEPVKVGHTVGMDCQECTCEAATWTLTCRPKLCPLPPACPLPGFVPVPAAPQAGQCCPQYSCACNTSRCPAPVGCPEGARAIPTYQEGACCPVQNCSWTVCSINGTLYQPGAVVSSSLCETCRCELPGGPPSDAFVVSCETQICNTHCPVGFEYQEQSGQCCGTCVQVACVTNTSKSPAHLFYPGETWSDAGNHCVTHQCEKHQDGLVVVTTKKACPPLSCSLDEARMSKDGCCRFCPPPPPPYQNQSTCAVYHRSLIIQQQGCSSSEPVRLAYCRGNCGDSSSMYSLEGNTVEHRCQCCQELRTSLRNVTLHCTDGSSRAFSYTEVEECGCMGRRCPAPGDTQHSEEAEPEPSQEAESGSWERGVPVSPMH

SEQ ID NO: 29 - protein, 314 aa, Artificial Sequence
  Synthetic Construct
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 30 - protein, 27 aa, Artificial Sequence
  Synthetic Construct
CACCACAACNNNCTTNNNTGCCACGTG

SEQ ID NO: 31 - protein, 27 aa, Artificial Sequence
  Synthetic Construct
CACGTGGCANNNAAGNNNGTTGTGGTG

SEQ ID NO: 32 - protein, 27 aa, Artificial Sequence
  Synthetic Construct
ACAGCTGGGGTCNNNTCCACCAACCTG

SEQ ID NO: 33 - protein, 27 aa, Artificial Sequence
  Synthetic Construct
CAGGTTGGTGGANNNGACCCCAGCTGT

SEQ ID NO: 34 - protein, 27 aa, Artificial Sequence
  Synthetic Construct
CAGATCNNNGTGNNNCTGGAAATGACC

SEQ ID NO: 35 - protein, 27 aa, Artificial Sequence
  Synthetic Construct
GGTCATTTCCAGNNNCACNNNGATCTG

SEQ ID NO: 36 - protein, 14 aa, Artificial Sequence
  Synthetic Construct
CAVCTLYNFNKFYF

SEQ ID NO: 37 - protein, 17 aa, Artificial Sequence
  Synthetic Construct
CAVAPYDRGSTLGRLYF

SEQ ID NO: 38 - protein, 13 aa, Artificial Sequence
  Synthetic Construct
CAGVKDSNYQLIW

SEQ ID NO: 39 - protein, 17 aa, Artificial Sequence
  Synthetic Construct
CALSDLSYGGATNKLIF

SEQ ID NO: 40 - protein, 14 aa, Artificial Sequence
  Synthetic Construct
CAVEVNSGTYKYIF

SEQ ID NO: 41 - protein, 12 aa, Artificial Sequence
  Synthetic Construct
CALSVGTYKYIF

SEQ ID NO: 42 - protein, 16 aa, Artificial Sequence
  Synthetic Construct
CAFMKRAETSGSRLTF

SEQ ID NO: 43 - protein, 12 aa, Artificial Sequence
  Synthetic Construct
CASLTDNNEQFF

SEQ ID NO: 44 - protein, 14 aa, Artificial Sequence
  Synthetic Construct
CASSTGQGLETQYF

SEQ ID NO: 45 - protein, 11 aa, Artificial Sequence
  Synthetic Construct
CAWSSYNEQFF

SEQ ID NO: 46 - protein, 16 aa, Artificial Sequence
  Synthetic Construct
CASSKGQGLGNQPQHF

SEQ ID NO: 47 - protein, 11 aa, Artificial Sequence
  Synthetic Construct
CASRRDLAAFF

SEQ ID NO: 48 - protein, 15 aa, Artificial Sequence
  Synthetic Construct
CASSPGTGGRETQYF

SEQ ID NO: 49 - protein, 14 aa, Artificial Sequence
  Synthetic Construct
CASSYWPTRETQYF

SEQ ID NO: 50 - protein, 20 aa, Artificial Sequence
  Synthetic Construct
EVLAPVILMLLNSFFNAIST

SEQ ID NO: 51 - protein, 20 aa, Artificial Sequence
  Synthetic Construct
EGEISDPFRFTTFYIHFALV

SEQ ID NO: 52 - protein, 20 aa, Artificial Sequence
  Synthetic Construct
KALLATFGSSFLISACFKLI

SEQ ID NO: 53 - protein, 20 aa, Artificial Sequence
  Synthetic Construct
FKLIQDLLSFINPQLLSILI

SEQ ID NO: 54 - protein, 20 aa, Artificial Sequence
  Synthetic Construct
GCEDVRVSGWLQTEFLSFQD

SEQ ID NO: 55 - protein, 20 aa, Artificial Sequence
  Synthetic Construct
TVTKLSQKFTKVNFTEIQKL

SEQ ID NO: 56 - protein, 20 aa, Artificial Sequence
  Synthetic Construct
TDEELDRFKFFLSDEFNIAT

SEQ ID NO: 57 - protein, 20 aa, Artificial Sequence
  Synthetic Construct
RIIIIARYYRHSGFLEVNSA

SEQ ID NO: 58 - protein, 20 aa, Artificial Sequence
  Synthetic Construct
RTIPIDGNFFTYTRHEPIGV

SEQ ID NO: 59 - protein, 20 aa, Artificial Sequence
  Synthetic Construct
DELDFLMEALIISKFNHQNI

SEQ ID NO: 60 - protein, 20 aa, Artificial Sequence
  Synthetic Construct
VYGNTALHYAVYSEILSVVA

SEQ ID NO: 61 - protein, 20 aa, Artificial Sequence
  Synthetic Construct
RIRPLWKHYFQNTQGLIFVV

SEQ ID NO: 62 - protein, 20 aa, Artificial Sequence
  Synthetic Construct
RIRPLWKHYFQNTQGLIFVV

SEQ ID NO: 63 - protein, 20 aa, Artificial Sequence
  Synthetic Construct
AVFLALSAQLLQARLMKEES

SEQ ID NO: 64 - protein, 20 aa, Artificial Sequence
  Synthetic Construct
RTIMGWTLDFLRERLLGWIQ

SEQ ID NO: 65 - protein, 20 aa, Artificial Sequence
  Synthetic Construct
LSYKLSQKGYSWSQFSDVEE

SEQ ID NO: 66 - protein, 20 aa, Artificial Sequence
  Synthetic Construct
LSVKKQFEELTLGEFLKLDR

SEQ ID NO: 67 - protein, 20 aa, Artificial Sequence
  Synthetic Construct
DPWTEHAKWFPSCQFLLRSK

SEQ ID NO: 68 - protein, 20 aa, Artificial Sequence
  Synthetic Construct
YPSSQDSSSAAAPQLLIVLL

SEQ ID NO: 69 - protein, 20 aa, Artificial Sequence
  Synthetic Construct
LTTPPCAQGVIWTVFNQTVM

SEQ ID NO: 70 - protein, 20 aa, Artificial Sequence
  Synthetic Construct
MGFQKFSPFLALSILVLLQA

SEQ ID NO: 71 - protein, 20 aa, Artificial Sequence
  Synthetic Construct
VLQQQLESFQALRMQTLQNV

SEQ ID NO: 72 - protein, 20 aa, Artificial Sequence
  Synthetic Construct
KYVAELSLLEADPFLKYLPS

SEQ ID NO: 73 - protein, 20 aa, Artificial Sequence
  Synthetic Construct
QQAIREKYKASKYLCVSLME

SEQ ID NO: 74 - protein, 20 aa, Artificial Sequence
  Synthetic Construct
SPNNFLSYYRLTRFLSRVIK

SEQ ID NO: 75 - protein, 20 aa, Artificial Sequence
  Synthetic Construct
MRIFAVFIFMTYWHLLNAFT

SEQ ID NO: 76 - protein, 20 aa, Artificial Sequence
  Synthetic Construct
PSKYINASFIMSYWKPEVMI

SEQ ID NO: 77 - protein, 20 aa, Artificial Sequence
  Synthetic Construct
PLKETIGDFWQMIFQRKVKV

SEQ ID NO: 78 - protein, 20 aa, Artificial Sequence
  Synthetic Construct
KARPGMVSTFEQYQFLYDVI

SEQ ID NO: 79 - protein, 20 aa, Artificial Sequence
  Synthetic Construct
DHKPKFTQDTFRGSVLEGVL

SEQ ID NO: 80 - protein, 20 aa, Artificial Sequence
  Synthetic Construct
DFVTETPLEGDFAWERVRGL

SEQ ID NO: 81 - protein, 20 aa, Artificial Sequence
  Synthetic Construct
PAQYSWFVNGTFQQSTQELF

SEQ ID NO: 82 - protein, 20 aa, Artificial Sequence
  Synthetic Construct
RSLRTNFLRYWTLTYLALPT

SEQ ID NO: 83 - protein, 20 aa, Artificial Sequence
  Synthetic Construct
CGKEGKYIHFTPNFLLNDNL

SEQ ID NO: 84 - protein, 20 aa, Artificial Sequence
  Synthetic Construct
ISRSDEGKYTCFAENFMGKA

SEQ ID NO: 85 - protein, 20 aa, Artificial Sequence
  Synthetic Construct
QQCTDDVRLFAFVRFTTGDA

SEQ ID NO: 86 - protein, 20 aa, Artificial Sequence
  Synthetic Construct
NIIDLQFLHGYYEPTLLILF

SEQ ID NO: 87 - protein, 20 aa, Artificial Sequence
  Synthetic Construct
ANAAVGEPAFLSEEFQNSPE

SEQ ID NO: 88 - protein, 20 aa, Artificial Sequence
  Synthetic Construct
GVKRKDQGFLEKEFYHKTNI

SEQ ID NO: 89 - protein, 20 aa, Artificial Sequence
  Synthetic Construct
HDKACVRTFYETPLQLLEKV

SEQ ID NO: 90 - protein, 20 aa, Artificial Sequence
  Synthetic Construct
QRSEHDVLFQVTQFPSRGQL

SEQ ID NO: 91 - protein, 20 aa, Artificial Sequence
  Synthetic Construct
FQIDQGEVVFAFTNFSSSHD

SEQ ID NO: 92 - protein, 20 aa, Artificial Sequence
  Synthetic Construct
IDQGEVVFAFTNFSSSHDHF

SEQ ID NO: 93 - protein, 20 aa, Artificial Sequence
  Synthetic Construct
SSILCALIVFWKYRRFQRNT

SEQ ID NO: 94 - protein, 20 aa, Artificial Sequence
  Synthetic Construct
AGQSRCGGFLVREDFVLTAA

SEQ ID NO: 95 - protein, 20 aa, Artificial Sequence
  Synthetic Construct
EQLNRNFSNFILDKFLRHCE

SEQ ID NO: 96 - protein, 20 aa, Artificial Sequence
  Synthetic Construct
STALQWLLLLFTRYPDVQTR

SEQ ID NO: 97 - protein, 20 aa, Artificial Sequence
  Synthetic Construct
DPWPLNPLSIQQTTLLLLLS

SEQ ID NO: 98 - protein, 20 aa, Artificial Sequence
  Synthetic Construct
PQFANCSVYDFFVWLHYYSV

SEQ ID NO: 99 - protein, 20 aa, Artificial Sequence
  Synthetic Construct
ANCSVYDFFVWLHYYSVRDT

SEQ ID NO: 100 - protein, 20 aa, Artificial Sequence
  Synthetic Construct
QKFDNPPFFQNSTFSFRNAL

SEQ ID NO: 101 - protein, 20 aa, Artificial Sequence
  Synthetic Construct
IATPGRLNDLQMSNFVNLKN

SEQ ID NO: 102 - protein, 20 aa, Artificial Sequence
  Synthetic Construct
CASGLCCARHFWSKICKPVL

SEQ ID NO: 103 - protein, 20 aa, Artificial Sequence
  Synthetic Construct
EEGCEAISFLLSLIDRLVLY

SEQ ID NO: 104 - protein, 20 aa, Artificial Sequence
  Synthetic Construct
GSKEDANVFASAMMHALEVL

SEQ ID NO: 105 - protein, 20 aa, Artificial Sequence
  Synthetic Construct
TRYQLDPKFITSILYENNVI

SEQ ID NO: 106 - protein, 20 aa, Artificial Sequence
  Synthetic Construct
ESDLDYGTNFQKRLFTKIDT

SEQ ID NO: 107 - protein, 20 aa, Artificial Sequence
  Synthetic Construct
ESDDDHGVKFREHQFTKIDT

SEQ ID NO: 108 - protein, 20 aa, Artificial Sequence
  Synthetic Construct
PEGASAASFEYTILDPSSQL

SEQ ID NO: 109 - protein, 20 aa, Artificial Sequence
  Synthetic Construct
EDLGPASPLDSTFYRSLLED

SEQ ID NO: 110 - protein, 20 aa, Artificial Sequence
  Synthetic Construct
DLGPASPLDSTFYRSLLEDD

SEQ ID NO: 111 - protein, 20 aa, Artificial Sequence
  Synthetic Construct
AASQHVFRFYRESLQRRYSK

SEQ ID NO: 112 - protein, 20 aa, Artificial Sequence
  Synthetic Construct
HTLFCRRCFKYDCFLHPFHA

SEQ ID NO: 113 - protein, 20 aa, Artificial Sequence
  Synthetic Construct
MSLSFLLLLFFSHLILSAWA

SEQ ID NO: 114 - protein, 20 aa, Artificial Sequence
  Synthetic Construct
VYETYELVEKFYDPMFKYHL

SEQ ID NO: 115 - protein, 20 aa, Artificial Sequence
  Synthetic Construct
VAQTGILWLLMNNCFLNLSP

SEQ ID NO: 116 - protein, 20 aa, Artificial Sequence
  Synthetic Construct
MNLSRGKSTYYWPRPRRYVQ

SEQ ID NO: 117 - protein, 20 aa, Artificial Sequence
  Synthetic Construct
MKQDFSVPQLPHSSSHWLRL

SEQ ID NO: 118 - protein, 20 aa, Artificial Sequence
  Synthetic Construct
PSLTPQAFEFVGEFFTDVSL

SEQ ID NO: 119 - protein, 20 aa, Artificial Sequence
  Synthetic Construct
AMWIQLLYSACFWWLFCYAV

SEQ ID NO: 120 - protein, 20 aa, Artificial Sequence
  Synthetic Construct
TGPIRNGDWTFQTVVMLEMT

SEQ ID NO: 121 - protein, 20 aa, Artificial Sequence
  Synthetic Construct
PGVYTKVSDFREWIFQAIKT

SEQ ID NO: 122 - protein, 20 aa, Artificial Sequence
  Synthetic Construct
GTVAYLALRISYSLFTALRV

SEQ ID NO: 123 - protein, 20 aa, Artificial Sequence
  Synthetic Construct
VFVQSVLPYFVATKLAKIRK

SEQ ID NO: 124 - protein, 20 aa, Artificial Sequence
  Synthetic Construct
EGIDFYTSITRARFEELCSD

SEQ ID NO: 125 - protein, 20 aa, Artificial Sequence
  Synthetic Construct
SFRDGDCSKGFFLVSLLVEI

SEQ ID NO: 126 - protein, 20 aa, Artificial Sequence
  Synthetic Construct
SSENKPIRSSYFTFQLQNIV

SEQ ID NO: 127 - protein, 20 aa, Artificial Sequence
  Synthetic Construct
DDAAPRVEGVPVAVHKHALH

SEQ ID NO: 128 - protein, 20 aa, Artificial Sequence
  Synthetic Construct
TFGEMADAFKSDYFNMPVHM

SEQ ID NO: 129 - protein, 20 aa, Artificial Sequence
  Synthetic Construct
IIILVGTAVIAMFFWLLLVI

SEQ ID NO: 130 - protein, 20 aa, Artificial Sequence
  Synthetic Construct
RFSIWISFFEIYNELLYDLL

SEQ ID NO: 131 - protein, 20 aa, Artificial Sequence
  Synthetic Construct
NQPCYRKLGLEVYVTFFEIY

SEQ ID NO: 132 - protein, 20 aa, Artificial Sequence
  Synthetic Construct
SVRHPEYNRPLLANDLMLIK

SEQ ID NO: 133 - protein, 20 aa, Artificial Sequence
  Synthetic Construct
QKKTLQALEFHTVPFQLLAR

SEQ ID NO: 134 - protein, 20 aa, Artificial Sequence
  Synthetic Construct
VMVNGILFVQYFHRVPFHRV

SEQ ID NO: 135 - protein, 20 aa, Artificial Sequence
  Synthetic Construct
IAKRQLSHLQASGFSLLSAF

SEQ ID NO: 136 - protein, 20 aa, Artificial Sequence
  Synthetic Construct
TGVKEVARKYNYIASFFIET

SEQ ID NO: 137 - protein, 20 aa, Artificial Sequence
  Synthetic Construct
YNYIASFFIETGFCDSGILS

SEQ ID NO: 138 - protein, 20 aa, Artificial Sequence
  Synthetic Construct
QAMAKNRLQFVRFEATDLHG

SEQ ID NO: 139 - protein, 20 aa, Artificial Sequence
  Synthetic Construct
QCFVCAQCFQQFPEGLFYEF

SEQ ID NO: 140 - protein, 20 aa, Artificial Sequence
  Synthetic Construct
EKRFLLEEPMPFFYLKCCKI

SEQ ID NO: 141 - protein, 20 aa, Artificial Sequence
  Synthetic Construct
DEKVTDLVQFLLFKYQMKEP

SEQ ID NO: 142 - protein, 20 aa, Artificial Sequence
  Synthetic Construct
ALSRKMAELVHFLLLKYRAR

SEQ ID NO: 143 - protein, 20 aa, Artificial Sequence
  Synthetic Construct
PRKLLTQDLVQEKYLEYRQV

SEQ ID NO: 144 - protein, 20 aa, Artificial Sequence
  Synthetic Construct
AISRKMVELVHFLLLKYRAR

SEQ ID NO: 145 - protein, 20 aa, Artificial Sequence
  Synthetic Construct
RKMVELVHFLLLKYRAREPV

SEQ ID NO: 146 - protein, 20 aa, Artificial Sequence
  Synthetic Construct
YEFLWGPRALAETSYVKVLE

SEQ ID NO: 147 - protein, 20 aa, Artificial Sequence
  Synthetic Construct
LGSVVGNWQYFFPVIFSKAS

SEQ ID NO: 148 - protein, 20 aa, Artificial Sequence
  Synthetic Construct
PKKLLTQYFVQENYLEYRQV

SEQ ID NO: 149 - protein, 20 aa, Artificial Sequence
  Synthetic Construct
YFPVIFGKASEFMQVIFGTD

SEQ ID NO: 150 - protein, 20 aa, Artificial Sequence
  Synthetic Construct
PRKFITQDLVQEKYLKYEQV

SEQ ID NO: 151 - protein, 20 aa, Artificial Sequence
  Synthetic Construct
PWKLITKDLVQEKYLEYKQV

SEQ ID NO: 152 - protein, 20 aa, Artificial Sequence
  Synthetic Construct
QSPLQNPASSFFSSALLSIF

SEQ ID NO: 153 - protein, 20 aa, Artificial Sequence
  Synthetic Construct
QSPLQIPVSRSFSSTLLSIF

SEQ ID NO: 154 - protein, 20 aa, Artificial Sequence
  Synthetic Construct
SPLQIPGSPSFSSTLLSLFQ

SEQ ID NO: 155 - protein, 20 aa, Artificial Sequence
  Synthetic Construct
SPLQIPMTSSFSSTLLSILQ

SEQ ID NO: 156 - protein, 20 aa, Artificial Sequence
  Synthetic Construct
PLSSCCSSFSWSSFSEESSS

SEQ ID NO: 157 - protein, 20 aa, Artificial Sequence
  Synthetic Construct
ILTVILGVLLLIGCWYCRRR

SEQ ID NO: 158 - protein, 20 aa, Artificial Sequence
  Synthetic Construct
AVDRYISIFYALRYHSIVTL

SEQ ID NO: 159 - protein, 20 aa, Artificial Sequence
  Synthetic Construct
ILLGIFFLCWGPFFLHLTLI

SEQ ID NO: 160 - protein, 20 aa, Artificial Sequence
  Synthetic Construct
MQHRGFLLLTLLALLALTSA

SEQ ID NO: 161 - protein, 20 aa, Artificial Sequence
  Synthetic Construct
TYTMKEVLFYLGQYIMTKRL

SEQ ID NO: 162 - protein, 20 aa, Artificial Sequence
  Synthetic Construct
VSRISAIFSTFIGEHYVHVN

SEQ ID NO: 163 - protein, 20 aa, Artificial Sequence
  Synthetic Construct
RSAMCAFPIKYVNDFFNKIV

SEQ ID NO: 164 - protein, 20 aa, Artificial Sequence
  Synthetic Construct
KLGFFLVTFGFIWGMMLLHF

SEQ ID NO: 165 - protein, 20 aa, Artificial Sequence
  Synthetic Construct
AQIRGEIFFFKDRFIWRTVT

SEQ ID NO: 166 - protein, 20 aa, Artificial Sequence
  Synthetic Construct
APQEEKAVFFAGNEYWIYSA

SEQ ID NO: 167 - protein, 20 aa, Artificial Sequence
  Synthetic Construct
SKNKKTYIFAGDKFWRYNEV

SEQ ID NO: 168 - protein, 20 aa, Artificial Sequence
  Synthetic Construct
LQGGGHSYFFKGAYYLKLEN

SEQ ID NO: 169 - protein, 20 aa, Artificial Sequence
  Synthetic Construct
ELQWEQAQDYLKRFYLYDSE

SEQ ID NO: 170 - protein, 20 aa, Artificial Sequence
  Synthetic Construct
QPYTEYISTRWYRAPECLLT

SEQ ID NO: 171 - protein, 20 aa, Artificial Sequence
  Synthetic Construct
KGRVGPLLACIIGTQFRKLR

SEQ ID NO: 172 - protein, 20 aa, Artificial Sequence
  Synthetic Construct
DPGAAEQVPDFVTFLYQITR

SEQ ID NO: 173 - protein, 20 aa, Artificial Sequence
  Synthetic Construct
QMDRVNAIPFTYEQLDVLKH

SEQ ID NO: 174 - protein, 20 aa, Artificial Sequence
  Synthetic Construct
QLSTGVSFFFLSFHISNLQF

SEQ ID NO: 175 - protein, 20 aa, Artificial Sequence
  Synthetic Construct
SMPANFETTGFEAEPFSHLT

SEQ ID NO: 176 - protein, 20 aa, Artificial Sequence
  Synthetic Construct
SLPSSTPVPFSSSTFTTTDS

SEQ ID NO: 177 - protein, 20 aa, Artificial Sequence
  Synthetic Construct
AKTTTTFNTLAGSLFTPLTT

SEQ ID NO: 178 - protein, 20 aa, Artificial Sequence
  Synthetic Construct
STKAISASSFQSTGFTETPE

SEQ ID NO: 179 - protein, 20 aa, Artificial Sequence
  Synthetic Construct
ILLTIKDDTIYLTRHLAVLN

SEQ ID NO: 180 - protein, 20 aa, Artificial Sequence
  Synthetic Construct
SGWGDPHYITFDGTYYTFLD

SEQ ID NO: 181 - protein, 20 aa, Artificial Sequence
  Synthetic Construct
MRALQIVYGIRLEHFYMMPV

SEQ ID NO: 182 - protein, 20 aa, Artificial Sequence
  Synthetic Construct
LVMGVDVMFISLSYFLIIRT

SEQ ID NO: 183 - protein, 20 aa, Artificial Sequence
  Synthetic Construct
DPSVEPPLSQETFSDLWKLL

SEQ ID NO: 184 - protein, 20 aa, Artificial Sequence
  Synthetic Construct
QIAAVCRECLQALEFLHANQ

SEQ ID NO: 185 - protein, 20 aa, Artificial Sequence
  Synthetic Construct
VEKRGSAKELLQHPFLKLAK

SEQ ID NO: 186 - protein, 20 aa, Artificial Sequence
  Synthetic Construct
PRHVDRSDFFTSFYDKLKLQ

SEQ ID NO: 187 - protein, 20 aa, Artificial Sequence
  Synthetic Construct
MFVDSDSTYCSSTVFLDTMP

SEQ ID NO: 188 - protein, 20 aa, Artificial Sequence
  Synthetic Construct
RKQRRSRTTFTAEQLEELER

SEQ ID NO: 189 - protein, 20 aa, Artificial Sequence
  Synthetic Construct
LTGMVPGSEFSGSPYSHPQY

SEQ ID NO: 190 - protein, 20 aa, Artificial Sequence
  Synthetic Construct
VWNGPVGVFEWEAFARGTKA

SEQ ID NO: 191 - protein, 20 aa, Artificial Sequence
  Synthetic Construct
QAGAQEAQPLQPSHFLDISE

SEQ ID NO: 192 - protein, 20 aa, Artificial Sequence
  Synthetic Construct
NLPLVSSTYDLMSSAYLSTK

SEQ ID NO: 193 - protein, 20 aa, Artificial Sequence
  Synthetic Construct
NERFRCPEALFQPCFLGMES

SEQ ID NO: 194 - protein, 20 aa, Artificial Sequence
  Synthetic Construct
LATGEKGFGYKNSKFHRVIK

SEQ ID NO: 195 - protein, 20 aa, Artificial Sequence
  Synthetic Construct
SLQCLQALYVDSLFFLRGRL

SEQ ID NO: 196 - protein, 20 aa, Artificial Sequence
  Synthetic Construct
YQCKVCPAKFTQFVHLKLHK

SEQ ID NO: 197 - protein, 20 aa, Artificial Sequence
  Synthetic Construct
EVAAKTLPFYKDYFNVPYPL

SEQ ID NO: 198 - protein, 20 aa, Artificial Sequence
  Synthetic Construct
KLNLGTVGFYRTQYSSAMLE

SEQ ID NO: 199 - protein, 20 aa, Artificial Sequence
  Synthetic Construct
SHTDFYEEIQEFVKDVFSPI

SEQ ID NO: 200 - protein, 20 aa, Artificial Sequence
  Synthetic Construct
ELYNRYQGGFLISRLIKLSV

SEQ ID NO: 201 - protein, 20 aa, Artificial Sequence
  Synthetic Construct
CKLSSARWGVCWVNFEALII

SEQ ID NO: 202 - protein, 20 aa, Artificial Sequence
  Synthetic Construct
SRLGPTLMCLLSTQFKRLRD

SEQ ID NO: 203 - protein, 20 aa, Artificial Sequence
  Synthetic Construct
QGKDVLMNNGLKMDQFCKEH

SEQ ID NO: 204 - protein, 20 aa, Artificial Sequence
  Synthetic Construct
NTKFSEEELCSWYQSFLKDC

SEQ ID NO: 205 - protein, 20 aa, Artificial Sequence
  Synthetic Construct
MEKDSLPRFVRSEFYQELIK

SEQ ID NO: 206 - protein, 20 aa, Artificial Sequence
  Synthetic Construct
YGLASFKSFLKSEFSEENLE

SEQ ID NO: 207 - protein, 20 aa, Artificial Sequence
  Synthetic Construct
CGKTCLLIVFSKDQFPEVYV

SEQ ID NO: 208 - protein, 20 aa, Artificial Sequence
  Synthetic Construct
PDYDVWILMTVVGTIFVIIL

SEQ ID NO: 209 - protein, 20 aa, Artificial Sequence
  Synthetic Construct
YLTPDLWKETVFTKSPYQEF

SEQ ID NO: 210 - protein, 20 aa, Artificial Sequence
  Synthetic Construct
KVKRQFVEFTIKEAARFKKV

SEQ ID NO: 211 - protein, 20 aa, Artificial Sequence
  Synthetic Construct
MDQEFGVSTLVEEEFGQRRQ

SEQ ID NO: 212 - protein, 20 aa, Artificial Sequence
  Synthetic Construct
RQKMSEIFPLTEELWLEWLH

SEQ ID NO: 213 - protein, 20 aa, Artificial Sequence
  Synthetic Construct
LSNVEVFMVISFSSYKLFKS

SEQ ID NO: 214 - protein, 20 aa, Artificial Sequence
  Synthetic Construct
TLRDKASGVCIDSEFFLTTA

SEQ ID NO: 215 - protein, 20 aa, Artificial Sequence
  Synthetic Construct
VGLIFAVCLVGFMLYRMKKK

SEQ ID NO: 216 - protein, 20 aa, Artificial Sequence
  Synthetic Construct
HHHLLQEYEIERSFFLRMKC

SEQ ID NO: 217 - protein, 20 aa, Artificial Sequence
  Synthetic Construct
LLVPLLLSLFVLGLFLWFLK

SEQ ID NO: 218 - protein, 20 aa, Artificial Sequence
  Synthetic Construct
VQRLWVSRLLRHRKAQLLLV

SEQ ID NO: 219 - protein, 20 aa, Artificial Sequence
  Synthetic Construct
SQIAYTSLSLPHYGSAFPSI

SEQ ID NO: 220 - protein, 20 aa, Artificial Sequence
  Synthetic Construct
NFESMSLGSFSSSSALDRDL

SEQ ID NO: 221 - protein, 20 aa, Artificial Sequence
  Synthetic Construct
REQPDNIPAFAAAYFESLLE

SEQ ID NO: 222 - protein, 20 aa, Artificial Sequence
  Synthetic Construct
KAFDDIAKYFSKEEWEKMKA

SEQ ID NO: 223 - protein, 20 aa, Artificial Sequence
  Synthetic Construct
KAFDDIAKYFSKKEWEKMKS

SEQ ID NO: 224 - protein, 20 aa, Artificial Sequence
  Synthetic Construct
LLGTIHALIFAWNKWIDIKQ

SEQ ID NO: 225 - protein, 20 aa, Artificial Sequence
  Synthetic Construct
FLPIVVLIFKSILFLPCLRK

SEQ ID NO: 226 - protein, 20 aa, Artificial Sequence
  Synthetic Construct
PIKIAAIIASLTFLYTLLRE

SEQ ID NO: 227 - protein, 20 aa, Artificial Sequence
  Synthetic Construct
KIAAIIASLTFLYTLLREVI

SEQ ID NO: 228 - protein, 20 aa, Artificial Sequence
  Synthetic Construct
LALGLFVCFYAYNFVRDVLQ

SEQ ID NO: 229 - protein, 20 aa, Artificial Sequence
  Synthetic Construct
AVTDHPDRLWAWEKFVYLDE

SEQ ID NO: 230 - protein, 20 aa, Artificial Sequence
  Synthetic Construct
LRSFFYVTETTFQKNRLFFY

SEQ ID NO: 231 - protein, 20 aa, Artificial Sequence
  Synthetic Construct
RSFFYVTETTFQKNRLFFYR

SEQ ID NO: 232 - protein, 20 aa, Artificial Sequence
  Synthetic Construct
LDSLGQWNYTFASTEGQYLL

SEQ ID NO: 233 - protein, 20 aa, Artificial Sequence
  Synthetic Construct
LSNNSLVSLTYVSFRNLTHL

SEQ ID NO: 234 - protein, 20 aa, Artificial Sequence
  Synthetic Construct
APKSQFGRIIHTPFMKFIIH

SEQ ID NO: 235 - protein, 20 aa, Artificial Sequence
  Synthetic Construct
IIHGASYFTFLLLLNLYSLV

SEQ ID NO: 236 - protein, 20 aa, Artificial Sequence
  Synthetic Construct
NQLSFVMNSLYLATFALKVV

SEQ ID NO: 237 - protein, 20 aa, Artificial Sequence
  Synthetic Construct
QQSNDTFHSFIGTCFALFWY

SEQ ID NO: 238 - protein, 20 aa, Artificial Sequence
  Synthetic Construct
PLLTTKRVFWKGVLEELLWF

SEQ ID NO: 239 - protein, 20 aa, Artificial Sequence
  Synthetic Construct
DPIFLLHHAFVDSIFEQWLR

SEQ ID NO: 240 - protein, 20 aa, Artificial Sequence
  Synthetic Construct
QRCLRDILDGFFPSELQRLY

SEQ ID NO: 241 - protein, 20 aa, Artificial Sequence
  Synthetic Construct
LAAASGQNRMTQGQHFLQKV

SEQ ID NO: 242 - protein, 20 aa, Artificial Sequence
  Synthetic Construct
RRCDRVTRLEFLPFHFLLAT

SEQ ID NO: 243 - protein, 20 aa, Artificial Sequence
  Synthetic Construct
CATWKVICKSCISQTPGINL

SEQ ID NO: 244 - protein, 20 aa, Artificial Sequence
  Synthetic Construct
LQIQSLISCWAFWTTWTQSC

SEQ ID NO: 245 - protein, 20 aa, Artificial Sequence
  Synthetic Construct
VSTANVGDILSAARLLEIPA

SEQ ID NO: 246 - protein, 24 aa, Artificial Sequence
  Synthetic Construct
LPKPPKPVSKMRMATPLLMQALPM

SEQ ID NO: 247 - protein, 16 aa, Artificial Sequence
  Synthetic Construct
KKLLTQHFVQENYLEY

SEQ ID NO: 248 - protein, 21 aa, Artificial Sequence
  Synthetic Construct
PGCNKRYFKLSHLQMHSRKHT

SEQ ID NO: 249 - protein, 14 aa, Artificial Sequence
  Synthetic Construct
SLLMWITQCFLPVF

SEQ ID NO: 250 - protein, 20 aa, Artificial Sequence
  Synthetic Construct
YLAMPFATPMEAELARRSLA

SEQ ID NO: 251 - protein, 13 aa, Artificial Sequence
  Synthetic Construct
PKYVKQNTLKLAT

SEQ ID NO: 252 - protein, 20 aa, Artificial Sequence
  Synthetic Construct
FRDYVDRFYKTLRAEQASQE

SEQ ID NO: 253 - protein, 20 aa, Artificial Sequence
  Synthetic Construct
TGSNCPPHIENFSDIDMGEI

SEQ ID NO: 254 - protein, 12 aa, Artificial Sequence
  Synthetic Construct
ETLLRAVESYLL

SEQ ID NO: 255 - protein, 16 aa, Artificial Sequence
  Synthetic Construct
RGYFKMRTGKSSIMRS

SEQ ID NO: 256 - protein, 282 aa, Artificial Sequence
  Synthetic Construct
MMRPIVLVLLFATSALARATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKGGGGSLEIEAAFLERENTALETRVAELRQRVQRLRNRVSQYRTRYGPLGGGKGSGLNDIFEAQKIEWHE

SEQ ID NO: 257 - protein, 849 aa, Artificial Sequence
  Synthetic Construct
ATGATGAGGCCCATCGTGCTGGTGCTGCTGTTCGCCACATCTGCCCTGGCCAGAGCCACCCCCGAGAACTACCTGTTTCAGGGCCGGCAGGAATGCTACGCCTTCAACGGCACCCAGCGGTTTCTGGAACGGTACATCTACAACCGGGAAGAGTTCGCCAGATTCGACAGCGACGTGGGCGAGTTCAGAGCCGTGACAGAGCTGGGCAGACCTGCCGCCGAGTACTGGAACAGCCAGAAGGACATCCTGGAAGAGAAGCGGGCCGTGCCCGACCGGATGTGCAGACACAATTACGAGCTGGGAGGCCCCATGACCCTGCAGAGAAGAGTGCAGCCCAGAGTGAACGTGTCCCCCAGCAAGAAGGGCCCCCTGCAGCACCACAACTTGCTTGTCTGCCACGTGACCGACTTCTACCCCGGCTCTATCCAAGTGCGGTGGTTCCTGAACGGCCAGGAAGAGACAGCCGGCGTGGTGTCCACCAACCTGATCAGAAACGGCGACTGGACCTTCCAGATCCTCGTGATGCTGGAAATGACCCCCCAGCAGGGCGACGTGTACACCTGTCAGGTGGAACACACCAGCCTGGACAGCCCCGTGACCGTGGAATGGAAGGCCCAGAGCGATAGCGCCAGAAGCAAAGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 258 - protein, 282 aa, Artificial Sequence
  Synthetic Construct
MMRPIVLVLLFATSALARATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNWLVCHVTDFYPGSIQVRWFLNGQEETAGVMSTNLIRNGDWTFQILVMLEMTPQQGDVYTCQVEHTSLDSPVTVEWKAQSDSARSKGGGGSLEIEAAFLERENTALETRVAELRQRVQRLRNRVSQYRTRYGPLGGGKGSGLNDIFEAQKIEWHE

SEQ ID NO: 259 - protein, 849 aa, Artificial Sequence
  Synthetic Construct
ATGATGAGGCCCATCGTGCTGGTGCTGCTGTTCGCCACATCTGCCCTGGCCAGAGCCACCCCCGAGAACTACCTGTTTCAGGGCCGGCAGGAATGCTACGCCTTCAACGGCACCCAGCGGTTTCTGGAACGGTACATCTACAACCGGGAAGAGTTCGCCAGATTCGACAGCGACGTGGGCGAGTTCAGAGCCGTGACAGAGCTGGGCAGACCTGCCGCCGAGTACTGGAACAGCCAGAAGGACATCCTGGAAGAGAAGCGGGCCGTGCCCGACCGGATGTGCAGACACAATTACGAGCTGGGAGGCCCCATGACCCTGCAGAGAAGAGTGCAGCCCAGAGTGAACGTGTCCCCCAGCAAGAAGGGCCCCCTGCAGCACCACAACTGGCTTGTCTGCCACGTGACCGACTTCTACCCCGGCTCTATCCAAGTGCGGTGGTTCCTGAACGGCCAGGAAGAGACAGCCGGCGTGATGTCCACCAACCTGATCAGAAACGGCGACTGGACCTTCCAGATCCTCGTGATGCTGGAAATGACCCCCCAGCAGGGCGACGTGTACACCTGTCAGGTGGAACACACCAGCCTGGACAGCCCCGTGACCGTGGAATGGAAGGCCCAGAGCGATAGCGCCAGAAGCAAAGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 260 - protein, 273 aa, Artificial Sequence
  Synthetic Construct
MMRPIVLVLLFATSALAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETGGGGSLEIRAAFLRQRNTALRTEVAELEQEVQRLENEVSQYETRYGPLGGGKGSHHHHHHHHHH

SEQ ID NO: 261 - protein, 822 aa, Artificial Sequence
  Synthetic Construct
ATGATGAGGCCCATCGTGCTGGTGCTGCTGTTCGCCACATCTGCCCTGGCCATCAAGGCCGACCACGTGTCCACATACGCCGCCTTCGTGCAGACCCACAGACCCACCGGCGAGTTCATGTTCGAGTTCGACGAGGACGAGATGTTCTACGTGGACCTGGACAAGAAAGAAACCGTGTGGCACCTGGAAGAGTTCGGCCAGGCCTTCAGCTTTGAGGCCCAGGGCGGACTGGCCAATATCGCCATCCTGAACAACAACCTGAACACCCTGATCCAGCGGAGCAACCACACCCAGGCCACCAACGATCCCCCCGAAGTGACCGTGTTCCCCAAAGAACCCGTGGAACTGGGCCAGCCCAATACCCTGATCTGCCACATCGACAAGTTCTTCCCCCCCGTGCTGAACGTGACCTGGCTGTGCAATGGCGAGCTCGTGACAGAGGGCGTGGCCGAGTCTCTGTTCCTGCCCAGAACCGACTACAGCTTCCACAAGTTCCACTACCTGACCTTCGTGCCCAGCGCCGAGGACTTCTACGACTGCAGAGTGGAACACTGGGGCCTGGACCAGCCCCTGCTGAAACATTGGGAAGCCCAGGAACCCATCCAGATGCCCGAGACAACCGAGACAGGCGGCGGAGGCAGCCTGGAAATCAGAGCCGCCTTCCTGCGGCAGAGAAACACCGCCCTGAGAACCGAAGTGGCCGAGCTGGAACAGGAAGTGCAGCGGCTGGAAAACGAGGTGTCCCAGTACGAGACAAGATACGGCCCTCTGGGAGGCGGCAAGGGCTCTCACCACCACCATCACCATCATCATCACCATTGA

SEQ ID NO: 262 - protein, 1185 aa, Artificial Sequence
  Synthetic Construct
ATGATGCGGCCCATCGTGCTGGTGCTGCTGTTTGCCACATCTGCCCTGGCCAAGAAAGTGGTGCTGGGCAAAAAAGGGGATACAGTGGAACTGACCTGTACAGCTTCCCAGAAGAAGAGCATACAATTCCACTGGAAAAACTCCAACCAGATAAAGATTCTGGGAAATCAGGGCTCCTTCTTAACTAAAGGTCCATCCAAGCTGAATGATCGCGCTGACTCAAGAAGAAGCCTTTGGGACCAAGGAAACTTTCCCCTGATCATCAAGAATCTTAAGATAGAAGACTCAGATACTTACATCTGTGAAGTGGAGGACCAGAAGGAGGAGGTGCAATTGCTAGTGTTCGGATTGACTGCCAACTCTGACACCCACCTGCTTCAGGGGCAGAGCCTGACCCTGACCTTGGAGAGCCCCCCTGGTAGTAGCCCCTCAGTGCAATGTAGGAGTCCAAGGGGTAAAAACATACAGGGGGGGAAGACCCTCTCCGTGTCTCAGCTGGAGCTCCAGGATAGTGGCACCTGGACATGCACTGTCTTGCAGAACCAGAAGAAGGTGGAGTTCAAAATAGACATCGTGGTGCTAGCTTTCCAGAAGGCCTCCAGCATAGTCTATAAGAAAGAGGGGGAACAGGTGGAGTTCTCCTTCCCACTCGCCTTTACAGTTGAAAAGCTGACGGGCAGTGGCGAGCTGTGGTGGCAGGCGGAGAGGGCTTCCTCCTCCAAGTCTTGGATCACCTTTGACCTGAAGAACAAGGAAGTGTCTGTAAAACGGGTTACCCAGGACCCTAAGCTCCAGATGGGCAAGAAGCTCCCGCTCCACCTCACCCTGCCCCAGGCCTTGCCTCAGTATGCTGGCTCTGGAAACCTCACCCTGGCCCTTGAAGCGAAAACAGGAAAGTTGCATCAGGAAGTGAACCTGGTGGTGATGAGAGCCACTCAGCTCCAGAAAAATTTGACCTGTGAGGTGTGGGGACCCACCTCCCCTAAGCTGATGCTGAGCTTGAAACTGGAGAACAAGGAGGCAAAGGTCTCGAAGCGGGAGAAGGCGGTGTGGGTGCTGAACCCTGAGGCGGGGATGTGGCAGTGTCTGCTGAGTGACTCGGGACAGGTCCTGCTGGAATCCAACATCAAGGTTCTGCCCACATGGGGCAGCCACCACCACCATCACCATCATCATCACCATTGA

SEQ ID NO: 263 - protein, 394 aa, Artificial Sequence
  Synthetic Construct
MMRPIVLVLLFATSALAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWGSHHHHHHHHHH

SEQ ID NO: 264 - protein, 15 aa, Artificial Sequence
  Synthetic Construct
GLNDIFEAQKIEWHE

SEQ ID NO: 265 - protein, 198 aa, Artificial Sequence
  Synthetic Construct
RDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNWLICSVTDFYPSQIKVRWFRNDQEETAGVMSTPLIRNGDWTFQILVMLEMTPQRGDVYTCHVEHPSLQSPITVEWRAQSESAQSK

SEQ ID NO: 266 - protein, 267 aa, Artificial Sequence
  Synthetic Construct
MMRPIVLVLLFATSALARDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSKGGGGSLEIEAAFLERENTALETRVAELRQRVQRLRNRVSQYRTRYGPLGGGK

SEQ ID NO: 267 - protein, 267 aa, Artificial Sequence
  Synthetic Construct
MMRPIVLVLLFATSALARDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNWLICSVTDFYPSQIKVRWFRNDQEETAGVMSTPLIRNGDWTFQILVMLEMTPQRGDVYTCHVEHPSLQSPITVEWRAQSESAQSKGGGGSLEIEAAFLERENTALETRVAELRQRVQRLRNRVSQYRTRYGPLGGGK

SEQ ID NO: 268 - protein, 198 aa, Artificial Sequence
  Synthetic Construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCSVSGFYPGSIEVRWFRNGQEEKAGVMSTGLIQNGDWTFQTLVMLETVPRSGEVYTCQVEHPSVTSPLTVEWRARSESAQSK

SEQ ID NO: 269 - protein, 198 aa, Artificial Sequence
  Synthetic Construct
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEETAGVMSTNLIQNGDWTFQILVMLEMTPRSGEVYTCQVEHPSVTSPLTVEWRARSESAQSK

SEQ ID NO: 270 - protein, 284 aa, Artificial Sequence
  Synthetic Construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSLEIEAAFLERENTALETRVAELRQRVQRLRNRVSQYRTRYGPLGGGKGSGLNDIFEAQKIEWHE

SEQ ID NO: 271 - protein, 855 aa, Artificial Sequence
  Synthetic Construct
ATGATGCGGCCCATCGTGCTGGTGCTGCTGTTCGCCACATCTGCCCTGGCCGGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACCTCCTGGTCTGCTCTGTGAGTGGTTTCTATCCAGGCAGCATTGAAGTCAGGTGGTTCCGGAACGGCCAGGAAGAGAAGGCTGGGGTGGTGTCCACAGGCCTGATCCAGAATGGAGATTGGACCTTCCAGACCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAGGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 272 - protein, 284 aa, Artificial Sequence
  Synthetic Construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEEKAGVMSTGLIQNGDWTFQILVMLETVPRSGEVYTCQVEHPSVTSPLTVEWRARSESAQSKGGGGSLEIEAAFLERENTALETRVAELRQRVQRLRNRVSQYRTRYGPLGGGKGSGLNDIFEAQKIEWHE

SEQ ID NO: 273 - protein, 855 aa, Artificial Sequence
  Synthetic Construct
ATGATGCGGCCCATCGTGCTGGTGCTGCTGTTCGCCACATCTGCCCTGGCCGGGGACACCCGACCACGTTTCTTGTGGCAGCTTAAGTTTGAATGTCATTTCTTCAATGGGACGGAGCGGGTGCGGTTGCTGGAAAGATGCATCTATAACCAAGAGGAGTCCGTGCGCTTCGACAGCGACGTGGGGGAGTACCGGGCGGTGACGGAGCTGGGGCGGCCTGATGCCGAGTACTGGAACAGCCAGAAGGACCTCCTGGAGCAGAGGCGGGCCGCGGTGGACACCTACTGCAGACACAACTACGGGGTTGGTGAGAGCTTCACAGTGCAGCGGCGAGTTGAGCCTAAGGTGACTGTGTATCCTTCAAAGACCCAGCCCCTGCAGCACCACAACTGGCTGGTCTGCCATGTGAGTGGTTTCTATCCAGGCAGCATTGAAGTCAGGTGGTTCCGGAACGGCCAGGAAGAGAAGGCTGGGGTGATGTCCACAGGCCTGATCCAGAATGGAGATTGGACCTTCCAGATCCTGGTGATGCTGGAAACAGTTCCTCGGAGTGGAGAGGTTTACACCTGCCAAGTGGAGCACCCAAGTGTGACGAGCCCTCTCACAGTGGAATGGAGAGCACGGTCTGAATCTGCACAGAGCAAGGGCGGCGGAGGCAGCCTGGAAATCGAGGCCGCCTTCCTGGAAAGAGAGAACACCGCCCTGGAAACCCGGGTGGCCGAGCTGAGACAGAGAGTGCAGAGACTGCGGAACCGGGTGTCCCAGTACCGGACCAGATATGGCCCTCTGGGAGGCGGCAAAGGGTCCGGCTTGAACGACATTTTTGAGGCCCAGAAGATAGAGTGGCACGAGTGA

SEQ ID NO: 274 - protein, 267 aa, Artificial Sequence
  Synthetic Construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCSVSGFYPGSIEVRWFRNGQEEKAGVMSTGLIQNGDWTFQTLVMLETVPRSGEVYTCQVEHPSVTSPLTVEWRARSESAQSKGGGGSLEIEAAFLERENTALETRVAELRQRVQRLRNRVSQYRTRYGPLGGGK

SEQ ID NO: 275 - protein, 267 aa, Artificial Sequence
  Synthetic Construct
MMRPIVLVLLFATSALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNWLVCHVSGFYPGSIEVRWFRNGQEETAGVMSTNLIQNGDWTFQILVMLEMTPRSGEVYTCQVEHPSVTSPLTVEWRARSESAQSKGGGGSLEIEAAFLERENTALETRVAELRQRVQRLRNRVSQYRTRYGPLGGGK

SEQ ID NO: 276 - protein, 273 aa, Artificial Sequence
  Synthetic Construct
MMRPIVLVLLFATSALAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENGGGGSLEIRAAFLRQRNTALRTEVAELEQEVQRLENEVSQYETRYGPLGGGKGSHHHHHHHHHH

SEQ ID NO: 277 - protein, 822 aa, Artificial Sequence
  Synthetic Construct
ATGATGCGGCCCATCGTGCTGGTGCTGCTGTTCGCCACATCTGCCCTGGCCATCAAAGAAGAACATGTGATCATCCAGGCCGAGTTCTATCTGAATCCTGACCAATCAGGCGAGTTTATGTTTGACTTTGATGGTGATGAGATTTTCCATGTGGATATGGCAAAGAAGGAGACGGTCTGGCGGCTTGAAGAATTTGGACGATTTGCCAGCTTTGAGGCTCAAGGTGCATTGGCCAACATAGCTGTGGACAAAGCCAACCTGGAAATCATGACAAAGCGCTCCAACTATACTCCGATCACCAATGTACCTCCAGAGGTAACTGTGCTCACGAACAGCCCTGTGGAACTGAGAGAGCCCAACGTCCTCATCTGTTTCATCGACAAGTTCACCCCACCAGTGGTCAATGTCACGTGGCTTCGAAATGGAAAACCTGTCACCACAGGAGTGTCAGAGACAGTCTTCCTGCCCAGGGAAGACCACCTTTTCCGCAAGTTCCACTATCTCCCCTTCCTGCCCTCAACTGAGGACGTTTACGACTGCAGGGTGGAGCACTGGGGCTTGGATGAGCCTCTTCTCAAGCACTGGGAGTTTGATGCTCCAAGCCCTCTCCCAGAGACTACAGAGAACGGCGGCGGAGGCAGCCTGGAAATCAGAGCCGCCTTCCTGCGGCAGAGAAACACCGCCCTGAGAACCGAAGTGGCCGAGCTGGAACAGGAAGTGCAGCGGCTGGAAAACGAGGTGTCCCAGTACGAGACAAGATACGGCCCTCTGGGAGGCGGCAAGGGCTCTCACCACCACCATCACCATCATCATCACCATTGA

SEQ ID NO: 278 - protein, 17 aa, Artificial Sequence
  Synthetic Construct
CVVSGGVNGGATNKLIF

SEQ ID NO: 279 - protein, 15 aa, Artificial Sequence
  Synthetic Construct
CASSLTGGVSYEQYF

SEQ ID NO: 280 - protein, 15 aa, Artificial Sequence
  Synthetic Construct
CAASVRGSTLGRLYF

SEQ ID NO: 281 - protein, 15 aa, Artificial Sequence
  Synthetic Construct
CASSLGTGGTGELFF

SEQ ID NO: 282 - protein, 16 aa, Artificial Sequence
  Synthetic Construct
CAVSGGRGSTLGRLYF

SEQ ID NO: 283 - protein, 15 aa, Artificial Sequence
  Synthetic Construct
CSVQGGLDSNYGYTF

SEQ ID NO: 284 - protein, 15 aa, Artificial Sequence
  Synthetic Construct
CAASVRGSTLGRLYF

SEQ ID NO: 285 - protein, 15 aa, Artificial Sequence
  Synthetic Construct
CASSLGTGGTGELFF

SEQ ID NO: 286 - protein, 15 aa, Artificial Sequence
  Synthetic Construct
CAASVRGSTLGRLYF

SEQ ID NO: 287 - protein, 15 aa, Artificial Sequence
  Synthetic Construct
CASSLGTGGTGELFF

SEQ ID NO: 288 - protein, 13 aa, Artificial Sequence
  Synthetic Construct
CAYRSNNFNKFYF

SEQ ID NO: 289 - protein, 15 aa, Artificial Sequence
  Synthetic Construct
CASSLNTGAGYGYTF

SEQ ID NO: 290 - protein, 15 aa, Artificial Sequence
  Synthetic Construct
CAASVRGSTLGRLYF

SEQ ID NO: 291 - protein, 15 aa, Artificial Sequence
  Synthetic Construct
CASSLGTGGTGELFF

SEQ ID NO: 292 - protein, 15 aa, Artificial Sequence
  Synthetic Construct
CAASVRGSTLGRLYF

SEQ ID NO: 293 - protein, 15 aa, Artificial Sequence
  Synthetic Construct
CASSLGTGGTGELFF

SEQ ID NO: 294 - protein, 14 aa, Artificial Sequence
  Synthetic Construct
CAVEERTGGFKTIF

SEQ ID NO: 295 - protein, 18 aa, Artificial Sequence
  Synthetic Construct
CASSLPSGGAPGTGELFF

SEQ ID NO: 296 - protein, 16 aa, Artificial Sequence
  Synthetic Construct
CAVSGGRGSTLGRLYF

SEQ ID NO: 297 - protein, 15 aa, Artificial Sequence
  Synthetic Construct
CSVQGGLDSNYGYTF

SEQ ID NO: 298 - protein, 15 aa, Artificial Sequence
  Synthetic Construct
CAASVRGSTLGRLYF

SEQ ID NO: 299 - protein, 15 aa, Artificial Sequence
  Synthetic Construct
CASSLGTGGTGELFF

SEQ ID NO: 300 - protein, 16 aa, Artificial Sequence
  Synthetic Construct
CLVGDLGANAGNMLTF

SEQ ID NO: 301 - protein, 15 aa, Artificial Sequence
  Synthetic Construct
CASSIATTNTGELFF

SEQ ID NO: 302 - protein, 16 aa, Artificial Sequence
  Synthetic Construct
CLVGDLGANAGNMLTF

SEQ ID NO: 303 - protein, 15 aa, Artificial Sequence
  Synthetic Construct
CASSLETGTNYEQYF

SEQ ID NO: 304 - protein, 15 aa, Artificial Sequence
  Synthetic Construct
CAVALYGGATNKLIF

SEQ ID NO: 305 - protein, 14 aa, Artificial Sequence
  Synthetic Construct
CASSLDIGNNEQFF

SEQ ID NO: 306 - protein, 14 aa, Artificial Sequence
  Synthetic Construct
CAGRSGGSNYKLTF

SEQ ID NO: 307 - protein, 15 aa, Artificial Sequence
  Synthetic Construct
CASSIATTNTGELFF

SEQ ID NO: 308 - protein, 14 aa, Artificial Sequence
  Synthetic Construct
CAGRSGGSNYKLTF

SEQ ID NO: 309 - protein, 15 aa, Artificial Sequence
  Synthetic Construct
CASSIATTNTGELFF

SEQ ID NO: 310 - protein, 14 aa, Artificial Sequence
  Synthetic Construct
CAGRSGGSNYKLTF

SEQ ID NO: 311 - protein, 15 aa, Artificial Sequence
  Synthetic Construct
CASSIATTNTGELFF

SEQ ID NO: 312 - protein, 14 aa, Artificial Sequence
  Synthetic Construct
CAGRSGGSNYKLTF

SEQ ID NO: 313 - protein, 15 aa, Artificial Sequence
  Synthetic Construct
CASSIATTNTGELFF

SEQ ID NO: 314 - protein, 14 aa, Artificial Sequence
  Synthetic Construct
CAGRSGGSNYKLTF

SEQ ID NO: 315 - protein, 15 aa, Artificial Sequence
  Synthetic Construct
CASSIATTNTGELFF

SEQ ID NO: 316 - protein, 12 aa, Artificial Sequence
  Synthetic Construct
CALYTNAGKSTF

SEQ ID NO: 317 - protein, 15 aa, Artificial Sequence
  Synthetic Construct
CATSRDVSSTDTQYF

SEQ ID NO: 318 - protein, 13 aa, Artificial Sequence
  Synthetic Construct
CADLSGGYNKLIF

SEQ ID NO: 319 - protein, 14 aa, Artificial Sequence
  Synthetic Construct
CASSPTLGTDTQYF

SEQ ID NO: 320 - protein, 19 aa, Artificial Sequence
  Synthetic Construct
CAYRSFLNAGGTSYGKLTF

SEQ ID NO: 321 - protein, 18 aa, Artificial Sequence
  Synthetic Construct
CAASRESKWSSYNSPLHF

(19) United States
(12) Patent Application Publication
(10) Pub. No.: US 2022/0291215 A1
(43) Pub. Date: Sep. 15, 2022

(54) METHODS OF IDENTIFYING T CELL RECEPTORS

(71) Applicant: University Health Network, Toronto (CA)

(72) Inventors: Naoto HIRANO, Toronto (CA); Munehide NAKATSUGAWA, Toronto (CA); Yuki YAMASHITA, Toronto (CA); Kenji SUGATA, Toronto (CA); Muhammed Aashiq RAHMAN, Toronto (CA)

(73) Assignee: University Health Network, Toronto, ON (CA)

(21) Appl. No.: 17/631,818

(22) PCT Filed: Jul. 29, 2020
(86) PCT No.: PCT/IB2020/057176

§ 371 Date: Jan. 31, 2022

Related U.S. Application Data
(60) Provisional application No. 63/029,103, filed on May 22, 2020.
(60) Provisional application No. 62/880,492, filed on Jul. 30, 2019.

(51) Int. Cl. G01N 33/569; G01N 33/554

(52) U.S. Cl. CPC G01N 33/56977; G01N 33/554; G01N 2333/7051; G01N 2333/70539

(57) ABSTRACT

The present disclosure is directed to methods of identifying MHC class II-specific T cell receptors (TCRs). In certain aspects, the method comprises contacting a T cell with a complex comprising an (i) MHC class II molecule having a higher affinity for CD4 than naturally occurring MHC class II molecules and (ii) a peptide, e.g., an epitope. In certain aspects, the HLA class II molecule comprises a beta chain having one or more mutations relative to a wild-type beta chain sequence.

### CROSS REFERENCE TO RELATED APPLICATIONS

This PCT application claims the priority benefit of U.S. Provisional Application Nos. 62/880,492, filed Jul. 30, 2019, and 63/029,103, filed May 22, 2020, each of which is incorporated herein by reference in its entirety.

### REFERENCE TO SEQUENCE LISTING SUBMITTED ELECTRONICALLY VIA EFS-WEB

The content of the electronically submitted sequence listing (Name: 4285.009PC02_SL_ST25.txt, Size: 291,794 bytes; and Date of Creation: Jul. 28, 2020) is incorporated herein by reference in its entirety.

### FIELD OF THE DISCLOSURE

The present disclosure provides methods of identifying MHC class II-specific T cell receptors (“TCRs”).

### BACKGROUND OF THE DISCLOSURE

Immunotherapy has emerged as a critical tool in the battle against a variety of diseases, including cancer. T cell therapies are at the forefront of immunotherapeutic development, and adoptive transfer of antitumor T cells has been shown to induce clinical responses in cancer patients. Though many T cell therapies target mutated tumor antigens, the vast majority of neoantigens are not shared and are unique to each patient.

Potential non-mutated antigens outnumber mutated antigens by multiple orders of magnitude. The elucidation of T cell epitopes derived from shared antigens may facilitate the robust development of efficacious and safe adoptive T cell therapies that are readily available to a larger cohort of cancer patients. However, the sheer number of non-mutated antigens and the high polymorphism of HLA genes may have hampered comprehensive analyses of the specificity of antitumor T cell responses toward non-mutated antigens.

### SUMMARY OF THE DISCLOSURE

Certain aspects of the present disclosure are directed to a method of identifying an MHC class II-specific T cell receptor (TCR) comprising contacting a T cell with a complex comprising an MHC class II molecule and a peptide; wherein the T cell expresses CD4 and one or more TCRs; wherein the MHC class II molecule comprises an alpha chain and a beta chain, wherein the MHC class II molecule has a higher affinity for CD4 than a naturally occurring MHC class II molecule has for the CD4; and wherein the MHC class II-specific TCR specifically binds the complex comprising the MHC class II molecule and the peptide.

In some aspects, the beta chain of the MHC class II molecule comprises an amino acid sequence having one or more mutations relative to a wild-type beta chain of a MHC class II molecule. In some aspects, the alpha chain of the MHC class II molecule comprises an amino acid sequence having one or more mutations relative to a wild-type alpha chain of a MHC class II molecule. In some aspects, the one or more mutations comprise a substitution mutation.

In some aspects, the MHC class II molecule is an HLA-DP, HLA-DQ, or HLA-DR allele, or any combination thereof. In some aspects, (i) the beta chain of the HLA class II molecule is an HLA-DP allele, (ii) the alpha chain of the HLA class II molecule is an HLA-DP allele, or (iii) both (i) and (ii). In some aspects, the beta chain of the HLA class II molecule is a DP1, DP2, DP3, DP4, DP5, DP6, DP8, or DP9 allele.

In some aspects, the beta chain of the MHC class II molecule comprises an HLA allele selected from the group consisting of DPB1*01, DPB1*02, DPB1*03, DPB1*04, DPB1*05, DPB1*06, DPB1*08, DPB1*09, DPB1*10, DPB1*100, DPB1*101, DPB1*102, DPB1*103, DPB1*104, DPB1*105, DPB1*106, DPB1*107, DPB1*108, DPB1*109, DPB1*110, DPB1*111, DPB1*112, DPB1*113, DPB1*114, DPB1*115, DPB1*116, DPB1*117, DPB1*118, DPB1*119, DPB1*11, DPB1*120, DPB1*121, DPB1*122, DPB1*123, DPB1*124, DPB1*125, DPB1*126, DPB1*127, DPB1*128, DPB1*129, DPB1*130, DPB1*131, DPB1*132, DPB1*133, DPB1*134, DPB1*135, DPB1*136, DPB1*137, DPB1*138, DPB1*139, DPB1*13, DPB1*140, DPB1*141, DPB1*142, DPB1*143, DPB1*144, DPB1*145, DPB1*146, DPB1*147, DPB1*148, DPB1*149, DPB1*14, DPB1*150, DPB1*151, DPB1*152, DPB1*153, DPB1*154, DPB1*155, DPB1*156, DPB1*157, DPB1*158, DPB1*159, DPB1*15, DPB1*160, DPB1*161, DPB1*162, DPB1*163, DPB1*164, DPB1*165, DPB1*166, DPB1*167, DPB1*168, DPB1*169, DPB1*16, DPB1*170, DPB1*171, DPB1*172, DPB1*173, DPB1*174, DPB1*175, DPB1*176, DPB1*177, DPB1*178, DPB1*179, DPB1*17, DPB1*180, DPB1*181, DPB1*182, DPB1*183, DPB1*184, DPB1*185, DPB1*186, DPB1*187, DPB1*188, DPB1*189, DPB1*18, DPB1*190, DPB1*191, DPB1*192, DPB1*193, DPB1*194, DPB1*195, DPB1*196, DPB1*197, DPB1*198, DPB1*199, DPB1*19, DPB1*200, DPB1*201, DPB1*202, DPB1*203, DPB1*204, DPB1*205, DPB1*206, DPB1*207, DPB1*208, DPB1*209, DPB1*20, DPB1*210, DPB1*211, DPB1*212, DPB1*213, DPB1*214, DPB1*215, DPB1*216, DPB1*217, DPB1*218, DPB1*219, DPB1*21, DPB1*220, DPB1*221, DPB1*222, DPB1*223, DPB1*224, DPB1*225, DPB1*226, DPB1*227, DPB1*228, DPB1*229, DPB1*22, DPB1*230, DPB1*231, DPB1*232, DPB1*233, DPB1*234, DPB1*235, DPB1*236, DPB1*237, DPB1*238, DPB1*239, DPB1*23, DPB1*240, DPB1*241, DPB1*242, DPB1*243, DPB1*244, DPB1*245, DPB1*246, DPB1*247, DPB1*248, DPB1*249, DPB1*24, DPB1*250, DPB1*251, DPB1*252, DPB1*253, DPB1*254, DPB1*255, DPB1*256, DPB1*257, DPB1*258, DPB1*259, DPB1*25, DPB1*260, DPB1*261, DPB1*262, DPB1*263, DPB1*264, DPB1*265, DPB1*266, DPB1*267, DPB1*268, DPB1*269, DPB1*26, DPB1*270, DPB1*271, DPB1*272, DPB1*273, DPB1*274, DPB1*275, DPB1*276, DPB1*277, DPB1*278, DPB1*279, DPB1*27, DPB1*280, DPB1*281, DPB1*282, DPB1*283, DPB1*284, DPB1*285, DPB1*286, DPB1*287, DPB1*288, DPB1*289, DPB1*28, DPB1*290, DPB1*291, DPB1*292, DPB1*293, DPB1*294, DPB1*295, DPB1*296, DPB1*297, DPB1*298, DPB1*299, DPB1*29, DPB1*300, DPB1*301, DPB1*302, DPB1*303, DPB1*304, DPB1*305, DPB1*306, DPB1*307, DPB1*308, DPB1*309, DPB1*30, DPB1*310, DPB1*311, DPB1*312, DPB1*313, DPB1*314, DPB1*315, DPB1*316, DPB1*317, DPB1*318, DPB1*319, DPB1*31, DPB1*320, DPB1*321, DPB1*322, DPB1*323, DPB1*324, DPB1*325, DPB1*326, DPB1*327, DPB1*328, DPB1*329, DPB1*32, DPB1*330, DPB1*331, DPB1*332, DPB1*333, DPB1*334, DPB1*335, DPB1*336, DPB1*337, DPB1*338, DPB1*339, DPB1*33, DPB1*340, DPB1*341, DPB1*342, DPB1*343, DPB1*344, DPB1*345, DPB1*346, DPB1*347, DPB1*348, DPB1*349, DPB1*34, DPB1*350, DPB1*351, DPB1*352, DPB1*353, DPB1*354, DPB1*355, DPB1*356, DPB1*357, DPB1*358, DPB1*359, DPB1*35, DPB1*360, DPB1*361, DPB1*362, DPB1*363, DPB1*364, DPB1*365, DPB1*366, DPB1*367, DPB1*368, DPB1*369, DPB1*36, DPB1*370, DPB1*371, DPB1*372, DPB1*373, DPB1*374, DPB1*375, DPB1*376, DPB1*377, DPB1*378, DPB1*379, DPB1*37, DPB1*380, DPB1*381, DPB1*382, DPB1*383, DPB1*384, DPB1*385, DPB1*386, DPB1*387, DPB1*388, DPB1*389, DPB1*38, DPB1*390, DPB1*391, DPB1*392, DPB1*393, DPB1*394, DPB1*395, DPB1*396, DPB1*397, DPB1*398, DPB1*399, DPB1*39, DPB1*400, DPB1*401, DPB1*402, DPB1*403, DPB1*404, DPB1*405, DPB1*406, DPB1*407, DPB1*408, DPB1*409, DPB1*40, DPB1*410, DPB1*411, DPB1*412, DPB1*413, DPB1*414, DPB1*415, DPB1*416, DPB1*417, DPB1*418, DPB1*419, DPB1*41, DPB1*420, DPB1*421, DPB1*422, DPB1*423, DPB1*424, DPB1*425, DPB1*426, DPB1*427, DPB1*428, DPB1*429, DPB1*430, DPB1*431, DPB1*432, DPB1*433, DPB1*434, DPB1*435, DPB1*436, DPB1*437, DPB1*438, DPB1*439, DPB1*440, DPB1*441, DPB1*442, DPB1*443, DPB1*444, DPB1*445, DPB1*446, DPB1*447, DPB1*448, DPB1*449, DPB1*44, DPB1*450, DPB1*451, DPB1*452, DPB1*453, DPB1*454, DPB1*455, DPB1*456, DPB1*457, DPB1*458, DPB1*459, DPB1*45, DPB1*460, DPB1*461, DPB1*462, DPB1*463, DPB1*464, DPB1*465, DPB1*466, DPB1*467, DPB1*468, DPB1*469, DPB1*46, DPB1*470, DPB1*471, DPB1*472, DPB1*473, DPB1*474, DPB1*475, DPB1*476, DPB1*477, DPB1*478, DPB1*479, DPB1*47, DPB1*480, DPB1*481, DPB1*482, DPB1*483, DPB1*484, DPB1*485, DPB1*486, DPB1*487, DPB1*488, DPB1*489, DPB1*48, DPB1*490, DPB1*491, DPB1*492, DPB1*493, DPB1*494, DPB1*495, DPB1*496, DPB1*497, DPB1*498, DPB1*499, DPB1*49, DPB1*500, DPB1*501, DPB1*502, DPB1*503, DPB1*504, DPB1*505, DPB1*506, DPB1*507, DPB1*508, DPB1*509, DPB1*50, DPB1*510, DPB1*511, DPB1*512, DPB1*513, DPB1*514, DPB1*515, DPB1*516, DPB1*517, DPB1*518, DPB1*519, DPB1*51, DPB1*520, DPB1*521, DPB1*522, DPB1*523, DPB1*524, DPB1*525, DPB1*526, DPB1*527, DPB1*528, DPB1*529, DPB1*52, DPB1*530, DPB1*531, DPB1*532, DPB1*533, DPB1*534, DPB1*535, DPB1*536, DPB1*537, DPB1*538, DPB1*539, DPB1*53, DPB1*540, DPB1*541, DPB1*542, DPB1*543, DPB1*544, DPB1*545, DPB1*546, DPB1*547, DPB1*548, DPB1*549, DPB1*54, DPB1*550, DPB1*551, DPB1*552, DPB1*553, DPB1*554, DPB1*555, DPB1*556, DPB1*557, DPB1*558, DPB1*559, DPB1*55, DPB1*560, DPB1*561, DPB1*562, DPB1*563, DPB1*564, DPB1*565, DPB1*566, DPB1*567, DPB1*568, DPB1*569, DPB1*56, DPB1*570, DPB1*571, DPB1*572, DPB1*573, DPB1*574, DPB1*575, DPB1*576, DPB1*577, DPB1*578, DPB1*579, DPB1*57, DPB1*580, DPB1*581, DPB1*582, DPB1*583, DPB1*584, DPB1*585, DPB1*586, DPB1*587, DPB1*588, DPB1*589, DPB1*58, DPB1*590, DPB1*591, DPB1*592, DPB1*593, DPB1*594, DPB1*595, DPB1*596, DPB1*597, DPB1*598, DPB1*599, DPB1*59, DPB1*600, DPB1*601, DPB1*602, DPB1*603, DPB1*604, DPB1*605, DPB1*606, DPB1*607, DPB1*608, DPB1*609, DPB1*60, DPB1*610, DPB1*611, DPB1*612, DPB1*613, DPB1*614, DPB1*615, DPB1*616, DPB1*617, DPB1*618, DPB1*619, DPB1*61, DPB1*620, DPB1*621, DPB1*622, DPB1*623, DPB1*624, DPB1*625, DPB1*626, DPB1*627, DPB1*628, DPB1*629, DPB1*62, DPB1*630, DPB1*631, DPB1*632, DPB1*633, DPB1*634, DPB1*635, DPB1*636, DPB1*637, DPB1*638, DPB1*639, DPB1*63, DPB1*640, DPB1*641, DPB1*642, DPB1*643, DPB1*644, DPB1*645, DPB1*646, DPB1*647, DPB1*648, DPB1*649, DPB1*64, DPB1*650, DPB1*651, DPB1*652, DPB1*653, DPB1*654, DPB1*655, DPB1*656, DPB1*657, DPB1*658, DPB1*659, DPB1*65, DPB1*660, DPB1*661, DPB1*662, DPB1*663, DPB1*664, DPB1*665, DPB1*666, DPB1*667, DPB1*668, DPB1*669, DPB1*66, DPB1*670, DPB1*671, DPB1*672, DPB1*673, DPB1*674, DPB1*675, DPB1*676, DPB1*677, DPB1*678, DPB1*679, DPB1*67, DPB1*680, DPB1*681, DPB1*682, DPB1*683, DPB1*684, DPB1*685, DPB1*686, DPB1*687, DPB1*688, DPB1*689, DPB1*68, DPB1*690, DPB1*691, DPB1*692, DPB1*693, DPB1*694, DPB1*695, DPB1*696, DPB1*697, DPB1*698, DPB1*699, DPB1*69, DPB1*700, DPB1*701, DPB1*702, DPB1*703, DPB1*704, DPB1*705, DPB1*706, DPB1*707, DPB1*708, DPB1*709, DPB1*70, DPB1*710, DPB1*711, DPB1*712, DPB1*713, DPB1*714, DPB1*715, DPB1*716, DPB1*717, DPB1*718, DPB1*719, DPB1*71, DPB1*720, DPB1*721, DPB1*722, DPB1*723, DPB1*724, DPB1*725, DPB1*726, DPB1*727, DPB1*728, DPB1*729, DPB1*72, DPB1*730, DPB1*731, DPB1*732, DPB1*733, DPB1*734, DPB1*735, DPB1*736, DPB1*737, DPB1*738, DPB1*739, DPB1*73, DPB1*740, DPB1*741, DPB1*742, DPB1*743, DPB1*744, DPB1*745, DPB1*746, DPB1*747, DPB1*748, DPB1*749, DPB1*74, DPB1*750, DPB1*751, DPB1*752, DPB1*753, DPB1*754, DPB1*755, DPB1*756, DPB1*757, DPB1*758, DPB1*759, DPB1*75, DPB1*760, DPB1*761, DPB1*762, DPB1*763, DPB1*764, DPB1*765, DPB1*766, DPB1*767, DPB1*768, DPB1*769, DPB1*76, DPB1*770, DPB1*771, DPB1*772, DPB1*773, DPB1*774, DPB1*775, DPB1*776, DPB1*777, DPB1*778, DPB1*779, DPB1*77, DPB1*780, DPB1*781, DPB1*782, DPB1*783, DPB1*784, DPB1*785, DPB1*786, DPB1*787, DPB1*788, DPB1*789, DPB1*78, DPB1*790, DPB1*791, DPB1*792, DPB1*794, DPB1*795, DPB1*796, DPB1*797, DPB1*798, DPB1*799, DPB1*79, DPB1*800, DPB1*801, DPB1*802, DPB1*803, DPB1*804, DPB1*805, DPB1*806, DPB1*807, DPB1*808, DPB1*809, DPB1*80, DPB1*810, DPB1*811, DPB1*812, DPB1*813, DPB1*814, DPB1*815, DPB1*816, DPB1*817, DPB1*818, DPB1*819, DPB1*81, DPB1*820, DPB1*821, DPB1*822, DPB1*823, DPB1*824, DPB1*825, DPB1*826, DPB1*827, DPB1*828, DPB1*829, DPB1*82, DPB1*830, DPB1*831, DPB1*832, DPB1*833, DPB1*834, DPB1*835, DPB1*836, DPB1*837, DPB1*838, DPB1*839, DPB1*83, DPB1*840, DPB1*841, DPB1*842, DPB1*843, DPB1*844, DPB1*845, DPB1*846, DPB1*847, DPB1*848, DPB1*849, DPB1*84, DPB1*850, DPB1*851, DPB1*852, DPB1*853, DPB1*854, DPB1*855, DPB1*856, DPB1*857, DPB1*858, DPB1*859, DPB1*85, DPB1*860, DPB1*861, DPB1*862, DPB1*863, DPB1*864, DPB1*865, DPB1*866, DPB1*867, DPB1*868, DPB1*869, DPB1*86, DPB1*870, DPB1*871, DPB1*872, DPB1*873, DPB1*874, DPB1*875, DPB1*876, DPB1*877, DPB1*878, DPB1*879, DPB1*87, DPB1*880, DPB1*881, DPB1*882, DPB1*883, DPB1*884, DPB1*885, DPB1*886, DPB1*887, DPB1*888, DPB1*889, DPB1*88, DPB1*890, DPB1*891, DPB1*892, DPB1*893, DPB1*894, DPB1*895, DPB1*896, DPB1*897, DPB1*898, DPB1*899, DPB1*89, DPB1*900, DPB1*901, DPB1*902, DPB1*903, DPB1*904, DPB1*905, DPB1*906, DPB1*907, DPB1*908, DPB1*909, DPB1*90, DPB1*910, DPB1*911, DPB1*912, DPB1*913, DPB1*914, DPB1*915, DPB1*916, DPB1*917, DPB1*918, DPB1*919, DPB1*91, DPB1*920, DPB1*921, DPB1*922, DPB1*923, DPB1*924, DPB1*925, DPB1*926, DPB1*927, DPB1*928, DPB1*929, DPB1*92, DPB1*930, DPB1*931, DPB1*932, DPB1*933, DPB1*934, DPB1*935, DPB1*936, DPB1*937, DPB1*938, DPB1*939, DPB1*93, DPB1*940, DPB1*941, DPB1*942, DPB1*943, DPB1*944, DPB1*945, DPB1*946, DPB1*947, DPB1*948, DPB1*949, DPB1*94, DPB1*950, DPB1*951, DPB1*952, DPB1*953, DPB1*954, DPB1*955, DPB1*956, DPB1*957, DPB1*958, DPB1*959, DPB1*95, DPB1*960, DPB1*961, DPB1*962, DPB1*963, DPB1*964, DPB1*965, DPB1*96, DPB1*97, DPB1*98, and DPB1*99 allele.

In some aspects, the alpha chain of the MHC class II molecule comprises an HLA-DPA1*01, HLA-DPA1*02, HLA-DPA1*03, or HLA-DPA1*04 allele.

In some aspects, the beta chain of the MHC class II molecule comprises an amino acid other than leucine at a position corresponding to amino acid residue 112 of SEQ ID NO: 1. In some aspects, the beta chain of the MHC class II molecule comprises an amino acid other than valine at a position corresponding to amino acid residue 141 of SEQ ID NO: 1.

Certain aspects of the present disclosure are directed to a method of identifying a MHC class II-specific T cell receptor (TCR) comprising contacting a T cell with a complex comprising an MHC class II molecule and a peptide; wherein the T cell expresses CD4 and one or more TCRs; wherein the MHC class II molecule comprises an alpha chain and a beta chain, wherein the beta chain of the MHC class II molecule comprises (i) an amino acid other than leucine at a position corresponding to amino acid residue 112 of SEQ ID NO: 1, (ii) an amino acid other than valine at a position corresponding to amino acid residue 141 of SEQ ID NO: 1, or (iii) both (i) and (ii); and wherein the MHC class II-specific TCR specifically binds the complex comprising the MHC class II molecule and the peptide.

In some aspects, the MHC class II molecule has a higher affinity for CD4 than a naturally occurring MHC class II molecule has for CD4.

In some aspects, the amino acid other than leucine at a position corresponding to amino acid residue 112 of SEQ ID NO: 1 comprises a hydrophobic side chain. In some aspects, the amino acid other than leucine at a position corresponding to amino acid residue 112 of SEQ ID NO: 1 is selected from the group consisting of an alanine, a valine, an isoleucine, a methionine, a phenylalanine, a tyrosine, and a tryptophan. In some aspects, the amino acid other than leucine at a position corresponding to amino acid residue 112 of SEQ ID NO: 1 is a tryptophan.

In some aspects, the amino acid other than valine at a position corresponding to amino acid residue 141 of SEQ ID NO: 1 comprises a hydrophobic side chain. In some aspects, the amino acid other than valine at a position corresponding to amino acid residue 141 of SEQ ID NO: 1 is selected from an alanine, an isoleucine, a leucine, a methionine, a phenylalanine, a tyrosine, and a tryptophan. In some aspects, the amino acid other than valine at a position corresponding to amino acid residue 141 of SEQ ID NO: 1 is a methionine.

In some aspects, (i) the beta chain of the HLA class II molecule is an HLA-DQ allele, (ii) the alpha chain of the HLA class II molecule is an HLA-DQ allele, or (iii) both (i) and (ii). In some aspects, the beta chain of the HLA class II molecule comprises a DQ2, DQ3, DQ4, DQ5, or DQ6 allele. In some aspects, the beta chain of the MHC class II molecule comprises an HLA-DQB1*02, HLA-DQB1*03, HLA-DQB1*04, HLA-DQB1*05, or HLA-DQB1*06 allele. In some aspects, the alpha chain of the MHC class II molecule comprises an HLA-DQA1*01, HLA-DQA1*02, HLA-DQA1*03, HLA-DQA1*04, HLA-DQA1*05, or HLA-DQA1*06 allele.

In some aspects, the beta chain of the MHC class II molecule comprises (a) an amino acid other than leucine at a position corresponding to amino acid residue 114 of SEQ ID NO: 11; (b) an amino acid other than valine at a position corresponding to amino acid residue 143 of SEQ ID NO: 11; and (c) at least three of: (i) an amino acid other than asparagine at a position corresponding to amino acid residue 110 of SEQ ID NO: 11, (ii) an amino acid other than isoleucine at a position corresponding to amino acid residue 116 of SEQ ID NO: 11, (iii) an amino acid other than serine at a position corresponding to amino acid residue 118 of SEQ ID NO: 11, and (iv) an amino acid other than proline at a position corresponding to amino acid residue 146 of SEQ ID NO: 11.

In some aspects, the beta chain of the MHC class II molecule comprises (a) an amino acid other than leucine at a position corresponding to amino acid residue 114 of SEQ ID NO: 11; (b) an amino acid other than valine at a position corresponding to amino acid residue 143 of SEQ ID NO: 11; (c) an amino acid other than asparagine at a position corresponding to amino acid residue 110 of SEQ ID NO: 11; (d) an amino acid other than isoleucine at a position corresponding to amino acid residue 116 of SEQ ID NO: 11; (e) an amino acid other than serine at a position corresponding to amino acid residue 118 of SEQ ID NO: 11; and (f) an amino acid other than proline at a position corresponding to amino acid residue 146 of SEQ ID NO: 11.

In some aspects, the amino acid other than leucine at a position corresponding to amino acid residue 114 of SEQ ID NO: 11 comprises a hydrophobic side chain. In some aspects, the amino acid other than leucine at a position corresponding to amino acid residue 114 of SEQ ID NO: 11 is selected from the group consisting of an alanine, a valine, an isoleucine, a methionine, a phenylalanine, a tyrosine, and a tryptophan. In some aspects, the amino acid other than leucine at a position corresponding to amino acid residue 114 of SEQ ID NO: 11 is a tryptophan.

In some aspects, the amino acid other than valine at a position corresponding to amino acid residue 143 of SEQ ID NO: 11 comprises a hydrophobic side chain. In some aspects, the amino acid other than valine at a position corresponding to amino acid residue 143 of SEQ ID NO: 11 is selected from an alanine, an isoleucine, a leucine, a methionine, a phenylalanine, a tyrosine, and a tryptophan. In some aspects, the amino acid other than valine at a position corresponding to amino acid residue 143 of SEQ ID NO: 11 is a methionine.

In some aspects, the beta chain of the MHC class II molecule comprises an amino acid other than asparagine at a position corresponding to amino acid residue 110 of SEQ ID NO: 11. In some aspects, the amino acid other than asparagine at a position corresponding to amino acid residue 110 of SEQ ID NO: 11 is selected from a serine, a threonine, and a glutamine. In some aspects, the amino acid other than asparagine at a position corresponding to amino acid residue 110 of SEQ ID NO: 11 is a glutamine.

In some aspects, the beta chain of the MHC class II molecule comprises an amino acid other than isoleucine at a position corresponding to amino acid residue 116 of SEQ ID NO: 11. In some aspects, the amino acid other than isoleucine at a position corresponding to amino acid residue 116 of SEQ ID NO: 11 is selected from an alanine, a valine, a leucine, a methionine, a phenylalanine, a tyrosine, and a tryptophan. In some aspects, the amino acid other than isoleucine at a position corresponding to amino acid residue 116 of SEQ ID NO: 11 is valine.

In some aspects, the beta chain of the MHC class II molecule comprises an amino acid other than serine at a position corresponding to amino acid residue 118 of SEQ ID NO: 11. In some aspects, the amino acid other than serine at a position corresponding to amino acid residue 118 of SEQ ID NO: 11 is selected from an arginine, a histidine, and a lysine. In some aspects, the amino acid other than serine at a position corresponding to amino acid residue 118 of SEQ ID NO: 11 is a histidine.

In some aspects, the beta chain of the MHC class II molecule comprises an amino acid other than proline at a position corresponding to amino acid residue 146 of SEQ ID NO: 11. In some aspects, the amino acid other than proline at a position corresponding to amino acid residue 146 of SEQ ID NO: 11 is selected from a serine, a threonine, an asparagine, and a glutamine. In some aspects, the amino acid other than proline at a position corresponding to amino acid residue 146 of SEQ ID NO: 11 is a glutamine.

In some aspects, (i) the beta chain of the HLA class II molecule is an HLA-DR allele, (ii) the alpha chain of the HLA class II molecule is an HLA-DR allele, of (iii) both (i) and (ii).

In some aspects, the beta chain of the HLA class II molecule comprises a DR2, DR3, DR4, DR5, DR6, DR7, DR8, DR9, DR10, DR11, DR12, DR13, DR14, DR15, or DR16 allele. In some aspects, the beta chain of the MHC class II molecule comprises an HLA allele selected from the group consisting of DRB1*01, DRB1*03, DRB1*04, DRB1*07, DRB1*08, DRB1*09, DRB1*10, DRB1*11, DRB1*12, DRB1*13, DRB1*14, DRB1*15, and DRB1*16. In some aspects, the alpha chain of the MHC class II molecule comprises an HLA-DRA1*01 allele.

In some aspects, the beta chain comprises: (a) an amino acid other than leucine at a position corresponding to amino acid residue 114 of SEQ ID NO: 19; (b) an amino acid other than valine at a position corresponding to amino acid residue 143 of SEQ ID NO: 19; and (c) at least two of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In some aspects, the beta chain comprises: (c) at least three of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In some aspects, the beta chain comprises: (c) at least four of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In some aspects, the beta chain comprises: (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, and (d) an amino acid other than threonine at a position corresponding to amino acid residue 157 of SEQ ID NO: 19.

In some aspects, the beta chain comprises: (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, (d) an amino acid other than lysine at a position corresponding to amino acid residue 139 of SEQ ID NO: 19, (e) an amino acid other than glycine at a position corresponding to amino acid residue 146 of SEQ ID NO: 19, (f) an amino acid other than threonine at a position corresponding to amino acid residue 157 of SEQ ID NO: 19, (g) an amino acid other than threonine at a position corresponding to amino acid residue 163 of SEQ ID NO: 19, and (h) an amino acid other than valine at a position corresponding to amino acid residue 164 of SEQ ID NO: 19.

In some aspects, the amino acid other than leucine at a position corresponding to amino acid residue 114 of SEQ ID NO: 19 comprises a hydrophobic side chain. In some aspects, the amino acid other than leucine at a position corresponding to amino acid residue 114 of SEQ ID NO: 19 is selected from the group consisting of an alanine, a valine, an isoleucine, a methionine, a phenylalanine, a tyrosine, and a tryptophan. In some aspects, the amino acid other than leucine at a position corresponding to amino acid residue 114 of SEQ ID NO: 19 is a tryptophan.

In some aspects, the amino acid other than valine at a position corresponding to amino acid residue 143 of SEQ ID NO: 19 comprises a hydrophobic side chain. In some aspects, the amino acid other than valine at a position corresponding to amino acid residue 143 of SEQ ID NO: 19 is selected from an alanine, an isoleucine, a leucine, a methionine, a phenylalanine, a tyrosine, and a tryptophan. In some aspects, the amino acid other than valine at a position corresponding to amino acid residue 143 of SEQ ID NO: 19 is a methionine.

In some aspects, the beta chain of the MHC class II molecule comprises an amino acid other than serine at a position corresponding to amino acid residue 118 of SEQ ID NO: 19. In some aspects, the amino acid other than serine at a position corresponding to amino acid residue 118 of SEQ ID NO: 19 is selected from an arginine, a histidine, and a lysine. In some aspects, the amino acid other than serine at a position corresponding to amino acid residue 118 of SEQ ID NO: 19 is a histidine.

In some aspects, the beta chain of the MHC class II molecule comprises an amino acid other than lysine at a position corresponding to amino acid residue 139 of SEQ ID NO: 19. In some aspects, the amino acid other than lysine at a position corresponding to amino acid residue 139 of SEQ ID NO: 19 is selected from a serine, a threonine, and a glutamine. In some aspects, the amino acid other than lysine at a position corresponding to amino acid residue 139 of SEQ ID NO: 19 is a threonine.

In some aspects, the beta chain of the MHC class II molecule comprises an amino acid other than glycine at a position corresponding to amino acid residue 146 of SEQ ID NO: 19. In some aspects, the amino acid other than glycine at a position corresponding to amino acid residue 146 of SEQ ID NO: 19 is selected from a serine, an asparagine, a threonine, and a glutamine. In some aspects, the amino acid other than glycine at a position corresponding to amino acid residue 146 of SEQ ID NO: 19 is a glutamine.

In some aspects, the beta chain of the MHC class II molecule comprises an amino acid other than threonine at a position corresponding to amino acid residue 157 of SEQ ID NO: 19. In some aspects, the amino acid other than threonine at a position corresponding to amino acid residue 157 of SEQ ID NO: 19 is selected from an alanine, a valine, an isoleucine, a leucine, a methionine, a phenylalanine, a tyrosine, and a tryptophan. In some aspects, the amino acid other than threonine at a position corresponding to amino acid residue 157 of SEQ ID NO: 19 is an isoleucine.

In some aspects, the beta chain of the MHC class II molecule comprises an amino acid other than threonine at a position corresponding to amino acid residue 163 of SEQ ID NO: 19. In some aspects, the amino acid other than threonine at a position corresponding to amino acid residue 163 of SEQ ID NO: 19 is selected from an alanine, a valine, an isoleucine, a leucine, a methionine, a phenylalanine, a tyrosine, and a tryptophan. In some aspects, the amino acid other than threonine at a position corresponding to amino acid residue 163 of SEQ ID NO: 19 is a methionine.

In some aspects, the beta chain of the MHC class II molecule comprises an amino acid other than valine at a position corresponding to amino acid residue 164 of SEQ ID NO: 19. In some aspects, the amino acid other than valine at a position corresponding to amino acid residue 164 of SEQ ID NO: 19 is selected from a serine, an asparagine, a threonine, and a glutamine. In some aspects, the amino acid other than valine at a position corresponding to amino acid residue 164 of SEQ ID NO: 19 is a threonine.

In some aspects, the beta chain comprises: (a) a tryptophan at a position corresponding to amino acid residue 114 of SEQ ID NO: 19, (b) a methionine at a position corresponding to amino acid residue 143 of SEQ ID NO: 19, (c) a histidine at a position corresponding to amino acid residue 118 of SEQ ID NO: 19, and (d) an isoleucine at a position corresponding to amino acid residue 157 of SEQ ID NO: 19.

In some aspects, the naturally occurring MHC class II molecule comprises: (a) a leucine at a position corresponding to amino acid residue 112 of SEQ ID NO: 1 or amino acid residue 114 of SEQ ID NO: 11 or 19, (b) a valine at a position corresponding to amino acid residue 141 of SEQ ID NO: 1 or amino acid residue 143 of SEQ ID NO: 11 or 19, or (c) both (a) and (b).

In some aspects, the naturally occurring MHC class II molecule comprises: (a) a leucine at a position corresponding to amino acid residue 112 of SEQ ID NO: 1 or amino acid residue 114 of SEQ ID NO: 11 or 19, (b) a valine at a position corresponding to amino acid residue 141 of SEQ ID NO: 1 or amino acid residue 143 of SEQ ID NO: 11 or 19, (c) an asparagine at a position corresponding to amino acid residue 110 of SEQ ID NO: 11; (d) an isoleucine at a position corresponding to amino acid residue 116 of SEQ ID NO: 11; (e) a serine at a position corresponding to amino acid residue 118 of SEQ ID NO: 11 or 19; and (f) a proline at a position corresponding to amino acid residue 146 of SEQ ID NO: 11 (g) a lysine at a position corresponding to amino acid residue 139 of SEQ ID NO: 19, (h) a glycine at a position corresponding to amino acid residue 146 of SEQ ID NO: 19, (i) a threonine at a position corresponding to amino acid residue 157 of SEQ ID NO: 19, (j) a threonine at a position corresponding to amino acid residue 163 of SEQ ID NO: 19, (k) a valine at a position corresponding to amino acid residue 164 of SEQ ID NO: 19, or (1) any combination of (a) to (k).

In some aspects, the MHC class II molecule is a dimer. In some aspects, the MHC class II molecule is a trimer. In some aspects, the MHC class II molecule is a tetramer. In some aspects, the peptide comprises a fragment of a protein. In some aspects, the protein is expressed by a diseased cell. In some aspects, the protein is expressed by a tumor cell.

In some aspects, the peptide comprises at least about 10 amino acids. In some aspects, the peptide comprises about 10 to about 100 amino acids, about 10 to about 90 amino acids, about 10 to about 80 amino acids, about 10 to about 70 amino acids, about 10 to about 60 amino acids, about 10 to about 50 amino acids, about 10 to about 40 amino acids, about 10 to about 30 amino acids, about 10 to about 25 amino acids, about 10 to about 20 amino acids, about 10 to about 15 amino acids, about 15 to about 100 amino acids, 20 to about 100 amino acids, 25 to about 100 amino acids, 30 to about 100 amino acids, 35 to about 100 amino acids, 40 to about 100 amino acids, 50 to about 100 amino acids, 60 to about 100 amino acids, 70 to about 100 amino acids, 80 to about 100 amino acids, or 90 to about 100 amino acids.

In some aspects, the peptide comprises about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, or about 100 amino acids.

In some aspects, the MHC class II molecule is expressed on the surface of an antigen presenting cell.

In some aspects, the T cell is obtained from a human subject. In some aspects, the T cell is a tumor infiltrating lymphocyte (TIL).

In some aspects, the MHC class II molecule has an affinity for CD4 that is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, or at least about 100-fold higher than the binding affinity of a naturally occurring MHC class II molecule to CD4.

In some aspects, the method further comprises selecting the T cell that is bound by the MHC class II molecule. In some aspects, the method further comprises isolating the TCR that is bound to the MHC class II molecule. In some aspects, the method further comprises sequencing the TCR. In some aspects, the method further comprises cloning the TCR. In some aspects, the method further comprises recombinantly expressing the TCR in a host cell.

In some aspects, the MHC class II molecule binds CD4 with a K_{D} of less than about 100 μM, less than about 50 μM, less than about 20 μM, or less than about 10 μM. In some aspects, the MHC class II molecule binds CD4 with a K_{D} of about 14 μM or less. In some aspects, the MHC class II molecule binds CD4 with a K_{D} of about 8.9 μM or less.

### BRIEF DESCRIPTION OF THE DRAWINGS

FIGS. 1A-1V are graphical representations of data illustrating that affinity-matured DP4^{L112W/V141M} molecules exhibit an enhanced CD4 binding ability. FIGS. 1A-1F are histograms showing the results of HLA class II-null K562 cells stably expressing the wild-type DPα chain (DPA1*01:03) transduced with blank, wild-type, or mutant DPβ chain (DPB1*04:01) harboring L112W, V114M, V141M, and M158I substitutions (DP4^{L112W/V114M/V141M/M158I}) and stained with an anti-class II mAb and soluble CD4 (sCD4). FIG. 1G is a bar graph summarizing the binding affinity for sCD4 (MFI; y-axis) of all possible DP4 reversion mutants, which were similarly expressed and stained with sCD4 as FIGS. 1A-1F. FIG. 1H shows the affinity between DP4^{L112W/V141M} and CD4 as quantified by steady state analysis. FIG. 1I shows the results of an IL-2 EPISPOT assay of DP4/WT1 TCR, clone 9-transduced Jurkat 76 and Jurkat 76/CD4 cells stimulated by wild-type DP4 or DP4^{L112W/V141M}-expressing aAPCs pulsed with graded concentrations of the DP4/WT1 peptide. FIGS. 1J-1W are histograms representing staining of K562 cells expressing DP^{L112W/V141M} alleles (as indicated) with an anti-class II mAb and sCD4. Open histograms represent the isotype control staining. *P<0.05 by Student's t-test. Bars and error bars represent the mean±SD of results in triplicate experiments. At least 2 independent experiments were performed. FIGS. 1X-IAA are histograms showing wild-type DP4 and DP4L112W/V141M molecules on the surface of K562 cells that were detected with the indicated anti-HLA class II antibodies. Staining of control cells devoid of Class II expression is shown in solid gray. FIGS. 1AB-1BH are histograms showing aAPCs expressing the indicated DP4 or class II parental cells that were stained with sCD4 at the indicated concentrations. FIG. 1BI shows the quantification of aAPCs expressing wild-type DP4 or DP4^{L112W/V141M} at the indicated concentrations. Error bars represent the mean±standard deviation of experiments performed in triplicate. FIG. 1BJ is a biolayer interferometry sensogram showing the interaction of biotinylated wild-type DP4 (ligand) with sCD4 (analyte) over a range of concentrations. FIG. 1BK is a biolayer interferometry sensogram showing the interaction of biotinylated DP4^{L112W/V141M} (ligand) with sCD4 (analyte) over a range of concentrations. Experiments in FIGS. 1BJ and 1BI were performed in parallel. All data are representative of two independent experiments.

FIGS. 2A-2D are ribbon diagrams of a model structure of DP4^{L112W/V141M} and the human CD4 complex. FIGS. 2A-2B are two orientations of the ternary complex model structure of DPA1*01:03, DPB1*04:01, and CD4, as indicated. The DPB1*04:01-CD4 binding interface is enclosed in a dashed square (FIG. 2B). FIGS. 2C-2D provide close-up views of the CD4 binding interface of wild-type DP4 (FIG. 2C) and DP4^{L112W/V141M} (FIG. 2D). The side chains of interacting residues are shown as ball-and-stick representations (FIGS. 2C-2D).

FIGS. 3A-3P are graphical representations of data illustrating that DP4^{L112W/V141M} dimers stain cognate TCRs expressed in human primary CD4⁺ T cells. Primary T cells were transduced with either DP4/MAGE-A3₂₄₃₋₂₅₈ (R12C9; FIGS. 3E-3H), DP4/WT1₃₂₈₋₃₄₈ (clone 9; FIGS. 3I-3L), or DP4/NY-ESO-1₁₅₇₋₁₇₀ (5B8; FIGS. 3M-3P) TCR and stained with the indicated DP4^{L112W/V141M} dimers (FIGS. 3B-3D, 3F-3H, 3J-3L, and 3N-3P).

FIGS. 4A-4D are scatter plots illustrating costaining of R12C9-transduced CD4⁺ T cells stained with DP4^{L112W/V141M} dimer and an anti-V022 mAb. Note that R12C9 expresses V022. FIGS. 4E-4H are scatter plots illustrating costaining of Clone 9-transduced CD4⁺ T cells double-stained with DP4^{L112W/V141M} dimer and an anti-NGFR mAb. Note that clone 9 and ΔNGFR genes are fused with P2A.

FIGS. 5A-5P are scatter plots illustrating costaining of Clone 9- (FIGS. 5A-5H) and 5B8- (FIGS. 5I-5P) transduced primary T cells stained with 5 μg/ml conventional wild-type DP4 tetramers and DP4^{L112W/V141M} dimers. At least 2 independent experiments were performed.

FIGS. 6A-6F are bar graphs illustrating the results of comprehensive screening with DP4^{L112W/V141M} dimers, which identified an array of novel DP4-restricted tumor-associated antigens. Peripheral CD4⁺ T cells were purified from six DP4⁺ melanoma patients and stimulated with DP4-expressing aAPCs individually pulsed with 196 distinct peptides derived from tumor-associated antigens and stained with cognate DP4^{L112W/V141M} dimers. The results using the 30 peptides with the highest positivity values are shown in FIGS. 6A-6B. The results for the remaining 166 peptides are shown in FIGS. 6C-6F. Each gating was set so that control dimer staining showed <0.2% positivity. Positive dimer staining was defined as staining exceeding the control dimer staining by 3 standard deviations, as shown by the dashed line (>0.6%).

FIGS. 7A-7L are graphical representations of DP4^{L112W/V141M} dimer staining of peptide-specific CD4⁺ T cells from melanoma patients. Primary CD4⁺ T cells were purified from six DP4⁺ melanoma patients and stimulated with DP4-expressing aAPCs individually pulsed with 196 distinct peptides derived from tumor-associated antigens and stained with cognate DP4^{L112W/V141M} dimers as shown in FIGS. 6A-6F. Examples of DP4^{L112W/V141M} dimer staining are shown. *P<0.05 by Student's t-test. n.s., not significant. At least 2 independent experiments were performed.

FIGS. 8A-8X are graphical representations of data illustrating that DP4-restricted TCRs isolated from DP4^{L112W/V141M} dimer-positive cells and reconstituted in human TCR-defective CD4⁺ T cells were functional in a DP4-restricted and antigen-specific manner. 03-CCND1₂₁₉₋₂₃₈ (FIGS. 8A-8D), 05-HSD17B12₂₂₅₋₂₄₄ and 09-HSD17B12₂₂₅₋₂₄₄ (FIGS. 8E-8J), 05-LGSN₂₉₆₋₃₁₅ (FIGS. 8K-8N), 03-MAGE-A2₁₀₈₋₁₂₇ and 06-MAGE-A2₁₀₈₋₁₂₇ (FIGS. 80-8T), and 05-MUC5AC₄₉₂₂₋₄₉₄₁ (FIGS. 8U-8X) were cloned from DP4^{L112W/V141M} dimer-positive cells, reconstituted in TCR-defective Jurkat 76/CD4 cells, and stained by the respective DP4^{L112W/V141M} dimers.

FIGS. 9A-9G are bar graphs illustrating the results of IL-2 EPISPOT assays of 03-CCND1₂₁₉₋₂₃₈ (FIG. 9A), 05-HSD17B12₂₂₅₋₂₄₄ (FIG. 9B), 09-HSD17B12₂₂₅₋₂₄₄ (FIG. 9C), 05-LGSN₂₉₆₋₃₁₅ (FIG. 9D), 03-MAGE-A2₁₀₈₋₁₂₇ (FIG. 9E), 06-MAGE-A2₁₀₈₋₁₂₇ (FIG. 9F), and 05-MUC5AC₄₉₂₂₋₄₉₄₁ (FIG. 9G) were stimulated by aAPCs pulsed with the respective peptides in IL-2 ELISPOT assays. DP4/WT1 (clone 9) TCR was used as a negative control. At least 2 independent experiments were performed. *, P<0.05 by Student's t-test. Bars and error bars represent the mean±SD of results in triplicate experiments.

FIGS. 10A-10Q are graphical representations of data showing that DP4-restricted TCRs isolated from DP4^{L112W/V141M} dimer-positive cells and reconstituted in human primary CD4⁺ T cells were functional in a DP4-restricted and antigen-specific manner. 03-CCND1₂₁₉₋₂₃₈ (FIGS. 10A-10D and 10O), 03-MAGE-A2₁₀₈₋₁₂₇ and 06-MAGE-A2₁₀₈₋₁₂₇ (FIGS. 10E-10J and 10P) and 05-MUC5AC₄₉₂₂₋₄₉₄₁ (FIGS. 10K-10N and 10Q) were retrovirally transduced into human primary CD4⁺ T cells and stained with the respective DP4^{L112W/V141M} dimers (FIGS. 10A-10N). *P<0.05 by Student's t-test. n.s., not significant. At least 2 independent experiments were performed. *, P<0.05 by Student's t-test. Bars and error bars represent the mean±SD of results in triplicate experiments.

FIGS. 11A-11E present data showing that DP4-restricted TCRs cloned from melanoma patients recognized peptides endogenously processed and presented by K562-based aAPCs. FIGS. 11A-11B are images of gel chromatography showing CCDN1 (FIG. 11A) and MAGE-A2 (FIG. 11B) endogenously expressed in K562-derived aAPC cells. FIGS. 11C-11D are bar graphs showing the results of IFN-7 ELISPOT assays of human primary T cells retrovirally transduced with 03-CCND1₂₁₉₋₂₃₈ (FIG. 11C) or 06-MAGE-A2₁₀₈₋₁₂₇ (FIG. 11D) and stimulated with peptide-unpulsed HLA-null or DP4-aAPCs (FIGS. 11C-11D). FIG. 11E is a bar graph showing the results of an IFN-7 ELISPOT assay of human primary T cells retrovirally transduced with 05-MUC5AC₄₉₂₂₋₄₉₄₁ TCR and stimulated with MUC5AC₄₉₁₄₋₄₉₄₉ minigene-transduced and peptide-unpulsed HLA-null or DP4-aAPCs. At least 2 independent experiments were performed. *, P<0.05 by Student's t-test. Bars and error bars represent the mean±SD of results in triplicate experiments.

FIGS. 12A-12E present data showing that 06-MAGE-A2₁₀₈₋₁₂₇ TCR recognizes melanoma cell lines in a DP4- and MAGE-A2-dependent manner. FIG. 12A is an image of western blot showing endogenous MAGE-A2 expression in K562 cells and the indicated melanoma cell lines. FIGS. 12B-12E are bar graphs showing data from IFN-7 ELISPOT assays of primary human T cells transduced with 06-MAGE-A2₁₀₈₋₁₂₇ TCR stimulated with SK-MEL-21 (DP4⁺ MAGE-A2-; FIG. 12B) or SK-MEL-37 (DP4⁺ MAGE-A2+; FIG. 12C) and SK-MEL-28 (DP4- MAGE-A2⁺; FIG. 12D) and Me275 (DP4- MAGE-A2⁺; FIG. 12E) transduced with DP4. *, P<0.05 by Student's t-test. Bars and error bars represent the mean±SD of results in triplicate experiments. At least 2 independent experiments were performed.

FIGS. 13A-13Q are histograms comparing expression levels of wild-type HLADP*04:01 and derivatives thereof in K562 cells stained with the anti-HLA class II mAb clone 9-49. Open histograms represent the isotype control staining.

FIGS. 14A-14F provide data illustrating the enhanced CD4 binding ability of modified DQ molecules. FIG. 14A is a table comparing the amino acid sequences of DPB1*04:01, DQB1*05:01, and DQB1*05:01^{L114W/V143M+4reps}, with mutated amino acids underlined. FIGS. 14B and 14C are graphical representations of data of class II-deficient K562 cells stably expressing wild-type DQ5 (DQA1*01:01/DQB1*05:01), DQ5^{L114W/V143M}, DQ5^{L114W/V143M+4reps}, wild-type DP4, or DP4^{L112W/V141M} stained with sCD4, as shown in FIG. 14A. FIG. 14D shows the CD4 binding ability of a series of K562 derivatives individually expressing DQ5^{L114W/V143M+4reps} mutants with a single amino acid reversal at one of the four positions, similarly stained with sCD4. FIG. 14E is a table listing the amino acid sequences of DPB1*04:01, DQB1*02:01, DQB1*04:02 and DQB1*06:01 with replaced amino acids underlined. Note that unlike DQB1*05:01, DQB1*02:01, DQB1*04:02 and DQB1*06:01 encode Val at position 116, similar to DPB1*04:01, which codes for Val at position 114. FIG. 14F provides graphical representations of data showing that the L114W/V143M+3reps replacements in the R chains enhanced the binding of DQ2, DQ4, and DQ6 to CD4. At least 2 independent experiments were performed. *, P<0.05 by Student's t-test. Bars and error bars represent the mean±SD of results in triplicate experiments.

FIGS. 15A-15B are graphical representations illustrating that affinity-matured DQ dimers detected cognate TCRs expressed in human primary CD4⁺ T cells. DQ5 (DQA1*01:01-DQB1*05:01)-restricted DDX3Y-specific TCR (E6) (FIG. 15A) and DQ6 (DQA1*01:02-DQB1*06:02)-restricted influenza virus HA-specific TCR (DM2) (FIG. 15B) were reconstituted in human primary CD4⁺ T cells and stained by DQ5^{L114W/V143M+4reps} and DQ6^{L114W/V143M+3reps} dimers, respectively. At least 2 independent experiments were performed.

FIGS. 16A-16Q are graphical representations of histograms illustrating the comparable expression levels of HLA class II genes. HLA-DQ and their derivatives were reconstituted in K562 cells and stained with anti-HLA class II monoclonal antibodies. The surface expression of each DQ2, DQ5, and DQ6 allele was detected using the anti-HLA class II monoclonal antibody clone 9-49(I3) (DQ5 and DQ6) or the anti-class II monoclonal antibody clone T639 (DQ2 and DQ4). Open histograms represent the isotype control staining.

FIGS. 17A-17F provide data illustrating the enhanced CD4 binding ability of modified DR molecules. FIG. 17A is a table comparing the amino acid sequences of DPB1*04:01, DRB1*01:01, and DRB1*01:01^{L114W/V143M+6reps}, with mutated amino acids underlined. FIGS. 17B and 17C are graphical representations of data of class II-deficient K562 cells stably transduced with wild-type DR1 (DRA1*01:01/DRB1*01:01), DR1^{L114W/V143M}, DR1^{L114W/V143M+6reps}, wild-type DP4, or DP4^{L112W/V141M} and stained with sCD4. FIGS. 17D-17E show the CD4 binding ability of a series of K562 derivatives individually expressing DR1^{L114W/V143M+6reps} mutants with a single amino acid reversal at one of the six positions (FIG. 17D), similarly stained with sCD4 and DRB1^{L114W/V143M+2reps}, which carries S118H and T157I along with L114W/V143M (FIG. 17E). FIG. 17F is a table listing the amino acid sequences of DPB1*04:01 and DRB1 alleles of DR3, DR4, DR7, DR10, DR11, and DR13 were compared along with those of DRB1^{L114W/V143M+6reps} and DRB1^{L114W/V143M+2reps}, with mutated amino acids underlined. FIGS. 17G-17L are graphical representations of data showing that the L114W/V143M+2reps mutations enhanced the binding of DR3, DR4, DR7, DR10, DR11, and DR13 to CD4 better than the L114W/V143M+6reps mutations. At least 2 independent experiments were performed. *, P<0.05 by Student's t-test. Bars and error bars represent the mean±SD of results in triplicate experiments. FIGS. 17M-17N are biolayer interferometry sensorgrams showing the interaction of biotinylated HLA-DR1 (ligand) with soluble CD4 (analyte) over a range of concentrations. Binding experiments for wild-type DR1 (FIG. 17M) and DR1^{L114W/V143M+2reps} (FIG. 17N) were performed in parallel, and binding was not detected for wild-type DR1 (FIG. 17M). FIG. 17O is a graph showing the affinity between DR1^{L114W/V143M+2reps} and CD4 as quantified by steady-state analysis. All data are representative of two independent experiments.

FIGS. 18A-18D are graphical representations illustrating that affinity-matured DR dimers detected cognate TCRs are expressed in human primary CD4⁺ T cells. DR1-restricted TCRs (HA1.7 and SB95) (FIG. 18A), DR7-restricted TCR (SD334) (FIG. 18B) and DR11-restricted TCR (F24) (FIG. 18C) were reconstituted in primary human T cells and stained by respective DR^{L114W/V143M+2reps} dimers. DR11-restricted F24-transduced CD4⁺ T cells were stained with the DR11^{L114W/V143M+2reps} dimer and an anti-Vβ 22 mAb (FIG. 18D). Note that F24 expresses Vβ22. At least 2 independent experiments were performed.

FIGS. 19A-19D are drawings of model structures of HLA-DR1^{L114W/V143M+2reps} and the human CD4 complex. FIG. 19A provides an overview ribbon model of the ternary complex model structure of DRA1*01:01, DRB1*01:01, and CD4, as indicated. FIGS. 19B-19D provide close-up views of four mutated residues: L114W and V143M (FIG. 19B), S118H (FIG. 19C) and T157I (FIG. 19D) in wild-type DR1 (left) and mutated DR1^{L114W/V143M+2reps} (right), as illustrated using ball-and-stick representation.

FIGS. 20A-20II are graphical representations of histograms illustrating the comparable expression levels of HLA class II genes. HLA-DR and their derivatives were reconstituted in K562 cells and stained with anti-HLA class II monoclonal antibodies. The surface expression of all DR alleles was detected using the anti-HLA class II monoclonal antibody clone 9-49(I3). Open histograms represent the isotype control staining.

FIGS. 21A-21D are graphical representations of data showing comparison of DP4^{L112W/V141M} dimers and dextramers for the staining of endogenous TRPC1₅₇₈₋₅₉₇-specific CD4⁺ T cells. Endogenous (non-transduced) TRPC1₅₇₈₋₅₉₇-specific CD4⁺ T cells were expanded from a melanoma patient by stimulation with peptide-pulsed and irradiated DP4⁺ artificial APCs and stained with DP4^{L112W/V141M} TRPC1₅₇₈₋₅₉₇ dimers (FIG. 21B) or a TRPC1₅₇₈₋₅₉₇ dextramer (FIG. 21D). The corresponding CLIP multimers were used as controls (FIGS. 21A and 21C).

FIGS. 22A-22F are graphical representations of data showing comparison of DP4^{L112W/V141M} dimers and conventional DP4 tetramers and dextramers for the staining of endogenous NY-ESO-1₁₅₇₋₁₇₀-specific T cells. CD4⁺ T cells were purified from DP4⁺ healthy donor No. 4 and stimulated once with NY-ESO-1₁₅₇₋₁₇₀-pulsed and irradiated DP4⁺ artificial APCs. Expanded CD4⁺ T cells were individually stained as indicated by three different DP4 multimers (DP4^{L112W/V141M} dimers (FIG. 22B), DP4 tetramers (FIG. 22D), or DP4 dextramers (FIG. 22F)).

FIGS. 23A-23Y are graphical representations of data showing pathogen-specific CD4⁺ T cells subjected to ex vivo staining with DP4^{L112W/V141M} dimers. Memory CD4⁺ T cells were purified from five DP4⁺ donors and subjected to ex vivo staining with the DP4^{L112W/V141M} dimers for the following pathogen-associated peptides without in vitro stimulation: TT₉₄₈₋₉₆₈ (FIGS. 23F-23J), HSV-2-UL21₂₈₃₋₃₀₂ (FIGS. 23K-230), Flu-HA527-546 (FIGS. 23P-23T), and RSV-GP₁₆₂₋₁₇₅ (FIGS. 23U-23Y). The CLIP peptide was used as a negative control (FIGS. 23A-23E).

FIGS. 24A-24W are graphical representations of data showing endogenous RSV-GP₁₆₂₋₁₇₅-specific CD4⁺ T cell clones successfully established from DP4^{L112W/V141M} dimer⁺ cells. Memory CD4⁺ T cells were purified from DP4⁺ Donor No. 06 and subjected to ex vivo staining with DP4^{L112W/V141M} RSV-GP₁₆₂₋₁₇₅ dimers without in vitro stimulation. Dimer⁺ CD4⁺ T cells were then cloned by limiting dilution. FIGS. 24A-24V are graphical representations of representative dimer staining data of 10 dimer-positive and 1 dimer-negative single-cell clones. Seventy-seven out of 84 clones (91.7%) were successfully stained with DP4^{L112W/V141M} RSV-GP₁₆₂₋₁₇₅ dimers. FIG. 24W is a bar graph showing antigen-specific IL-2 production in RSV-GP₁₆₂₋₁₇₅ dimer⁺ single-cell clones.

FIGS. 25A-25S are graphical representations of data showing endogenous DP4 TT₉₄₈₋₉₆₈-specific CD4⁺ T cell clones successfully established from DP4^{L112W/V141M} dimer⁺ cells. Memory CD4⁺ T cells were purified from DP4⁺ Donor No. 04 and subjected to ex vivo staining with DP4^{L112W/V141M} TT₉₄₈₋₉₆₈ dimers without in vitro stimulation. Dimer⁺ CD4⁺ T cells were then cloned by limiting dilution. FIGS. 25A-25R are graphical representations of representative dimer staining data of 8 dimer-positive and 1 dimer-negative single-cell clones. Twenty-six out of 29 clones (89.7%) were successfully stained with DP4^{L112W/V141M} TT₉₄₈₋₉₆₈ dimers. FIG. 25S is a bar graph showing antigen-specific IL-2 production in TT₉₄₈₋₉₆₈ dimer⁺ single-cell clones.

FIGS. 26A-26NN are graphical representations of DP4 multimer staining of RSV-GP (FIGS. 26A-26P) and TT (FIGS. 26O-26NN) dimer⁺ single-cell clones. RSV-GP dimer⁺ single-cell clones (c6, c12, c26, and c39) were stained with either DP4^{L112W/V141M} RSV-GP₁₆₂₋₁₇₅ dimers (FIGS. 26B, 26D, 26F, and 26H) or wild-type DP4 dextramers (FIGS. 26J, 26L, 26N, and 26P). TT dimer⁺ single-cell clones (c2, c4, c6, and c9) were individually stained with three different DP4 TT₉₄₈₋₉₆₈ multimers (DP4^{L112W/V141M} dimers (FIGS. 26R, 26T, 26V, and 26X), wild-type DP4 tetramers (FIGS. 26Z, 26BB, 26DD, and 26FF), and wild-type DP4 dextramers (FIGS. 26HH, 26JJ, 26LL, and 26NN).

FIGS. 27A-27L are graphical representations showing that DQ5^{L114W/V143M+4reps} dimers robustly stained E6-transduced CD4⁺ T cells. E6 was reconstituted in CD4⁺ T cells, which were then stained with wild-type DQ5 (FIGS. 27D and 27J), DQ5^{L114W/V143M} (FIGS. 27E and 27K), and DQ5^{L114W/V143M+4reps} (FIGS. 27F and 27L) CLIP control dimers (FIGS. 4D-4F) and dimers specific to DDX3Y₁₇₁₋₁₉₀ (FIGS. 27J-27L). Control cells not transduced with a TCR are shown in FIGS. 27A-27C and 27G-27I.

FIGS. 28A-28H are graphical representations showing cloning of DQ5-restricted TCR using affinity matured dimer. Primary CD4⁺ T cells were purified from a DQ5.1⁺ melanoma patient and stimulated with irradiated GPC3₁₃₈₋₁₅₇-pulsed aAPCs expressing DQ5.1. Two weeks later, stimulated CD4⁺ T cells were stained with cognate GPC3₁₃₈₋₁₅₇-DQ5^{L114W/V143M+4reps} dimers (FIGS. 28A-28B). The GPC3 specific TCR was reconstituted in TCR-defective Jurkat 76/CD4 cells, and stained by the respective DQ5^{L114W/V143M+4reps} dimers (FIG. 28C (E6/Control); FIG. 28D (E6/GPC3₁₃₈₋₁₅₇); FIG. 28E (DQ5-06-GPC3₁₃₈₋₁₅₇/Control); and FIG. 28F (DQ5-06-GPC3₁₃₈₋₁₅₇/GPC3₁₃₈₋₁₅₇)). Jurkat 76/CD4 cells expressing the GPC3 specific TCR were stimulated by DQ5-K562 cells pulsed with the respective peptides in IL-2 ELISPOT assays (FIG. 28G).

FIGS. 29A-29L are graphical representations showing influenza virus hemagglutinin-specific peripheral CD4⁺ T cells subjected to ex vivo staining with DR1^{L114W/V143M+2reps} dimers. Memory CD4⁺ T cells were purified from two DR1⁺ donors (No. 07 (FIGS. 29A-29F) and No. 08 (FIGS. 29G-29L)) and stained with DR1^{L114W/V143M+2reps} dimers specific to Flu-HA₅₋₂₄ (FIGS. 29B and 29H), Flu-HA₁₁₇₋₁₃₆ (FIGS. 29C and 29I), Flu-HA₂₃₂₋₂₅₁ (FIGS. 29D and 29J), Flu-HA₂₆₈₋₂₈₇ (FIGS. 29E and 29K), and Flu-HA₃₀₆₋₃₁₈ (FIGS. 29F and 29L) influenza virus hemagglutinin (Flu-HA) peptides without in vitro stimulation. The CLIP peptide was used as a negative control (FIGS. 29A and 29G).

FIGS. 30A-30X are graphical representations showing DR1^{L114W/V143M+6reps} and DR1^{L114W/V143M+2reps} dimers robustly stained HA1.7-transduced CD4⁺ T cells. HA1.7 was reconstituted in primary CD4⁺ T cells, which were then stained with wild-type DR1 (FIGS. 30I and 30M), DR1^{L114W/V143M} (FIGS. 30J and 30N), DR1^{L114W/V143M+6reps} (FIGS. 30K and 30O), and DR1^{L114W/V143M+2reps} (FIGS. 30L and 30P) dimers without a transduced TCR (FIGS. 30I-30L) and transduced with an HA1.7 TCR (FIGS. 30M-30P), with CLIP dimers used as negative controls (FIGS. 30A-30H). In addition, HA1.7 was reconstituted in primary CD4⁺ T cells, which were then stained with DR1^{L114W/V143M+2reps} dimer (FIGS. 30O-30T) or a wild-type DR1 dextramer (FIGS. 30U-30X) specific to Flu-HA₃₀₆₋₃₁₈.

FIGS. 31A-31P are graphical representations showing data for cloning of DR1-restricted TCRs using affinity matured dimer. Primary CD4⁺ T cells were purified from two DR1⁺ melanoma patients and stimulated with irradiated HSD17B12₂₂₅₋₂₄₄-pulsed (FIG. 31B) and LY6K₉₉₋₁₁₈-pulsed (FIG. 31D) aAPCs expressing DR1. Two weeks later, stimulated CD4⁺ T cells were stained with cognate DR1^{L114W/V143M+2reps} dimers (FIGS. 31A-31D). The DR1-restricted TCRs were reconstituted in primary CD4⁺ T cells, and stained by the respective dimer (FIGS. 31E-31M). Primary CD4⁺ T cells expressing the DR1-restricted DR1-07-HSD17B12₂₂₅₋₂₄₄ (FIG. 31N) and DR1-08-LY6K₉₉₋₁₁₈ (FIG. 31O) TCRs were stimulated by DR1-K562 cells pulsed with HSD17B12₂₂₅₋₂₄₄ (FIG. 31N) and LY6K₉₉₋₁₁₈ (FIG. 31O) peptides, respectively, in IL-2 ELISPOT assays.

### DETAILED DESCRIPTION OF THE DISCLOSURE

The present disclosure is directed to methods of identifying MHC class II-specific TCRs comprising contacting a T cell with a complex comprising an MHC class II molecule and a peptide, wherein the MHC class II molecule has a higher affinity for CD4 than a naturally occurring MHC class II molecule has for CD4. In some aspects, the MHC class II molecule comprises an alpha chain and a beta chain, wherein the beta chain of the MHC class II molecule comprises an amino acid sequence having one or more mutations relative to a wild-type beta chain of a MHC class II molecule.

### L. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs; full-length TCRs and antigen binding portions thereof, chimeric TCRs; TCR fusion constructs; and synthetic TCRs. In human, TCRs are expressed on the surface of T cells, and they are responsible for T cell recognition and targeting of antigen presenting cells. Antigen presenting cells (APCs) display fragments of foreign proteins (antigens) complexed with the major histocompatibility complex (MHC; also referred to herein as complexed with an HLA molecule, e.g., an HLA class II molecule). A TCR recognizes and binds to the peptide:HLA complex and recruits CD8 (for MHC Class I molecules) or CD4 (for MHC class II molecules), activating the TCR. The activated TCR initiates downstream signaling and an immune response, including the destruction of the EPC.

In general, a TCR can comprise two chains, an alpha chain and a beta chain (or less commonly a gamma chain and a delta chain), interconnected by disulfide bonds. Each chain comprises a variable domain (alpha chain variable domain and beta chain variable domain) and a constant region (alpha chain constant region and beta chain constant region). The variable domain is located distal to the cell membrane, and the variable domain interacts with an antigen. The constant region is located proximal to the cell membrane. A TCR can further comprises a transmembrane region and a short cytoplasmic tail. As used herein, the term “constant region” encompasses the transmembrane region and the cytoplasmic tail, when present, as well as the traditional “constant region.”

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region, while CDR1 and CDR2 are believed to primarily recognize the HLA molecule.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD4, as described herein), or a TCR described herein that is free from a cell membrane (e.g., an isolated TCR or a soluble TCR).

An “antigen binding molecule,” “portion of a TCR,” or “TCR fragment” refers to any portion of an TCR less than the whole. An antigen binding molecule can include the antigenic CDRs.

An “antigen” refers to any molecule, e.g., a peptide, that provokes an immune response or is capable of being bound by a TCR. An “epitope,” as used herein, refers to a portion of a polypeptide that provokes an immune response or is capable of being bound by a TCR. The immune response may involve either antibody production, or the activation of specific immunologically-competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or an epitope can be specific to a certain tissue, such as a diseased cell, e.g., a cancer cell, or it can be broadly expressed. In addition, fragments of larger molecules can act as antigens. In one aspect, antigens are tumor antigens. An epitope can be present in a longer polypeptide (e.g., in a protein), or an epitope can be present as a fragment of a longer polypeptide. In some aspects, an epitope is complexed with a major histocompatibility complex (MHC; also referred to herein as complexed with an HLA molecule, e.g., an HLA class 1 molecule).

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

“CCND1,” “G1/S-specific cyclin-D1,” “B-cell lymphoma 1 protein,” “BCL-1,” or “PRAD1,” as used herein, refers to a human regulatory component of the cyclin D1-CDK4 (DC) complex that phosphorylates and inhibits members of the retinoblastoma (RB) protein family including RB1 and regulates the cell-cycle during G1/S transition. Phosphorylation of RB1 allows dissociation of the transcription factor E2F from the RB/E2F complex and the subsequent transcription of E2F target genes which are responsible for the progression through the G1 phase. CCND1 is also involved in hypophosphorylation of RB1 in early G1 phase. Cyclin D-CDK4 complexes are major integrators of various mitogenenic and antimitogenic signals. CCND1 is also a substrate for SMAD3, phosphorylating SMAD3 in a cell-cycle-dependent manner and repressing its transcriptional activity. CCND1 is also a component of the ternary complex, cyclin D1/CDK4/CDKN1B, required for nuclear translocation and activity of the cyclin D-CDK4 complex, and CCND1 exhibits transcriptional corepressor activity with INSM1 on the NEUROD1 and INS promoters in a cell cycle-independent manner. Mutations, amplification, and overexpression of CCND1, which alter cell cycle progression, are observed frequently in a variety of tumors and may contribute to tumorigenesis.

As used herein, CCND1 refers to not only the full-length canonical sequence, but also variants and fragments thereof. The amino acid sequence of CCND1 (SEQ ID NO: 27) is provided in Table 1A (UniProtKB-P24385).

TABLE 1A
CCND1 Amino Acid Sequence

CCND1 Amino Acid Sequence
MEHQLLCCEVETIRRAYPDANLLNDRVLRAMLKAEETCAPSVSYFKCVQK
EVLPSMRKIVATWMLEVCEEQKCEEEVFPLAMNYLDRFLSLEPVKKSRLQ
LLGATCMFVASKMKETIPLTAEKLCIYTDNSIRPEELLQMELLLVNKLKW
NLAAMTPHDFIEHFLSKMPEAEENKQIIRKHAQTFVALCATDVKFISNPP
SMVAAGSVVAAVQGLNLRSPNNFLSYYRLTRFLSRVIKCDPDCLRACQEQ
IEALLESSLRQAQQNMDPKAAEEEEEEEEEVDLACTPTDVRDVDI(SEQ
ID NO: 27)

“MUC5AC” or “mucin 5AC,” as used herein, refers to a human gel-forming glycoprotein of gastric and respiratory tract epithelia that protects the mucosa from infection and chemical damage by binding to inhaled microorganisms and particles that are subsequently removed by the mucocilary system.

As used herein, MUC5AC refers to not only the full-length canonical sequence, but also variants and fragments thereof. The amino acid sequence of MUC5AC (SEQ TD NO: 28) is provided in Table 1B (UniProtKB-P98088).

TABLE 1B
MUC5AC Amino Acid Sequence

MUC5AC Amino Acid Sequence
MSVGRRKLALLWALALALACTRHTGHAQDGSSESSYKHHPALSPIARGPSGVPLRGATVFPSLRTIPVVRASNPAHN
GRVCSTWGSFHYKTFDGDVFRFPGLCNYVFSEHCGAAYEDFNIQLRRSQESAAPTLSRVLMKVDGVVIQLTKGSVLV
NGHPVLLPFSQSGVLIQQSSSYTKVEARLGLVLMWNHDDSLLLELDTKYANKTCGLCGDFKGMPVVSELLSHNTKLT
PMEFGNLQKMDDPTDQCQDPVPEPPRNCSTGFGICEELLHGQLFSGCVALVDVGSYLEACRQDLCFCEDTDLLSCVC
HTLAEYSRQCTHAGGLPQDWRGPDFCPQKCPNNMQYHECRSPCADTCSNQEHSRACEDHCVAGCFCPEGTVLDDIGQ
TGCVPVSKCACVYNGAAYAPGATYSTDCTNCTCSGGRWSCQEVPCPGTCSVLGGAHFSTFDGKQYTVHGDCSYVLTK
PCDSSAFTVLAELRRCGLTDSETCLKSVTLSLDGAQTVVVIKASGEVFLNQIYTQLPISAANVTIFRPSTFFIIAQT
SLGLQLNLQLVPTMQLFMQLAPKLRGQTCGLCGNFNSIQADDFRTLSGVVEATAAAFFNTFKTQAACPNIRNSFEDP
CSLSVENEKYAQHWCSQLTDADGPFGRCHAAVKPGTYYSNCMFDTCNCERSEDCLCAALSSYVHACAAKGVQLGGWR
DGVCTKPMTTCPKSMTYHYHVSTCQPTCRSLSEGDITCSVGFIPVDGCICPKGTFLDDTGKCVQASNCPCYHRGSMI
PNGESVHDSGAICTCTHGKLSCIGGQAPAPVCAAPMVFFDCRNATPGDTGAGCQKSCHTLDMTCYSPQCVPGCVCPD
GLVADGEGGCITAEDCPCVHNEASYRAGQTIRVGCNTCTCDSRMWRCTDDPCLATCAVYGDGHYLTFDGQSYSFNGD
CEYTLVQNHCGGKDSTQDSFRVVTENVPCGTTGTTCSKAIKIFLGGFELKLSHGKVEVIGTDESQEVPYTIRQMGIY
LVVDTDIGLVLLWDKKTSIFINLSPEFKGRVCGLCGNFDDIAVNDFATRSRSVVGDVLEFGNSWKLSPSCPDALAPK
DPCTANPFRKSWAQKQCSILHGPTFAACHAHVEPARYYEACVNDACACDSGGDCECFCTAVAAYAQACHEVGLCVSW
RTPSICPLFCDYYNPEGQCEWHYQPCGVPCLRTCRNPRGDCLRDVRGLEGCYPKCPPEAPIFDEDKMQCVATCPTPP
LPPRCHVHGKSYRPGAVVPSDKNCQSCLCTERGVECTYKAEACVCTYNGQRFHPGDVIYHTTDGTGGCISARCGANG
TIERRVYPCSPTTPVPPTTFSFSTPPLVVSSTHTPSNGPSSAHTGPPSSAWPTTAGTSPRTRLPTASASLPPVCGEK
CLWSPWMDVSRPGRGTDSGDFDTLENLRAHGYRVCESPRSVECRAEDAPGVPLRALGQRVQCSPDVGLTCRNREQAS
GLCYNYQIRVQCCTPLPCSTSSSPAQTTPPTTSKTTETRASGSSAPSSTPGTVSLSTARTTPAPGTATSVKKTFSTP
SPPPVPATSTSSMSTTAPGTSVVSSKPTPTEPSTSSCLQELCTWTEWIDGSYPAPGINGGDFDTFQNLRDEGYTFCE
SPRSVQCRAESFPNTPLADLGQDVICSHTEGLICLNKNQLPPICYNYEIRIQCCETVNVCRDITRLPKTVATTRPTP
HPTGAQTQTTFTTHMPSASTEQPTATSRGGPTATSVTQGTHTTLVTRNCHPRCTWTKWFDVDFPSPGPHGGDKETYN
NIIRSGEKICRRPEEITRLQCPAKSHPEVSIEHLGQVVQCSREEGLVCRNQDQQGPFKMCLNYEVRVLCCETPRGCH
MTSTPGSTSSSPAQTTPSTTSKTTETQASGSSAPSSTPGTVSLSTARTTPAPGTATSVKKTFSTPSPPPVPATSTSS
MSTTAPGTSVVSSKPTPTEPSTSSCLQELCTWTEWIDGSYPAPGINGGDFDTFQNLRDEGYTFCESPPSVQCRAESF
PNTPLADLGQDVICSHTEGLICLNKNQLPPICYNYEIRIQCCETVNVCRDITRPPKTVATTRPTPHPTGAQTQTTFT
THMPSASTEQPTATSRGGPTATSVTQGTHTTPVTRNCHPRCTWTTWFDVDFPSPGPHGGDKETYNNIIRSGEKICRR
PEEITRLQCRAKSHPEVSIEHLGQVVQCSREEGLVCRNQDQQGPFKMCLNYEVRVLCCETPKGCPVTSTPVTAPSTP
SGRATSPTQSTSSWQKSRTTTLVTTSTTSTPQTSTTYAHTTSTTSAPTARTTSAPTTRTTSASPASTTSGPGNTPSP
VPTTSTISAPTTSITSAPTTSTTSAPTSSTTSGPGTTPSPVPTTSITSAPTTSTTSAPTTSTTSARTSSTTSATTTS
RISGPETTPSPVPTTSTTSATTTSTTSAPTTSTTSAPTSSTTSSPQTSTTSAPTTSTTSGPGTTPSPVPTTSTTSAP
TTRTTSAPKSSTTSAATTSTTSGPETTPRPVPTTSTTSSPTTSTTSAPTTSTTSASTTSTTSGAGTTPSPVPTTSTT
SAPTTSTTSAPISSTTSATTTSTTSGPGTTPSPVPTTSTTSAPTTSTTSGPGTTPSAVPTTSITSAPTTSTNSAPIS
STTSATTTSRTSGPETTPSPVPTASTTSASTTSTTSGPGTTPSPVPTTSTISVPTTSTTSASTTSTTSASTTSTTSG
PGTTPSPVPTTSTTSAPTTSTTSAPTTSTISAPTTSTTSATTTSTTSAPTPPRTSAPTTSTISASTTSTTSATTTST
TSATTTSTISAPTTSTTLSPTTSTTSTTITSTTSAPISSTTSTPQTSTTSAPTTSTTSGPGTTSSPVPTTSTTSAPT
TSTTSAPTTRTTSVPTS3TTSTATTSTTSGPGTTPSPVPTTSTTSAPTTRTTSAPTTSTTSAPTTSTTSAPTSSTTS
ATTTSTISVPTTSTTSVPGTTPSPVPTTSTISVPTTSTTSASTTSTTSGPGTTPSPVPTTSTTSAPTTSTTSAPTTS
TISAPTTSTPSAPTTSTTLAPTTSTTSAPTTSTTSTPTSSTTSSPQTSTTSASTTSITSGPGTTPSPVPTTSTTSAP
TTSTTSAATTSTISAPTTSTTSAPTTSTTSASTASKTSGLGTTPSPIPTTSTTSPPTTSTTSASTASKTSGPGTTPS
PVPTTSTIFAPRTSTTSASTTSTTPGPGTTPSPVPTTSTASVSKTSTSHVSISKTTHSQPVTRDCHLRCTWTKWFDI
DFPSPGPHGGDKETYNNIIRSGEKICRRPEEITRLQCRAESHPEVSIEHLGQVVQCSREEGLVCRNQDQQGPFKMCL
NYEVPVLCCETPKGCPVTSTPVTAPSTPSGRATSPTQSTSSWQKSRTTTLVTTSTTSTPQTSTTSAPTTSTTSAPTT
STTSAPTTSTTSTPQTSISSAPTSSTTSAPTSSTISARTTSIISAPTTSTTSSPTTSTTSATTTSTTSAPTSSTTST
PQTSKTSAATSSTTSGSGTTPSPVTTTSTASVSKTSTSHVSVSKTTHSQPVTRDCHPRCTWTKWFDVDFPSPGPHGG
DKETYNNIIRSGEKICRRPEEITRLQCRAKSHPEVSIEHLGQVVQCSREEGLVCRNQDQQGPFKMCLNYEVRVLCCE
TPKGCPVTSTSVTAPSTPSGRATSPTQSTSSWQKSRTTTLVTSSITSTTQTSTTSAPTTSTTPASIPSTTSAPTTST
TSAPTTSTTSAPTTSTTSTPQTTTSSAPTSSTTSAPTTSTISAPTTSTISAPTTSTTSAPTASTTSAPTSTSSAPTT
NTTSAPTTSTTSAPITSTISAPTTSTTSTPQTSTISSPTTSTTSTPQTSTTSSPTTSTTSAPTTSTTSAPTTSTTST
PQTSISSAPTSSTTSAPTASTISAPTTSTTSFHTTSTTSPPTSSTSSTPQTSKTSAATSSTTSGSGTTPSPVPTTST
ASVSKTSTSHVSVSKTTHSQPVTRDCHPRCTWTKWEDVDFPSPGPHGGDKETYNNIIRSGEKICRRPEEITRLQCRA
ESHPEVSIEHLGQVVQCSREEGLVCRNQDQQGPFKMCLNYEVRVLCCETPKGCPVTSTPVTAPSTPSGRATSPTQST
SSWQKSRTTTLVTTSTTSTPQTSTTSAPTTSTIPASTPSTTSAPTTSTTSAPTTSTTSAPTHRTTSGPTTSTTLAPT
TSTTSAPTTSTNSAPTTSTISASTTSTTSAPTTSTISSPTSSTTSTPQTSKTSAATSSTTSGSGTTPSPVPTTSTTS
ASTTSTTSAPTTSTTSGPGTTPSPVPSTSTTSAATTSTTSAPTTRTTSAPTSSMTSGPGTTPSPVPTTSTTSAPTTS
TTSGPGTTPSPVPTTSTTSAPITSTTSGPGSTPSPVPTTSTTSAPTTSTTSASTASTTSGPGTTPSPVPTTSTTSAP
TTRTTSASTASTTSGPGSTPSPVPTTSTTSAPTTRTTPASTASTTSGPGTTPSPVPTTSTTSASTTSTISLPTTSTT
SAPITSMTSGPGTTPSPVPTTSTTSAPTTSTTSASTASTTSGPGTTPSPVPTTSTTSAPTTSTTSASTASTTSGPGT
SLSPVPTTSTTSAPTTSTTSGPGTTPSPVPTTSTTSAPTTSTTSGPGTTPSPVPTTSTTPVSKTSTSHLSVSKTTHS
QPVTSDCHPLCAWTKWFDVDFPSPGPHGGDKETYNNIIRSGEKICRRPEEITRLQCRAESHPEVNIEHLGQVVQCSR
EEGLVCRNQDQQGPFKMCLNYEVRVLCCETPRGCPVTSVTPYGTSPTNALYPSLSTSMVSASVASTSVASSSVASSS
VAYSTQTCFCNVADRLYPAGSTIYRHRDLAGHCYYALCSQDCQVVRGVDSDCPSTTLPPAPATSPSISTSEPVTELG
CPNAVPPRKKGETWATPNCSEATCEGNNVISLRPRTCPRVEKPTCANGYPAVKVADQDGCCHHYQCQCVCSGWGDPH
YITFDGTYYTFLDNCTYVLVQQIVPVYGHFRVLVDNYFCGAEDGLSCPRSIILEYHQDRVVLTRKPVHGVMTNEIIF
NNKVVSPGFRKNGIVVSRIGVKMYATIPELGVQVMFSGLIFSVEVPFSKFANNTEGQCGTCTNDRKDECRTPRGTVV
ASCSEMSGLWNVSIPDCPACHRPHPTPTTVGPTTVGSTTVGPTTVGSTTVGPTTPPAPCLPSPICQLILSKVFEPCH
TVIPPLLEYEGCVFDRCHMTDLDVVCSSLELYAALCASHDICIDWRGRTGHMCPFTCPADKVIOPCGPSNPSYCYGN
DSASLGALPEAGPITEGCFCPEGMTLFSTSAQVCVPTGCPRCLGPHGEPVKVGHTVGMDCQECTCEAATWTLTCRPK
LCPLPPACPLPGFVPVPAPPQAGQCCPQYSCACNTSRCPAPVGCPEGARAIPTYQEGACCPVQNCSWTVCSINGTLY
QPGAVVSSSLCETCRCELPGGPPSDAFVVSCETQICNTHCPVGFEYQEQSGQCCGTCVQVACVTNTSKSPAHLFYPG
ETWSDAGNHCVTHQCEKHQDGLVVVTTKKACPPLSCSLDEARMSKDGCCRECPPPPPPYQNQSTCAVYHRSLIIQQQ
GCSSSEPVRLAYCRGNCGDSSSMYSLEGNTVEHRCQCCQELRTSLRNVTLHCTDGSSRAFSYTEVEECGCMGRRCPA
PGDTCHSEEAEPEPSQEAESGSWERGVPVSPMH (SEQ ID NO: 28)

“MAGE-A2,” “melanoma-associated antigen 2,” or “cancer/testis antigen 1.2,” as used herein, refers to a human protein primarily expressed by tumor cells. MAGE-A2 reduces p53/TP53 transactivation function through recruitment of HDAC3 to p53/TP53 transcription sites. MAGE-A2 represses p73/TP73 activity. In vitro, MAGE-A2 promotes cell viability in melanoma cell lines. MAGE-A2 is expressed in many tumors of several types, such as melanoma, head and neck squamous cell carcinoma, lung carcinoma, and breast carcinoma. However, in healthy tissue, MAGE-A2 is only expressed in the testes.

As used herein, MAGE-A2 refers to not only the full-length sequence, but also variants and fragments thereof. The amino acid sequence of MAGE-A2 (SEQ TD NO: 29) is provided in Table 1C (UniProtKB-P43356).

TABLE 1C
MAGE-A2 Amino Acid Sequence

MAGE-A2 Amino Acid Sequence
MPLEQRSQHCKPEEGLEARGEALGLVGAQAPATEEQQTASSSSTLVEVTL
GEVPAADSPSPPHSPQGASSFSTTINYTLWRQSDEGSSNQEEEGPRMFPD
LESEFQAAISRKMVELVHFLLLKYRAREPVTKAEMLESVLRNCQDFFPVI
FSKASEYLQLVFGIEVVEVVPISHLYILVTCLGLSYDGLLGDNQVMPKTG
LLIIVLAIIAIEGDCAPEEKIWEELSMLEVFEGREDSVFAHPRKLLMQDL
VQENYLEYRQVPGSDPACYEFLWGPRALIETSYVKVLHHTLKIGGEPHIS
YPPLHERALREGEE (SEQ ID NO: 29)

The term “HLA,” as used herein, refers to the human leukocyte antigen. HLA genes encode the major histocompatibility complex (MHC) proteins in humans. MHC proteins are expressed on the surface of cells, and are involved in activation of the immune response. HLA class II genes encode MHC class II proteins which are expressed on the surface of professional antigen presenting cells (APCs). Non-limiting examples of professional APCs include monocytes, macrophages, dendritic cells (DCs), and B lymphocytes. Some endothelial and epithelial cells can also express MHC class II molecules after inflammatory signals are activated. Humans lacking functional MHC class II molecules are extremely susceptible to an array of infectious diseases and typically die at a young age.

As used herein, an “HLA class II molecule” or “MHC class II molecule” refers to a protein product of a wild-type or variant HLA class II gene encoding an MHC class II molecule. Accordingly, “HLA class II molecule” and “MHC class II molecule” are used interchangeably herein. A typical MHC Class II molecule comprises two protein chains: an alpha chain and a beta chain. In general, naturally occurring alpha chains and beta chains each comprise a transmembrane domain, which anchors the alpha/beta chain to the cell surface, and an extracellular domain, which carries the antigen and interacts with a TCR and/or CD4 expressed on a T cell.

Both the MHC Class II alpha and beta chains are encoded by the HLA gene complex. The HLA complex is located within the 6p21.3 region on the short arm of human chromosome 6 and contains more than 220 genes of diverse function. The HLA gene complex is highly variant, with over 20,000 HLA alleles and related alleles, including over 250 MHC class II alpha chain alleles and 5,000 MHC class II beta chain alleles, known in the art, encoding thousands of MHC class II proteins (see, e.g., hla.alleles.org, last visited May 20, 2019, which is incorporated by reference herein in its entirety). For example one such HLA-DP allele, DP4 is the most frequently found allele in many ethnic groups.

Three loci in the HLA complex encode MHC Class II proteins: HLA-DP, HLA-DQ, and HLA-DR. HLA-DO and HLA-DM encode proteins that associate with the MHC class II molecule and support its configuration and function.

When the MHC class II molecule is complexed with an antigen peptide, the 10-30 amino acid long antigen peptide binds the peptide-binding groove and is presented extracellularly to CD4⁺ cells. Both the alpha- and beta-chains fold into two separate domains; alpha-1 and alpha-2 for the alpha polypeptide, and beta-1 and beta-2 for the beta polypeptide. The open-ended peptide-binding groove which holds the presented antigen is found between the alpha-1 and beta-1 domains. Upon interaction with a CD4⁺ T cell, the MHC class II complex interacts with a T cell receptor (TCR) expressed on the surface of the T cell. In addition, the beta chain of the MHC class II molecule weakly interacts (K_{D}>2 mM) with CD4 expressed on the surface of the T cell. The canonical CD4 amino acid sequence (UniProt-P01730) is provided in Table 2 (SEQ ID NO: 10).

TABLE 2
Human CD4 Amino Acid Sequence

MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI (SEQ ID NO: 10)

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some aspects, the methods of the present invention can be used to reduce the tumor size of a tumor derived from, for example, bone cancer, renal cancer, prostate cancer, breast cancer, colon cancer, lung cancer, cutaneous or intraocular malignant melanoma, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. The particular cancer can be responsive to chemo- or radiation therapy or the cancer can be refractory.

A refractory cancer refers to a cancer that is not amendable to surgical intervention, and the cancer is either initially unresponsive to chemo- or radiation therapy or the cancer becomes unresponsive over time.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” or “progressive disease,” which can be abbreviated as PD, as used herein, refers to a worsening of one or more symptom associated with a particular disease. For example, disease progression for a subject afflicted with a cancer can include an increase in the number or size of one or more malignant lesions, tumor metastasis, and death.

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

Other examples of analytes and cytokines of the present invention include, but are not limited to chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM), CD154, lymphotoxin (LT) beta, 4-1BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “infection,” as used herein refers to any type of invasion of one or more tissue of the body by a foreign agent. The term “infection” includes without limitation infection by a virus (including viroids and prions), a bacterium, a fungus, a parasite, and any combination thereof.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). T-cell receptors (TCR) differentiate T cells from other lymphocyte types. The thymus, a specialized organ of the immune system, is primarily responsible for the T cell's maturation. There are six types of T-cells, namely: Helper T-cells (e.g., CD4⁺ cells), Cytotoxic T-cells (also known as TC, cytotoxic T lymphocyte, CTL, T-killer cell, cytolytic T cell, CD8⁺ T-cells or killer T cell), Memory T-cells ((i) stem memory T_{SCM} cells, like naive cells, are CD45RO−, CCR7⁺, CD45RA+, CD62L+(L-selectin), CD27⁺, CD28⁺ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM}cells, however, do not express L-selectin or CCR7 but produce effector cytokines like IFNγ and IL-4), Regulatory T-cells (Tregs, suppressor T cells, or CD4⁺CD25⁺ regulatory T cells), Natural Killer T-cells (NKT) and Gamma Delta T-cells. B-cells, on the other hand, play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

The terms “modified” and “mutated,” when used herein to refer to a nucleotide or amino acid sequence, refers to a change in the sequence relative to a wild-type sequence or a specified reference sequence. The terms “modified” and “mutated” do not require a step in a process for making the modified or mutated sequence (e.g., the modified beta chain sequence), unless otherwise specified. Rather, these terms indicate that there is a variation in the modified or mutated sequence relative to a reference sequence, e.g., a wild-type sequence. For example, a DP beta chain comprising a substitution mutation at a position corresponding to amino acid residue 112 of SEQ ID NO: 1 does not require that a wild-type DP beta chain has been physically altered to arrive at the recited DP beta chain; but rather that, when properly aligned, the recited DP beta chain comprises an amino acid residue at the recited position (residue 112) that is different from the amino acid residue at the corresponding position in a wild-type or reference DP beta chain.

The term “any amino acid,” as used herein, means any known amino acid. Amino acids are organic compounds comprising (i) an amine (—NH₂) functional group, (ii) a carboxyl (—COOH)_functional group, and (iii) a side chain (R group), wherein the side chain is specific to each amino acid. This includes but is not limited to any naturally occurring amino acid, as well as any modifications and variants thereof. There are about 500 naturally occurring amino acids, 20 of which are encoded by the genetic code. Amino acids with positively charged side chains include arginine (Arg; R), histidine (His, H), and lysine (Lys; K). Amino acids with a negatively charged side chain include aspartic acid (Asp; D) and glutamic acid (Glu; E). Amino acids with a polar uncharged side chain include serine (Ser; S), threonine (Thr; T), glutamine (Gln; Q), and asparagine (Asn; N). Amino acids with a hydrophobic side chain include alanine (Ala; A), isoleucine (Ile; I), leucine (Leu; L), methionine (Met; M), phenylalanine (Phe; F), valine (Val; V), Tryptophan (Trp; W), Tyrosine (Tyr; Y). Tryptophan (Trp; W), tyrosine (Tyr; Y), and methionine (Met; M) can also be classified as polar and/or amphipathic, in that these amino acids can often be found at the surface of proteins or lipid membranes. Additional amino acids include cysteine (Cys; C), selenocysteine (Sec; U), glycine (Gly; G) and proline (Pro; P).

As used herein “at a position corresponding to” is used as a means to identify a particular amino acid residue, e.g., a specific amino acid position, in a polynucleotide or a particular nucleic acid, e.g., a specific nucleic acid position, in a polypeptide. The position can be determined by properly aligning the sequence in question with the referenced sequence. A person of skill in the art would readily understand how to align to sequences to determine the relative position. For example, various alignment tools are available online, including, without limitation, “Clustal Omega Multiple Sequence Alignment,” available at www.ebi.ac.uk (last visited May 25, 2019).

The term “genetically engineered” or “engineered” refers to a method of modifying the genome of a cell, including, but not limited to, deleting a coding or non-coding region or a portion thereof or inserting a coding region or a portion thereof. In some aspects, the cell that is modified is a lymphocyte, e.g., a T cell or a modified cell that expresses CD4, which can either be obtained from a patient or a donor. The cell can be modified to express an exogenous construct, such as, e.g., a T cell receptor (TCR) disclosed herein, which is incorporated into the cell's genome. In some aspects, the cell is modified to express CD4.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation.

Cells used in an immunotherapy described herein can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety. An immunotherapy can also comprise administering a modified cell to a subject, wherein the modified cell expresses CD4 and a TCR disclosed herein. In some aspects, the modified cell is not a T cell.

A “patient” as used herein includes any human who is afflicted with a cancer (e.g., a lymphoma or a leukemia). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD4 complex, that specifically binds with a cognate stimulatory ligand present on an antigen present cell. A “stimulatory ligand” is a ligand that when present on an antigen presenting cell (e.g., an aAPC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class II molecule loaded with a peptide, an anti-CD4 antibody, a superagonist anti-CD2 antibody, a superagonist anti-CD28 antibody, and a superagonist anti-CD3 antibody.

The terms “conditioning” and “pre-conditioning” are used interchangeably herein and indicate preparing a patient in need of a T cell therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy. In one aspect, “conditioning” comprises increasing a serum level of one or more cytokines, e.g., interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof. In another aspect, “conditioning” comprises increasing a serum level of IL-7, IL-15, IP-10, MCP-1, PLGF, CRP, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Various aspects of the invention are described in further detail in the following subsections.

### II. Methods of the Disclosure

The present disclosure is directed to methods of identifying MHC class II-specific TCRs comprising contacting a T cell with a complex comprising (i) an HLA class II molecule with enhanced CD4 binding and (ii) a peptide, e.g., an epitope. In certain aspects, the T cell expresses CD4. In certain aspects, the T cell expresses one or more TCRs. In some aspects, the MHC class II-specific TCR specifically binds the complex comprising the MHC class II molecule and the peptide.

In some aspects, the MHC class II molecule comprises an alpha chain and a beta chain, wherein the alpha chain, the beta chain, or both the alpha chain and the beta chain comprises an amino acid sequence having one or more mutations relative to a wild-type alpha chain and/or beta chain of a MHC class II molecule. In some aspects, the alpha chain comprises an amino acid sequence having one or more mutations relative to a wild-type alpha chain of a MHC class II molecule. In some aspects, the beta chain comprises an amino acid sequence having one or more mutations relative to a wild-type beta chain of a MHC class II molecule. In some aspects, the alpha chain comprises an amino acid sequence having one or more mutations relative to a wild-type alpha chain of a MHC class II molecule, and the beta chain comprises an amino acid sequence having one or more mutations relative to a wild-type beta chain of a MHC class II molecule.

In some aspects, the one or more mutations comprises a substitution mutation. In some aspects, the one or more mutations comprises a deletion mutation. In some aspects, the one or more mutations comprises an insertion mutation. In some aspects, the one or more mutations comprises a substitution of a single amino acid with one or more heterologous amino acids. In some aspects, the one or more mutations comprises the substitution of a single amino acid with a different amino acid. In some aspects, the one or more mutations comprises the substation of a single amino acid with 2 different amino acids, 3 different amino acids, 4 different amino acids, 5 different amino acids, or more than 5 different amino acids.

In some aspects, the MHC class II molecule is a dimer. In some aspects, the MHC class II molecule is a trimer. In some aspects, the MHC class II molecule is a tetramer.

Certain aspects of the present disclosure are directed to methods of enriching a target population of T cells obtained from a human subject. In some aspects, the method comprises contacting the T cells with an HLA class II molecule disclosed herein. In some aspects, the method comprises contacting the T cells with a cell, e.g., an APC, disclosed herein. In some aspects, following the contacting, the enriched population of T cells comprises a higher number of T cells capable of binding the HLA class II molecule relative to the number of T cells capable of binding the HLA class II molecule prior to the contacting.

Some aspects of the present disclosure are directed to a method of selecting a T cell capable of targeting a diseased cell, e.g., a tumor cell. In some aspects, the method comprises contacting a population of isolated T cells in vitro with a complex comprising an MHC class II molecule disclosed herein and a fragment of a polypeptide, e.g. an antigen expressed by a diseased cell, e.g., a tumor-expressed polypeptide, e.g., an epitope.

In some aspects, the T cells used in the methods disclosed herein are obtained from a human subject. The T cells obtained from the human subject can be any T cells disclosed herein. In some aspects, the T cells obtained from the human subject are tumor infiltrating lymphocytes (TIL).

In some aspects, the method further comprises selecting the T cell that is bound by the MHC class II molecule. In some aspects, the method further comprises administering to the human subject the enriched T cells. In some aspects, the subject is preconditioned prior to receiving the T cells, as described herein.

In some aspects, the method further comprises isolating the TCR that is bound to the MHC class II molecule. In some aspects, the method further comprises sequencing the TCR. In some aspects, the method further comprises cloning the TCR. In some aspects, the method further comprises recombinantly expressing the TCR, or a modified variant thereof, in a host cell. In some aspects, the host cell is an immune cell, e.g., a T cell. In some aspects, the method further comprises administering the host cell to a subject. In some aspects, the subject has a cancer, and the host cell comprising the TCR treats the cancer in the subject.

II.A. MHC Class II Molecules

The human leukocyte antigen (HLA) system (the major histocompatibility complex [MHC] in humans) is an important part of the immune system and is controlled by genes located on chromosome 6. It encodes cell surface molecules specialized to present antigenic peptides to the TCR on T cells. (See also Overview of the Immune System.) MHC molecules that present antigen (Ag) are divided into 2 main classes: Class I MHC molecules and Class II MHC molecules.

Class II MHC molecules are present as transmembrane glycoproteins on the surface of professional antigen presenting cells (APCs). Intact class II molecules consist of an alpha chain and a beta chain. Three loci in the HLA complex encode MHC class II proteins: HLA-DP, HLA-DQ, and HLA-DR. T cells that express CD4 molecules react with class II MHC molecules. These lymphocytes often have effector and helper functions and activate a response to eliminate self-cells infected with intracellular pathogens or to destroy extracellular parasites and help other T cells such as CD8 T cells. Because only professional APCs express class II MHC molecules, only these cells present antigen for CD4 T cells (CD4 binds to the nonpolymorphic part of the alpha-2 and beta-2 domains of the alpha and beta chains of an MHC class II molecule respectively).

In some aspects, the HLA class II alpha and beta chains are selected from an HLA-DP, HLA-DQ, and HLA-DR allele. In certain aspects, the HLA class II beta chain is an HLA-DP allele. In certain aspects, the HLA class II alpha chain is an HLA-DP allele. In certain aspects, the HLA class II beta chain is an HLA-DQ allele. In certain aspects, the HLA class II alpha chain is an HLA-DQ allele. In certain aspects, the HLA class II beta chain is an HLA-DR allele. In certain aspects, the HLA class II alpha chain is an HLA-DR allele.

II.A.1. HLA-DP Molecules

Many HLA-DP alleles are known in the art, and any of the known alleles can be used in the methods of present disclosure. Examples of HLA-DP alpha chain and beta chain alleles are shown in Table 3. An updated list of HLA alleles is available at hla.alleles.org/(last visited on Feb. 27, 2019).

TABLE 3
DP Beta chain and alpha chain amino acid and nucleotide sequences.
Beta Chain
DPB1*04: 01 Extracellular Domain (SEQ ID NO: 1)
RATPENYLFQGRQECYAFNGTQRFLERYIYNREEFARFDSDVGEFRAVTELGRPAAEYWNSQKDILEEKRAVPD
RMCRHNYELGGPMTLQRRVQPRVNVSPSKKGPLQHHNLLVCHVTDFYPGSIQVRWELNGQEETAGVVSTNLIRN
GDWTFQILVMLEMTPQQGDVYTCQVEHTSLDSPVTVEWKAQSDSARSK
DPB1*04: 01 Extracellular Domain (SEQ ID NO: 2)
AGGGCCACTCCAGAGAATTACCTTTTCCAGGGACGGCAGGAATGCTACGCGTTTAATGGGACACAGCGCTTCCT
GGAGAGATACATCTACAACCGGGAGGAGTTCGCGCGCTTCGACAGCGACGTGGGGGAGTTCCGGGCGGTGACGG
AGCTGGGGCGGCCTGCTGCGGAGTACTGGAACAGCCAGAAGGACATCCTGGAGGAGAAGCGGGCAGTGCCGGAC
AGGATGTGCAGACACAACTACGAGCTGGGCGGGCCCATGACCCTGCAGCGCCGAGTCCAGCCTAGGGTGAATGT
TTCCCCCTCCAAGAAGGGGCCCTTGCAGCACCACAACCTGCTTGTCTGCCACGTGACGGATTTCTACCCAGGCA
GCATTCAAGTCCGATGGTTCCTGAATGGACAGGAGGAAACAGCTGGGGTCGTGTCCACCAACCTGATCCGTAAT
GGAGACTGGACCTTCCAGATCCTGGTGATGCTGGAAATGACCCCCCAGCAGGGAGATGTCTACACCTGCCAAGT
GGAGCACACCAGCCTGGATAGTCCTGTCACCGTGGAGTGGAAGGCACAGTCTGATTCTGCCCGGAGTAAG
DPB1*04: 01 L112W/V141M Extracellular Domain (SEQ ID NO: 3)
RATPENYLFQGRQECYAFNGTQRFLERYIYNREEFARFDSDVGEFRAVTELGRPAAEYWNSQKDILEEKRAVPD
RMCRHNYELGGPMTLQRRVQPRVNVSPSKKGPLQHHNWLVCHVTDFYPGSIQVRWFLNGQEETAGVMSTNLIRN
GDWTFQILVMLEMTPQQGDVYTCQVEHTSLDSPVTVEWKAQSDSARSK
Signal Peptide; DPB1*04: 01 L112W/V141M Extracellular Domain; Gly/Ser
Linker; Zip Sequences and His tag sequences) (SEQ ID NO: 4)
MMRPIVLVLLFATSALARATPENYLFQGRQECYAFNGTQRFLERYIYNREEFARFDSDVGEFRAVTELGRPAAE
YWNSQKDILEEKRAVPDRMCRHNYELGGPMTLQRRVQPRVNVSPSKKGPLQHHNWLVCHVTDFYPGSIQVRWFL
NGQEETAGVMSTNLIRNGDWTFQILVMLEMTPQQGDVYTCQVEHTSLDSPVTVEWKAQSDSARSKGGGGSLEIE
AAFLERENTALETRVAELRQRVQRLRNRVSQYRTRYGPLGGGK
Full-length wild-type DPB1*04: 01 (SEQ ID NO: 5)
MMVLQVSAAPRTVALTALLMVLLTSVVQGRATPENYLFQGRQECYAFNGTQRFLERYIYNREEFARFDSDVGEF
RAVTELGRPAAEYWNSQKDILEEKRAVPDRMCRHNYELGGPMTLQRRVQPRVNVSPSKKGPLQHHNLLVCHVTD
FYPGSIQVRWFLNGQEETAGVVSTNLIRNGDWTFQILVMLEMTPQQGDVYTCQVEHTSLDSPVTVEWKAQSDSA
RSKTLTGAGGFVLGLIICGVGIFMHRRSKKVQRGSA
Signal Peptide; DPB1*04: 01 Extracellular Domain; and Gly/Ser Linker, Zip
Sequences, GS linker, and biotinylation sequences) (SEQ ID NO: 256)
MMRPIVLVLLFATSALARATPENYLFQGRQECYAFNGTQRFLERYIYNREEFARFDSDVGEFRAVTELGRPAAE
YWNSQKDILEEKRAVPDRMCRHNYELGGPMTLQRRVQPRVNVSPSEKGPLQHHNLLVCHVTDFYPGSIQVRWFL
NGQEETAGVVSTNLIRNGDWTFQILVMLEMTPQQGDVYTCQVEHTSLDSPVTVEWEAQSDSARSKGGGGSLEIE
AAFLERENTALETRVAELRQRVQRLRNRVSQYRTRYGPLGGGKGSGLNDIFEAQKIEWHE
Signal Peptide; DPB1*04: 01 Extracellular Domain; Gly/Ser Linker; Zip
Sequences; GSlinker and biotinylation sequences) (SEQ ID NO: 257)
ATGATGAGGCCCATCGTGCTGGTGCTGCTGTTCGCCACATCTGCCCTGGCCAGAGCCACCCCCGAGAACTACCT
GTTTCAGGGCCGGCAGGAATGCTACGCCTTCAACGGCACCCAGCGGTTTCTGGAACGGTACATCTACAACCGGG
AAGAGTTCGCCAGATTCGACAGCGACGTGGGCGAGTTCAGAGCCGTGACAGAGCTGGGCAGACCTGCCGCCGAG
TACTGGAACAGCCAGAAGGACATCCTGGAAGAGAAGCGGGCCGTGCCCGACCGGATGTGCAGACACAATTACGA
GCTGGGAGGCCCCATGACCCTGCAGAGAAGAGTGCAGCCCAGAGTGAACGTGTCCCCCAGCAAGAAGGGCCCCC
TGCAGCACCACAACTTGCTTGTCTGCCACGTGACCGACTTCTACCCCGGCTCTATCCAAGTGCGGTGGTTCCTG
AACGGCCAGGAAGAGACAGCCGGCGTGGTGTCCACCAACCTGATCAGAAACGGCGACTGGACCTTCCAGATCCT
CGTGATGCTGGAAATGACCCCCCAGCAGGGCGACGTGTACACCTGTCAGGTGGAACACACCAGCCTGGACAGCC
CCGTGACCGTGGAATGGAAGGCCCAGAGCGATAGCGCCAGAAGCAAAGGCGGCGGAGGCAGCCTGGAAATCGAG
GCCGCCTTCCTGGAAAGAGAGAACACCGCCCTGGAAACCCGGGTGGCCGAGCTGAGACAGAGAGTGCAGAGACT
GCGGAACCGGGTGTCCCAGTACCGGACCAGATATGGCCCTCTGGGAGGCGGCAAAGGGTCCGGCTTGAACGACA
TTTTTGAGGCCCAGAAGATAGAGTGGCACGAGTGA
Signal Peptide; DPB1*04: 01 [symbol] Extracellular Domain; Gly/Ser
Linker; Zip Sequences; GSlinker and biotinylation sequences) (SEQ ID NO:
258)
MMRPIVLVLLFATSALARATPENYLFQGRQECYAFNGTQRFLERYIYNREEFARFDSDVGEFRAVTELGRPAAE
YWNSQKDILEEKRAVPDRMCRHNYELGGPMTLQRRVQPRVNVSPSKKGPLQHHNWLVCHVTDFYPGSIQVRWFL
NGQEETAGVMSTNLIRNGDWTFQILVMLEMTPQQGDVYTCQVEHTSLDSPVTVEWKAQSDSARSKGGGGSLEIE
AAFLERENTALETRVAELRQRVQRLRNRVSQYRTRYGPLGGGKGSGLNDIFEAQKIEWHE
Signal Peptide; DPB1*04: 01 [symbol] Extracellular Domain; Gly/Ser
Linker; Zip Sequences; GS linker and biotinylation sequences) (SEQ ID NO:
259)
ATGATGAGGCCCATCGTGCTGGTGCTGCTGTTCGCCACATCTGCCCTGGCCAGAGCCACCCCCGAGAACTACCT
GTTTCAGGGCCGGCAGGAATGCTACGCCTTCAACGGCACCCAGCGGTTTCTGGAACGGTACATCTACAACCGGG
AAGAGTTCGCCAGATTCGACAGCGACGTGGGCGAGTTCAGAGCCGTGACAGAGCTGGGCAGACCTGCCGCCGAG
TACTGGAACAGCCAGAAGGACATCCTGGAAGAGAAGCGGGCCGTGCCCGACCGGATGTGCAGACACAATTACGA
GCTGGGAGGCCCCATGACCCTGCAGAGAAGAGTGCAGCCCAGAGTGAACGTGTCCCCCAGCAAGAAGGGCCCCC
TGCAGCACCACAAC [symbol] CTTGTCTGCCACGTGACCGACTTCTACCCCGGCTCTATCCAAGTGCGGTGGTTCCTG
AACGGCCAGGAAGAGACAGCCGGCGTG [symbol] TCCACCAACCTGATCAGAAACGGCGACTGGACCTTCCAGATCCT
CGTGATGCTGGAAATGACCCCCCAGCAGGGCGACGTGTACACCTGTCAGGTGGAACACACCAGCCTGGACAGCC
CCGTGACCGTGGAATGGAAGGCCCAGAGCGATAGCGCCAGAAGCAAAGGCGGCGGAGGCAGCCTGGAAATCGAG
GCCGCCTTCCTGGAAAGAGAGAACACCGCCCTGGAAACCCGGGTGGCCGAGCTGAGACAGAGAGTGCAGAGACT
GCGGAACCGGGTGTCCCAGTACCGGACCAGATATGGCCCTCTGGGAGGCGGCAAAGGGTCCGGCTTGAACGACA
TTTTTGAGGCCCAGAAGATAGAGTGGCACGAGTGA
Alpha Chain
DPA1*01: 03 Extracellular Domain (SEQ ID NO: 6)
IKADHVSTYAAFVQTHRPTGEFMFEFDEDEMFYVDLDKKETVWHLEEFGQAFSFEAQGGLANIAILNNNLNTLI
QRSNHTQATNDPPEVTVFPKEPVELGQPNTLICHIDKFFPPVLNVTWLCNGELVTEGVAESLFLPRTDYSFHKF
HYLTFVPSAEDFYDCRVEHWGLDQPLLKHWEAQEPIQMPETTET
DPA1*01: 03 Extracellular Domain (SEQ ID NO: 7)
ATCAAGGCCGACCACGTGTCCACATACGCCGCCTTCGTGCAGACCCACAGACCCACCGGCGAGTTCATGTTCGA
GTTCGACGAGGACGAGATGTTCTACGTGGACCTGGACAAGAAAGAAACCGTGTGGCACCTGGAAGAGTTCGGCC
AGGCCTTCAGCTTTGAGGCCCAGGGCGGACTGGCCAATATCGCCATCCTGAACAACAACCTGAACACCCTGATC
CAGCGGAGCAACCACACCCAGGCCACCAACGATCCCCCCGAAGTGACCGTGTTCCCCAAAGAACCCGTGGAACT
GGGCCAGCCCAATACCCTGATCTGCCACATCGACAAGTTCTTCCCCCCCGTGCTGAACGTGACCTGGCTGTGCA
ATGGCGAGCTCGTGACAGAGGGCGTGGCCGAGTCTCTGTTCCTGCCCAGAACCGACTACAGCTTCCACAAGTTC
CACTACCTGACCTTCGTGCCCAGCGCCGAGGACTTCTACGACTGCAGAGTGGAACACTGGGGCCTGGACCAGCC
CCTGCTGAAACATTGGGAAGCCCAGGAACCCATCCAGATGCCCGAGACAACCGAGACA
Signal Peptide; DPA1*01: 03 Extracellular Domain; Gly/Ser Linker, Zip
Sequences and His tag sequences)(SEQ ID NO: 8)
MMRPIVLVLLFATSALAIKADHVSTYAAFVQTHRPTGEFMFEFDEDEMFYVDLDKKETVWHLEEFGQAFSFEAQ
GGLANIAILNNNLNTLIQRSNHTQATNDPPEVTVFPKEPVELGQPNTLICHIDKFFPPVLNVTWLCNGELVTEG
VAESLFLPRTDYSFHKFHYLTFVPSAEDFYDCRVEHWGLDQPLLKHWEAQEPIQMPETTETGGGGSLEIRAAFL
RQRNTALRTEVAELEQEVQRLENEVSQYETRYGPLGGGKGSHHHHHH
Signal Peptide; DPA1*01: 03 Extracellular Domain; Gly/Ser Linker, Zip
Sequences, and His tag sequences (10X) (SEQ ID NO: 260)
MMRPIVLVLLFATSALAIKADHVSTYAAFVQTHRPTGEFMFEFDEDEMFYVDLDKKETVWHLEEFGQAFSFEAQ
GGLANIAILNNNLNTLIQRSNHTQATNDPPEVTVFPKEPVELGQPNTLICHIDKFFPPVLNVTWLCNGELVTEG
VAESLFLPRTDYSFHKEHYLTFVPSAEDFYDCRVEHWGLDQPLLKHWEAQEPIQMPETTETGGGGSLEIRAAFL
RQRNTALRTEVAELEQEVQRLENEVSQYETRYGPLGGGKGSHHHHHHHHHH
Signal Peptide; DPA1*01: 03 Extracellular Domain; Gly/Ser Linker, Zip
Sequences, and His tag sequences (10X) (SEQ ID NO: 261)
ATGATGAGGCCCATCGTGCTGGTGCTGCTGTTCGCCACATCTGCCCTGGCCATCAAGGCCGACCACGTGTCCAC
ATACGCCGCCTTCGTGCAGACCCACAGACCCACCGGCGAGTTCATGTTCGAGTTCGACGAGGACGAGATGTTCT
ACGTGGACCTGGACAAGAAAGAAACCGTGTGGCACCTGGAAGAGTTCGGCCAGGCCTTCAGCTTTGAGGCCCAG
GGCGGACTGGCCAATATCGCCATCCTGAACAACAACCTGAACACCCTGATCCAGCGGAGCAACCACACCCAGGC
CACCAACGATCCCCCCGAAGTGACCGTGTTCCCCAAAGAACCCGTGGAACTGGGCCAGCCCAATACCCTGATCT
GCCACATCGACAAGTTCTTCCCCCCCGTGCTGAACGTGACCTGGCTGTGCAATGGCGAGCTCGTGACAGAGGGC
GTGGCCGAGTCTCTGTTCCTGCCCAGAACCGACTACAGCTTCCACAAGTTCCACTACCTGACCTTCGTGCCCAG
CGCCGAGGACTTCTACGACTGCAGAGTGGAACACTGGGGCCTGGACCAGCCCCTGCTGAAACATTGGGAAGCCC
AGGAACCCATCCAGATGCCCGAGACAACCGAGACAGGCGGCGGAGGCAGCCTGGAAATCAGAGCCGCCTTCCTG
CGGCAGAGAAACACCGCCCTGAGAACCGAAGTGGCCGAGCTGGAACAGGAAGTGCAGCGGCTGGAAAACGAGGT
GTCCCAGTACGAGACAAGATACGGCCCTCTGGGAGGCGGCAAGGGCTCTCACCACCACCATCACCATCATCATC
ACCATTGA
Signal Peptide (Fibroin light chain-derived)
MMRPIVLVLLFATSALA (SEQ ID NO: 9)

II.A.1.a. HLA-DP Beta Chain

In certain aspects, the HLA class II molecule comprises a DP beta chain, wherein the DP beta chain comprises an amino acid other than leucine at a position corresponding to amino acid residue 112 of SEQ ID NO: 1. Any amino acid other than leucine can be present at the position corresponding to amino acid residue 112 of SEQ ID NO: 1. In some aspects, the amino acid other than leucine is an amino acid comprising a hydrophobic side chain. In certain aspects, the amino acid other than leucine at the position corresponding to amino acid residue 112 of SEQ ID NO: 1 is an amino acid selected from an alanine, a valine, an isoleucine, a methionine, a phenylalanine, a tyrosine, and a tryptophan. In certain aspects, the amino acid other than leucine at the position corresponding to amino acid residue 112 of SEQ ID NO: 1 is an alanine. In certain aspects, the amino acid other than leucine at the position corresponding to amino acid residue 112 of SEQ ID NO: 1 is a valine. In certain aspects, the amino acid other than leucine at the position corresponding to amino acid residue 112 of SEQ ID NO: 1 is an isoleucine. In certain aspects, the amino acid other than leucine at the position corresponding to amino acid residue 112 of SEQ ID NO: 1 is a methionine. In certain aspects, the amino acid other than leucine at the position corresponding to amino acid residue 112 of SEQ ID NO: 1 is a phenylalanine. In certain aspects, the amino acid other than leucine at the position corresponding to amino acid residue 112 of SEQ ID NO: 1 is a tyrosine. In certain aspects, the amino acid other than leucine at the position corresponding to amino acid residue 112 of SEQ ID NO: 1 is a tryptophan.

In some embodiments, the amino acid other than leucine at the position corresponding to amino acid residue 112 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than leucine at the position corresponding to amino acid residue 112 of SEQ ID NO: 1 consists of a series, e.g., at least 2, at least 3, at least 4, or at least 5, amino acids, wherein each of the series of amino acids comprises a hydrophobic side chain.

In certain aspects, the HLA class II molecule comprises a DP beta chain, wherein the DP beta chain comprises an amino acid other than valine at a position corresponding to amino acid residue 141 of SEQ ID NO: 1. Any amino acid other than valine can be present at the position corresponding to amino acid residue 141 of SEQ ID NO: 1. In some aspects, the amino acid other than valine is an amino acid comprising a hydrophobic side chain. In certain aspects, the amino acid other than valine at the position corresponding to amino acid residue 141 of SEQ ID NO: 1 is an amino acid selected from an alanine, an isoleucine, a leucine, a methionine, a phenylalanine, a tyrosine, and a tryptophan. In certain aspects, the amino acid other than valine at the position corresponding to amino acid residue 141 of SEQ ID NO: 1 is an alanine. In certain aspects, the amino acid other than valine at the position corresponding to amino acid residue 141 of SEQ ID NO: 1 is an isoleucine. In certain aspects, the amino acid other than valine at the position corresponding to amino acid residue 141 of SEQ ID NO: 1 is a leucine. In certain aspects, the amino acid other than valine at the position corresponding to amino acid residue 141 of SEQ ID NO: 1 is a methionine. In certain aspects, the amino acid other than valine at the position corresponding to amino acid residue 141 of SEQ ID NO: 1 is a phenylalanine. In certain aspects, the amino acid other than valine at the position corresponding to amino acid residue 141 of SEQ ID NO: 1 is a tyrosine. In certain aspects, the amino acid other than valine at the position corresponding to amino acid residue 141 of SEQ ID NO: 1 is a tryptophan.

In some aspects, the amino acid other than valine at the position corresponding to amino acid residue 141 of SEQ ID NO: 1 consists of more than one amino acid, e.g., two amino acids, three amino acids, four amino acids, five amino acids, or more. In some aspects at least one of the more than one amino acids comprises a hydrophobic side chain. In certain aspects, the amino acid other than valine at the position corresponding to amino acid residue 141 of SEQ ID NO: 1 consists of a series, e.g., at least 2, at least 3, at least 4, or at least 5, amino acids, wherein each of the series of amino acids comprises a hydrophobic side chain.

In certain aspects of the present disclosure, the MHC class II molecule comprises a DP beta chain comprising more than one substitution mutation relative to the wild-type DP beta chain. In certain aspects, the DP beta chain comprises at least two mutations, at least three mutations, at least four mutations, at least five mutations, at least six mutations, at least seven mutations, at least eight mutations, at least nine mutations, or at least ten mutations relative to the wild-type DP beta chain.

In certain aspects, the DP beta chain comprises an amino acid other than leucine at a position corresponding to amino acid residue 112 of SEQ ID NO: 1 and an amino acid other than valine at a position corresponding to amino acid residue 141 of SEQ ID NO: 1. In some aspects, (i) the amino acid other than leucine at the position corresponding to amino acid residue 112 of SEQ ID NO: 1, (ii) the amino acid other than valine at a position corresponding to amino acid residue 141 of SEQ ID NO: 1, or each of the amino acid other than leucine at the position corresponding to amino acid residue 112 of SEQ ID NO: 1 and the amino acid other than valine at a position corresponding to amino acid residue 141 of SEQ ID NO: 1 is an amino acid comprising a hydrophobic side chain. In some aspects, (i) the amino acid other than leucine at the position corresponding to amino acid residue 112 of SEQ ID NO: 1 is selected from an alanine, a valine, an isoleucine, a methionine, a phenylalanine, a tyrosine, and a tryptophan; and (ii) the amino acid other than valine at a position corresponding to amino acid residue 141 of SEQ ID NO: 1 is selected from an alanine, an isoleucine, a leucine, a methionine, a phenylalanine, a tyrosine, and a tryptophan.

In some aspects, (i) the amino acid other than leucine at the position corresponding to amino acid residue 112 of SEQ ID NO: 1 is a tryptophan; and (ii) the amino acid other than valine at a position corresponding to amino acid residue 141 of SEQ ID NO: 1 is selected from an alanine, an isoleucine, a leucine, a methionine, a phenylalanine, a tyrosine, and a tryptophan. In some aspects, (i) the amino acid other than leucine at the position corresponding to amino acid residue 112 of SEQ ID NO: 1 is selected from an alanine, a valine, an isoleucine, a methionine, a phenylalanine, a tyrosine, and a tryptophan; and (ii) the amino acid other than valine at a position corresponding to amino acid residue 141 of SEQ ID NO: 1 is a methionine. In some aspects, (i) the amino acid other than leucine at the position corresponding to amino acid residue 112 of SEQ ID NO: 1 is a tryptophan; and (ii) the amino acid other than valine at a position corresponding to amino acid residue 141 of SEQ ID NO: 1 is a methionine.

In certain aspects, the DP beta chain further comprises an amino acid other than valine at a position corresponding to amino acid residue 114 of SEQ ID NO: 1. In some aspects, the amino acid other than valine at the position corresponding to amino acid residue 114 of SEQ ID NO: 1 is selected from an alanine, an isoleucine, a leucine, a methionine, a phenylalanine, a tyrosine, and a tryptophan. In certain aspects, the amino acid other than valine at a position corresponding to amino acid residue 114 of SEQ ID NO: 1 is a methionine.

In certain aspects, the DP beta chain further comprises an amino acid other than methionine at a position corresponding to amino acid residue 158 of SEQ ID NO: 1. In some aspects, the amino acid other than methionine at the position corresponding to amino acid residue 158 of SEQ ID NO: 1 is selected from an alanine, a valine, an isoleucine, a methionine, a phenylalanine, a tyrosine, and a tryptophan. In certain aspects, the amino acid other than methionine at a position corresponding to amino acid residue 158 of SEQ ID NO: 1 is an isoleucine.

In some aspects, the DP beta chain comprises (i) an amino acid other than leucine at a position corresponding to amino acid residue 112 of SEQ ID NO: 1, and (ii) an amino acid other than valine at a position corresponding to amino acid residue 114 of SEQ ID NO: 1. In some aspects, the DP beta chain comprises (i) an amino acid other than leucine at a position corresponding to amino acid residue 112 of SEQ ID NO: 1, and (ii) an amino acid other than methionine at a position corresponding to amino acid residue 158 of SEQ ID NO: 1.

In some aspects, the DP beta chain comprises (i) a tryptophan at a position corresponding to amino acid residue 112 of SEQ ID NO: 1, and (ii) a methionine at a position corresponding to amino acid residue 114 of SEQ ID NO: 1. In some aspects, the DP beta chain comprises (i) a tryptophan at a position corresponding to amino acid residue 112 of SEQ ID NO: 1, and (ii) a isoleucine at a position corresponding to amino acid residue 158 of SEQ ID NO: 1.

In some aspects, the DP beta chain comprises (i) an amino acid other than valine at a position corresponding to amino acid residue 141 of SEQ ID NO: 1, and (ii) an amino acid other than valine at a position corresponding to amino acid residue 114 of SEQ ID NO: 1. In some aspects, the DP beta chain comprises (i) an amino acid other than valine at a position corresponding to amino acid residue 141 of SEQ ID NO: 1, and (ii) an amino acid other than methionine at a position corresponding to amino acid residue 158 of SEQ ID NO: 1.

In some aspects, the DP beta chain comprises (i) a methionine at a position corresponding to amino acid residue 141 of SEQ ID NO: 1, and (ii) an amino acid other than valine at a position corresponding to amino acid residue 114 of SEQ ID NO: 1. In some aspects, the DP beta chain comprises (i) a methionine at a position corresponding to amino acid residue 141 of SEQ ID NO: 1, and (ii) a isoleucine at a position corresponding to amino acid residue 158 of SEQ ID NO: 1.

In some aspects, the DP beta chain comprises (i) an amino acid other than leucine at a position corresponding to amino acid residue 112 of SEQ ID NO: 1, (ii) an amino acid other than valine at a position corresponding to amino acid residue 141 of SEQ ID NO: 1, (iii) an amino acid other than valine at a position corresponding to amino acid residue 114 of SEQ ID NO: 1, and (iv) an amino acid other than methionine at a position corresponding to amino acid residue 158 of SEQ ID NO: 1.

In some aspects, the DP beta chain comprises (i) a tryptophan at a position corresponding to amino acid residue 112 of SEQ ID NO: 1, (ii) a methionine at a position corresponding to amino acid residue 141 of SEQ ID NO: 1, (iii) a methionine at a position corresponding to amino acid residue 114 of SEQ ID NO: 1, and (iv) a isoleucine at a position corresponding to amino acid residue 158 of SEQ ID NO: 1.

In some aspects, the DP beta chain comprises a valine at a position corresponding to amino acid residue 114 SEQ ID NO: 1. In some aspects, the DP beta chain comprises a methionine at a position corresponding to amino acid residue 158 corresponding to SEQ ID NO: 1. In some aspects, the DP beta chain comprises (i) an amino acid other than leucine at a position corresponding to amino acid residue 112 of SEQ ID NO: 1, (ii) an amino acid other than valine at a position corresponding to amino acid residue 141 of SEQ ID NO: 1, and (iii) a valine at a position corresponding to amino acid residue 114 SEQ ID NO: 1. In some aspects, the DP beta chain comprises (i) an amino acid other than leucine at a position corresponding to amino acid residue 112 of SEQ ID NO: 1, (ii) an amino acid other than valine at a position corresponding to amino acid residue 141 of SEQ ID NO: 1, and (iii) a methionine at a position corresponding to amino acid residue 158 corresponding to SEQ ID NO: 1. In some aspects, the DP beta chain comprises (i) an amino acid other than leucine at a position corresponding to amino acid residue 112 of SEQ ID NO: 1, (ii) an amino acid other than valine at a position corresponding to amino acid residue 141 of SEQ ID NO: 1, (iii) a valine at a position corresponding to amino acid residue 114 SEQ ID NO: 1, and (iv) a methionine at a position corresponding to amino acid residue 158 corresponding to SEQ ID NO: 1.

In some aspects, the DP beta chain comprises (i) a tryptophan at a position corresponding to amino acid residue 112 of SEQ ID NO: 1, (ii) a methionine at a position corresponding to amino acid residue 141 of SEQ ID NO: 1, and (iii) a valine at a position corresponding to amino acid residue 114 SEQ ID NO: 1. In some aspects, the DP beta chain comprises (i) a tryptophan at a position corresponding to amino acid residue 112 of SEQ ID NO: 1, (ii) a methionine at a position corresponding to amino acid residue 141 of SEQ ID NO: 1, and (iii) a methionine at a position corresponding to amino acid residue 158 corresponding to SEQ ID NO: 1. In some aspects, the DP beta chain comprises (i) a tryptophan at a position corresponding to amino acid residue 112 of SEQ ID NO: 1, (ii) a methionine at a position corresponding to amino acid residue 141 of SEQ ID NO: 1, (iii) a valine at a position corresponding to amino acid residue 114 SEQ ID NO: 1, and (iv) a methionine at a position corresponding to amino acid residue 158 corresponding to SEQ ID NO: 1.

In some aspects, the DP beta chain comprises (i) a tryptophan at a position corresponding to amino acid residue 112 of SEQ ID NO: 1, (ii) a methionine at a position corresponding to amino acid residue 141 of SEQ ID NO: 1, (iii) a methionine at a position corresponding to amino acid residue 114 of SEQ ID NO: 1, and (iv) a isoleucine at a position corresponding to amino acid residue 158 of SEQ ID NO: 1.

In certain aspects, a DP beta chain described herein has an increased affinity for a CD4 protein as compared to a reference HLA class II molecule. In some aspects, the reference HLA class II molecule is an HLA class II molecule having a wild-type DP beta chain. In some aspects, the reference HLA class II molecule is an HLA class II molecule having a DP beta chain comprising (i) a leucine at a position corresponding to amino acid residue 112 of SEQ ID NO: 1 and/or (ii) a valine at a position corresponding to amino acid residue 141 of SEQ ID NO: 1.

In some aspects, the increased affinity for CD4 is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, at least about 4000-fold, at least about 4500-fold, or at least about 4000-fold greater than the affinity of the reference HLA class II molecule for CD4.

In some aspects, the increased affinity for CD4 is at least about 1.5-fold to at least about 5000-fold, 1.5-fold to at least about 4000-fold, 1.5-fold to at least about 3000-fold, 1.5-fold to at least about 2000-fold, 1.5-fold to at least about 1000-fold, 10-fold to at least about 5000-fold, 10-fold to at least about 4000-fold, 10-fold to at least about 3000-fold, 10-fold to at least about 2000-fold, 10-fold to at least about 1000-fold, 10-fold to at least about 900-fold, 10-fold to at least about 800-fold, 10-fold to at least about 700-fold, 10-fold to at least about 600-fold, 10-fold to at least about 500-fold, 10-fold to at least about 400-fold, 10-fold to at least about 300-fold, 10-fold to at least about 200-fold, 10-fold to at least about 100-fold, 100-fold to at least about 5000-fold, 100-fold to at least about 4000-fold, 100-fold to at least about 3000-fold, 100-fold to at least about 2000-fold, 100-fold to at least about 1000-fold, 100-fold to at least about 900-fold, 100-fold to at least about 800-fold, 100-fold to at least about 700-fold, 100-fold to at least about 600-fold, 100-fold to at least about 500-fold, 100-fold to at least about 400-fold, 100-fold to at least about 300-fold, or 100-fold to at least about 200-fold greater than the affinity of the reference HLA class II molecule for CD4.

In certain aspects, the DP beta chain comprises an allele selected from DPB1*01, DPB1*02, DPB1*03, DPB1*04, DPB1*05, DPB1*06, DPB1*08, DPB1*09, DPB1*10, DPB1*100, DPB1*101, DPB1*102, DPB1*103, DPB1*104, DPB1*105, DPB1*106, DPB1*107, DPB1*108, DPB1*109, DPB1*11, DPB1*110, DPB1*111, DPB1*112, DPB1*113, DPB1*114, DPB1*115, DPB1*116, DPB1*117, DPB1*118, DPB1*119, DPB1*120, DPB1*121, DPB1*122, DPB1*123, DPB1*124, DPB1*125, DPB1*126, DPB1*127, DPB1*128, DPB1*129, DPB1*13, DPB1*130, DPB1*131, DPB1*132, DPB1*133, DPB1*134, DPB1*135, DPB1*136, DPB1*137, DPB1*138, DPB1*139, DPB1*14, DPB1*140, DPB1*141, DPB1*142, DPB1*143, DPB1*144, DPB1*145, DPB1*146, DPB1*147, DPB1*148, DPB1*149, DPB1*15, DPB1*150, DPB1*151, DPB1*152, DPB1*153, DPB1*154, DPB1*155, DPB1*156, DPB1*157, DPB1*158, DPB1*159, DPB1*16, DPB1*160, DPB1*161, DPB1*162, DPB1*163, DPB1*164, DPB1*165, DPB1*166, DPB1*167, DPB1*168, DPB1*169, DPB1*17, DPB1*170, DPB1*171, DPB1*172, DPB1*173, DPB1*174, DPB1*175, DPB1*176, DPB1*177, DPB1*178, DPB1*179, DPB1*18, DPB1*180, DPB1*181, DPB1*182, DPB1*183, DPB1*184, DPB1*185, DPB1*186, DPB1*187, DPB1*188, DPB1*189, DPB1*19, DPB1*190, DPB1*191, DPB1*192, DPB1*193, DPB1*194, DPB1*195, DPB1*196, DPB1*197, DPB1*198, DPB1*199, DPB1*20, DPB1*200, DPB1*201, DPB1*202, DPB1*203, DPB1*204, DPB1*205, DPB1*206, DPB1*207, DPB1*208, DPB1*209, DPB1*21, DPB1*210, DPB1*211, DPB1*212, DPB1*213, DPB1*214, DPB1*215, DPB1*216, DPB1*217, DPB1*218, DPB1*219, DPB1*22, DPB1*220, DPB1*221, DPB1*222, DPB1*223, DPB1*224, DPB1*225, DPB1*226, DPB1*227, DPB1*228, DPB1*229, DPB1*23, DPB1*230, DPB1*231, DPB1*232, DPB1*233, DPB1*234, DPB1*235, DPB1*236, DPB1*237, DPB1*238, DPB1*239, DPB1*24, DPB1*240, DPB1*241, DPB1*242, DPB1*243, DPB1*244, DPB1*245, DPB1*246, DPB1*247, DPB1*248, DPB1*249, DPB1*25, DPB1*250, DPB1*251, DPB1*252, DPB1*253, DPB1*254, DPB1*255, DPB1*256, DPB1*257, DPB1*258, DPB1*259, DPB1*26, DPB1*260, DPB1*261, DPB1*262, DPB1*263, DPB1*264, DPB1*265, DPB1*266, DPB1*267, DPB1*268, DPB1*269, DPB1*27, DPB1*270, DPB1*271, DPB1*272, DPB1*273, DPB1*274, DPB1*275, DPB1*276, DPB1*277, DPB1*278, DPB1*279, DPB1*28, DPB1*280, DPB1*281, DPB1*282, DPB1*283, DPB1*284, DPB1*285, DPB1*286, DPB1*287, DPB1*288, DPB1*289, DPB1*29, DPB1*290, DPB1*291, DPB1*292, DPB1*293, DPB1*294, DPB1*295, DPB1*296, DPB1*297, DPB1*298, DPB1*299, DPB1*30, DPB1*300, DPB1*301, DPB1*302, DPB1*303, DPB1*304, DPB1*305, DPB1*306, DPB1*307, DPB1*308, DPB1*309, DPB1*31, DPB1*310, DPB1*311, DPB1*312, DPB1*313, DPB1*314, DPB1*315, DPB1*316, DPB1*317, DPB1*318, DPB1*319, DPB1*32, DPB1*320, DPB1*321, DPB1*322, DPB1*323, DPB1*324, DPB1*325, DPB1*326, DPB1*327, DPB1*328, DPB1*329, DPB1*33, DPB1*330, DPB1*331, DPB1*332, DPB1*333, DPB1*334, DPB1*335, DPB1*336, DPB1*337, DPB1*338, DPB1*339, DPB1*34, DPB1*340, DPB1*341, DPB1*342, DPB1*343, DPB1*344, DPB1*345, DPB1*346, DPB1*347, DPB1*348, DPB1*349, DPB1*35, DPB1*350, DPB1*351, DPB1*352, DPB1*353, DPB1*354, DPB1*355, DPB1*356, DPB1*357, DPB1*358, DPB1*359, DPB1*36, DPB1*360, DPB1*361, DPB1*362, DPB1*363, DPB1*364, DPB1*365, DPB1*366, DPB1*367, DPB1*368, DPB1*369, DPB1*37, DPB1*370, DPB1*371, DPB1*372, DPB1*373, DPB1*374, DPB1*375, DPB1*376, DPB1*377, DPB1*378, DPB1*379, DPB1*38, DPB1*380, DPB1*381, DPB1*382, DPB1*383, DPB1*384, DPB1*385, DPB1*386, DPB1*387, DPB1*388, DPB1*389, DPB1*39, DPB1*390, DPB1*391, DPB1*392, DPB1*393, DPB1*394, DPB1*395, DPB1*396, DPB1*397, DPB1*398, DPB1*399, DPB1*40, DPB1*400, DPB1*401, DPB1*402, DPB1*403, DPB1*404, DPB1*405, DPB1*406, DPB1*407, DPB1*408, DPB1*409, DPB1*41, DPB1*410, DPB1*411, DPB1*412, DPB1*413, DPB1*414, DPB1*415, DPB1*416, DPB1*417, DPB1*418, DPB1*419, DPB1*420, DPB1*421, DPB1*422, DPB1*423, DPB1*424, DPB1*425, DPB1*426, DPB1*427, DPB1*428, DPB1*429, DPB1*430, DPB1*431, DPB1*432, DPB1*433, DPB1*434, DPB1*435, DPB1*436, DPB1*437, DPB1*438, DPB1*439, DPB1*44, DPB1*440, DPB1*441, DPB1*442, DPB1*443, DPB1*444, DPB1*445, DPB1*446, DPB1*447, DPB1*448, DPB1*449, DPB1*45, DPB1*450, DPB1*451, DPB1*452, DPB1*453, DPB1*454, DPB1*455, DPB1*456, DPB1*457, DPB1*458, DPB1*459, DPB1*46, DPB1*460, DPB1*461, DPB1*462, DPB1*463, DPB1*464, DPB1*465, DPB1*466, DPB1*467, DPB1*468, DPB1*469, DPB1*47, DPB1*470, DPB1*471, DPB1*472, DPB1*473, DPB1*474, DPB1*475, DPB1*476, DPB1*477, DPB1*478, DPB1*479, DPB1*48, DPB1*480, DPB1*481, DPB1*482, DPB1*483, DPB1*484, DPB1*485, DPB1*486, DPB1*487, DPB1*488, DPB1*489, DPB1*49, DPB1*490, DPB1*491, DPB1*492, DPB1*493, DPB1*494, DPB1*495, DPB1*496, DPB1*497, DPB1*498, DPB1*499, DPB1*50, DPB1*500, DPB1*501, DPB1*502, DPB1*503, DPB1*504, DPB1*505, DPB1*506, DPB1*507, DPB1*508, DPB1*509, DPB1*51, DPB1*510, DPB1*511, DPB1*512, DPB1*513, DPB1*514, DPB1*515, DPB1*516, DPB1*517, DPB1*518, DPB1*519, DPB1*52, DPB1*520, DPB1*521, DPB1*522, DPB1*523, DPB1*524, DPB1*525, DPB1*526, DPB1*527, DPB1*528, DPB1*529, DPB1*53, DPB1*530, DPB1*531, DPB1*532, DPB1*533, DPB1*534, DPB1*535, DPB1*536, DPB1*537, DPB1*538, DPB1*539, DPB1*54, DPB1*540, DPB1*541, DPB1*542, DPB1*543, DPB1*544, DPB1*545, DPB1*546, DPB1*547, DPB1*548, DPB1*549, DPB1*55, DPB1*550, DPB1*551, DPB1*552, DPB1*553, DPB1*554, DPB1*555, DPB1*556, DPB1*557, DPB1*558, DPB1*559, DPB1*56, DPB1*560, DPB1*561, DPB1*562, DPB1*563, DPB1*564, DPB1*565, DPB1*566, DPB1*567, DPB1*568, DPB1*569, DPB1*57, DPB1*570, DPB1*571, DPB1*572, DPB1*573, DPB1*574, DPB1*575, DPB1*576, DPB1*577, DPB1*578, DPB1*579, DPB1*58, DPB1*580, DPB1*581, DPB1*582, DPB1*583, DPB1*584, DPB1*585, DPB1*586, DPB1*587, DPB1*588, DPB1*589, DPB1*59, DPB1*590, DPB1*591, DPB1*592, DPB1*593, DPB1*594, DPB1*595, DPB1*596, DPB1*597, DPB1*598, DPB1*599, DPB1*60, DPB1*600, DPB1*601, DPB1*602, DPB1*603, DPB1*604, DPB1*605, DPB1*606, DPB1*607, DPB1*608, DPB1*609, DPB1*61, DPB1*610, DPB1*611, DPB1*612, DPB1*613, DPB1*614, DPB1*615, DPB1*616, DPB1*617, DPB1*618, DPB1*619, DPB1*62, DPB1*620, DPB1*621, DPB1*622, DPB1*623, DPB1*624, DPB1*625, DPB1*626, DPB1*627, DPB1*628, DPB1*629, DPB1*63, DPB1*630, DPB1*631, DPB1*632, DPB1*633, DPB1*634, DPB1*635, DPB1*636, DPB1*637, DPB1*638, DPB1*639, DPB1*64, DPB1*640, DPB1*641, DPB1*642, DPB1*643, DPB1*644, DPB1*645, DPB1*646, DPB1*647, DPB1*648, DPB1*649, DPB1*65, DPB1*650, DPB1*651, DPB1*652, DPB1*653, DPB1*654, DPB1*655, DPB1*656, DPB1*657, DPB1*658, DPB1*659, DPB1*66, DPB1*660, DPB1*661, DPB1*662, DPB1*663, DPB1*664, DPB1*665, DPB1*666, DPB1*667, DPB1*668, DPB1*669, DPB1*67, DPB1*670, DPB1*671, DPB1*672, DPB1*673, DPB1*674, DPB1*675, DPB1*676, DPB1*677, DPB1*678, DPB1*679, DPB1*68, DPB1*680, DPB1*681, DPB1*682, DPB1*683, DPB1*684, DPB1*685, DPB1*686, DPB1*687, DPB1*688, DPB1*689, DPB1*69, DPB1*690, DPB1*691, DPB1*692, DPB1*693, DPB1*694, DPB1*695, DPB1*696, DPB1*697, DPB1*698, DPB1*699, DPB1*70, DPB1*700, DPB1*701, DPB1*702, DPB1*703, DPB1*704, DPB1*705, DPB1*706, DPB1*707, DPB1*708, DPB1*709, DPB1*71, DPB1*710, DPB1*711, DPB1*712, DPB1*713, DPB1*714, DPB1*715, DPB1*716, DPB1*717, DPB1*718, DPB1*719, DPB1*72, DPB1*720, DPB1*721, DPB1*722, DPB1*723, DPB1*724, DPB1*725, DPB1*726, DPB1*727, DPB1*728, DPB1*729, DPB1*73, DPB1*730, DPB1*731, DPB1*732, DPB1*733, DPB1*734, DPB1*735, DPB1*736, DPB1*737, DPB1*738, DPB1*739, DPB1*74, DPB1*740, DPB1*741, DPB1*742, DPB1*743, DPB1*744, DPB1*745, DPB1*746, DPB1*747, DPB1*748, DPB1*749, DPB1*75, DPB1*750, DPB1*751, DPB1*752, DPB1*753, DPB1*754, DPB1*755, DPB1*756, DPB1*757, DPB1*758, DPB1*759, DPB1*76, DPB1*760, DPB1*761, DPB1*762, DPB1*763, DPB1*764, DPB1*765, DPB1*766, DPB1*767, DPB1*768, DPB1*769, DPB1*77, DPB1*770, DPB1*771, DPB1*772, DPB1*773, DPB1*774, DPB1*775, DPB1*776, DPB1*777, DPB1*778, DPB1*779, DPB1*78, DPB1*780, DPB1*781, DPB1*782, DPB1*783, DPB1*784, DPB1*785, DPB1*786, DPB1*787, DPB1*788, DPB1*789, DPB1*79, DPB1*790, DPB1*791, DPB1*792, DPB1*794, DPB1*795, DPB1*796, DPB1*797, DPB1*798, DPB1*799, DPB1*80, DPB1*800, DPB1*801, DPB1*802, DPB1*803, DPB1*804, DPB1*805, DPB1*806, DPB1*807, DPB1*808, DPB1*809, DPB1*81, DPB1*810, DPB1*811, DPB1*812, DPB1*813, DPB1*814, DPB1*815, DPB1*816, DPB1*817, DPB1*818, DPB1*819, DPB1*82, DPB1*820, DPB1*821, DPB1*822, DPB1*823, DPB1*824, DPB1*825, DPB1*826, DPB1*827, DPB1*828, DPB1*829, DPB1*83, DPB1*830, DPB1*831, DPB1*832, DPB1*833, DPB1*834, DPB1*835, DPB1*836, DPB1*837, DPB1*838, DPB1*839, DPB1*84, DPB1*840, DPB1*841, DPB1*842, DPB1*843, DPB1*844, DPB1*845, DPB1*846, DPB1*847, DPB1*848, DPB1*849, DPB1*85, DPB1*850, DPB1*851, DPB1*852, DPB1*853, DPB1*854, DPB1*855, DPB1*856, DPB1*857, DPB1*858, DPB1*859, DPB1*86, DPB1*860, DPB1*861, DPB1*862, DPB1*863, DPB1*864, DPB1*865, DPB1*866, DPB1*867, DPB1*868, DPB1*869, DPB1*87, DPB1*870, DPB1*871, DPB1*872, DPB1*873, DPB1*874, DPB1*875, DPB1*876, DPB1*877, DPB1*878, DPB1*879, DPB1*88, DPB1*880, DPB1*881, DPB1*882, DPB1*883, DPB1*884, DPB1*885, DPB1*886, DPB1*887, DPB1*888, DPB1*889, DPB1*89, DPB1*890, DPB1*891, DPB1*892, DPB1*893, DPB1*894, DPB1*895, DPB1*896, DPB1*897, DPB1*898, DPB1*899, DPB1*90, DPB1*900, DPB1*901, DPB1*902, DPB1*903, DPB1*904, DPB1*905, DPB1*906, DPB1*907, DPB1*908, DPB1*909, DPB1*91, DPB1*910, DPB1*911, DPB1*912, DPB1*913, DPB1*914, DPB1*915, DPB1*916, DPB1*917, DPB1*918, DPB1*919, DPB1*92, DPB1*920, DPB1*921, DPB1*922, DPB1*923, DPB1*924, DPB1*925, DPB1*926, DPB1*927, DPB1*928, DPB1*929, DPB1*93, DPB1*930, DPB1*931, DPB1*932, DPB1*933, DPB1*934, DPB1*935, DPB1*936, DPB1*937, DPB1*938, DPB1*939, DPB1*94, DPB1*940, DPB1*941, DPB1*942, DPB1*943, DPB1*944, DPB1*945, DPB1*946, DPB1*947, DPB1*948, DPB1*949, DPB1*95, DPB1*950, DPB1*951, DPB1*952, DPB1*953, DPB1*954, DPB1*955, DPB1*956, DPB1*957, DPB1*958, DPB1*959, DPB1*96, DPB1*960, DPB1*961, DPB1*962, DPB1*963, DPB1*964, DPB1*965, DPB1*97, DPB1*98, and DPB1*99. In some aspects, the DP beta chain comprises an HLA-DPB1*01, HLA-DPB1*02, HLA-DPB1*03, HLA-DPB1*04, HLA-DPB1*05, HLA-DPB1*06, HLA-DPB1*08, or HLA-DPB1*09 allele. In certain aspects, the DP beta chain comprises an HLA-DPB1*04 allele. In particular aspects, the DP beta chain comprises an HLA-DPB1*04:01 allele.

In certain aspects, the MHC class II molecule comprises a DP beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 3, wherein the DP beta chain comprises a tryptophan at a position corresponding to amino acid residue 112 of SEQ ID NO: 1, and wherein the DP beta chain comprises a methionine at a position corresponding to amino acid residue 141 of SEQ ID NO: 1. In certain aspects, the MHC class II molecule comprises a DP beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 3, wherein the DP beta chain comprises (i) a tryptophan at a position corresponding to amino acid residue 112 of SEQ ID NO: 1, (ii) a methionine at a position corresponding to amino acid residue 141 of SEQ ID NO: 1, (iii) a valine at a position corresponding to amino acid residue 114 SEQ ID NO: 1, and (iv) a methionine at a position corresponding to amino acid residue 158 corresponding to SEQ ID NO: 1. In certain aspects, the MHC class II molecule comprises a DP beta chain comprising an amino acid sequence set forth in SEQ ID NO: 3.

II.A.1.a. HLA-DP Alpha Chain

In some aspects of the present disclosure, the MHC class II molecule further comprises an alpha chain. In some aspects, the alpha chain is a wild-type alpha chain. In some aspects, the alpha chain is a DP alpha chain. Any DP alpha chain can be used in the compositions and methods of the present disclosure. In some aspects, the DP alpha chain comprises an HLA-DPA1*01, HLA-DPA1*02, HLA-DPA1*03, or HLA-DPA1*04 allele. In certain aspects, the DP alpha chain comprises an HLA-DPA1*01 allele. In certain aspects, the DP alpha chain comprises an HLA-DPA1*02 allele. In certain aspects, the DP alpha chain comprises an HLA-DPA1*03 allele. In certain aspects, the DP alpha chain comprises an HLA-DPA1*04 allele.

In certain aspects, the DP alpha chain is selected from DPA1*01:03:01:01, DPA1*01:03:01:02, DPA1*01:03:01:03, DPA1*01:03:01:04, DPA1*01:03:01:05, DPA1*01:03:01:06, DPA1*01:03:01:07, DPA1*01:03:01:08, DPA1*01:03:01:09, DPA1*01:03:01:10, DPA1*01:03:01:11, DPA1*01:03:01:12, DPA1*01:03:01:13, DPA1*01:03:01:14, DPA1*01:03:01:15, DPA1*01:03:01:16, DPA1*01:03:01:17, DPA1*01:03:01:18Q, DPA1*01:03:01:19, DPA1*01:03:01:20, DPA1*01:03:01:21, DPA1*01:03:01:22, DPA1*01:03:01:23, DPA1*01:03:02, DPA1*01:03:03, DPA1*01:03:04, DPA1*01:03:05, DPA1*01:03:06, DPA1*01:03:07, DPA1*01:03:08, DPA1*01:03:09, DPA1*01:04, DPA1*01:05, DPA1*01:06:01, DPA1*01:06:02, DPA1*01:07, DPA1*01:08, DPA1*01:09, DPA1*01:10, DPA1*01:11, DPA1*01:12, DPA1*01:13, DPA1*01:14, DPA1*01:15, DPA1*01:16, DPA1*01:17, DPA1*01:18, DPA1*01:19, DPA1*02:01:01:01, DPA1*02:01:01:02, DPA1*02:01:01:03, DPA1*02:01:01:04, DPA1*02:01:01:05, DPA1*02:01:01:06, DPA1*02:01:01:07, DPA1*02:01:01:08, DPA1*02:01:01:09, DPA1*02:01:01:10, DPA1*02:01:01:11, DPA1*02:01:02:01, DPA1*02:01:02:02, DPA1*02:01:03, DPA1*02:01:04, DPA1*02:01:05, DPA1*02:01:06, DPA1*02:01:07, DPA1*02:01:08:01, DPA1*02:01:08:02, DPA1*02:02:02:01, DPA1*02:02:02:02, DPA1*02:02:02:03, DPA1*02:02:02:04, DPA1*02:02:02:05, DPA1*02:02:03, DPA1*02:02:04, DPA1*02:02:05, DPA1*02:02:06, DPA1*02:03, DPA1*02:04, DPA1*02:05, DPA1*02:06, DPA1*02:07:01:01, DPA1*02:07:01:02, DPA1*02:07:01:03, DPA1*02:08, DPA1*02:09, DPA1*02:10, DPA1*02:11, DPA1*02:12, DPA1*02:13N, DPA1*02:14, DPA1*02:15, DPA1*02:16, DPA1*03:01:01:01, DPA1*03:01:01:02, DPA1*03:01:01:03, DPA1*03:01:01:04, DPA1*03:01:01:05, DPA1*03:01:02, DPA1*03:02, DPA1*03:03, DPA1*03:04, DPA1*04:01:01:01, DPA1*04:01:01:02, DPA1*04:01:01:03, DPA1*04:02, or any combination thereof.

In certain aspects, the MHC class II molecule comprises a DP alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 6. In certain aspects, the MHC class II molecule comprises a DP alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 8. In certain aspects, the MHC class II molecule comprises a DP alpha chain comprising an amino acid sequence set forth in SEQ ID NO: 6. In certain aspects, the MHC class II molecule comprises a DP alpha chain comprising an amino acid sequence set forth in SEQ ID NO: 8.

II.A.2. HLA-DQ Molecules

Many HLA-DQ alleles are known in the art, and any of the known alleles can be used in the present disclosure. Examples of HLA-DQ alpha chain and beta chain alleles are shown in Table 4. An updated list of HLA alleles is available at hla.alleles.org/(last visited on Jul. 10, 2019).

TABLE 4
DQ Beta chain and alpha chain amino acid and nucleotide sequences.
Beta Chain
DQB1*05: 01 Extracellular Domain (SEQ ID NO: 11)
RDSPEDFVYQFKGLCYFTNGTERVRGVTRHIYNREEYVRFDSDVGVYRAVTPQGRPVAEYWNSQKEVLEGARAS
VDRVCRHNYEVAYRGILQRRVEPTVTISPSRTEALNHHNLLICSVTDFYPSQIKVRWFRNDQEETAGVVSTPLI
RNGDWTFQILVMLEMTPQRGDVYTCHVEHPSLQSPITVEWRAQSESAQSK
DQB1*05: 01 Extracellular Domain (SEQ ID NO: 12)
AGAGACTCTCCCGAGGATTTCGTGTACCAGTTTAAGGGCCTGTGCTACTTCACCAACGGGACGGAGCGCGTGCG
GGGTGTGACCAGACACATCTATAACCGAGAGGAGTACGTGCGCTTCGACAGCGACGTGGGGGTGTACCGGGCAG
TGACGCCGCAGGGGCGGCCTGTTGCCGAGTACTGGAACAGCCAGAAGGAAGTCCTGGAGGGGGCCCGGGCGTCG
GTGGACAGGGTGTGCAGACACAACTACGAGGTGGCGTACCGCGGGATCCTGCAGAGGAGAGTGGAGCCCACAGT
GACCATCTCCCCATCCAGGACAGAGGCCCTCAACCACCACAACCTGCTGATCTGCTCGGTGACAGATTTCTATC
CAAGCCAGATCAAAGTCCGGTGGTTTCGGAATGATCAGGAGGAGACAGCCGGCGTTGTGTCCACCCCCCTCATT
AGGAACGGTGACTGGACCTTCCAGATCCTGGTGATGCTGGAAATGACTCCCCAGCGTGGAGATGTCTACACCTG
CCACGTGGAGCACCCCAGCCTCCAGAGCCCCATCACCGTGGAGTGGCGGGCTCAGTCTGAATCTGCCCAGAGCA
AG
DQB1*05: 01 L114W/V143M Extracellular Domain (SEQ ID NO: 265)
RDSPEDFVYQFKGLCYFTNGTERVRGVTRHIYNREEYVRFDSDVGVYRAVTPQGRPVAEYWNSQKEVLEGARAS
VDRVCRHNYEVAYRGILQRRVEPTVTISPSRTEALNHHNWLICSVTDFYPSQIKVRWFRNDQEETAGVMSTPLI
RNGDWTFQILVMLEMTPQRGDVYTCHVEHPSLQSPITVEWRAQSESAQSK
DQB1*05: 01 L114W/V143M + 4 Reps Extracellular Domain (SEQ ID NO: 13)
RDSPEDEVYQFKGLCYFTNGTERVRGVTRHIYNREEYVRFDSDVGVYRAVTPQGRPVAEYWNSQKEVLEGARAS
VDRVCRHNYEVAYRGILQRRVEPTVTISPSRTEALQHHNWLVCHVTDFYPSQIKVRWFRNDQEETAGVMSTNLI
RNGDWTFQILVMLEMTPQRGDVYTCHVEHPSLQSPITVEWRAQSESAQSK
Signal Peptide; DQB1*05: 01 Domain; Gly/Ser Linker; Zip Sequences and His tag
sequences) (SEQ ID NO: 266)
MMRPIVLVLLFATSALARDSPEDFVYQFKGLCYFTNGTERVRGVTRHIYNREEYVRFDSDVGVYRAVTPQGRPV
AEYWNSQKEVLEGARASVDRVCRHNYEVAYRGILQRRVEPTVTISPSRTEALNHHNLLICSVTDFYPSQIKVRW
FRNDQEETAGVVSTPLIRNGDWTFQILVMLEMTPQRGDVYTCHVEHPSLQSPITVEWRAQSESAQSKGGGGSLE
IEAAFLERENTALETRVAELRQRVQRLRNRVSQYRTRYGPLGGGK
Signal Peptide; DQB1*05: 01 L114W/V143M Domain; Gly/Ser Linker; Zip Sequences
and His tag sequences) (SEQ ID NO: 267)
MMRPIVLVLLFATSALARDSPEDFVYQFKGLCYFTNGTERVRGVTRHIYNREEYVRFDSDVGVYRAVTPQGRPV
AEYWNSQKEVLEGARASVDRVCRHNYEVAYRGILQRRVEPTVTISPSRTEALNHHNWLICSVTDFYPSQIKVRW
FRNDQEETAGVMSTPLIRNGDWTFQILVMLEMTPQRGDVYTCHVEHPSLQSPITVEWRAQSESAQSKGGGGSLE
IEAAFLERENTALETRVAELRQRVQRLRNRVSQYRTRYGPLGGGK
Signal Peptide; DQB1*05: 01 L114W/V143M + 4 Reps Extracellular Domain; Gly/Ser
Linker; Zip Sequences and His tag sequences) (SEQ ID NO: 14)
MMRPIVLVLLFATSALARDSPEDFVYQFKGLCYFTNGTERVRGVTRHIYNREEYVRRDSDVGVYRAVTPQGRPV
AEYWNSQKEVLEGARASVDRVCRHNYEVAYRGILQRRVEPTVTISPSRTEALQHHNWLVCHVTDFYPSQIKVRW
FRNDQEETAGVMSTNLIRNGDWTFQILVMLEMTPQRGDVYTCHVEHPSLQSPITVEWRAQSESAQSKGGGGSLE
IEAAFLERENTALETRVAELRQRVQRLRNRVSQYRTRYGPLGGGK
Full-length wild-type DQB1*05: 01 (SEQ ID NO: 15)
MSWKKSLRIPGDLRVATVTLMLAILSSSLAEGRDSPEDFVYQFKGLCYFTNGTERVRGVTRHIYNREEYVRFDS
DVGVYRAVTPQGRPVAEYWNSQKEVLEGARASVDRVCRHNYEVAYRGILQRRVEPTVTISPSRTEALNHHNLLI
CSVTDFYPSQIKVRWFRNDQEETAGVVSTPLIRNGDWTFQILVMLEMTPQRGDVYTCHVEHPSLQSPITVEWRA
QSESAQSKMLSGVGGFVLGLIFLGLGLIIRQRSRKGLLH
Alpha Chain
DQA1*01: 01 Extracellular Domain (SEQ ID NO: 16)
EDIVADHVASCGVNLYQFYGPSGQYTHEFDGDEEFYVDLERKETAWRWPEFSKFGGFDPQGALRNMAVAKHNLN
IMIKRYNSTAATNEVPEVTVFSKSPVTLGQPNTLICLVDNIFPPVVNITWLSNGQSVTEGVSETSFLSKSDHSF
FKISYLTFLPSADEIYDCKVEHWGLDQPLLKHWEPEIPAPMSELTET
DQA1*01: 01 Extracellular Domain (SEQ ID NO: 17)
GAGGACATCGTGGCCGATCACGTGGCAAGCTGCGGCGTGAACCTGTACCAGTTCTACGGCCCCTCTGGCCAGTA
CACCCATGAATTTGATGGAGATGAGGAGTTCTACGTGGACCTGGAGAGGAAGGAGACTGCCTGGCGGTGGCCTG
AGTTCAGCAAATTTGGAGGTTTTGACCCGCAGGGTGCACTGAGAAACATGGCTGTGGCAAAACACAACTTGAAC
ATCATGATTAAACGCTACAACTCTACCGCTGCTACCAATGAGGTTCCTGAGGTCACAGTGTTTTCCAAGTCTCC
CGTGACACTGGGTCAGCCCAACACCCTCATTTGTCTTGTGGACAACATCTTTCCTCCTGTGGTCAACATCACAT
GGCTGAGCAATGGGCAGTCAGTCACAGAAGGTGTTTCTGAGACCAGCTTCCTCTCCAAGAGTGATCATTCCTTC
TTCAAGATCAGTTACCTCACCTTCCTCCCTTCTGCTGATGAGATTTATGACTGCAAGGTGGAGCACTGGGGCCT
GGACCAGCCTCTTCTGAAACACTGGGAGCCTGAGATTCCAGCCCCTATGTCAGAGCTCACAGAGACT
Signal Peptide; DQA1*01: 01 Extracellular Domain; Gly/Ser Linker, Zip
Sequences and His tag sequences)(SEQ ID NO: 18)
MMRPIVLVLLFATSALAEDIVADHVASCGVNLYQFYGPSGQYTHEFDGDEEFYVDLERKETAWRWPEFSKEGGF
DPQGALRNMAVAKHNLNIMIKRYNSTAATNEVPEVTVFSKSPVTLGQPNTLICLVDNIFPPVVNITWLSNGQSV
TEGVSETSFLSKSDHSFFKISYLTFLPSADEIYDCKVEHWGLDQPLLKHWEPEIPAPMSELTETGGGGSLEIRA
AFLRQRNTALRTEVAELEQEVQRLENEVSQYETRYGPLGGGKGSHHHHHH

II.A.2.a. HLA-DQ Beta Chain

In certain aspects, the HLA class II molecule comprises a DQ beta chain, wherein the DQ beta chain comprises an amino acid other than leucine at a position corresponding to amino acid residue 114 of SEQ ID NO: 11. Any amino acid other than leucine can be present at the position corresponding to amino acid residue 114 of SEQ ID NO: 11. In some aspects, the amino acid other than leucine is an amino acid comprising a hydrophobic side chain. In certain aspects, the amino acid other than leucine at the position corresponding to amino acid residue 114 of SEQ ID NO: 11 is an amino acid selected from an alanine, a valine, an isoleucine, a methionine, a phenylalanine, a tyrosine, and a tryptophan. In certain aspects, the amino acid other than leucine at the position corresponding to amino acid residue 114 of SEQ ID NO: 11 is an alanine. In certain aspects, the amino acid other than leucine at the position corresponding to amino acid residue 114 of SEQ ID NO: 11 is a valine. In certain aspects, the amino acid other than leucine at the position corresponding to amino acid residue 114 of SEQ ID NO: 11 is an isoleucine. In certain aspects, the amino acid other than leucine at the position corresponding to amino acid residue 114 of SEQ ID NO: 11 is a methionine. In certain aspects, the amino acid other than leucine at the position corresponding to amino acid residue 114 of SEQ ID NO: 11 is a phenylalanine. In certain aspects, the amino acid other than leucine at the position corresponding to amino acid residue 114 of SEQ ID NO: 11 is a tyrosine. In certain aspects, the amino acid other than leucine at the position corresponding to amino acid residue 114 of SEQ ID NO: 11 is a tryptophan.

In some embodiments, the amino acid other than leucine at the position corresponding to amino acid residue 114 of SEQ ID NO: 11 consists of more than one amino acid, e.g., two amino acids, three amino acids, four amino acids, five amino acids, or more. In some aspects at least one of the more than one amino acids comprises a hydrophobic side chain. In certain aspects, the amino acid other than leucine at the position corresponding to amino acid residue 114 of SEQ ID NO: 11 consists of a series, e.g., at least 2, at least 3, at least 4, or at least 5, amino acids, wherein each of the series of amino acids comprises a hydrophobic side chain.

In certain aspects, the HLA class II molecule comprises a DQ beta chain, wherein the DQ beta chain comprises an amino acid other than valine at a position corresponding to amino acid residue 143 of SEQ ID NO: 11. Any amino acid other than valine can be present at the position corresponding to amino acid residue 143 of SEQ ID NO: 11. In some aspects, the amino acid other than valine is an amino acid comprising a hydrophobic side chain. In certain aspects, the amino acid other than valine at the position corresponding to amino acid residue 143 of SEQ ID NO: 11 is an amino acid selected from an alanine, an isoleucine, a leucine, a methionine, a phenylalanine, a tyrosine, and a tryptophan. In certain aspects, the amino acid other than valine at the position corresponding to amino acid residue 143 of SEQ ID NO: 11 is an alanine. In certain aspects, the amino acid other than valine at the position corresponding to amino acid residue 143 of SEQ ID NO: 11 is an isoleucine. In certain aspects, the amino acid other than valine at the position corresponding to amino acid residue 143 of SEQ ID NO: 11 is a leucine. In certain aspects, the amino acid other than valine at the position corresponding to amino acid residue 143 of SEQ ID NO: 11 is a methionine. In certain aspects, the amino acid other than valine at the position corresponding to amino acid residue 143 of SEQ ID NO: 11 is a phenylalanine. In certain aspects, the amino acid other than valine at the position corresponding to amino acid residue 143 of SEQ ID NO: 11 is a tyrosine. In certain aspects, the amino acid other than valine at the position corresponding to amino acid residue 143 of SEQ ID NO: 11 is a tryptophan.

In some aspects, the amino acid other than valine at the position corresponding to amino acid residue 143 of SEQ ID NO: 11 consists of more than one amino acid, e.g., two amino acids, three amino acids, four amino acids, five amino acids, or more. In some aspects at least one of the more than one amino acids comprises a hydrophobic side chain. In certain aspects, the amino acid other than valine at the position corresponding to amino acid residue 143 of SEQ ID NO: 11 consists of a series, e.g., at least 2, at least 3, at least 4, or at least 5, amino acids, wherein each of the series of amino acids comprises a hydrophobic side chain.

In certain aspects, the HLA class II molecule comprises a DQ beta chain, wherein the DQ beta chain comprises an amino acid other than asparagine at a position corresponding to amino acid residue 110 of SEQ ID NO: 11. Any amino acid other than asparagine can be present at the position corresponding to amino acid residue 110 of SEQ ID NO: 11. In some aspects, the amino acid other than asparagine is an amino acid comprising a polar uncharged side chain. In certain aspects, the amino acid other than asparagine at the position corresponding to amino acid residue 110 of SEQ ID NO: 11 is an amino acid selected from a serine, a threonine, and a glutamine. In certain aspects, the amino acid other than asparagine at the position corresponding to amino acid residue 110 of SEQ ID NO: 11 is a serine. In certain aspects, the amino acid other than asparagine at the position corresponding to amino acid residue 110 of SEQ ID NO: 11 is a threonine. In certain aspects, the amino acid other than asparagine at the position corresponding to amino acid residue 110 of SEQ ID NO: 11 is a glutamine.

In some aspects, the amino acid other than asparagine at the position corresponding to amino acid residue 110 of SEQ ID NO: 11 consists of more than one amino acid, e.g., two amino acids, three amino acids, four amino acids, five amino acids, or more. In some aspects at least one of the more than one amino acids comprises a polar uncharged side chain. In certain aspects, the amino acid other than asparagine at the position corresponding to amino acid residue 110 of SEQ ID NO: 11 consists of a series, e.g., at least 2, at least 3, at least 4, or at least 5, amino acids, wherein each of the series of amino acids comprises a polar uncharged side chain.

In certain aspects, the HLA class II molecule comprises a DQ beta chain, wherein the DQ beta chain comprises an amino acid other than isoleucine at a position corresponding to amino acid residue 116 of SEQ ID NO: 11. Any amino acid other than isoleucine can be present at the position corresponding to amino acid residue 116 of SEQ ID NO: 11. In some aspects, the amino acid other than isoleucine is an amino acid comprising a hydrophobic side chain. In certain aspects, the amino acid other than isoleucine at the position corresponding to amino acid residue 116 of SEQ ID NO: 11 is an amino acid selected from an alanine, a valine, a leucine, a methionine, a phenylalanine, a tyrosine, and a tryptophan. In certain aspects, the amino acid other than isoleucine at the position corresponding to amino acid residue 116 of SEQ ID NO: 11 is an alanine. In certain aspects, the amino acid other than isoleucine at the position corresponding to amino acid residue 116 of SEQ ID NO: 11 is a valine. In certain aspects, the amino acid other than isoleucine at the position corresponding to amino acid residue 116 of SEQ ID NO: 11 is a leucine. In certain aspects, the amino acid other than isoleucine at the position corresponding to amino acid residue 116 of SEQ ID NO: 11 is a methionine. In certain aspects, the amino acid other than isoleucine at the position corresponding to amino acid residue 116 of SEQ ID NO: 11 is a phenylalanine. In certain aspects, the amino acid other than isoleucine at the position corresponding to amino acid residue 116 of SEQ ID NO: 11 is a tyrosine. In certain aspects, the amino acid other than isoleucine at the position corresponding to amino acid residue 116 of SEQ ID NO: 11 is a tryptophan.

In some aspects, the amino acid other than isoleucine at the position corresponding to amino acid residue 116 of SEQ ID NO: 11 consists of more than one amino acid, e.g., two amino acids, three amino acids, four amino acids, five amino acids, or more. In some aspects at least one of the more than one amino acids comprises a hydrophobic side chain. In certain aspects, the amino acid other than isoleucine at the position corresponding to amino acid residue 116 of SEQ ID NO: 11 consists of a series, e.g., at least 2, at least 3, at least 4, or at least 5, amino acids, wherein each of the series of amino acids comprises a hydrophobic side chain.

In certain aspects, the HLA class II molecule comprises a DQ beta chain, wherein the DQ beta chain comprises an amino acid other than serine at a position corresponding to amino acid residue 118 of SEQ ID NO: 11. Any amino acid other than serine can be present at the position corresponding to amino acid residue 118 of SEQ ID NO: 11. In some aspects, the amino acid other than serine is an amino acid comprising an electrically charged side chain. In certain aspects, the amino acid other than serine at the position corresponding to amino acid residue 118 of SEQ ID NO: 11 is an amino acid selected from an arginine, a histidine, and a lysine. In certain aspects, the amino acid other than serine at the position corresponding to amino acid residue 118 of SEQ ID NO: 11 is an arginine. In certain aspects, the amino acid other than serine at the position corresponding to amino acid residue 118 of SEQ ID NO: 11 is a histidine. In certain aspects, the amino acid other than serine at the position corresponding to amino acid residue 118 of SEQ ID NO: 11 is a lysine.

In some aspects, the amino acid other than serine at the position corresponding to amino acid residue 118 of SEQ ID NO: 11 consists of more than one amino acid, e.g., two amino acids, three amino acids, four amino acids, five amino acids, or more. In some aspects at least one of the more than one amino acids comprises an electrically charged side chain. In certain aspects, the amino acid other than serine at the position corresponding to amino acid residue 118 of SEQ ID NO: 11 consists of a series, e.g., at least 2, at least 3, at least 4, or at least 5, amino acids, wherein each of the series of amino acids comprises an electrically charged side chain.

In certain aspects, the HLA class II molecule comprises a DQ beta chain, wherein the DQ beta chain comprises an amino acid other than proline at a position corresponding to amino acid residue 146 of SEQ ID NO: 11. Any amino acid other than proline can be present at the position corresponding to amino acid residue 146 of SEQ ID NO: 11. In some aspects, the amino acid other than proline is an amino acid comprising a polar uncharged side chain. In certain aspects, the amino acid other than proline at the position corresponding to amino acid residue 146 of SEQ ID NO: 11 is an amino acid selected from a serine, a threonine, an asparagine, and a glutamine. In certain aspects, the amino acid other than proline at the position corresponding to amino acid residue 146 of SEQ ID NO: 11 is a serine. In certain aspects, the amino acid other than proline at the position corresponding to amino acid residue 146 of SEQ ID NO: 11 is a threonine. In certain aspects, the amino acid other than proline at the position corresponding to amino acid residue 146 of SEQ ID NO: 11 is an asparagine. In certain aspects, the amino acid other than proline at the position corresponding to amino acid residue 146 of SEQ ID NO: 11 is a glutamine.

In some aspects, the amino acid other than proline at the position corresponding to amino acid residue 146 of SEQ ID NO: 11 consists of more than one amino acid, e.g., two amino acids, three amino acids, four amino acids, five amino acids, or more. In some aspects at least one of the more than one amino acids comprises a polar uncharged side chain. In certain aspects, the amino acid other than proline at the position corresponding to amino acid residue 146 of SEQ ID NO: 11 consists of a series, e.g., at least 2, at least 3, at least 4, or at least 5, amino acids, wherein each of the series of amino acids comprises a polar uncharged side chain.

In certain aspects of the present disclosure, the MHC class II molecule comprises a DQ beta chain comprising more than one substitution mutation relative to the wild-type DQ beta chain. In certain aspects, the DQ beta chain comprises at least two mutations, at least three mutations, at least four mutations, at least five mutations, at least six mutations, at least seven mutations, at least eight mutations, at least nine mutations, or at least ten mutations relative to the wild-type DQ beta chain.

In certain aspects, the DQ beta chain comprises an amino acid other than leucine at a position corresponding to amino acid residue 114 of SEQ ID NO: 11 and an amino acid other than valine at a position corresponding to amino acid residue 143 of SEQ ID NO: 11. In certain aspects, the DQ beta chain comprises an amino acid other than leucine at a position corresponding to amino acid residue 114 of SEQ ID NO: 11; an amino acid other than valine at a position corresponding to amino acid residue 143 of SEQ ID NO: 11; and at least three of: (i) an amino acid other than asparagine at a position corresponding to amino acid residue 110 of SEQ ID NO: 11, (ii) an amino acid other than isoleucine at a position corresponding to amino acid residue 116 of SEQ ID NO: 11, (iii) an amino acid other than serine at a position corresponding to amino acid residue 118 of SEQ ID NO: 11, and (iv) an amino acid other than proline at a position corresponding to amino acid residue 146 of SEQ ID NO: 11.

In some aspects, the DQ beta chain comprises (i) an amino acid other than leucine at a position corresponding to amino acid residue 114 of SEQ ID NO: 11; (ii) an amino acid other than valine at a position corresponding to amino acid residue 143 of SEQ ID NO: 11; (iii) an amino acid other than asparagine at a position corresponding to amino acid residue 110 of SEQ ID NO: 11; (iv) an amino acid other than isoleucine at a position corresponding to amino acid residue 116 of SEQ ID NO: 11; (v) an amino acid other than serine at a position corresponding to amino acid residue 118 of SEQ ID NO: 11; and (vi) an amino acid other than proline at a position corresponding to amino acid residue 146 of SEQ ID NO: 11.

In some aspects, (i) the amino acid other than leucine at the position corresponding to amino acid residue 114 of SEQ ID NO: 11, (ii) the amino acid other than valine at a position corresponding to amino acid residue 143 of SEQ ID NO: 11, or each of the amino acid other than leucine at the position corresponding to amino acid residue 114 of SEQ ID NO: 11 and the amino acid other than valine at a position corresponding to amino acid residue 143 of SEQ ID NO: 11 is an amino acid comprising a hydrophobic side chain.

In some aspects, (i) the amino acid other than leucine at the position corresponding to amino acid residue 114 of SEQ ID NO: 11 is selected from an alanine, a valine, an isoleucine, a methionine, a phenylalanine, a tyrosine, and a tryptophan; (ii) the amino acid other than valine at a position corresponding to amino acid residue 143 of SEQ ID NO: 11 is selected from an alanine, an isoleucine, a leucine, a methionine, a phenylalanine, a tyrosine, and a tryptophan; (iii) the amino acid other than asparagine at a position corresponding to amino acid residue 110 of SEQ ID NO: 11 is selected from a serine, a threonine, and a glutamine; (iv) the amino acid other than isoleucine at a position corresponding to amino acid residue 116 of SEQ ID NO: 11 is selected from an alanine, a valine, a leucine, a methionine, a phenylalanine, a tyrosine, and a tryptophan; (v) the amino acid other than serine at a position corresponding to amino acid residue 118 of SEQ ID NO: 11 is selected from an arginine, a histidine, and a lysine; and (vi) the amino acid other than proline at a position corresponding to amino acid residue 146 of SEQ ID NO: 11 is selected from a serine, a threonine, an asparagine, and a glutamine.

In some aspects, (i) the amino acid other than leucine at the position corresponding to amino acid residue 114 of SEQ ID NO: 11 is a tryptophan; (ii) the amino acid other than valine at a position corresponding to amino acid residue 143 of SEQ ID NO: 11 is selected from an alanine, an isoleucine, a leucine, a methionine, a phenylalanine, a tyrosine, and a tryptophan. In some aspects, (i) the amino acid other than leucine at the position corresponding to amino acid residue 114 of SEQ ID NO: 11 is selected from an alanine, a valine, an isoleucine, a methionine, a phenylalanine, a tyrosine, and a tryptophan; and (ii) the amino acid other than valine at a position corresponding to amino acid residue 143 of SEQ ID NO: 11 is a methionine. In some aspects, (i) the amino acid other than leucine at the position corresponding to amino acid residue 114 of SEQ ID NO: 11 is a tryptophan; and (ii) the amino acid other than valine at a position corresponding to amino acid residue 143 of SEQ ID NO: 11 is a methionine. In some aspects, (i) the amino acid other than leucine at the position corresponding to amino acid residue 114 of SEQ ID NO: 11 is a tryptophan; (ii) the amino acid other than valine at a position corresponding to amino acid residue 143 of SEQ ID NO: 11 is a methionine; (iii) the amino acid other than asparagine at a position corresponding to amino acid residue 110 of SEQ ID NO: 11 is a glutamine; (iv) the amino acid other than isoleucine at a position corresponding to amino acid residue 116 of SEQ ID NO: 11 is a valine; (v) the amino acid other than serine at a position corresponding to amino acid residue 118 of SEQ ID NO: 11 is a histidine; and (vi) the amino acid other than proline at a position corresponding to amino acid residue 146 of SEQ ID NO: 11 is a glutamine.

In certain aspects, a DQ beta chain described herein has an increased affinity for a CD4 protein as compared to a reference HLA class II molecule. In some aspects, the reference HLA class II molecule is an HLA class II molecule having a wild-type DQ beta chain. In some aspects, the reference HLA class II molecule is an HLA class II molecule having a DQ beta chain comprising (i) a leucine at a position corresponding to amino acid residue 114 of SEQ ID NO: 11 and/or (ii) a valine at a position corresponding to amino acid residue 143 of SEQ ID NO: 11. In some aspects, the reference HLA class II molecule is an HLA class II molecule having a DQ beta chain comprising (i) a leucine at a position corresponding to amino acid residue 114 of SEQ ID NO: 11, (ii) a valine at a position corresponding to amino acid residue 143 of SEQ ID NO: 11, (iii) an asparagine at a position corresponding to amino acid residue 110 of SEQ ID NO: 11, (iv) an isoleucine at a position corresponding to amino acid residue 116 of SEQ ID NO: 11, (iii) a serine at a position corresponding to amino acid residue 118 of SEQ ID NO: 11, and/or (iv) a proline at a position corresponding to amino acid residue 146 of SEQ ID NO: 11.

In some aspects, the increased affinity for CD4 is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, at least about 4000-fold, at least about 4500-fold, or at least about 4000-fold greater than the affinity of the reference HLA class II molecule for CD4.

In some aspects, the increased affinity for CD4 is at least about 1.5-fold to at least about 5000-fold, 1.5-fold to at least about 4000-fold, 1.5-fold to at least about 3000-fold, 1.5-fold to at least about 2000-fold, 1.5-fold to at least about 1000-fold, 10-fold to at least about 5000-fold, 10-fold to at least about 4000-fold, 10-fold to at least about 3000-fold, 10-fold to at least about 2000-fold, 10-fold to at least about 1000-fold, 10-fold to at least about 900-fold, 10-fold to at least about 800-fold, 10-fold to at least about 700-fold, 10-fold to at least about 600-fold, 10-fold to at least about 500-fold, 10-fold to at least about 400-fold, 10-fold to at least about 300-fold, 10-fold to at least about 200-fold, 10-fold to at least about 100-fold, 100-fold to at least about 5000-fold, 100-fold to at least about 4000-fold, 100-fold to at least about 3000-fold, 100-fold to at least about 2000-fold, 100-fold to at least about 1000-fold, 100-fold to at least about 900-fold, 100-fold to at least about 800-fold, 100-fold to at least about 700-fold, 100-fold to at least about 600-fold, 100-fold to at least about 500-fold, 100-fold to at least about 400-fold, 100-fold to at least about 300-fold, or 100-fold to at least about 200-fold greater than the affinity of the reference HLA class II molecule for CD4.

In certain aspects, the DQ beta chain comprises an allele selected from an HLA-DQB1*02, an HLA-DQB1*03, an HLA-DQB1*04, an HLA-DQB1*05, and an HLA-DQB1*06 allele. In certain aspects, the DQ beta chain comprises an HLA-DQB1*05 allele. In particular aspects, the DQ beta chain comprises an HLA-DQB1*05:01 allele.

In certain aspects, the DQ beta chain comprises an allele selected from DQB1*02:01:01, DQB1*02:01:02, DQB1*02:01:03, DQB1*02:01:04, DQB1*02:01:05, DQB1*02:01:06, DQB1*02:01:07, DQB1*02:01:08, DQB1*02:01:09, DQB1*02:01:10, DQB1*02:01:11, DQB1*02:01:12, DQB1*02:01:13, DQB1*02:01:14, DQB1*02:01:15, DQB1*02:01:16, DQB1*02:01:17, DQB1*02:01:18, DQB1*02:01:19, DQB1*02:01:20, DQB1*02:01:21, DQB1*02:01:22, DQB1*02:01:23, DQB1*02:01:24, DQB1*02:01:25, DQB1*02:01:26, DQB1*02:01:27, DQB1*02:01:28, DQB1*02:01:29, DQB1*02:01:30, DQB1*02:01:31, DQB1*02:02:01:01, DQB1*02:02:01:02, DQB1*02:02:01:03, DQB1*02:02:01:04, DQB1*02:02:02, DQB1*02:02:03, DQB1*02:02:04, DQB1*02:02:05, DQB1*02:02:06, DQB1*02:02:07, DQB1*02:02:08, DQB1*02:02:09, DQB1*02:03:01, DQB1*02:03:02, DQB1*02:04, DQB1*02:05, DQB1*02:06, DQB1*02:07:01, DQB1*02:07:02, DQB1*02:08, DQB1*02:09, DQB1*02:10, DQB1*02:100, DQB1*02:101, DQB1*02:102, DQB1*02:103, DQB1*02:104, DQB1*02:105, DQB1*02:106, DQB1*02:107, DQB1*02:108, DQB1*02:109, DQB1*02:11, DQB1*02:110, DQB1*02:111, DQB1*02:112, DQB1*02:113, DQB1*02:114, DQB1*02:115, DQB1*02:116, DQB1*02:117, DQB1*02:118, DQB1*02:119, DQB1*02:12, DQB1*02:120, DQB1*02:121, DQB1*02:122, DQB1*02:123, DQB1*02:124, DQB1*02:125, DQB1*02:126, DQB1*02:127, DQB1*02:128, DQB1*02:129N, DQB1*02:13, DQB1*02:130, DQB1*02:131, DQB1*02:132N, DQB1*02:133, DQB1*02:134N, DQB1*02:135, DQB1*02:136, DQB1*02:137, DQB1*02:138, DQB1*02:139, DQB1*02:140, DQB1*02:141, DQB1*02:142, DQB1*02:14:01, DQB1*02:14:02, DQB1*02:15, DQB1*02:16, DQB1*02:17, DQB1*02:18N, DQB1*02:19, DQB1*02:20N, DQB1*02:21, DQB1*02:22, DQB1*02:23, DQB1*02:24, DQB1*02:25, DQB1*02:26, DQB1*02:27, DQB1*02:28, DQB1*02:29, DQB1*02:30, DQB1*02:31, DQB1*02:32, DQB1*02:33, DQB1*02:34, DQB1*02:35, DQB1*02:36, DQB1*02:37, DQB1*02:38, DQB1*02:39, DQB1*02:40, DQB1*02:41, DQB1*02:42, DQB1*02:43, DQB1*02:44, DQB1*02:45, DQB1*02:46, DQB1*02:47, DQB1*02:48, DQB1*02:49, DQB1*02:50, DQB1*02:51, DQB1*02:52, DQB1*02:53Q, DQB1*02:54, DQB1*02:55, DQB1*02:56, DQB1*02:57, DQB1*02:58N, DQB1*02:59, DQB1*02:60, DQB1*02:61, DQB1*02:62, DQB1*02:63, DQB1*02:64, DQB1*02:65, DQB1*02:66, DQB1*02:67NX, DQB1*02:68, DQB1*02:69, DQB1*02:70, DQB1*02:71, DQB1*02:72, DQB1*02:73, DQB1*02:74, DQB1*02:75, DQB1*02:76, DQB1*02:77, DQB1*02:78, DQB1*02:79, DQB1*02:80, DQB1*02:81, DQB1*02:82, DQB1*02:83, DQB1*02:84, DQB1*02:85, DQB1*02:86, DQB1*02:87, DQB1*02:88, DQB1*02:89:01, DQB1*02:89:02, DQB1*02:90, DQB1*02:91, DQB1*02:92, DQB1*02:93, DQB1*02:94, DQB1*02:95, DQB1*02:96N, DQB1*02:97, DQB1*02:98, DQB1*02:99, DQB1*03:01:01:01, DQB1*03:01:01:02, DQB1*03:01:01:03, DQB1*03:01:01:04, DQB1*03:01:01:05, DQB1*03:01:01:06, DQB1*03:01:01:07, DQB1*03:01:01:08, DQB1*03:01:01:09, DQB1*03:01:01:10, DQB1*03:01:01:11, DQB1*03:01:01:12, DQB1*03:01:01:14, DQB1*03:01:01:15, DQB1*03:01:01:16, DQB1*03:01:01:17, DQB1*03:01:01:18, DQB1*03:01:01:19, DQB1*03:01:01:20, DQB1*03:01:02, DQB1*03:01:03, DQB1*03:01:04, DQB1*03:01:05, DQB1*03:01:06, DQB1*03:01:07, DQB1*03:01:08, DQB1*03:01:09, DQB1*03:01:10, DQB1*03:01:11, DQB1*03:01:12, DQB1*03:01:13, DQB1*03:01:14, DQB1*03:01:15, DQB1*03:01:16, DQB1*03:01:17, DQB1*03:01:18, DQB1*03:01:19, DQB1*03:01:20, DQB1*03:01:21, DQB1*03:01:22, DQB1*03:01:23, DQB1*03:01:24, DQB1*03:01:25, DQB1*03:01:26, DQB1*03:01:27, DQB1*03:01:28, DQB1*03:01:29, DQB1*03:01:30, DQB1*03:01:31, DQB1*03:01:32, DQB1*03:01:33, DQB1*03:01:34, DQB1*03:01:35, DQB1*03:01:36, DQB1*03:01:37, DQB1*03:01:38, DQB1*03:01:39, DQB1*03:01:40, DQB1*03:01:41, DQB1*03:01:42, DQB1*03:01:43, DQB1*03:01:44, DQB1*03:01:45, DQB1*03:01:46, DQB1*03:02:01:01, DQB1*03:02:01:02, DQB1*03:02:01:03, DQB1*03:02:01:04, DQB1*03:02:01:05, DQB1*03:02:01:06, DQB1*03:02:01:07, DQB1*03:02:01:08, DQB1*03:02:02, DQB1*03:02:03, DQB1*03:02:04, DQB1*03:02:05, DQB1*03:02:06, DQB1*03:02:07, DQB1*03:02:08, DQB1*03:02:09, DQB1*03:02:10, DQB1*03:02:11, DQB1*03:02:12, DQB1*03:02:13, DQB1*03:02:14, DQB1*03:02:15, DQB1*03:02:16, DQB1*03:02:17, DQB1*03:02:18, DQB1*03:02:19, DQB1*03:02:20, DQB1*03:02:21, DQB1*03:02:22, DQB1*03:02:23, DQB1*03:02:24, DQB1*03:02:25, DQB1*03:02:26, DQB1*03:02:27, DQB1*03:02:28, DQB1*03:02:29, DQB1*03:02:30, DQB1*03:03:02:01, DQB1*03:03:02:02, DQB1*03:03:02:03, DQB1*03:03:02:04, DQB1*03:03:02:05, DQB1*03:03:03, DQB1*03:03:04, DQB1*03:03:05, DQB1*03:03:06, DQB1*03:03:07, DQB1*03:03:08, DQB1*03:03:09, DQB1*03:03:10, DQB1*03:03:11, DQB1*03:03:12, DQB1*03:03:13, DQB1*03:03:14, DQB1*03:03:15, DQB1*03:03:16, DQB1*03:03:17, DQB1*03:03:18, DQB1*03:03:19, DQB1*03:03:20, DQB1*03:03:21, DQB1*03:04:01, DQB1*03:04:02, DQB1*03:04:03, DQB1*03:04:04, DQB1*03:05:01, DQB1*03:05:02, DQB1*03:05:03, DQB1*03:05:04, DQB1*03:06, DQB1*03:07, DQB1*03:08, DQB1*03:09, DQB1*03:100, DQB1*03:101, DQB1*03:102, DQB1*03:103, DQB1*03:104, DQB1*03:105, DQB1*03:106, DQB1*03:107, DQB1*03:108, DQB1*03:109, DQB1*03:10:01, DQB1*03:10:02:01, DQB1*03:10:02:02, DQB1*03:11, DQB1*03:110, DQB1*03:111, DQB1*03:112, DQB1*03:113, DQB1*03:114, DQB1*03:115, DQB1*03:116, DQB1*03:117, DQB1*03:118N, DQB1*03:119, DQB1*03:12, DQB1*03:120, DQB1*03:121, DQB1*03:122, DQB1*03:123, DQB1*03:124, DQB1*03:125, DQB1*03:126, DQB1*03:127, DQB1*03:128, DQB1*03:129, DQB1*03:13, DQB1*03:130, DQB1*03:131, DQB1*03:132, DQB1*03:133, DQB1*03:134, DQB1*03:135, DQB1*03:136, DQB1*03:137, DQB1*03:138, DQB1*03:139, DQB1*03:140, DQB1*03:141, DQB1*03:142, DQB1*03:143, DQB1*03:144, DQB1*03:145, DQB1*03:146, DQB1*03:147, DQB1*03:148, DQB1*03:149, DQB1*03:14:01, DQB1*03:14:02, DQB1*03:15, DQB1*03:150, DQB1*03:151, DQB1*03:152, DQB1*03:153, DQB1*03:154, DQB1*03:155, DQB1*03:156, DQB1*03:157, DQB1*03:158, DQB1*03:159, DQB1*03:16, DQB1*03:160, DQB1*03:161, DQB1*03:162, DQB1*03:163, DQB1*03:164, DQB1*03:165, DQB1*03:166, DQB1*03:167, DQB1*03:168, DQB1*03:169, DQB1*03:170, DQB1*03:171, DQB1*03:172, DQB1*03:173, DQB1*03:174, DQB1*03:175, DQB1*03:176, DQB1*03:177, DQB1*03:178, DQB1*03:179, DQB1*03:17:01, DQB1*03:17:02, DQB1*03:18, DQB1*03:180, DQB1*03:181, DQB1*03:182, DQB1*03:183, DQB1*03:184, DQB1*03:185, DQB1*03:186, DQB1*03:187, DQB1*03:188, DQB1*03:189, DQB1*03:190, DQB1*03:191, DQB1*03:192, DQB1*03:193, DQB1*03:194, DQB1*03:195, DQB1*03:196, DQB1*03:197Q, DQB1*03:198:01, DQB1*03:198:02, DQB1*03:199, DQB1*03:19:01, DQB1*03:19:02, DQB1*03:19:03, DQB1*03:19:04, DQB1*03:20, DQB1*03:200, DQB1*03:201, DQB1*03:202, DQB1*03:203, DQB1*03:204, DQB1*03:205, DQB1*03:206, DQB1*03:207, DQB1*03:208, DQB1*03:209, DQB1*03:21, DQB1*03:210, DQB1*03:211, DQB1*03:212, DQB1*03:213NX, DQB1*03:214, DQB1*03:215, DQB1*03:216, DQB1*03:217, DQB1*03:218, DQB1*03:219, DQB1*03:220, DQB1*03:221, DQB1*03:222, DQB1*03:223, DQB1*03:224, DQB1*03:225, DQB1*03:226, DQB1*03:227, DQB1*03:228, DQB1*03:229, DQB1*03:22:01, DQB1*03:22:02, DQB1*03:230, DQB1*03:231, DQB1*03:232, DQB1*03:233, DQB1*03:234, DQB1*03:235, DQB1*03:236, DQB1*03:237N, DQB1*03:238, DQB1*03:239, DQB1*03:23:01, DQB1*03:23:02, DQB1*03:23:03, DQB1*03:24, DQB1*03:240, DQB1*03:241, DQB1*03:242, DQB1*03:243, DQB1*03:244, DQB1*03:245, DQB1*03:246, DQB1*03:247, DQB1*03:248, DQB1*03:249, DQB1*03:250, DQB1*03:251, DQB1*03:252, DQB1*03:253, DQB1*03:254, DQB1*03:255, DQB1*03:256, DQB1*03:257, DQB1*03:258, DQB1*03:259, DQB1*03:25:01, DQB1*03:25:02, DQB1*03:26, DQB1*03:260, DQB1*03:261, DQB1*03:262, DQB1*03:263, DQB1*03:264, DQB1*03:265, DQB1*03:266, DQB1*03:267, DQB1*03:268, DQB1*03:269N, DQB1*03:27, DQB1*03:270, DQB1*03:271, DQB1*03:272, DQB1*03:273, DQB1*03:274, DQB1*03:275, DQB1*03:277, DQB1*03:278, DQB1*03:279, DQB1*03:28, DQB1*03:280, DQB1*03:281, DQB1*03:282N, DQB1*03:283, DQB1*03:284, DQB1*03:285, DQB1*03:286, DQB1*03:287, DQB1*03:288, DQB1*03:289, DQB1*03:29, DQB1*03:290, DQB1*03:291, DQB1*03:292, DQB1*03:293, DQB1*03:294, DQB1*03:295, DQB1*03:296, DQB1*03:297, DQB1*03:298, DQB1*03:299, DQB1*03:30, DQB1*03:300, DQB1*03:301, DQB1*03:302, DQB1*03:303N, DQB1*03:304, DQB1*03:305, DQB1*03:306, DQB1*03:307, DQB1*03:308, DQB1*03:309, DQB1*03:31, DQB1*03:310N, DQB1*03:311, DQB1*03:312, DQB1*03:313, DQB1*03:314, DQB1*03:315, DQB1*03:316, DQB1*03:317:01, DQB1*03:317:02, DQB1*03:318, DQB1*03:319, DQB1*03:32, DQB1*03:320, DQB1*03:321, DQB1*03:322, DQB1*03:323, DQB1*03:324, DQB1*03:326, DQB1*03:327, DQB1*03:328, DQB1*03:329, DQB1*03:33, DQB1*03:330, DQB1*03:331, DQB1*03:332, DQB1*03:333, DQB1*03:334N4 bp, DQB1*03:335, DQB1*03:336, DQB1*03:337, DQB1*03:338N, DQB1*03:339N, DQB1*03:34, DQB1*03:340N, DQB1*03:341, DQB1*03:342, DQB1*03:343, DQB1*03:344, DQB1*03:345, DQB1*03:346, DQB1*03:347, DQB1*03:348, DQB1*03:349, DQB1*03:35, DQB1*03:350, DQB1*03:351, DQB1*03:352, DQB1*03:353, DQB1*03:354N, DQB1*03:355, DQB1*03:356NX, DQB1*03:357N, DQB1*03:358N, DQB1*03:36, DQB1*03:37, DQB1*03:38:01, DQB1*03:38:02, DQB1*03:39, DQB1*03:40, DQB1*03:41, DQB1*03:42, DQB1*03:43, DQB1*03:44, DQB1*03:45, DQB1*03:46, DQB1*03:47, DQB1*03:48, DQB1*03:49, DQB1*03:50, DQB1*03:51, DQB1*03:52, DQB1*03:53, DQB1*03:54, DQB1*03:55, DQB1*03:56, DQB1*03:57, DQB1*03:58, DQB1*03:59, DQB1*03:60, DQB1*03:61, DQB1*03:62, DQB1*03:63, DQB1*03:64, DQB1*03:65, DQB1*03:66N, DQB1*03:67, DQB1*03:68, DQB1*03:69, DQB1*03:70, DQB1*03:71, DQB1*03:72, DQB1*03:73, DQB1*03:74, DQB1*03:75, DQB1*03:76, DQB1*03:77, DQB1*03:78, DQB1*03:79, DQB1*03:80, DQB1*03:81, DQB1*03:82, DQB1*03:83, DQB1*03:84N, DQB1*03:85, DQB1*03:86, DQB1*03:87, DQB1*03:88, DQB1*03:89, DQB1*03:90N, DQB1*03:91Q, DQB1*03:92, DQB1*03:93, DQB1*03:94, DQB1*03:95N, DQB1*03:96, DQB1*03:97, DQB1*03:98, DQB1*03:99Q, DQB1*04:01:01:01, DQB1*04:01:01:02, DQB1*04:01:02, DQB1*04:01:03, DQB1*04:01:04, DQB1*04:01:05, DQB1*04:02:01:01, DQB1*04:02:01:04, DQB1*04:02:01:05, DQB1*04:02:01:06, DQB1*04:02:01:07, DQB1*04:02:01:08, DQB1*04:02:01:09, DQB1*04:02:01:10, DQB1*04:02:02, DQB1*04:02:03, DQB1*04:02:04, DQB1*04:02:05, DQB1*04:02:06, DQB1*04:02:07, DQB1*04:02:08, DQB1*04:02:09, DQB1*04:02:10, DQB1*04:02:11, DQB1*04:02:12, DQB1*04:02:13, DQB1*04:02:14, DQB1*04:02:15, DQB1*04:02:16, DQB1*04:02:17, DQB1*04:02:18, DQB1*04:03:01, DQB1*04:03:02, DQB1*04:03:03, DQB1*04:04, DQB1*04:05, DQB1*04:06, DQB1*04:07, DQB1*04:08, DQB1*04:09, DQB1*04:10, DQB1*04:11, DQB1*04:12, DQB1*04:13, DQB1*04:14, DQB1*04:15, DQB1*04:16, DQB1*04:17, DQB1*04:18, DQB1*04:19, DQB1*04:20, DQB1*04:21, DQB1*04:22, DQB1*04:23, DQB1*04:24, DQB1*04:25N, DQB1*04:26, DQB1*04:27, DQB1*04:28, DQB1*04:29, DQB1*04:30, DQB1*04:31, DQB1*04:32, DQB1*04:33, DQB1*04:34, DQB1*04:35, DQB1*04:36N, DQB1*04:37, DQB1*04:38, DQB1*04:39, DQB1*04:40, DQB1*04:41N, DQB1*04:42, DQB1*04:43, DQB1*04:44, DQB1*04:45, DQB1*04:46N, DQB1*04:47, DQB1*04:48, DQB1*04:49, DQB1*04:50, DQB1*04:51, DQB1*04:52, DQB1*04:53, DQB1*04:54, DQB1*04:55, DQB1*04:56, DQB1*04:57, DQB1*04:58, DQB1*04:59N, DQB1*04:60, DQB1*04:61, DQB1*04:62, DQB1*05:01:01:01, DQB1*05:01:01:02, DQB1*05:01:01:03, DQB1*05:01:01:04, DQB1*05:01:01:05, DQB1*05:01:02, DQB1*05:01:03, DQB1*05:01:04, DQB1*05:01:05, DQB1*05:01:06, DQB1*05:01:07, DQB1*05:01:08, DQB1*05:01:09, DQB1*05:01:10, DQB1*05:01:11, DQB1*05:01:12, DQB1*05:01:13, DQB1*05:01:14, DQB1*05:01:15, DQB1*05:01:16, DQB1*05:01:17, DQB1*05:01:18, DQB1*05:01:19, DQB1*05:01:20, DQB1*05:01:21, DQB1*05:01:22, DQB1*05:01:23, DQB1*05:01:24:01, DQB1*05:01:24:02, DQB1*05:01:25, DQB1*05:01:26, DQB1*05:01:27, DQB1*05:01:28, DQB1*05:01:29, DQB1*05:01:30, DQB1*05:01:31, DQB1*05:01:32, DQB1*05:01:33, DQB1*05:01:34, DQB1*05:02:01:01, DQB1*05:02:01:02, DQB1*05:02:01:03, DQB1*05:02:01:04, DQB1*05:02:01:05, DQB1*05:02:01:06, DQB1*05:02:02, DQB1*05:02:03, DQB1*05:02:04, DQB1*05:02:05, DQB1*05:02:06, DQB1*05:02:07, DQB1*05:02:08, DQB1*05:02:09, DQB1*05:02:10, DQB1*05:02:11, DQB1*05:02:12, DQB1*05:02:13, DQB1*05:02:14, DQB1*05:02:15, DQB1*05:02:16, DQB1*05:02:17, DQB1*05:02:18, DQB1*05:02:19, DQB1*05:03:01:01, DQB1*05:03:01:02, DQB1*05:03:01:03, DQB1*05:03:02, DQB1*05:03:03, DQB1*05:03:04, DQB1*05:03:05, DQB1*05:03:06, DQB1*05:03:07, DQB1*05:03:08, DQB1*05:03:09, DQB1*05:03:10, DQB1*05:03:11, DQB1*05:03:12, DQB1*05:03:13, DQB1*05:03:14, DQB1*05:03:15, DQB1*05:03:16, DQB1*05:03:17, DQB1*05:03:18, DQB1*05:03:19, DQB1*05:03:20, DQB1*05:04, DQB1*05:05:01, DQB1*05:05:02, DQB1*05:06:01, DQB1*05:06:02, DQB1*05:07, DQB1*05:08, DQB1*05:09, DQB1*05:10, DQB1*05:100, DQB1*05:101, DQB1*05:102, DQB1*05:103, DQB1*05:104, DQB1*05:105, DQB1*05:106, DQB1*05:107, DQB1*05:108, DQB1*05:109, DQB1*05:110N, DQB1*05:111, DQB1*05:112, DQB1*05:113, DQB1*05:114, DQB1*05:115, DQB1*05:116, DQB1*05:117, DQB1*05:118, DQB1*05:119, DQB1*05:11:01, DQB1*05:11:02, DQB1*05:12, DQB1*05:120, DQB1*05:121, DQB1*05:122, DQB1*05:123, DQB1*05:124, DQB1*05:125, DQB1*05:126, DQB1*05:127, DQB1*05:128N, DQB1*05:129, DQB1*05:13, DQB1*05:130, DQB1*05:131, DQB1*05:132Q, DQB1*05:133, DQB1*05:134, DQB1*05:135, DQB1*05:136, DQB1*05:137, DQB1*05:138, DQB1*05:139, DQB1*05:14, DQB1*05:140, DQB1*05:141, DQB1*05:142, DQB1*05:143, DQB1*05:144, DQB1*05:145, DQB1*05:146, DQB1*05:147, DQB1*05:148, DQB1*05:149, DQB1*05:15, DQB1*05:150, DQB1*05:151, DQB1*05:152, DQB1*05:153, DQB1*05:154, DQB1*05:155, DQB1*05:156, DQB1*05:157, DQB1*05:158, DQB1*05:159, DQB1*05:16, DQB1*05:160, DQB1*05:161, DQB1*05:162, DQB1*05:163, DQB1*05:164, DQB1*05:165, DQB1*05:166, DQB1*05:167, DQB1*05:168, DQB1*05:169, DQB1*05:17, DQB1*05:170, DQB1*05:171, DQB1*05:172, DQB1*05:173, DQB1*05:174, DQB1*05:175, DQB1*05:176, DQB1*05:177, DQB1*05:178, DQB1*05:179, DQB1*05:18, DQB1*05:180, DQB1*05:181, DQB1*05:182, DQB1*05:183, DQB1*05:184, DQB1*05:185N, DQB1*05:186, DQB1*05:187, DQB1*05:188, DQB1*05:189, DQB1*05:19, DQB1*05:190, DQB1*05:191, DQB1*05:192, DQB1*05:193, DQB1*05:194, DQB1*05:195, DQB1*05:196, DQB1*05:197, DQB1*05:198, DQB1*05:199, DQB1*05:20, DQB1*05:200, DQB1*05:201, DQB1*05:202, DQB1*05:203, DQB1*05:204, DQB1*05:205, DQB1*05:206N, DQB1*05:207, DQB1*05:208N5 bp, DQB1*05:209, DQB1*05:21, DQB1*05:210, DQB1*05:211, DQB1*05:212, DQB1*05:213, DQB1*05:214, DQB1*05:215N, DQB1*05:216, DQB1*05:217, DQB1*05:22, DQB1*05:23, DQB1*05:24, DQB1*05:25, DQB1*05:26, DQB1*05:27, DQB1*05:28, DQB1*05:29, DQB1*05:30, DQB1*05:31, DQB1*05:32, DQB1*05:33, DQB1*05:34, DQB1*05:35, DQB1*05:36, DQB1*05:37, DQB1*05:38, DQB1*05:39, DQB1*05:40, DQB1*05:41N, DQB1*05:42, DQB1*05:43:01, DQB1*05:43:02, DQB1*05:44, DQB1*05:45, DQB1*05:46, DQB1*05:47, DQB1*05:48, DQB1*05:49, DQB1*05:50, DQB1*05:51, DQB1*05:52, DQB1*05:53, DQB1*05:54, DQB1*05:55, DQB1*05:56, DQB1*05:57, DQB1*05:58, DQB1*05:59, DQB1*05:60, DQB1*05:61, DQB1*05:62, DQB1*05:63, DQB1*05:64, DQB1*05:65, DQB1*05:66:01, DQB1*05:66:02, DQB1*05:67, DQB1*05:68, DQB1*05:69, DQB1*05:70, DQB1*05:71, DQB1*05:72, DQB1*05:73, DQB1*05:74, DQB1*05:75, DQB1*05:76, DQB1*05:77, DQB1*05:78, DQB1*05:79, DQB1*05:80, DQB1*05:81, DQB1*05:82, DQB1*05:83, DQB1*05:84, DQB1*05:85, DQB1*05:86, DQB1*05:87Q, DQB1*05:88, DQB1*05:89:01, DQB1*05:89:02, DQB1*05:90N, DQB1*05:91, DQB1*05:92, DQB1*05:93, DQB1*05:94, DQB1*05:95, DQB1*05:96, DQB1*05:97, DQB1*05:98, DQB1*05:99, DQB1*06:01:01:01, DQB1*06:01:01:02, DQB1*06:01:02, DQB1*06:01:03, DQB1*06:01:04, DQB1*06:01:05, DQB1*06:01:06, DQB1*06:01:07, DQB1*06:01:08, DQB1*06:01:09, DQB1*06:01:10, DQB1*06:01:11, DQB1*06:01:12, DQB1*06:01:13, DQB1*06:01:14, DQB1*06:01:15, DQB1*06:01:16, DQB1*06:01:17, DQB1*06:01:18, DQB1*06:01:19, DQB1*06:01:20, DQB1*06:01:21, DQB1*06:02:01:01, DQB1*06:02:01:02, DQB1*06:02:01:03, DQB1*06:02:01:04, DQB1*06:02:02, DQB1*06:02:03, DQB1*06:02:04, DQB1*06:02:05, DQB1*06:02:06, DQB1*06:02:07, DQB1*06:02:08, DQB1*06:02:09, DQB1*06:02:10, DQB1*06:02:11, DQB1*06:02:12, DQB1*06:02:13, DQB1*06:02:14, DQB1*06:02:15, DQB1*06:02:16, DQB1*06:02:17, DQB1*06:02:18, DQB1*06:02:19, DQB1*06:02:20, DQB1*06:02:21, DQB1*06:02:22, DQB1*06:02:23, DQB1*06:02:24, DQB1*06:02:25, DQB1*06:02:26, DQB1*06:02:27, DQB1*06:02:28, DQB1*06:02:29, DQB1*06:02:30, DQB1*06:02:31, DQB1*06:02:32, DQB1*06:02:33, DQB1*06:02:34, DQB1*06:02:35, DQB1*06:02:36, DQB1*06:02:37, DQB1*06:02:38, DQB1*06:03:01:01, DQB1*06:03:01:02, DQB1*06:03:01:03, DQB1*06:03:02, DQB1*06:03:03, DQB1*06:03:04, DQB1*06:03:05, DQB1*06:03:06, DQB1*06:03:07, DQB1*06:03:08, DQB1*06:03:09, DQB1*06:03:10, DQB1*06:03:11, DQB1*06:03:12, DQB1*06:03:13, DQB1*06:03:14, DQB1*06:03:15, DQB1*06:03:16, DQB1*06:03:17, DQB1*06:03:18, DQB1*06:03:19, DQB1*06:03:20, DQB1*06:03:21, DQB1*06:03:22, DQB1*06:03:23, DQB1*06:03:24, DQB1*06:03:25, DQB1*06:03:26, DQB1*06:03:27, DQB1*06:03:28, DQB1*06:03:29, DQB1*06:03:30, DQB1*06:03:31, DQB1*06:03:32, DQB1*06:03:33, DQB1*06:03:34, DQB1*06:03:35, DQB1*06:04:01, DQB1*06:04:02, DQB1*06:04:03, DQB1*06:04:04, DQB1*06:04:05, DQB1*06:04:06, DQB1*06:04:07, DQB1*06:04:08, DQB1*06:04:09, DQB1*06:04:10, DQB1*06:04:11, DQB1*06:04:12, DQB1*06:05:01, DQB1*06:05:02, DQB1*06:06, DQB1*06:07:01, DQB1*06:07:02, DQB1*06:08:01, DQB1*06:08:02, DQB1*06:08:03, DQB1*06:09:01:01, DQB1*06:09:01:02, DQB1*06:09:02, DQB1*06:09:03, DQB1*06:09:04, DQB1*06:09:05, DQB1*06:09:06, DQB1*06:09:07, DQB1*06:09:08, DQB1*06:09:09, DQB1*06:09:10, DQB1*06:10, DQB1*06:100, DQB1*06:101, DQB1*06:102N, DQB1*06:103, DQB1*06:104, DQB1*06:105, DQB1*06:106, DQB1*06:107, DQB1*06:108, DQB1*06:109, DQB1*06:110, DQB1*06:111, DQB1*06:112N, DQB1*06:113, DQB1*06:114, DQB1*06:115, DQB1*06:116, DQB1*06:117, DQB1*06:118:01, DQB1*06:118:02, DQB1*06:118:03, DQB1*06:119, DQB1*06:11:01, DQB1*06:11:02, DQB1*06:11:03, DQB1*06:11:04, DQB1*06:12, DQB1*06:120, DQB1*06:121, DQB1*06:122, DQB1*06:123, DQB1*06:124, DQB1*06:125, DQB1*06:126, DQB1*06:127, DQB1*06:128, DQB1*06:129, DQB1*06:130, DQB1*06:131, DQB1*06:132, DQB1*06:133, DQB1*06:134, DQB1*06:135, DQB1*06:136, DQB1*06:137, DQB1*06:138, DQB1*06:139, DQB1*06:13:01, DQB1*06:13:02, DQB1*06:13:03, DQB1*06:140, DQB1*06:141, DQB1*06:142, DQB1*06:143, DQB1*06:144N, DQB1*06:145, DQB1*06:146:01, DQB1*06:146:02, DQB1*06:147, DQB1*06:148, DQB1*06:149, DQB1*06:14:01, DQB1*06:14:02, DQB1*06:14:03, DQB1*06:150, DQB1*06:151, DQB1*06:152, DQB1*06:153:01, DQB1*06:153:02, DQB1*06:154, DQB1*06:155, DQB1*06:156, DQB1*06:157, DQB1*06:158N, DQB1*06:159, DQB1*06:15:01, DQB1*06:15:02, DQB1*06:16, DQB1*06:160, DQB1*06:161, DQB1*06:162, DQB1*06:163, DQB1*06:164, DQB1*06:165, DQB1*06:166, DQB1*06:167, DQB1*06:168, DQB1*06:169, DQB1*06:17, DQB1*06:170, DQB1*06:171, DQB1*06:172, DQB1*06:173, DQB1*06:174, DQB1*06:175, DQB1*06:176, DQB1*06:177, DQB1*06:178, DQB1*06:179N, DQB1*06:180, DQB1*06:181, DQB1*06:182, DQB1*06:183, DQB1*06:184, DQB1*06:185, DQB1*06:186, DQB1*06:187, DQB1*06:188, DQB1*06:189, DQB1*06:18:01, DQB1*06:18:02, DQB1*06:190:01, DQB1*06:190:02, DQB1*06:191, DQB1*06:192, DQB1*06:193N, DQB1*06:194, DQB1*06:195, DQB1*06:196, DQB1*06:197, DQB1*06:198, DQB1*06:199, DQB1*06:19:01, DQB1*06:19:02, DQB1*06:20, DQB1*06:200, DQB1*06:201, DQB1*06:202, DQB1*06:203, DQB1*06:204, DQB1*06:205, DQB1*06:206:01, DQB1*06:206:02, DQB1*06:207, DQB1*06:208, DQB1*06:209, DQB1*06:21, DQB1*06:210, DQB1*06:211, DQB1*06:212, DQB1*06:213, DQB1*06:214, DQB1*06:215, DQB1*06:216N, DQB1*06:217, DQB1*06:218, DQB1*06:219, DQB1*06:221, DQB1*06:222, DQB1*06:223, DQB1*06:224, DQB1*06:225, DQB1*06:226, DQB1*06:227, DQB1*06:228, DQB1*06:229, DQB1*06:22:01, DQB1*06:22:02, DQB1*06:22:03, DQB1*06:23, DQB1*06:230, DQB1*06:231, DQB1*06:232, DQB1*06:233, DQB1*06:234, DQB1*06:235, DQB1*06:236, DQB1*06:237, DQB1*06:238, DQB1*06:239, DQB1*06:24, DQB1*06:240, DQB1*06:241, DQB1*06:242, DQB1*06:243, DQB1*06:244, DQB1*06:245, DQB1*06:246, DQB1*06:247, DQB1*06:248, DQB1*06:249, DQB1*06:25, DQB1*06:250, DQB1*06:251, DQB1*06:252N, DQB1*06:253, DQB1*06:254, DQB1*06:255, DQB1*06:256, DQB1*06:257, DQB1*06:258, DQB1*06:259, DQB1*06:260, DQB1*06:261, DQB1*06:262, DQB1*06:263, DQB1*06:264, DQB1*06:265, DQB1*06:266, DQB1*06:267, DQB1*06:268, DQB1*06:269, DQB1*06:26N, DQB1*06:270:01, DQB1*06:270:02, DQB1*06:271, DQB1*06:272, DQB1*06:273, DQB1*06:274, DQB1*06:275, DQB1*06:276, DQB1*06:277, DQB1*06:278, DQB1*06:279, DQB1*06:27:01, DQB1*06:27:02, DQB1*06:28, DQB1*06:280, DQB1*06:281, DQB1*06:282, DQB1*06:283, DQB1*06:284, DQB1*06:285, DQB1*06:286, DQB1*06:287, DQB1*06:288, DQB1*06:289, DQB1*06:29, DQB1*06:290, DQB1*06:291, DQB1*06:292, DQB1*06:293, DQB1*06:294, DQB1*06:295, DQB1*06:296, DQB1*06:297, DQB1*06:298, DQB1*06:299, DQB1*06:30, DQB1*06:300, DQB1*06:301, DQB1*06:302, DQB1*06:303N, DQB1*06:304N, DQB1*06:305, DQB1*06:306N, DQB1*06:307, DQB1*06:308N, DQB1*06:309, DQB1*06:31, DQB1*06:310, DQB1*06:311, DQB1*06:312, DQB1*06:313, DQB1*06:314, DQB1*06:315, DQB1*06:316, DQB1*06:317N, DQB1*06:318, DQB1*06:319, DQB1*06:320, DQB1*06:321, DQB1*06:322, DQB1*06:323, DQB1*06:324, DQB1*06:325, DQB1*06:326, DQB1*06:32:01, DQB1*06:32:02, DQB1*06:33, DQB1*06:34, DQB1*06:35, DQB1*06:36, DQB1*06:37, DQB1*06:38, DQB1*06:39, DQB1*06:40, DQB1*06:41, DQB1*06:42, DQB1*06:43, DQB1*06:44, DQB1*06:45, DQB1*06:46, DQB1*06:47, DQB1*06:48:01, DQB1*06:48:02, DQB1*06:49, DQB1*06:50, DQB1*06:51:01, DQB1*06:51:02, DQB1*06:52, DQB1*06:53:01, DQB1*06:53:02, DQB1*06:54N, DQB1*06:55, DQB1*06:56, DQB1*06:57, DQB1*06:58, DQB1*06:59, DQB1*06:60, DQB1*06:61, DQB1*06:62, DQB1*06:63, DQB1*06:64, DQB1*06:65, DQB1*06:66, DQB1*06:67, DQB1*06:68, DQB1*06:69:01, DQB1*06:69:02, DQB1*06:70, DQB1*06:71, DQB1*06:72, DQB1*06:73, DQB1*06:74, DQB1*06:75NX, DQB1*06:76, DQB1*06:77N, DQB1*06:78, DQB1*06:79:01, DQB1*06:79:02, DQB1*06:80, DQB1*06:81, DQB1*06:82, DQB1*06:83, DQB1*06:84, DQB1*06:85, DQB1*06:86, DQB1*06:87, DQB1*06:88, DQB1*06:89, DQB1*06:90, DQB1*06:91, DQB1*06:92:01, DQB1*06:92:02, DQB1*06:93, DQB1*06:94, DQB1*06:95, DQB1*06:96:01, DQB1*06:96:02, DQB1*06:97, DQB1*06:98, DQB1*06:99:01, DQB1*06:99:02, and any combination thereof.

In certain aspects, the MHC class II molecule comprises a DQ beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 13, wherein the DQ beta chain comprises a tryptophan at a position corresponding to amino acid residue 114 of SEQ ID NO: 11, and wherein the DQ beta chain comprises a methionine at a position corresponding to amino acid residue 143 of SEQ ID NO: 11. In certain aspects, the MHC class II molecule comprises a DQ beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 13, wherein the DQ beta chain comprises (i) a tryptophan at a position corresponding to amino acid residue 114 of SEQ ID NO: 11, (ii) a methionine at a position corresponding to amino acid residue 143 of SEQ ID NO: 11, (iii) a glutamine at a position corresponding to amino acid residue 110 of SEQ ID NO: 11; (iv) a valine at a position corresponding to amino acid residue 116 of SEQ ID NO: 11; (v) a histidine at a position corresponding to amino acid residue 118 of SEQ ID NO: 11; and (vi) a glutamine at a position corresponding to amino acid residue 146 of SEQ ID NO: 11. In certain aspects, the MHC class II molecule comprises a DQ beta chain comprising an amino acid sequence set forth in SEQ ID NO: 13.

II.A.2.b. HLA-DQ Alpha Chain

In some aspects of the present disclosure, the MHC class II molecule further comprises an alpha chain. In some aspects, the alpha chain is a wild-type alpha chain. In some aspects, the alpha chain is a DQ alpha chain. Any DQ alpha chain can be used in the compositions and methods of the present disclosure. In some aspects, the DQ alpha chain comprises an HLA-DQA1*01, HLA-DQA1*02, HLA-DQA1*03, HLA-DQA1*04, HLA-DQA1*05, or HLA-DQA1*06 allele. In certain aspects, the DQ alpha chain comprises an HLA-DQA1*01 allele. In certain aspects, the DQ alpha chain comprises an HLA-DQA1*02 allele. In certain aspects, the DQ alpha chain comprises an HLA-DQA1*03 allele. In certain aspects, the DQ alpha chain comprises an HLA-DQA1*04 allele. In certain aspects, the DQ alpha chain comprises an HLA-DQA1*05 allele. In certain aspects, the DQ alpha chain comprises an HLA-DQA1*06 allele.

In certain aspects, the DQ alpha chain is selected from DQA1*01:01:01:01, DQA1*01:01:01:02, DQA1*01:01:01:03, DQA1*01:01:01:05, DQA1*01:01:01:06, DQA1*01:01:02, DQA1*01:01:03, DQA1*01:01:04, DQA1*01:01:05, DQA1*01:02:01:01, DQA1*01:02:01:02, DQA1*01:02:01:03, DQA1*01:02:01:04, DQA1*01:02:01:05, DQA1*01:02:01:06, DQA1*01:02:01:07, DQA1*01:02:01:08, DQA1*01:02:01:09, DQA1*01:02:01:10, DQA1*01:02:01:11, DQA1*01:02:01:12, DQA1*01:02:02:01, DQA1*01:02:02:02, DQA1*01:02:02:03, DQA1*01:02:02:04, DQA1*01:02:03, DQA1*01:02:04, DQA1*01:03:01:01, DQA1*01:03:01:02, DQA1*01:03:01:03, DQA1*01:03:01:04, DQA1*01:03:01:05, DQA1*01:03:01:06, DQA1*01:03:01:07, DQA1*01:03:01:08, DQA1*01:03:01:09, DQA1*01:04:01:01, DQA1*01:04:01:02, DQA1*01:04:01:03, DQA1*01:04:01:04, DQA1*01:04:02, DQA1*01:05:01, DQA1*01:05:02, DQA1*01:06, DQA1*01:07Q, DQA1*01:08, DQA1*01:09, DQA1*01:10, DQA1*01:11, DQA1*01:12, DQA1*01:13, DQA1*01:14, DQA1*01:15N, DQA1*01:16N, DQA1*01:17, DQA1*01:18, DQA1*01:19, DQA1*01:20, DQA1*01:21, DQA1*01:22, DQA1*01:23, DQA1*01:24, DQA1*01:25, DQA1*01:26, DQA1*02:01:01:01, DQA1*02:01:01:02, DQA1*02:01:02, DQA1*02:02N, DQA1*02:03, DQA1*03:01:01, DQA1*03:01:03, DQA1*03:02:01:01, DQA1*03:02:01:02, DQA1*03:03:01:01, DQA1*03:03:01:02, DQA1*03:03:01:03, DQA1*03:03:01:04, DQA1*03:03:01:05, DQA1*03:03:01:06, DQA1*03:03:01:07, DQA1*03:03:02, DQA1*03:04, DQA1*03:05, DQA1*03:06, DQA1*03:07, DQA1*04:01:01:01, DQA1*04:01:01:02, DQA1*04:01:01:03, DQA1*04:01:01:04, DQA1*04:01:01:05, DQA1*04:01:01:06, DQA1*04:01:01:07, DQA1*04:01:01:08, DQA1*04:01:02:01, DQA1*04:01:02:02, DQA1*04:01:03, DQA1*04:02, DQA1*04:03N, DQA1*04:04, DQA1*04:05, DQA1*05:01:01:01, DQA1*05:01:01:02, DQA1*05:01:01:03, DQA1*05:01:01:04, DQA1*05:01:02, DQA1*05:01:04, DQA1*05:01:05, DQA1*05:01:06, DQA1*05:02, DQA1*05:03:01:01, DQA1*05:03:01:02, DQA1*05:04, DQA1*05:05:01:01, DQA1*05:05:01:02, DQA1*05:05:01:03, DQA1*05:05:01:04, DQA1*05:05:01:05, DQA1*05:05:01:06, DQA1*05:05:01:07, DQA1*05:05:01:08, DQA1*05:05:01:09, DQA1*05:05:01:10, DQA1*05:05:01:11, DQA1*05:05:01:12, DQA1*05:05:01:13, DQA1*05:05:01:14, DQA1*05:05:01:15, DQA1*05:05:01:16, DQA1*05:05:01:17, DQA1*05:05:01:18, DQA1*05:05:01:19, DQA1*05:05:01:20, DQA1*05:06:01:01, DQA1*05:06:01:02, DQA1*05:07, DQA1*05:08, DQA1*05:09, DQA1*05:10, DQA1*05:11, DQA1*05:12, DQA1*05:13, DQA1*05:14, DQA1*05:15N, DQA1*06:01:01:01, DQA1*06:01:01:02, DQA1*06:01:01:03, DQA1*06:01:01:04, DQA1*06:01:02, DQA1*06:02, and any combination thereof.

In certain aspects, the MHC class II molecule comprises a DQ alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 16. In certain aspects, the MHC class II molecule comprises a DQ alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 18. In certain aspects, the MHC class II molecule comprises a DQ alpha chain comprising an amino acid sequence set forth in SEQ ID NO: 16. In certain aspects, the MHC class II molecule comprises a DQ alpha chain comprising an amino acid sequence set forth in SEQ ID NO: 18.

II.A.3. HLA-DR Molecules

Many HLA-DR alleles are known in the art, and any of the known alleles can be used in the present disclosure. Examples of HLA-DR alpha chain and beta chain alleles are shown in Table 5. An updated list of HLA alleles is available at hla.alleles.org/(last visited on Jul. 10, 2019).

TABLE 5
DR Beta chain and alpha chain amino acid and nucleotide sequences.
Beta Chain
DRB1*01: 01 Extracellular Domain (SEQ ID NO: 19)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNLLVCSVSGFYPGSIEVRWFRNGQEEKAGVVSTGLI
QNGDWTFQTLVMLETVPRSGEVYTCQVEHPSVTSPLTVEWRARSESAQSK
DRB1*01: 01 Extracellular Domain (SEQ ID NO: 20)
GGGGACACCCGACCACGTTTCTTGTGGCAGCTTAAGTTTGAATGTCATTTCTTCAATGGGACGGAGCGGGTGCG
GTTGCTGGAAAGATGCATCTATAACCAAGAGGAGTCCGTGCGCTTCGACAGCGACGTGGGGGAGTACCGGGCGG
TGACGGAGCTGGGGCGGCCTGATGCCGAGTACTGGAACAGCCAGAAGGACCTCCTGGAGCAGAGGCGGGCCGCG
GTGGACACCTACTGCAGACACAACTACGGGGTTGGTGAGAGCTTCACAGTGCAGCGGCGAGTTGAGCCTAAGGT
GACTGTGTATCCTTCAAAGACCCAGCCCCTGCAGCACCACAACCTCCTGGTCTGCTCTGTGAGTGGTTTCTATC
CAGGCAGCATTGAAGTCAGGTGGTTCCGGAACGGCCAGGAAGAGAAGGCTGGGGTGGTGTCCACAGGCCTGATC
CAGAATGGAGATTGGACCTTCCAGACCCTGGTGATGCTGGAAACAGTTCCTCGGAGTGGAGAGGTTTACACCTG
CCAAGTGGAGCACCCAAGTGTGACGAGCCCTCTCACAGTGGAATGGAGAGCACGGTCTGAATCTGCACAGAGCA
AG
DRB1*01: 01 L114W/V143M Extracellular Domain (SEQ ID NO: 268)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCSVSGFYPGSIEVRWFRNGQEEKAGVMSTGLI
QNGDWTFQTLVMLETVPRSGEVYTCQVEHPSVTSPLTVEWRARSESAQSK
DRB1*01: 01 L114W/V143M + 6reps Extracellular Domain (SEQ ID NO: 269)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCHVSGFYPGSIEVRWFRNGQEETAGVMSTNLI
QNGDWTFQILVMLEMTPRSGEVYTCQVEHPSVTSPLTVEWRARSESAQSK
DRB1*01: 01 L114W/V143M/S118H/T157I Extracellular Domain (SEQ ID NO: 21)
GDTRPRFLWQLKFECHFFNGTERVRLLERCIYNQEESVRFDSDVGEYRAVTELGRPDAEYWNSQKDLLEQRRAA
VDTYCRHNYGVGESFTVQRRVEPKVTVYPSKTQPLQHHNWLVCHVSGFYPGSIEVRWFRNGQEEKAGVMSTGLI
QNGDWTFQILVMLETVPRSGEVYTCQVEHPSVTSPLTVEWRARSESAQSK
Signal Peptide; DRB1*01: 01 L114W/V143M/S118H/T157I Extracellular Domain;
Gly/Ser Linker; Zip Sequences and His tag sequences) (SEQ ID NO: 22)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCHVSGFYPGSIEVRW
FRNGQEEKAGVMSTGLIQNGDWTFQILVMLETVPRSGEVYTCQVEHPSVTSPLTVEWRARSESAQSKGGGGSLE
IEAAFLERENTALETRVAELRQRVQRLRNRVSQYRTRYGPLGGGK
Full-length wild-type DRB1*01: 01 (SEQ ID NO: 23)
MVCLKLPGGSCMTALTVTLMVLSSPLALAGDTRPRFLWQLKFECHFFNGTERVRLLERCIYNQEESVRFDSDVG
EYRAVTELGRPDAEYWNSQKDLLEQRRAAVDTYCRHNYGVGESFTVQRRVEPKVTVYPSKTQPLQHHNLLVCSV
SGFYPGSIEVRWFRNGQEEKAGVVSTGLIQNGDWTFQTLVMLETVPRSGEVYTCQVEHPSVTSPLTVEWRARSE
SAQSKMLSGVGGFVLGLLFLGAGLFIYFRNQKGHSGLQPTGFLS
Signal Peptide; DRB1*01: 01 Extracellular Domain; and Gly/Ser Linker, Zip
Sequences, and biotinylation sequences) (SEQ ID NO: 270)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNLLVCSVSGFYPGSIEVRW
FRNGQEEKAGVVSTGLIQNGDWTFQTLVMLETVPRSGEVYTCQVEHPSVTSPLTVEWRARSESAQSKGGGGSLE
IEAAFLERENTALETRVAELRQRVQRLRNRVSQYRTRYGPLGGGKGSGLNDIFEAQKIEWHE
Signal Peptide; DRB1*01: 01 Extracellular Domain; and Gly/Ser Linker, Zip
Sequences, and biotinylation sequences) (SEQ ID NO: 271)
ATGATGCGGCCCATCGTGCTGGTGCTGCTGTTCGCCACATCTGCCCTGGCCGGGGACACCCGACCACGTTTCTT
GTGGCAGCTTAAGTTTGAATGTCATTTCTTCAATGGGACGGAGCGGGTGCGGTTGCTGGAAAGATGCATCTATA
ACCAAGAGGAGTCCGTGCGCTTCGACAGCGACGTGGGGGAGTACCGGGCGGTGACGGAGCTGGGGCGGCCTGAT
GCCGAGTACTGGAACAGCCAGAAGGACCTCCTGGAGCAGAGGCGGGCCGCGGTGGACACCTACTGCAGACACAA
CTACGGGGTTGGTGAGAGCTTCACAGTGCAGCGGCGAGTTGAGCCTAAGGTGACTGTGTATCCTTCAAAGACCC
AGCCCCTGCAGCACCACAACCTCCTGGTCTGCTCTGTGAGTGGTTTCTATCCAGGCAGCATTGAAGTCAGGTGG
TTCCGGAACGGCCAGGAAGAGAAGGCTGGGGTGGTGTCCACAGGCCTGATCCAGAATGGAGATTGGACCTTCCA
GACCCTGGTGATGCTGGAAACAGTTCCTCGGAGTGGAGAGGTTTACACCTGCCAAGTGGAGCACCCAAGTGTGA
CGAGCCCTCTCACAGTGGAATGGAGAGCACGGTCTGAATCTGCACAGAGCAAGGGCGGCGGAGGCAGCCTGGAA
ATCGAGGCCGCCTTCCTGGAAAGAGAGAACACCGCCCTGGAAACCCGGGTGGCCGAGCTGAGACAGAGAGTGCA
GAGACTGCGGAACCGGGTGTCCCAGTACCGGACCAGATATGGCCCTCTGGGAGGCGGCAAAGGGTCCGGCTTGA
ACGACATTTTTGAGGCCCAGAAGATAGAGTGGCACGAGTGA
Signal Peptide; DRB1*01: 01 L114W/V143M Extracellular Domain; Gly/Ser Linker;
Zip Sequences and His tag sequences) (SEQ ID NO: 274)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCSVSGFYPGSIEVRW
FRNGQEEKAGVMSTGLIQNGDWTFQTLVMLETVPRSGEVYTCQVEHPSVTSPLTVEWRARSESAQSKGGGGSLE
IEAAFLERENTALETRVAELRQRVQRLRNRVSQYRTRYGPLGGGK
Signal Peptide; DRB1*01: 01 L114W/V143M/S118H/T157I Extracellular Domain;
Gly/Ser Linker, Zip Sequences, and biotinylation sequences) (SEQ ID NO: 272)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCHVSGFYPGSIEVRW
FRNGQEEKAGVMSTGLIQNGDWTFQILVMLETVPRSGEVYTCQVEHPSVTSPLTVEWRARSESAQSKGGGGSLE
IEAAFLERENTALETRVAELRQRVQRLRNRVSQYRTRYGPLGGGKGSGLNDIFEAQKIEWHE
Signal Peptide; DRB1*01: 01 L114W/V143M/S118H/T157I Extracellular Domain;
Gly/Ser Linker, Zip Sequences, and biotinylation sequences) (SEQ ID NO: 273)
ATGATGCGGCCCATCGTGCTGGTGCTGCTGTTCGCCACATCTGCCCTGGCCGGGGACACCCGACCACGTTTCTT
GTGGCAGCTTAAGTTTGAATGTCATTTCTTCAATGGGACGGAGCGGGTGCGGTTGCTGGAAAGATGCATCTATA
ACCAAGAGGAGTCCGTGCGCTTCGACAGCGACGTGGGGGAGTACCGGGCGGTGACGGAGCTGGGGCGGCCTGAT
GCCGAGTACTGGAACAGCCAGAAGGACCTCCTGGAGCAGAGGCGGGCCGCGGTGGACACCTACTGCAGACACAA
CTACGGGGTTGGTGAGAGCTTCACAGTGCAGCGGCGAGTTGAGCCTAAGGTGACTGTGTATCCTTCAAAGACCC
AGCCCCTGCAGCACCACAACTGGCTGGTCTGCCATGTGAGTGGTTTCTATCCAGGCAGCATTGAAGTCAGGTGG
TTCCGGAACGGCCAGGAAGAGAAGGCTGGGGTGATGTCCACAGGCCTGATCCAGAATGGAGATTGGACCTTCCA
GATCCTGGTGATGCTGGAAACAGTTCCTCGGAGTGGAGAGGTTTACACCTGCCAAGTGGAGCACCCAAGTGTGA
CGAGCCCTCTCACAGTGGAATGGAGAGCACGGTCTGAATCTGCACAGAGCAAGGGCGGCGGAGGCAGCCTGGAA
ATCGAGGCCGCCTTCCTGGAAAGAGAGAACACCGCCCTGGAAACCCGGGTGGCCGAGCTGAGACAGAGAGTGCA
GAGACTGCGGAACCGGGTGTCCCAGTACCGGACCAGATATGGCCCTCTGGGAGGCGGCAAAGGGTCCGGCTTGA
ACGACATTTTTGAGGCCCAGAAGATAGAGTGGCACGAGTGA
Signal Peptide; DRB1*01: 01 L114W/V143M + 6rps Extracellular Domain; Gly/Ser
Linker; Zip Sequences and His tag sequences) (SEQ ID NO: 275)
MMRPIVLVLLFATSALAGDTRPRFLWQLKFECHFFNGTERVRLLERCIYNQEESVRFDSDVGEYRAVTELGRPD
AEYWNSQKDLLEQRRAAVDTYCRHNYGVGESFTVQRRVEPKVTVYPSKTQPLQHHNWLVCHVSGFYPGSIEVRW
FRNGQEETAGVMSTNLIQNGDWTFQILVMLEMTPRSGEVYTCQVEHPSVTSPLTVEWRARSESAQSKGGGGSLE
IEAAFLERENTALETRVAELRQRVQRLRNRVSQYRTRYGPLGGGK
Alpha Chain
DRA1*01: 01 Extracellular Domain (SEQ ID NO: 24)
IKEEHVIIQAEFYLNPDQSGEFMFDFDGDEIFHVDMAKKETVWRLEEFGRFASFEAQGALANIAVDKANLEIMT
KRSNYTPITNVPPEVTVLTNSPVELREPNVLICFIDKFTPPVVNVTWLRNGKPVTTGVSETVFLPREDHLFRKF
HYLPFLPSTEDVYDCRVEHWGLDEPLLKHWEFDAPSPLPETTEN
DRA1*01: 01 Extracellular Domain (SEQ ID NO: 25)
ATCAAAGAAGAACATGTGATCATCCAGGCCGAGTTCTATCTGAATCCTGACCAATCAGGCGAGTTTATGTTTGA
CTTTGATGGTGATGAGATTTTCCATGTGGATATGGCAAAGAAGGAGACGGTCTGGCGGCTTGAAGAATTTGGAC
GATTTGCCAGCTTTGAGGCTCAAGGTGCATTGGCCAACATAGCTGTGGACAAAGCCAACCTGGAAATCATGACA
AAGCGCTCCAACTATACTCCGATCACCAATGTACCTCCAGAGGTAACTGTGCTCACAAACAGCCCTGTGGAACT
GAGAGAGCCCAACGTCCTCATCTGTTTCATAGACAAGTTCACCCCACCAGTGGTCAATGTCACGTGGCTTCGAA
ATGGAAAACCTGTCACCACAGGAGTGTCAGAGACAGTCTTCCTGCCCAGGGAAGACCACCTTTTCCGCAAGTTC
CACTATCTCCCCTTCCTGCCCTCAACTGAGGACGTTTACGACTGCAGGGTGGAGCACTGGGGCTTGGATGAGCC
TCTTCTCAAGCACTGGGAGTTTGATGCTCCAAGCCCTCTCCCAGAGACTACAGAGAAC
Signal Peptide; DRA1*01: 01 Extracellular Domain; Gly/Ser Linker, Zip
Sequences and His tag sequences)(SEQ ID NO: 26)
MMRPIVLVLLFATSALAIKEEHVIIQAEFYLNPDQSGEFMFDFDGDEIFHVDMAKKETVWRLEEFGRFASFEAQ
GALANIAVDKANLEIMTKRSNYTPITNVPPEVTVLTNSPVELREPNVLICFIDKFTPPVVNVTWLRNGKPVTTG
VSETVFLPREDHLFRKEHYLPFLPSTEDVYDCRVEHWGLDEPLLKHWEFDAPSPLPETTENGGGGSLEIRAAFL
RQRNTALRTEVAELEQEVQRLENEVSQYETRYGPLGGGKGSHHHHHH
Signal Peptide; DRA1*01: 01 Extracellular Domain; Gly/Ser Linker, Zip
Sequences and His tag sequences (10x) (SEQ ID NO: 276)
MMRPIVLVLLFATSALAIKEEHVIIQAEFYLNPDQSGEFMFDFDGDEIFHVDMAKKETVWRLEEFGRFASFEAQ
GALANIAVDKANLEIMTKRSNYTPITNVPPEVTVLTNSPVELREPNVLICFIDKFTPPVVNVTWLRNGKPVTTG
VSETVFLPREDHLFRKEHYLPFLPSTEDVYDCRVEHWGLDEPLLKHWEFDAPSPLPETTENGGGGSLEIRAAFL
RQRNTALRTEVAELEQEVQRLENEVSQYETRYGPLGGGKGSHHHHHHHHHH
Signal Peptide; DRA1*01: 01 Extracellular Domain; Gly/Ser Linker, Zip
Sequences and His tag sequences (10x) (SEQ ID NO: 277)
ATGATGCGGCCCATCGTGCTGGTGCTGCTGTTCGCCACATCTGCCCTGGCCATCAAAGAAGAACATGTGATCAT
CCAGGCCGAGTTCTATCTGAATCCTGACCAATCAGGCGAGTTTATGTTTGACTTTGATGGTGATGAGATTTTCC
ATGTGGATATGGCAAAGAAGGAGACGGTCTGGCGGCTTGAAGAATTTGGACGATTTGCCAGCTTTGAGGCTCAA
GGTGCATTGGCCAACATAGCTGTGGACAAAGCCAACCTGGAAATCATGACAAAGCGCTCCAACTATACTCCGAT
CACCAATGTACCTCCAGAGGTAACTGTGCTCACGAACAGCCCTGTGGAACTGAGAGAGCCCAACGTCCTCATCT
GTTTCATCGACAAGTTCACCCCACCAGTGGTCAATGTCACGTGGCTTCGAAATGGAAAACCTGTCACCACAGGA
GTGTCAGAGACAGTCTTCCTGCCCAGGGAAGACCACCTTTTCCGCAAGTTCCACTATCTCCCCTTCCTGCCCTC
AACTGAGGACGTTTACGACTGCAGGGTGGAGCACTGGGGCTTGGATGAGCCTCTTCTCAAGCACTGGGAGTTTG
ATGCTCCAAGCCCTCTCCCAGAGACTACAGAGAACGGCGGCGGAGGCAGCCTGGAAATCAGAGCCGCCTTCCTG
CGGCAGAGAAACACCGCCCTGAGAACCGAAGTGGCCGAGCTGGAACAGGAAGTGCAGCGGCTGGAAAACGAGGT
GTCCCAGTACGAGACAAGATACGGCCCTCTGGGAGGCGGCAAGGGCTCTCACCACCACCATCACCATCATCATC
ACCATTGA

II.A.3.a. HLA-DR Beta Chain

In certain aspects, the HLA class II molecule comprises a DR beta chain, wherein the DR beta chain comprises an amino acid other than leucine at a position corresponding to amino acid residue 114 of SEQ ID NO: 19. Any amino acid other than leucine can be present at the position corresponding to amino acid residue 114 of SEQ ID NO: 19. In some aspects, the amino acid other than leucine is an amino acid comprising a hydrophobic side chain. In certain aspects, the amino acid other than leucine at the position corresponding to amino acid residue 114 of SEQ ID NO: 19 is an amino acid selected from an alanine, a valine, an isoleucine, a methionine, a phenylalanine, a tyrosine, and a tryptophan. In certain aspects, the amino acid other than leucine at the position corresponding to amino acid residue 114 of SEQ ID NO: 19 is an alanine. In certain aspects, the amino acid other than leucine at the position corresponding to amino acid residue 114 of SEQ ID NO: 19 is a valine. In certain aspects, the amino acid other than leucine at the position corresponding to amino acid residue 114 of SEQ ID NO: 19 is an isoleucine. In certain aspects, the amino acid other than leucine at the position corresponding to amino acid residue 114 of SEQ ID NO: 19 is a methionine. In certain aspects, the amino acid other than leucine at the position corresponding to amino acid residue 114 of SEQ ID NO: 19 is a phenylalanine. In certain aspects, the amino acid other than leucine at the position corresponding to amino acid residue 114 of SEQ ID NO: 19 is a tyrosine. In certain aspects, the amino acid other than leucine at the position corresponding to amino acid residue 114 of SEQ ID NO: 19 is a tryptophan.

In some embodiments, the amino acid other than leucine at the position corresponding to amino acid residue 114 of SEQ ID NO: 19 consists of more than one amino acid, e.g., two amino acids, three amino acids, four amino acids, five amino acids, or more. In some aspects at least one of the more than one amino acids comprises a hydrophobic side chain. In certain aspects, the amino acid other than leucine at the position corresponding to amino acid residue 114 of SEQ ID NO: 19 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than valine at a position corresponding to amino acid residue 143 of SEQ ID NO: 19. Any amino acid other than valine can be present at the position corresponding to amino acid residue 143 of SEQ ID NO: 19. In some aspects, the amino acid other than valine is an amino acid comprising a hydrophobic side chain. In certain aspects, the amino acid other than valine at the position corresponding to amino acid residue 143 of SEQ ID NO: 19 is an amino acid selected from an alanine, an isoleucine, a leucine, a methionine, a phenylalanine, a tyrosine, and a tryptophan. In certain aspects, the amino acid other than valine at the position corresponding to amino acid residue 143 of SEQ ID NO: 19 is an alanine. In certain aspects, the amino acid other than valine at the position corresponding to amino acid residue 143 of SEQ ID NO: 19 is an isoleucine. In certain aspects, the amino acid other than valine at the position corresponding to amino acid residue 143 of SEQ ID NO: 19 is a leucine. In certain aspects, the amino acid other than valine at the position corresponding to amino acid residue 143 of SEQ ID NO: 19 is a methionine. In certain aspects, the amino acid other than valine at the position corresponding to amino acid residue 143 of SEQ ID NO: 19 is a phenylalanine. In certain aspects, the amino acid other than valine at the position corresponding to amino acid residue 143 of SEQ ID NO: 19 is a tyrosine. In certain aspects, the amino acid other than valine at the position corresponding to amino acid residue 143 of SEQ ID NO: 19 is a tryptophan.

In some aspects, the amino acid other than valine at the position corresponding to amino acid residue 143 of SEQ ID NO: 19 consists of more than one amino acid, e.g., two amino acids, three amino acids, four amino acids, five amino acids, or more. In some aspects at least one of the more than one amino acids comprises a hydrophobic side chain. In certain aspects, the amino acid other than valine at the position corresponding to amino acid residue 143 of SEQ ID NO: 19 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than serine at a position corresponding to amino acid residue 118 of SEQ ID NO: 19. Any amino acid other than serine can be present at the position corresponding to amino acid residue 118 of SEQ ID NO: 19. In some aspects, the amino acid other than serine is an amino acid comprising an electrically charged side chain. In certain aspects, the amino acid other than serine at the position corresponding to amino acid residue 118 of SEQ ID NO: 19 is an amino acid selected from an arginine, a histidine, and a lysine. In certain aspects, the amino acid other than serine at the position corresponding to amino acid residue 118 of SEQ ID NO: 19 is an arginine. In certain aspects, the amino acid other than serine at the position corresponding to amino acid residue 118 of SEQ ID NO: 19 is a histidine. In certain aspects, the amino acid other than serine at the position corresponding to amino acid residue 118 of SEQ ID NO: 19 is a lysine.

In some aspects, the amino acid other than serine at the position corresponding to amino acid residue 118 of SEQ ID NO: 19 consists of more than one amino acid, e.g., two amino acids, three amino acids, four amino acids, five amino acids, or more. In some aspects at least one of the more than one amino acids comprises an electrically charged side chain. In certain aspects, the amino acid other than serine at the position corresponding to amino acid residue 118 of SEQ ID NO: 19 consists of a series, e.g., at least 2, at least 3, at least 4, or at least 5, amino acids, wherein each of the series of amino acids comprises an electrically charged side chain.

In certain aspects, the HLA class II molecule comprises a DR beta chain, wherein the DR beta chain comprises an amino acid other than threonine at a position corresponding to amino acid residue 157 of SEQ ID NO: 19. Any amino acid other than threonine can be present at the position corresponding to amino acid residue 157 of SEQ ID NO: 19. In some aspects, the amino acid other than threonine is an amino acid comprising a hydrophobic side chain. In certain aspects, the amino acid other than threonine at the position corresponding to amino acid residue 157 of SEQ ID NO: 19 is an amino acid selected an alanine, a valine, an isoleucine, a leucine, a methionine, a phenylalanine, a tyrosine, and a tryptophan. In certain aspects, the amino acid other than threonine at the position corresponding to amino acid residue 157 of SEQ ID NO: 19 is an alanine. In certain aspects, the amino acid other than threonine at the position corresponding to amino acid residue 157 of SEQ ID NO: 19 is a valine. In certain aspects, the amino acid other than threonine at the position corresponding to amino acid residue 157 of SEQ ID NO: 19 is an isoleucine. In certain aspects, the amino acid other than threonine at the position corresponding to amino acid residue 157 of SEQ ID NO: 19 is a leucine. In certain aspects, the amino acid other than threonine at the position corresponding to amino acid residue 157 of SEQ ID NO: 19 is a methionine. In certain aspects, the amino acid other than threonine at the position corresponding to amino acid residue 157 of SEQ ID NO: 19 is a phenylalanine. In certain aspects, the amino acid other than threonine at the position corresponding to amino acid residue 157 of SEQ ID NO: 19 is a tyrosine. In certain aspects, the amino acid other than threonine at the position corresponding to amino acid residue 157 of SEQ ID NO: 19 is a tryptophan.

In some aspects, the amino acid other than threonine at the position corresponding to amino acid residue 157 of SEQ ID NO: 19 consists of more than one amino acid, e.g., two amino acids, three amino acids, four amino acids, five amino acids, or more. In some aspects at least one of the more than one amino acids comprises a hydrophobic side chain. In certain aspects, the amino acid other than threonine at the position corresponding to amino acid residue 157 of SEQ ID NO: 19 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than lysine at a position corresponding to amino acid residue 139 of SEQ ID NO: 19. Any amino acid other than lysine can be present at the position corresponding to amino acid residue 139 of SEQ ID NO: 19. In some aspects, the amino acid other than lysine is an amino acid comprising a polar uncharged side chain. In certain aspects, the amino acid other than lysine at the position corresponding to amino acid residue 139 of SEQ ID NO: 19 is an amino acid selected from a serine, a threonine, and a glutamine. In certain aspects, the amino acid other than lysine at the position corresponding to amino acid residue 139 of SEQ ID NO: 19 is a serine. In certain aspects, the amino acid other than lysine at the position corresponding to amino acid residue 139 of SEQ ID NO: 19 is a threonine. In certain aspects, the amino acid other than lysine at the position corresponding to amino acid residue 139 of SEQ ID NO: 19 is a glutamine.

In some aspects, the amino acid other than lysine at the position corresponding to amino acid residue 139 of SEQ ID NO: 19 consists of more than one amino acid, e.g., two amino acids, three amino acids, four amino acids, five amino acids, or more. In some aspects at least one of the more than one amino acids comprises a polar uncharged side chain. In certain aspects, the amino acid other than lysine at the position corresponding to amino acid residue 139 of SEQ ID NO: 19 consists of a series, e.g., at least 2, at least 3, at least 4, or at least 5, amino acids, wherein each of the series of amino acids comprises a polar uncharged side chain.

In certain aspects, the HLA class II molecule comprises a DR beta chain, wherein the DR beta chain comprises an amino acid other than glycine at a position corresponding to amino acid residue 146 of SEQ ID NO: 19. Any amino acid other than glycine can be present at the position corresponding to amino acid residue 146 of SEQ ID NO: 19. In some aspects, the amino acid other than glycine is an amino acid comprising a polar uncharged side chain. In certain aspects, the amino acid other than glycine at the position corresponding to amino acid residue 146 of SEQ ID NO: 19 is an amino acid selected from a serine, an asparagine, a threonine, and a glutamine. In certain aspects, the amino acid other than glycine at the position corresponding to amino acid residue 146 of SEQ ID NO: 19 is a serine. In certain aspects, the amino acid other than glycine at the position corresponding to amino acid residue 146 of SEQ ID NO: 19 is an asparagine. In certain aspects, the amino acid other than glycine at the position corresponding to amino acid residue 146 of SEQ ID NO: 19 is a threonine. In certain aspects, the amino acid other than glycine at the position corresponding to amino acid residue 146 of SEQ ID NO: 19 is a glutamine.

In some aspects, the amino acid other than glycine at the position corresponding to amino acid residue 146 of SEQ ID NO: 19 consists of more than one amino acid, e.g., two amino acids, three amino acids, four amino acids, five amino acids, or more. In some aspects at least one of the more than one amino acids comprises a polar uncharged side chain. In certain aspects, the amino acid other than glycine at the position corresponding to amino acid residue 146 of SEQ ID NO: 19 consists of a series, e.g., at least 2, at least 3, at least 4, or at least 5, amino acids, wherein each of the series of amino acids comprises a polar uncharged side chain.

In certain aspects, the HLA class II molecule comprises a DR beta chain, wherein the DR beta chain comprises an amino acid other than threonine at a position corresponding to amino acid residue 163 of SEQ ID NO: 19. Any amino acid other than threonine can be present at the position corresponding to amino acid residue 163 of SEQ ID NO: 19. In some aspects, the amino acid other than threonine is an amino acid comprising a hydrophobic side chain. In certain aspects, the amino acid other than threonine at the position corresponding to amino acid residue 163 of SEQ ID NO: 19 is an amino acid selected from an alanine, a valine, an isoleucine, a leucine, a methionine, a phenylalanine, a tyrosine, and a tryptophan. In certain aspects, the amino acid other than threonine at the position corresponding to amino acid residue 163 of SEQ ID NO: 19 is an alanine. In certain aspects, the amino acid other than threonine at the position corresponding to amino acid residue 163 of SEQ ID NO: 19 is a valine. In certain aspects, the amino acid other than threonine at the position corresponding to amino acid residue 163 of SEQ ID NO: 19 is an isoleucine. In certain aspects, the amino acid other than threonine at the position corresponding to amino acid residue 163 of SEQ ID NO: 19 is a leucine. In certain aspects, the amino acid other than threonine at the position corresponding to amino acid residue 163 of SEQ ID NO: 19 is a methionine. In certain aspects, the amino acid other than threonine at the position corresponding to amino acid residue 163 of SEQ ID NO: 19 is a phenylalanine. In certain aspects, the amino acid other than threonine at the position corresponding to amino acid residue 163 of SEQ ID NO: 19 is a tyrosine. In certain aspects, the amino acid other than threonine at the position corresponding to amino acid residue 163 of SEQ ID NO: 19 is a tryptophan.

In some aspects, the amino acid other than threonine at the position corresponding to amino acid residue 163 of SEQ ID NO: 19 consists of more than one amino acid, e.g., two amino acids, three amino acids, four amino acids, five amino acids, or more. In some aspects at least one of the more than one amino acids comprises a hydrophobic side chain. In certain aspects, the amino acid other than threonine at the position corresponding to amino acid residue 163 of SEQ ID NO: 19 consists of a series, e.g., at least 2, at least 3, at least 4, or at least 5, amino acids, wherein each of the series of amino acids comprises a hydrophobic side chain.

In certain aspects, the HLA class II molecule comprises a DR beta chain, wherein the DR beta chain comprises an amino acid other than valine at a position corresponding to amino acid residue 164 of SEQ ID NO: 19. Any amino acid other than valine can be present at the position corresponding to amino acid residue 164 of SEQ ID NO: 19. In some aspects, the amino acid other than valine is an amino acid comprising a polar uncharged side chain. In certain aspects, the amino acid other than valine at the position corresponding to amino acid residue 164 of SEQ ID NO: 19 is an amino acid selected from a serine, an asparagine, a threonine, and a glutamine. In certain aspects, the amino acid other than valine at the position corresponding to amino acid residue 164 of SEQ ID NO: 19 is a serine. In certain aspects, the amino acid other than valine at the position corresponding to amino acid residue 164 of SEQ ID NO: 19 is an asparagine. In certain aspects, the amino acid other than valine at the position corresponding to amino acid residue 164 of SEQ ID NO: 19 is a threonine. In certain aspects, the amino acid other than valine at the position corresponding to amino acid residue 164 of SEQ ID NO: 19 is a glutamine.

In some aspects, the amino acid other than valine at the position corresponding to amino acid residue 164 of SEQ ID NO: 19 consists of more than one amino acid, e.g., two amino acids, three amino acids, four amino acids, five amino acids, or more. In some aspects at least one of the more than one amino acids comprises a polar uncharged side chain. In certain aspects, the amino acid other than valine at the position corresponding to amino acid residue 164 of SEQ ID NO: 19 consists of a series, e.g., at least 2, at least 3, at least 4, or at least 5, amino acids, wherein each of the series of amino acids comprises a polar uncharged side chain.

In certain aspects of the present disclosure, the MHC class II molecule comprises a DR beta chain comprising more than one substitution mutation relative to the wild-type DR beta chain. In certain aspects, the DR beta chain comprises at least two mutations, at least three mutations, at least four mutations, at least five mutations, at least six mutations, at least seven mutations, at least eight mutations, at least nine mutations, or at least ten mutations relative to the wild-type DR beta chain.

In certain aspects, the DR beta chain comprises an amino acid other than leucine at a position corresponding to amino acid residue 114 of SEQ ID NO: 19 and an amino acid other than valine at a position corresponding to amino acid residue 143 of SEQ ID NO: 19.

In certain aspects, the DR beta chain comprises (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, and (d) an amino acid other than threonine at a position corresponding to amino acid residue 157 of SEQ ID NO: 19.

In certain aspects, the DR beta chain comprises an amino acid other than leucine at a position corresponding to amino acid residue 114 of SEQ ID NO: 19; an amino acid other than valine at a position corresponding to amino acid residue 143 of SEQ ID NO: 19; and at least two of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In certain aspects, the DR beta chain comprises an amino acid other than leucine at a position corresponding to amino acid residue 114 of SEQ ID NO: 19; an amino acid other than valine at a position corresponding to amino acid residue 143 of SEQ ID NO: 19; and at least three of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In certain aspects, the DR beta chain comprises an amino acid other than leucine at a position corresponding to amino acid residue 114 of SEQ ID NO: 19; an amino acid other than valine at a position corresponding to amino acid residue 143 of SEQ ID NO: 19; and at least four of: (i) an amino acid other than serine at a position corresponding to amino acid residue 118 of SEQ ID NO: 19, (ii) an amino acid other than lysine at a position corresponding to amino acid residue 139 of SEQ ID NO: 19, (iii) an amino acid other than glycine at a position corresponding to amino acid residue 146 of SEQ ID NO: 19, (iv) an amino acid other than threonine at a position corresponding to amino acid residue 157 of SEQ ID NO: 19, (v) an amino acid other than threonine at a position corresponding to amino acid residue 163 of SEQ ID NO: 19, and (vi) an amino acid other than valine at a position corresponding to amino acid residue 164 of SEQ ID NO: 19.

In certain aspects, the DR beta chain comprises (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, and (d) an amino acid other than threonine at a position corresponding to amino acid residue 157 of SEQ ID NO: 19; and at least one of: (i) an amino acid other than lysine at a position corresponding to amino acid residue 139 of SEQ ID NO: 19, (ii) an amino acid other than glycine at a position corresponding to amino acid residue 146 of SEQ ID NO: 19, (iii) an amino acid other than threonine at a position corresponding to amino acid residue 163 of SEQ ID NO: 19, and (iv) an amino acid other than valine at a position corresponding to amino acid residue 164 of SEQ ID NO: 19.

In certain aspects, the DR beta chain comprises (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, and (d) an amino acid other than threonine at a position corresponding to amino acid residue 157 of SEQ ID NO: 19; and at least two of: (i) an amino acid other than lysine at a position corresponding to amino acid residue 139 of SEQ ID NO: 19, (ii) an amino acid other than glycine at a position corresponding to amino acid residue 146 of SEQ ID NO: 19, (iii) an amino acid other than threonine at a position corresponding to amino acid residue 163 of SEQ ID NO: 19, and (iv) an amino acid other than valine at a position corresponding to amino acid residue 164 of SEQ ID NO: 19.

In certain aspects, the DR beta chain comprises (a) an amino acid other than leucine at a position corresponding to amino acid residue 114 of SEQ ID NO: 19, (b) an amino acid other than valine at a position corresponding to amino acid residue 143 of SEQ ID NO: 19, (c) an amino acid other than serine at a position corresponding to amino acid residue 118 of SEQ ID NO: 19, and (d) an amino acid other than threonine at a position corresponding to amino acid residue 157 of SEQ ID NO: 19; and at least three of: (i) an amino acid other than lysine at a position corresponding to amino acid residue 139 of SEQ ID NO: 19, (ii) an amino acid other than glycine at a position corresponding to amino acid residue 146 of SEQ ID NO: 19, (iii) an amino acid other than threonine at a position corresponding to amino acid residue 163 of SEQ ID NO: 19, and (iv) an amino acid other than valine at a position corresponding to amino acid residue 164 of SEQ ID NO: 19.

In certain aspects, the DR beta chain comprises (a) an amino acid other than leucine at a position corresponding to amino acid residue 114 of SEQ ID NO: 19; (b) an amino acid other than valine at a position corresponding to amino acid residue 143 of SEQ ID NO: 19; (c) an amino acid other than serine at a position corresponding to amino acid residue 118 of SEQ ID NO: 19, (d) an amino acid other than lysine at a position corresponding to amino acid residue 139 of SEQ ID NO: 19, (e) an amino acid other than glycine at a position corresponding to amino acid residue 146 of SEQ ID NO: 19, (f) an amino acid other than threonine at a position corresponding to amino acid residue 157 of SEQ ID NO: 19, (g) an amino acid other than threonine at a position corresponding to amino acid residue 163 of SEQ ID NO: 19, and (h) an amino acid other than valine at a position corresponding to amino acid residue 164 of SEQ ID NO: 19.

In certain aspects, the DR beta chain comprises (a) a tryptophan at a position corresponding to amino acid residue 114 of SEQ ID NO: 19, (b) a methionine at a position corresponding to amino acid residue 143 of SEQ ID NO: 19, (c) a histidine at a position corresponding to amino acid residue 118 of SEQ ID NO: 19, and (d) an isoleucine at a position corresponding to amino acid residue 157 of SEQ ID NO: 19.

In some aspects, (i) the amino acid other than leucine at the position corresponding to amino acid residue 114 of SEQ ID NO: 19, (ii) the amino acid other than valine at a position corresponding to amino acid residue 143 of SEQ ID NO: 19, or each of the amino acid other than leucine at the position corresponding to amino acid residue 114 of SEQ ID NO: 19 and the amino acid other than valine at a position corresponding to amino acid residue 143 of SEQ ID NO: 19 is an amino acid comprising a hydrophobic side chain.

In some aspects, (i) the amino acid other than leucine at the position corresponding to amino acid residue 114 of SEQ ID NO: 19 is selected from an alanine, a valine, an isoleucine, a methionine, a phenylalanine, a tyrosine, and a tryptophan; (ii) the amino acid other than valine at a position corresponding to amino acid residue 143 of SEQ ID NO: 19 is selected from an alanine, an isoleucine, a leucine, a methionine, a phenylalanine, a tyrosine, and a tryptophan; (iii) the amino acid other than serine at a position corresponding to amino acid residue 118 of SEQ ID NO: 19 is selected from an arginine, a histidine, and a lysine; and/or (iv) the amino acid other than threonine at a position corresponding to amino acid residue 157 of SEQ ID NO: 19 is selected from an alanine, a valine, an isoleucine, a leucine, a methionine, a phenylalanine, a tyrosine, and a tryptophan.

In some aspects, (i) the amino acid other than leucine at the position corresponding to amino acid residue 114 of SEQ ID NO: 19 is selected from an alanine, a valine, an isoleucine, a methionine, a phenylalanine, a tyrosine, and a tryptophan; (ii) the amino acid other than valine at a position corresponding to amino acid residue 143 of SEQ ID NO: 19 is selected from an alanine, an isoleucine, a leucine, a methionine, a phenylalanine, a tyrosine, and a tryptophan; (iii) the amino acid other than serine at a position corresponding to amino acid residue 118 of SEQ ID NO: 19 is selected from an arginine, a histidine, and a lysine; (iv) the amino acid other than threonine at a position corresponding to amino acid residue 157 of SEQ ID NO: 19 is selected from an alanine, a valine, an isoleucine, a leucine, a methionine, a phenylalanine, a tyrosine, and a tryptophan; (v) the amino acid other than lysine at a position corresponding to amino acid residue 139 of SEQ ID NO: 19 is selected from a serine, a threonine, and a glutamine; (vi) the amino acid other than glycine at a position corresponding to amino acid residue 146 of SEQ ID NO: 19 is selected from a serine, an asparagine, a threonine, and a glutamine; (vii) the amino acid other than threonine at a position corresponding to amino acid residue 163 of SEQ ID NO: 19 is selected from an alanine, a valine, an isoleucine, a leucine, a methionine, a phenylalanine, a tyrosine, and a tryptophan; and/or (viii) the amino acid other than valine at a position corresponding to amino acid residue 164 of SEQ ID NO: 19 is selected from a serine, an asparagine, a threonine, and a glutamine.

In certain aspects, a DR beta chain described herein has an increased affinity for a CD4 protein as compared to a reference HLA class II molecule. In some aspects, the reference HLA class II molecule is an HLA class II molecule having a wild-type DR beta chain. In some aspects, the reference HLA class II molecule is an HLA class II molecule having a DR beta chain comprising (i) a leucine at a position corresponding to amino acid residue 114 of SEQ ID NO: 19 and/or (ii) a valine at a position corresponding to amino acid residue 143 of SEQ ID NO: 19. In some aspects, the reference HLA class II molecule is an HLA class II molecule having a DR beta chain comprising (i) a leucine at a position corresponding to amino acid residue 114 of SEQ ID NO: 19, (ii) a valine at a position corresponding to amino acid residue 143 of SEQ ID NO: 19, (iii) a serine at a position corresponding to amino acid residue 118 of SEQ ID NO: 19, and (iv) a threonine at a position corresponding to amino acid residue 157 of SEQ ID NO: 19.

In some aspects, the increased affinity for CD4 is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, at least about 4000-fold, at least about 4500-fold, or at least about 4000-fold greater than the affinity of the reference HLA class II molecule for CD4.

In some aspects, the increased affinity for CD4 is at least about 1.5-fold to at least about 5000-fold, 1.5-fold to at least about 4000-fold, 1.5-fold to at least about 3000-fold, 1.5-fold to at least about 2000-fold, 1.5-fold to at least about 1000-fold, 10-fold to at least about 5000-fold, 10-fold to at least about 4000-fold, 10-fold to at least about 3000-fold, 10-fold to at least about 2000-fold, 10-fold to at least about 1000-fold, 10-fold to at least about 900-fold, 10-fold to at least about 800-fold, 10-fold to at least about 700-fold, 10-fold to at least about 600-fold, 10-fold to at least about 500-fold, 10-fold to at least about 400-fold, 10-fold to at least about 300-fold, 10-fold to at least about 200-fold, 10-fold to at least about 100-fold, 100-fold to at least about 5000-fold, 100-fold to at least about 4000-fold, 100-fold to at least about 3000-fold, 100-fold to at least about 2000-fold, 100-fold to at least about 1000-fold, 100-fold to at least about 900-fold, 100-fold to at least about 800-fold, 100-fold to at least about 700-fold, 100-fold to at least about 600-fold, 100-fold to at least about 500-fold, 100-fold to at least about 400-fold, 100-fold to at least about 300-fold, or 100-fold to at least about 200-fold greater than the affinity of the reference HLA class II molecule for CD4.

In certain aspects, the DR beta chain comprises an allele selected from an HLA-DRB1*01, an HLA-DRB1*03, an HLA-DRB1*04, an HLA-DRB1*06, an HLA-DRB1*07, an HLA-DRB1*08, an HLA-DRB1*09, an HLA-DRB1*10, an HLA-DRB1*11, an HLA-DRB1*12, an HLA-DRB1*13, an HLA-DRB1*14, an HLA-DRB1*15, or an HLA-DRB1*16 allele. In some aspects, the DR beta chain comprises an HLA-DRB1*01 allele. In particular aspects, the DR beta chain comprises an HLA-DRB1*01:01 allele.

In certain aspects, the DR beta chain comprises an allele selected from DRB1*01:01:01, DRB1*01:01:02, DRB1*01:01:03, DRB1*01:01:04, DRB1*01:01:05, DRB1*01:01:06, DRB1*01:01:07, DRB1*01:01:08, DRB1*01:01:09, DRB1*01:01:10, DRB1*01:01:11, DRB1*01:01:12, DRB1*01:01:13, DRB1*01:01:14, DRB1*01:01:15, DRB1*01:01:16, DRB1*01:01:17, DRB1*01:01:18, DRB1*01:01:19, DRB1*01:01:20, DRB1*01:01:21, DRB1*01:01:22, DRB1*01:01:23, DRB1*01:01:24, DRB1*01:01:25, DRB1*01:01:26, DRB1*01:01:27, DRB1*01:01:28, DRB1*01:01:29, DRB1*01:01:30, DRB1*01:01:31, DRB1*01:01:32, DRB1*01:01:33, DRB1*01:02:01:01, DRB1*01:02:01:02, DRB1*01:02:02, DRB1*01:02:03, DRB1*01:02:04, DRB1*01:02:05, DRB1*01:02:06, DRB1*01:02:07, DRB1*01:02:08, DRB1*01:02:09, DRB1*01:02:10, DRB1*01:02:11, DRB1*01:02:12, DRB1*01:02:13, DRB1*01:03:01, DRB1*01:03:02, DRB1*01:03:03, DRB1*01:03:04, DRB1*01:04, DRB1*01:05, DRB1*01:06, DRB1*01:07, DRB1*01:08, DRB1*01:09, DRB1*01:10, DRB1*01:100, DRB1*01:11:01, DRB1*01:11:02, DRB1*01:12, DRB1*01:13, DRB1*01:14, DRB1*01:15, DRB1*01:16, DRB1*01:17, DRB1*01:18:01, DRB1*01:18:02, DRB1*01:19, DRB1*01:20:01, DRB1*01:20:02, DRB1*01:21, DRB1*01:22, DRB1*01:23, DRB1*01:24:01, DRB1*01:24:02, DRB1*01:25, DRB1*01:26, DRB1*01:27, DRB1*01:28, DRB1*01:29:01, DRB1*01:29:02, DRB1*01:30, DRB1*01:31, DRB1*01:32, DRB1*01:33N, DRB1*01:34, DRB1*01:35, DRB1*01:36, DRB1*01:37, DRB1*01:38, DRB1*01:39N, DRB1*01:40N, DRB1*01:41, DRB1*01:42, DRB1*01:43, DRB1*01:44:01, DRB1*01:44:02, DRB1*01:45, DRB1*01:46, DRB1*01:47, DRB1*01:48, DRB1*01:49, DRB1*01:50, DRB1*01:51, DRB1*01:52N, DRB1*01:53, DRB1*01:54, DRB1*01:55, DRB1*01:56, DRB1*01:57, DRB1*01:58, DRB1*01:59, DRB1*01:60, DRB1*01:61, DRB1*01:62N, DRB1*01:63, DRB1*01:64, DRB1*01:65:01, DRB1*01:65:02, DRB1*01:66, DRB1*01:67, DRB1*01:68N, DRB1*01:69, DRB1*01:70, DRB1*01:71, DRB1*01:72, DRB1*01:73, DRB1*01:74, DRB1*01:75, DRB1*01:76, DRB1*01:77, DRB1*01:78, DRB1*01:79, DRB1*01:80, DRB1*01:81, DRB1*01:82, DRB1*01:83, DRB1*01:84, DRB1*01:85, DRB1*01:86, DRB1*01:87, DRB1*01:88, DRB1*01:89, DRB1*01:90, DRB1*01:91Q, DRB1*01:92, DRB1*01:93, DRB1*01:94, DRB1*01:95, DRB1*01:96, DRB1*01:97, DRB1*01:98, DRB1*01:99, DRB1*03:01:01:01, DRB1*03:01:01:02, DRB1*03:01:01:03, DRB1*03:01:02, DRB1*03:01:03, DRB1*03:01:04, DRB1*03:01:05, DRB1*03:01:06, DRB1*03:01:07, DRB1*03:01:08, DRB1*03:01:09, DRB1*03:01:10, DRB1*03:01:11, DRB1*03:01:12, DRB1*03:01:13, DRB1*03:01:14, DRB1*03:01:15, DRB1*03:01:16, DRB1*03:01:17, DRB1*03:01:18, DRB1*03:01:19, DRB1*03:01:20, DRB1*03:01:21, DRB1*03:01:22, DRB1*03:01:23, DRB1*03:01:24, DRB1*03:01:25, DRB1*03:01:26, DRB1*03:01:27, DRB1*03:01:28, DRB1*03:02:01, DRB1*03:02:02, DRB1*03:02:03, DRB1*03:03, DRB1*03:04:01, DRB1*03:04:02, DRB1*03:05:01, DRB1*03:05:02, DRB1*03:05:03, DRB1*03:06, DRB1*03:07:01, DRB1*03:07:02, DRB1*03:08, DRB1*03:09, DRB1*03:10, DRB1*03:100:01, DRB1*03:100:02, DRB1*03:101, DRB1*03:102, DRB1*03:103, DRB1*03:104, DRB1*03:105, DRB1*03:106, DRB1*03:107, DRB1*03:108, DRB1*03:109, DRB1*03:110, DRB1*03:111, DRB1*03:112, DRB1*03:113, DRB1*03:114, DRB1*03:115, DRB1*03:116, DRB1*03:117, DRB1*03:118, DRB1*03:119, DRB1*03:11:01, DRB1*03:12, DRB1*03:120, DRB1*03:121, DRB1*03:122, DRB1*03:123, DRB1*03:124, DRB1*03:125, DRB1*03:126, DRB1*03:127, DRB1*03:128, DRB1*03:129, DRB1*03:130, DRB1*03:131, DRB1*03:132, DRB1*03:133, DRB1*03:134, DRB1*03:135, DRB1*03:136, DRB1*03:137, DRB1*03:138, DRB1*03:139, DRB1*03:13:01, DRB1*03:13:02, DRB1*03:14, DRB1*03:140, DRB1*03:141, DRB1*03:142, DRB1*03:143, DRB1*03:144, DRB1*03:145, DRB1*03:146, DRB1*03:147, DRB1*03:148, DRB1*03:149, DRB1*03:150, DRB1*03:151, DRB1*03:152, DRB1*03:153, DRB1*03:154, DRB1*03:155, DRB1*03:156N, DRB1*03:157, DRB1*03:158, DRB1*03:15:01, DRB1*03:15:02, DRB1*03:16, DRB1*03:17, DRB1*03:18, DRB1*03:19, DRB1*03:20, DRB1*03:21, DRB1*03:22, DRB1*03:23, DRB1*03:24, DRB1*03:25:01, DRB1*03:25:02, DRB1*03:26, DRB1*03:27, DRB1*03:28, DRB1*03:29, DRB1*03:30, DRB1*03:31, DRB1*03:32, DRB1*03:33, DRB1*03:34, DRB1*03:35, DRB1*03:36, DRB1*03:37, DRB1*03:38, DRB1*03:39, DRB1*03:40, DRB1*03:41:01, DRB1*03:41:02, DRB1*03:42, DRB1*03:43, DRB1*03:44, DRB1*03:45, DRB1*03:46, DRB1*03:47, DRB1*03:48, DRB1*03:49, DRB1*03:50, DRB1*03:51, DRB1*03:52, DRB1*03:53, DRB1*03:54, DRB1*03:55, DRB1*03:56, DRB1*03:57, DRB1*03:58, DRB1*03:59, DRB1*03:60, DRB1*03:61, DRB1*03:62, DRB1*03:63, DRB1*03:64, DRB1*03:65, DRB1*03:66, DRB1*03:67N, DRB1*03:68N, DRB1*03:69, DRB1*03:70, DRB1*03:71:01, DRB1*03:71:02, DRB1*03:72, DRB1*03:73, DRB1*03:74, DRB1*03:75, DRB1*03:76, DRB1*03:77, DRB1*03:78, DRB1*03:79, DRB1*03:80, DRB1*03:81, DRB1*03:82, DRB1*03:83, DRB1*03:84, DRB1*03:85, DRB1*03:86, DRB1*03:87, DRB1*03:88, DRB1*03:89, DRB1*03:90, DRB1*03:91, DRB1*03:92, DRB1*03:93, DRB1*03:94, DRB1*03:95, DRB1*03:96, DRB1*03:97, DRB1*03:98, DRB1*03:99, DRB1*04:01:01:01, DRB1*04:01:01:02, DRB1*04:01:01:03, DRB1*04:01:02, DRB1*04:01:03, DRB1*04:01:04, DRB1*04:01:05, DRB1*04:01:06, DRB1*04:01:07, DRB1*04:01:08, DRB1*04:01:09, DRB1*04:01:10, DRB1*04:01:11, DRB1*04:01:12, DRB1*04:01:13, DRB1*04:01:14, DRB1*04:01:15, DRB1*04:01:16, DRB1*04:01:17, DRB1*04:01:18, DRB1*04:01:19, DRB1*04:01:20, DRB1*04:01:21, DRB1*04:02:01, DRB1*04:02:02, DRB1*04:02:03, DRB1*04:02:04, DRB1*04:02:05, DRB1*04:02:06, DRB1*04:03:01:01, DRB1*04:03:01:02, DRB1*04:03:02, DRB1*04:03:03, DRB1*04:03:04, DRB1*04:03:05, DRB1*04:03:06, DRB1*04:03:07, DRB1*04:03:08, DRB1*04:03:09, DRB1*04:03:10, DRB1*04:03:11, DRB1*04:03:12, DRB1*04:03:13, DRB1*04:03:14, DRB1*04:03:15, DRB1*04:04:01, DRB1*04:04:02, DRB1*04:04:03, DRB1*04:04:04, DRB1*04:04:05, DRB1*04:04:06, DRB1*04:04:07, DRB1*04:04:08, DRB1*04:04:09, DRB1*04:04:10, DRB1*04:04:11, DRB1*04:04:12, DRB1*04:04:13, DRB1*04:04:14, DRB1*04:04:15, DRB1*04:05:01:01, DRB1*04:05:01:02, DRB1*04:05:01:03, DRB1*04:05:02, DRB1*04:05:03, DRB1*04:05:04, DRB1*04:05:05, DRB1*04:05:06, DRB1*04:05:07, DRB1*04:05:08, DRB1*04:05:09, DRB1*04:05:10, DRB1*04:05:11, DRB1*04:05:13, DRB1*04:05:14, DRB1*04:05:15, DRB1*04:05:16, DRB1*04:05:17, DRB1*04:05:18, DRB1*04:05:19, DRB1*04:05:20, DRB1*04:06:01, DRB1*04:06:02, DRB1*04:06:03, DRB1*04:06:04, DRB1*04:06:05, DRB1*04:06:06, DRB1*04:06:07, DRB1*04:07:01:01, DRB1*04:07:01:02, DRB1*04:07:02, DRB1*04:07:03, DRB1*04:07:04, DRB1*04:07:05, DRB1*04:07:06, DRB1*04:08:01, DRB1*04:08:02, DRB1*04:08:03, DRB1*04:08:04, DRB1*04:09, DRB1*04:100, DRB1*04:101, DRB1*04:102, DRB1*04:103, DRB1*04:104, DRB1*04:105:01, DRB1*04:105:02, DRB1*04:106, DRB1*04:107, DRB1*04:108, DRB1*04:109, DRB1*04:10:01, DRB1*04:10:02, DRB1*04:10:03, DRB1*04:110, DRB1*04:111, DRB1*04:112, DRB1*04:113, DRB1*04:114, DRB1*04:115, DRB1*04:116, DRB1*04:117, DRB1*04:118, DRB1*04:119N, DRB1*04:11:01, DRB1*04:11:02, DRB1*04:11:03, DRB1*04:11:04, DRB1*04:11:05, DRB1*04:12, DRB1*04:120N, DRB1*04:121, DRB1*04:122, DRB1*04:123, DRB1*04:124, DRB1*04:125, DRB1*04:126, DRB1*04:127, DRB1*04:128, DRB1*04:129, DRB1*04:13, DRB1*04:130, DRB1*04:131:01, DRB1*04:131:02, DRB1*04:132, DRB1*04:133, DRB1*04:134, DRB1*04:135, DRB1*04:136, DRB1*04:137, DRB1*04:138, DRB1*04:139, DRB1*04:14, DRB1*04:140, DRB1*04:141, DRB1*04:142N, DRB1*04:143, DRB1*04:144, DRB1*04:145, DRB1*04:146, DRB1*04:147, DRB1*04:148, DRB1*04:149, DRB1*04:15, DRB1*04:150, DRB1*04:151, DRB1*04:152, DRB1*04:153, DRB1*04:154, DRB1*04:155, DRB1*04:156, DRB1*04:157N, DRB1*04:158N, DRB1*04:159, DRB1*04:16, DRB1*04:160, DRB1*04:161, DRB1*04:162, DRB1*04:163, DRB1*04:164, DRB1*04:165, DRB1*04:166, DRB1*04:167, DRB1*04:168, DRB1*04:169, DRB1*04:170, DRB1*04:171, DRB1*04:172, DRB1*04:173, DRB1*04:174, DRB1*04:175, DRB1*04:176, DRB1*04:177, DRB1*04:178N, DRB1*04:179, DRB1*04:17:01, DRB1*04:17:02, DRB1*04:18, DRB1*04:180, DRB1*04:181, DRB1*04:182, DRB1*04:183, DRB1*04:184, DRB1*04:185, DRB1*04:186N, DRB1*04:187, DRB1*04:188, DRB1*04:189, DRB1*04:19, DRB1*04:190, DRB1*04:191, DRB1*04:192, DRB1*04:193, DRB1*04:194, DRB1*04:195, DRB1*04:196, DRB1*04:197, DRB1*04:198, DRB1*04:199, DRB1*04:20, DRB1*04:200, DRB1*04:201, DRB1*04:202, DRB1*04:203, DRB1*04:204, DRB1*04:205, DRB1*04:206, DRB1*04:207, DRB1*04:208, DRB1*04:209, DRB1*04:21, DRB1*04:210, DRB1*04:211, DRB1*04:212N, DRB1*04:213, DRB1*04:214N, DRB1*04:215, DRB1*04:216, DRB1*04:217, DRB1*04:218, DRB1*04:219, DRB1*04:22, DRB1*04:220, DRB1*04:221, DRB1*04:222, DRB1*04:223, DRB1*04:224, DRB1*04:225, DRB1*04:226:01, DRB1*04:226:02, DRB1*04:227, DRB1*04:228, DRB1*04:229, DRB1*04:23, DRB1*04:230, DRB1*04:231, DRB1*04:232, DRB1*04:233, DRB1*04:234, DRB1*04:235, DRB1*04:236, DRB1*04:237, DRB1*04:238, DRB1*04:239, DRB1*04:24, DRB1*04:240, DRB1*04:241, DRB1*04:242, DRB1*04:243, DRB1*04:244, DRB1*04:245, DRB1*04:246, DRB1*04:247N, DRB1*04:248, DRB1*04:249, DRB1*04:25, DRB1*04:250, DRB1*04:251, DRB1*04:252, DRB1*04:253, DRB1*04:254, DRB1*04:255, DRB1*04:256, DRB1*04:257, DRB1*04:258, DRB1*04:259, DRB1*04:26, DRB1*04:260, DRB1*04:261, DRB1*04:262, DRB1*04:263, DRB1*04:264N, DRB1*04:265, DRB1*04:266N, DRB1*04:267N, DRB1*04:268, DRB1*04:269, DRB1*04:27, DRB1*04:270, DRB1*04:271, DRB1*04:272, DRB1*04:28, DRB1*04:29, DRB1*04:30, DRB1*04:31, DRB1*04:32, DRB1*04:33, DRB1*04:34, DRB1*04:35, DRB1*04:36, DRB1*04:37, DRB1*04:38, DRB1*04:39, DRB1*04:40, DRB1*04:41, DRB1*04:42, DRB1*04:43, DRB1*04:44:01, DRB1*04:44:02, DRB1*04:45, DRB1*04:46, DRB1*04:47, DRB1*04:48, DRB1*04:49, DRB1*04:50, DRB1*04:51, DRB1*04:52, DRB1*04:53:01, DRB1*04:53:02, DRB1*04:54, DRB1*04:55, DRB1*04:56:01, DRB1*04:56:02, DRB1*04:57, DRB1*04:58, DRB1*04:59, DRB1*04:60, DRB1*04:61, DRB1*04:62, DRB1*04:63, DRB1*04:64, DRB1*04:65, DRB1*04:66, DRB1*04:67, DRB1*04:68, DRB1*04:69, DRB1*04:70, DRB1*04:71, DRB1*04:72:01, DRB1*04:72:02, DRB1*04:73:01, DRB1*04:73:02, DRB1*04:74, DRB1*04:75, DRB1*04:76, DRB1*04:77, DRB1*04:78, DRB1*04:79, DRB1*04:80, DRB1*04:81N, DRB1*04:82, DRB1*04:83, DRB1*04:84, DRB1*04:85, DRB1*04:86, DRB1*04:87, DRB1*04:88, DRB1*04:89, DRB1*04:90, DRB1*04:91, DRB1*04:92, DRB1*04:93, DRB1*04:94:O1N, DRB1*04:95:01, DRB1*04:95:02, DRB1*04:96, DRB1*04:97, DRB1*04:98:01, DRB1*04:98:02, DRB1*04:99, DRB1*07:01:01:01, DRB1*07:01:01:02, DRB1*07:01:01:03, DRB1*07:01:01:04, DRB1*07:01:02, DRB1*07:01:03, DRB1*07:01:04, DRB1*07:01:05, DRB1*07:01:06, DRB1*07:01:07, DRB1*07:01:08, DRB1*07:01:09, DRB1*07:01:10, DRB1*07:01:11, DRB1*07:01:12, DRB1*07:01:13, DRB1*07:01:14, DRB1*07:01:15, DRB1*07:01:16, DRB1*07:01:17, DRB1*07:01:18, DRB1*07:01:19, DRB1*07:01:20, DRB1*07:01:21, DRB1*07:01:22, DRB1*07:03, DRB1*07:04, DRB1*07:05, DRB1*07:06, DRB1*07:07, DRB1*07:08, DRB1*07:09, DRB1*07:100, DRB1*07:101N, DRB1*07:10N, DRB1*07:11, DRB1*07:12, DRB1*07:13, DRB1*07:14, DRB1*07:15, DRB1*07:16, DRB1*07:17, DRB1*07:18, DRB1*07:19, DRB1*07:20, DRB1*07:21, DRB1*07:22, DRB1*07:23, DRB1*07:24, DRB1*07:25, DRB1*07:26N, DRB1*07:27, DRB1*07:28, DRB1*07:29, DRB1*07:30, DRB1*07:31, DRB1*07:32, DRB1*07:33, DRB1*07:34, DRB1*07:35, DRB1*07:36, DRB1*07:37, DRB1*07:38, DRB1*07:39, DRB1*07:40, DRB1*07:41, DRB1*07:42, DRB1*07:43, DRB1*07:44, DRB1*07:45, DRB1*07:46, DRB1*07:47, DRB1*07:48, DRB1*07:49, DRB1*07:50, DRB1*07:51, DRB1*07:52, DRB1*07:53, DRB1*07:54, DRB1*07:55, DRB1*07:56, DRB1*07:57, DRB1*07:58N, DRB1*07:59, DRB1*07:60, DRB1*07:61, DRB1*07:62, DRB1*07:63, DRB1*07:64, DRB1*07:65, DRB1*07:66, DRB1*07:67, DRB1*07:68N, DRB1*07:69, DRB1*07:70, DRB1*07:71, DRB1*07:72, DRB1*07:73, DRB1*07:74, DRB1*07:75, DRB1*07:76, DRB1*07:77, DRB1*07:78, DRB1*07:79, DRB1*07:80, DRB1*07:81, DRB1*07:82, DRB1*07:83, DRB1*07:84, DRB1*07:85, DRB1*07:86, DRB1*07:87N, DRB1*07:88, DRB1*07:89, DRB1*07:90, DRB1*07:91, DRB1*07:92, DRB1*07:93, DRB1*07:94, DRB1*07:95, DRB1*07:96, DRB1*07:97, DRB1*07:98, DRB1*07:99, DRB1*08:01:01, DRB1*08:01:02, DRB1*08:01:04, DRB1*08:01:05, DRB1*08:01:06, DRB1*08:01:07, DRB1*08:02:01:01, DRB1*08:02:01:02, DRB1*08:02:02, DRB1*08:02:03, DRB1*08:02:04, DRB1*08:03:02:01, DRB1*08:03:02:02, DRB1*08:03:03, DRB1*08:03:04, DRB1*08:03:05, DRB1*08:03:06, DRB1*08:03:07, DRB1*08:03:08, DRB1*08:03:09, DRB1*08:04:01, DRB1*08:04:02, DRB1*08:04:03, DRB1*08:04:04, DRB1*08:04:05, DRB1*08:04:06, DRB1*08:04:07, DRB1*08:05, DRB1*08:06, DRB1*08:07, DRB1*08:08, DRB1*08:09, DRB1*08:10, DRB1*08:11, DRB1*08:12, DRB1*08:13, DRB1*08:14, DRB1*08:15, DRB1*08:16, DRB1*08:17, DRB1*08:18, DRB1*08:19, DRB1*08:20, DRB1*08:21, DRB1*08:22, DRB1*08:23, DRB1*08:24:01, DRB1*08:24:02, DRB1*08:25, DRB1*08:26, DRB1*08:27, DRB1*08:28, DRB1*08:29, DRB1*08:30:01, DRB1*08:30:02, DRB1*08:30:03, DRB1*08:31, DRB1*08:32, DRB1*08:33, DRB1*08:34, DRB1*08:35, DRB1*08:36:01, DRB1*08:36:02, DRB1*08:37, DRB1*08:38, DRB1*08:39, DRB1*08:40, DRB1*08:41, DRB1*08:42, DRB1*08:43, DRB1*08:44, DRB1*08:45:01, DRB1*08:45:02, DRB1*08:46, DRB1*08:47, DRB1*08:48, DRB1*08:49, DRB1*08:50, DRB1*08:51, DRB1*08:52, DRB1*08:53, DRB1*08:54, DRB1*08:55, DRB1*08:56, DRB1*08:57, DRB1*08:58, DRB1*08:59, DRB1*08:60N, DRB1*08:61, DRB1*08:62, DRB1*08:63, DRB1*08:64, DRB1*08:65, DRB1*08:66, DRB1*08:67, DRB1*08:68:01, DRB1*08:68:02, DRB1*08:69, DRB1*08:70, DRB1*08:71, DRB1*08:72, DRB1*08:73, DRB1*08:74, DRB1*08:75, DRB1*08:76, DRB1*08:77, DRB1*08:78N, DRB1*08:79, DRB1*08:80, DRB1*08:81, DRB1*08:82, DRB1*08:83, DRB1*08:84, DRB1*08:85, DRB1*08:86, DRB1*08:87, DRB1*08:88, DRB1*08:89N, DRB1*08:90, DRB1*09:01:02:01, DRB1*09:01:02:02, DRB1*09:01:03, DRB1*09:01:04, DRB1*09:01:05, DRB1*09:01:06, DRB1*09:01:07, DRB1*09:01:08, DRB1*09:01:09, DRB1*09:01:10, DRB1*09:01:11, DRB1*09:02:01, DRB1*09:02:02, DRB1*09:03, DRB1*09:04, DRB1*09:05, DRB1*09:06, DRB1*09:07, DRB1*09:08, DRB1*09:09, DRB1*09:10, DRB1*09:11, DRB1*09:12, DRB1*09:13, DRB1*09:14, DRB1*09:15, DRB1*09:16, DRB1*09:17, DRB1*09:18, DRB1*09:19, DRB1*09:20, DRB1*09:21, DRB1*09:22, DRB1*09:23, DRB1*09:24, DRB1*09:25, DRB1*09:26, DRB1*09:27, DRB1*09:28, DRB1*09:29, DRB1*09:30, DRB1*09:31, DRB1*09:32, DRB1*09:33, DRB1*09:34, DRB1*09:35, DRB1*09:36, DRB1*09:37N, DRB1*09:38, DRB1*09:39, DRB1*09:40, DRB1*10:01:01:01, DRB1*10:01:01:02, DRB1*10:01:01:03, DRB1*10:01:02, DRB1*10:01:03, DRB1*10:01:04, DRB1*10:01:05, DRB1*10:01:06, DRB1*10:01:07, DRB1*10:01:08, DRB1*10:01:09, DRB1*10:01:10, DRB1*10:01:11, DRB1*10:01:12, DRB1*10:02, DRB1*10:03, DRB1*10:04, DRB1*10:05, DRB1*10:06, DRB1*10:07, DRB1*10:08, DRB1*10:09, DRB1*10:10, DRB1*10:11, DRB1*10:12, DRB1*10:13, DRB1*10:14, DRB1*10:15, DRB1*10:16, DRB1*10:17, DRB1*10:18, DRB1*10:19, DRB1*10:20, DRB1*10:21, DRB1*10:22, DRB1*10:23, DRB1*10:24, DRB1*10:25, DRB1*10:26, DRB1*10:27, DRB1*10:28, DRB1*10:29, DRB1*10:30, DRB1*10:31, DRB1*10:32, DRB1*10:33, DRB1*11:01:01:01, DRB1*11:01:01:02, DRB1*11:01:01:03, DRB1*11:01:01:04, DRB1*11:01:02, DRB1*11:01:03, DRB1*11:01:04, DRB1*11:01:05, DRB1*11:01:06, DRB1*11:01:07, DRB1*11:01:08, DRB1*11:01:09, DRB1*11:01:10, DRB1*11:01:11, DRB1*11:01:12, DRB1*11:01:13, DRB1*11:01:14, DRB1*11:01:15, DRB1*11:01:16, DRB1*11:01:17, DRB1*11:01:18, DRB1*11:01:19, DRB1*11:01:20, DRB1*11:01:21, DRB1*11:01:22, DRB1*11:01:23, DRB1*11:01:24, DRB1*11:01:25, DRB1*11:01:26, DRB1*11:01:27, DRB1*11:01:28, DRB1*11:01:29, DRB1*11:01:30, DRB1*11:01:31, DRB1*11:01:32, DRB1*11:01:33, DRB1*11:02:01, DRB1*11:02:02, DRB1*11:02:03, DRB1*11:02:04, DRB1*11:02:05, DRB1*11:03:01, DRB1*11:03:02, DRB1*11:03:03, DRB1*11:03:04, DRB1*11:04:01, DRB1*11:04:02, DRB1*11:04:03, DRB1*11:04:04, DRB1*11:04:05, DRB1*11:04:06, DRB1*11:04:07, DRB1*11:04:08, DRB1*11:04:09, DRB1*11:04:10, DRB1*11:04:11, DRB1*11:04:12, DRB1*11:04:13, DRB1*11:04:14, DRB1*11:04:15, DRB1*11:04:16, DRB1*11:04:17, DRB1*11:04:18, DRB1*11:05, DRB1*11:06:01, DRB1*11:06:02, DRB1*11:06:03, DRB1*11:07:01, DRB1*11:07:02, DRB1*11:08:01, DRB1*11:08:02, DRB1*11:08:03, DRB1*11:09, DRB1*11:100, DRB1*11:101:01, DRB1*11:101:02, DRB1*11:102:01, DRB1*11:102:02, DRB1*11:103:01, DRB1*11:103:02, DRB1*11:104, DRB1*11:105, DRB1*11:106, DRB1*11:107, DRB1*11:108, DRB1*11:109, DRB1*11:10:01, DRB1*11:10:02, DRB1*11:110, DRB1*11:111, DRB1*11:112, DRB1*11:113, DRB1*11:114, DRB1*11:115, DRB1*11:116, DRB1*11:117:01, DRB1*11:117:02, DRB1*11:118, DRB1*11:119, DRB1*11:11:01, DRB1*11:11:03, DRB1*11:120, DRB1*11:121, DRB1*11:122, DRB1*11:123, DRB1*11:124:01, DRB1*11:124:02, DRB1*11:125, DRB1*11:126, DRB1*11:127, DRB1*11:128, DRB1*11:129, DRB1*11:12:01, DRB1*11:12:02, DRB1*11:12:03, DRB1*11:130, DRB1*11:131, DRB1*11:132, DRB1*11:133, DRB1*11:134, DRB1*11:135, DRB1*11:136, DRB1*11:137, DRB1*11:138, DRB1*11:139, DRB1*11:13:01, DRB1*11:13:02, DRB1*11:140, DRB1*11:141, DRB1*11:142, DRB1*11:143, DRB1*11:144, DRB1*11:145, DRB1*11:146, DRB1*11:147:01, DRB1*11:147:02, DRB1*11:148, DRB1*11:149, DRB1*11:14:01, DRB1*11:14:02, DRB1*11:15, DRB1*11:150, DRB1*11:151, DRB1*11:152, DRB1*11:153, DRB1*11:154, DRB1*11:155, DRB1*11:156, DRB1*11:157, DRB1*11:158, DRB1*11:159, DRB1*11:16, DRB1*11:160, DRB1*11:161, DRB1*11:162, DRB1*11:163, DRB1*11:164, DRB1*11:165:01, DRB1*11:165:02, DRB1*11:166, DRB1*11:167, DRB1*11:168, DRB1*11:169N, DRB1*11:17, DRB1*11:170, DRB1*11:171, DRB1*11:172, DRB1*11:173, DRB1*11:174, DRB1*11:175, DRB1*11:176, DRB1*11:177, DRB1*11:178, DRB1*11:179, DRB1*11:18, DRB1*11:180, DRB1*11:181, DRB1*11:182, DRB1*11:183, DRB1*11:184, DRB1*11:185, DRB1*11:186, DRB1*11:187, DRB1*11:188, DRB1*11:189, DRB1*11:190, DRB1*11:191, DRB1*11:192, DRB1*11:193:01, DRB1*11:193:02, DRB1*11:194, DRB1*11:195, DRB1*11:196, DRB1*11:197, DRB1*11:198, DRB1*11:199, DRB1*11:19:01, DRB1*11:19:02, DRB1*11:19:03, DRB1*11:20, DRB1*11:200, DRB1*11:201, DRB1*11:202, DRB1*11:203, DRB1*11:204, DRB1*11:205, DRB1*11:206, DRB1*11:207, DRB1*11:208, DRB1*11:209, DRB1*11:21, DRB1*11:210, DRB1*11:211, DRB1*11:212, DRB1*11:213, DRB1*11:214, DRB1*11:215, DRB1*11:216, DRB1*11:217N, DRB1*11:218, DRB1*11:219, DRB1*11:22, DRB1*11:220, DRB1*11:221, DRB1*11:222, DRB1*11:223, DRB1*11:224, DRB1*11:225, DRB1*11:226, DRB1*11:227, DRB1*11:228, DRB1*11:229, DRB1*11:230, DRB1*11:231, DRB1*11:232, DRB1*11:233, DRB1*11:234, DRB1*11:235, DRB1*11:236, DRB1*11:237, DRB1*11:238, DRB1*11:239, DRB1*11:23:01, DRB1*11:23:02, DRB1*11:240, DRB1*11:241, DRB1*11:242, DRB1*11:243, DRB1*11:244, DRB1*11:245, DRB1*11:246N, DRB1*11:247, DRB1*11:248Q, DRB1*11:249, DRB1*11:24:01, DRB1*11:24:02, DRB1*11:25, DRB1*11:250N, DRB1*11:251, DRB1*11:252, DRB1*11:253, DRB1*11:254, DRB1*11:26, DRB1*11:27:01, DRB1*11:27:02, DRB1*11:27:03, DRB1*11:28:01, DRB1*11:28:02, DRB1*11:29:01, DRB1*11:29:02, DRB1*11:30, DRB1*11:31, DRB1*11:32, DRB1*11:33, DRB1*11:34, DRB1*11:35, DRB1*11:36, DRB1*11:37:01, DRB1*11:37:02, DRB1*11:38, DRB1*11:39, DRB1*11:40, DRB1*11:41, DRB1*11:42:01, DRB1*11:42:02, DRB1*11:43, DRB1*11:44, DRB1*11:45, DRB1*11:46:01, DRB1*11:46:02, DRB1*11:47, DRB1*11:48, DRB1*11:49:01, DRB1*11:49:02, DRB1*11:50, DRB1*11:51, DRB1*11:52, DRB1*11:53, DRB1*11:54:01, DRB1*11:54:02, DRB1*11:55, DRB1*11:56, DRB1*11:57, DRB1*11:58:01, DRB1*11:58:02, DRB1*11:59, DRB1*11:60, DRB1*11:61, DRB1*11:62:01, DRB1*11:62:02, DRB1*11:63:01, DRB1*11:63:02, DRB1*11:64, DRB1*11:65:01, DRB1*11:65:02, DRB1*11:66:01, DRB1*11:66:02, DRB1*11:67, DRB1*11:68, DRB1*11:69, DRB1*11:70, DRB1*11:72, DRB1*11:73, DRB1*11:74:01, DRB1*11:74:02, DRB1*11:75, DRB1*11:76, DRB1*11:77, DRB1*11:78, DRB1*11:79, DRB1*11:80, DRB1*11:81, DRB1*11:82, DRB1*11:83, DRB1*11:84:01, DRB1*11:84:02, DRB1*11:84:03, DRB1*11:85, DRB1*11:86, DRB1*11:87, DRB1*11:88, DRB1*11:89, DRB1*11:90, DRB1*11:91, DRB1*11:92, DRB1*11:93, DRB1*11:94, DRB1*11:95, DRB1*11:96, DRB1*11:97, DRB1*11:98, DRB1*11:99, DRB1*12:01:01:01, DRB1*12:01:01:02, DRB1*12:01:01:03, DRB1*12:01:01:04, DRB1*12:01:01:05, DRB1*12:01:01:06, DRB1*12:01:02, DRB1*12:01:03, DRB1*12:01:04, DRB1*12:01:05, DRB1*12:01:06, DRB1*12:01:07, DRB1*12:01:08, DRB1*12:01:09, DRB1*12:02:01:01, DRB1*12:02:01:02, DRB1*12:02:01:03, DRB1*12:02:01:04, DRB1*12:02:02, DRB1*12:02:03, DRB1*12:02:04, DRB1*12:02:05, DRB1*12:02:06, DRB1*12:02:07, DRB1*12:02:08, DRB1*12:02:09, DRB1*12:03:02, DRB1*12:03:03, DRB1*12:04, DRB1*12:05, DRB1*12:06, DRB1*12:07, DRB1*12:08, DRB1*12:09, DRB1*12:10, DRB1*12:11, DRB1*12:12, DRB1*12:13, DRB1*12:14, DRB1*12:15, DRB1*12:16:01, DRB1*12:16:02, DRB1*12:16:03, DRB1*12:17, DRB1*12:18, DRB1*12:19, DRB1*12:20, DRB1*12:21, DRB1*12:22, DRB1*12:23, DRB1*12:24N, DRB1*12:25, DRB1*12:26, DRB1*12:27, DRB1*12:28, DRB1*12:29, DRB1*12:30, DRB1*12:31N, DRB1*12:32, DRB1*12:33, DRB1*12:34, DRB1*12:35, DRB1*12:36, DRB1*12:37, DRB1*12:38, DRB1*12:39, DRB1*12:40, DRB1*12:41, DRB1*12:42, DRB1*12:43, DRB1*12:44, DRB1*12:45, DRB1*12:46, DRB1*12:47, DRB1*12:48, DRB1*12:49, DRB1*12:50, DRB1*12:51, DRB1*12:52, DRB1*12:53, DRB1*12:54, DRB1*12:55, DRB1*12:56, DRB1*12:57, DRB1*12:58, DRB1*12:59, DRB1*12:60N, DRB1*12:61, DRB1*12:62, DRB1*12:63, DRB1*12:64, DRB1*12:65, DRB1*12:66, DRB1*12:67, DRB1*12:68, DRB1*12:69, DRB1*12:70, DRB1*12:71, DRB1*12:72N, DRB1*12:73, DRB1*12:74N, DRB1*12:75, DRB1*13:01:01:01, DRB1*13:01:01:02, DRB1*13:01:02, DRB1*13:01:03, DRB1*13:01:04, DRB1*13:01:05, DRB1*13:01:06, DRB1*13:01:07, DRB1*13:01:08, DRB1*13:01:09, DRB1*13:01:10, DRB1*13:01:11, DRB1*13:01:12, DRB1*13:01:13, DRB1*13:01:14, DRB1*13:01:15, DRB1*13:01:16, DRB1*13:01:17, DRB1*13:01:18, DRB1*13:01:19, DRB1*13:01:20, DRB1*13:01:21, DRB1*13:01:22, DRB1*13:01:23, DRB1*13:01:24, DRB1*13:01:25, DRB1*13:01:26, DRB1*13:02:01:01, DRB1*13:02:01:02, DRB1*13:02:01:03, DRB1*13:02:02, DRB1*13:02:03, DRB1*13:02:04, DRB1*13:02:05, DRB1*13:02:06, DRB1*13:02:07, DRB1*13:02:08, DRB1*13:02:09, DRB1*13:02:10, DRB1*13:02:11, DRB1*13:02:12, DRB1*13:02:13, DRB1*13:02:14, DRB1*13:02:15, DRB1*13:02:16, DRB1*13:02:17, DRB1*13:03:01, DRB1*13:03:02, DRB1*13:03:03, DRB1*13:03:04, DRB1*13:03:05, DRB1*13:03:06, DRB1*13:03:07, DRB1*13:03:08, DRB1*13:03:09, DRB1*13:04, DRB1*13:05:01, DRB1*13:05:02, DRB1*13:05:03, DRB1*13:06, DRB1*13:07:01, DRB1*13:07:02, DRB1*13:08, DRB1*13:09, DRB1*13:10, DRB1*13:100, DRB1*13:101, DRB1*13:102, DRB1*13:103, DRB1*13:104, DRB1*13:105, DRB1*13:106, DRB1*13:107, DRB1*13:108, DRB1*13:109, DRB1*13:110, DRB1*13:111, DRB1*13:112, DRB1*13:113N, DRB1*13:114, DRB1*13:115, DRB1*13:116, DRB1*13:117, DRB1*13:118, DRB1*13:119, DRB1*13:11:01, DRB1*13:11:02, DRB1*13:120, DRB1*13:121, DRB1*13:122, DRB1*13:123, DRB1*13:124, DRB1*13:125, DRB1*13:126, DRB1*13:127, DRB1*13:128, DRB1*13:129, DRB1*13:12:01, DRB1*13:12:02, DRB1*13:12:03, DRB1*13:12:04, DRB1*13:13, DRB1*13:130, DRB1*13:131, DRB1*13:132, DRB1*13:133, DRB1*13:134, DRB1*13:135, DRB1*13:136, DRB1*13:137N, DRB1*13:138, DRB1*13:139, DRB1*13:140, DRB1*13:141, DRB1*13:142N, DRB1*13:143, DRB1*13:144, DRB1*13:145, DRB1*13:146, DRB1*13:147, DRB1*13:148, DRB1*13:149, DRB1*13:14:01, DRB1*13:14:02, DRB1*13:14:03, DRB1*13:15, DRB1*13:150, DRB1*13:151, DRB1*13:152, DRB1*13:153, DRB1*13:154, DRB1*13:155, DRB1*13:156, DRB1*13:157, DRB1*13:158, DRB1*13:159, DRB1*13:16, DRB1*13:160, DRB1*13:161, DRB1*13:162, DRB1*13:163, DRB1*13:164, DRB1*13:165, DRB1*13:166, DRB1*13:167, DRB1*13:168, DRB1*13:169, DRB1*13:17, DRB1*13:170, DRB1*13:171:01, DRB1*13:171:02, DRB1*13:172, DRB1*13:173, DRB1*13:174, DRB1*13:175, DRB1*13:176, DRB1*13:177, DRB1*13:178, DRB1*13:179, DRB1*13:18, DRB1*13:180, DRB1*13:181, DRB1*13:182, DRB1*13:183, DRB1*13:184, DRB1*13:185N, DRB1*13:186, DRB1*13:187, DRB1*13:188, DRB1*13:189, DRB1*13:19, DRB1*13:190, DRB1*13:191, DRB1*13:192, DRB1*13:193, DRB1*13:194, DRB1*13:195, DRB1*13:196, DRB1*13:197, DRB1*13:198, DRB1*13:199, DRB1*13:20, DRB1*13:200N, DRB1*13:201, DRB1*13:202, DRB1*13:203, DRB1*13:204, DRB1*13:205, DRB1*13:206, DRB1*13:207, DRB1*13:208, DRB1*13:209, DRB1*13:210, DRB1*13:211, DRB1*13:212, DRB1*13:213, DRB1*13:214, DRB1*13:215, DRB1*13:216, DRB1*13:217, DRB1*13:218, DRB1*13:219, DRB1*13:21:01, DRB1*13:21:02, DRB1*13:220, DRB1*13:221, DRB1*13:222, DRB1*13:223, DRB1*13:224, DRB1*13:225, DRB1*13:226, DRB1*13:227, DRB1*13:228, DRB1*13:229, DRB1*13:22:01, DRB1*13:22:02, DRB1*13:230, DRB1*13:231, DRB1*13:232, DRB1*13:233, DRB1*13:234, DRB1*13:235, DRB1*13:236, DRB1*13:237, DRB1*13:238, DRB1*13:239, DRB1*13:23:01, DRB1*13:23:02, DRB1*13:24, DRB1*13:240, DRB1*13:241, DRB1*13:242:01, DRB1*13:242:02, DRB1*13:243, DRB1*13:244, DRB1*13:245, DRB1*13:246, DRB1*13:247, DRB1*13:248, DRB1*13:249N, DRB1*13:25, DRB1*13:250, DRB1*13:251, DRB1*13:252N, DRB1*13:253, DRB1*13:254, DRB1*13:255N, DRB1*13:256, DRB1*13:257, DRB1*13:258, DRB1*13:259, DRB1*13:260, DRB1*13:261, DRB1*13:262, DRB1*13:263, DRB1*13:264, DRB1*13:265, DRB1*13:266, DRB1*13:267, DRB1*13:268N, DRB1*13:269, DRB1*13:26:01, DRB1*13:26:02, DRB1*13:27, DRB1*13:270, DRB1*13:271, DRB1*13:272, DRB1*13:273, DRB1*13:274, DRB1*13:275, DRB1*13:276, DRB1*13:277, DRB1*13:278Q, DRB1*13:279, DRB1*13:28:01, DRB1*13:28:02, DRB1*13:29, DRB1*13:30, DRB1*13:31, DRB1*13:32, DRB1*13:33:01, DRB1*13:33:02, DRB1*13:33:03, DRB1*13:34, DRB1*13:35, DRB1*13:36, DRB1*13:37, DRB1*13:38, DRB1*13:39, DRB1*13:40, DRB1*13:41, DRB1*13:42, DRB1*13:43, DRB1*13:44, DRB1*13:45, DRB1*13:46, DRB1*13:47, DRB1*13:48, DRB1*13:49, DRB1*13:50:01, DRB1*13:50:02, DRB1*13:50:03, DRB1*13:51, DRB1*13:52, DRB1*13:53, DRB1*13:54, DRB1*13:55, DRB1*13:56, DRB1*13:57, DRB1*13:58, DRB1*13:59, DRB1*13:60, DRB1*13:61:01, DRB1*13:61:02, DRB1*13:62, DRB1*13:63, DRB1*13:64, DRB1*13:65, DRB1*13:66:01, DRB1*13:66:02, DRB1*13:67, DRB1*13:68, DRB1*13:69, DRB1*13:70, DRB1*13:71, DRB1*13:72, DRB1*13:73, DRB1*13:74, DRB1*13:75, DRB1*13:76, DRB1*13:77, DRB1*13:78, DRB1*13:79, DRB1*13:80, DRB1*13:81, DRB1*13:82, DRB1*13:83, DRB1*13:84, DRB1*13:85, DRB1*13:86, DRB1*13:87, DRB1*13:88, DRB1*13:89:01, DRB1*13:89:02, DRB1*13:90, DRB1*13:91, DRB1*13:92, DRB1*13:93, DRB1*13:94:01, DRB1*13:94:02, DRB1*13:95, DRB1*13:96:01, DRB1*13:96:02, DRB1*13:97:01, DRB1*13:97:02, DRB1*13:98, DRB1*13:99, DRB1*14:01:01, DRB1*14:01:02, DRB1*14:01:03, DRB1*14:01:04, DRB1*14:02:01:01, DRB1*14:02:01:02, DRB1*14:02:02, DRB1*14:02:03, DRB1*14:02:04, DRB1*14:02:05, DRB1*14:02:06, DRB1*14:02:07, DRB1*14:03:01, DRB1*14:03:02, DRB1*14:04:01, DRB1*14:04:02, DRB1*14:04:03, DRB1*14:04:04, DRB1*14:04:05, DRB1*14:04:06, DRB1*14:05:01:01, DRB1*14:05:01:02, DRB1*14:05:02, DRB1*14:05:03, DRB1*14:05:04, DRB1*14:06:01, DRB1*14:06:02, DRB1*14:06:03, DRB1*14:06:04, DRB1*14:07:01, DRB1*14:07:02, DRB1*14:08, DRB1*14:09, DRB1*14:10, DRB1*14:100, DRB1*14:101, DRB1*14:102, DRB1*14:103, DRB1*14:104, DRB1*14:105, DRB1*14:106, DRB1*14:107, DRB1*14:108, DRB1*14:109, DRB1*14:11, DRB1*14:110, DRB1*14:111, DRB1*14:112, DRB1*14:113, DRB1*14:114, DRB1*14:115, DRB1*14:116, DRB1*14:117, DRB1*14:118, DRB1*14:119, DRB1*14:120, DRB1*14:121, DRB1*14:122, DRB1*14:123, DRB1*14:124, DRB1*14:125, DRB1*14:126:01, DRB1*14:126:02, DRB1*14:127:01, DRB1*14:127:02, DRB1*14:128, DRB1*14:129, DRB1*14:12:01, DRB1*14:12:02, DRB1*14:13, DRB1*14:130, DRB1*14:131, DRB1*14:132, DRB1*14:133, DRB1*14:134, DRB1*14:135, DRB1*14:136, DRB1*14:137N, DRB1*14:138, DRB1*14:139, DRB1*14:14, DRB1*14:140, DRB1*14:141, DRB1*14:142, DRB1*14:143, DRB1*14:144, DRB1*14:145, DRB1*14:146, DRB1*14:147, DRB1*14:148, DRB1*14:149, DRB1*14:15, DRB1*14:150, DRB1*14:151, DRB1*14:152N, DRB1*14:153, DRB1*14:154, DRB1*14:155, DRB1*14:156, DRB1*14:157, DRB1*14:158, DRB1*14:159, DRB1*14:16, DRB1*14:160, DRB1*14:161, DRB1*14:162, DRB1*14:163, DRB1*14:164, DRB1*14:165, DRB1*14:166N, DRB1*14:167, DRB1*14:168, DRB1*14:169, DRB1*14:17, DRB1*14:170, DRB1*14:171, DRB1*14:172, DRB1*14:173, DRB1*14:174, DRB1*14:175, DRB1*14:176, DRB1*14:177, DRB1*14:178, DRB1*14:179, DRB1*14:18, DRB1*14:180, DRB1*14:181, DRB1*14:182, DRB1*14:183, DRB1*14:184, DRB1*14:185, DRB1*14:186, DRB1*14:187, DRB1*14:188N, DRB1*14:189, DRB1*14:19, DRB1*14:190, DRB1*14:191, DRB1*14:192, DRB1*14:193, DRB1*14:194, DRB1*14:195N, DRB1*14:196, DRB1*14:197N, DRB1*14:198, DRB1*14:199, DRB1*14:20, DRB1*14:200, DRB1*14:201, DRB1*14:202, DRB1*14:203, DRB1*14:204, DRB1*14:205, DRB1*14:206, DRB1*14:207, DRB1*14:208, DRB1*14:209, DRB1*14:21, DRB1*14:210Q, DRB1*14:211, DRB1*14:22, DRB1*14:23:01, DRB1*14:23:02, DRB1*14:23:03, DRB1*14:23:04, DRB1*14:24, DRB1*14:25:01, DRB1*14:25:02, DRB1*14:26, DRB1*14:27:01, DRB1*14:27:02, DRB1*14:28, DRB1*14:29, DRB1*14:30, DRB1*14:31, DRB1*14:32:01, DRB1*14:32:02, DRB1*14:32:03, DRB1*14:33, DRB1*14:34, DRB1*14:35, DRB1*14:36, DRB1*14:37, DRB1*14:38:01, DRB1*14:38:02, DRB1*14:39, DRB1*14:40, DRB1*14:41, DRB1*14:42, DRB1*14:43, DRB1*14:44:01, DRB1*14:44:02, DRB1*14:44:03, DRB1*14:45, DRB1*14:46, DRB1*14:47, DRB1*14:48, DRB1*14:49, DRB1*14:50, DRB1*14:51, DRB1*14:52, DRB1*14:53, DRB1*14:54:01:01, DRB1*14:54:01:02, DRB1*14:54:01:03, DRB1*14:54:01:04, DRB1*14:54:02, DRB1*14:54:03, DRB1*14:54:04, DRB1*14:54:05, DRB1*14:54:06, DRB1*14:54:07, DRB1*14:55, DRB1*14:56, DRB1*14:57, DRB1*14:58, DRB1*14:59, DRB1*14:60, DRB1*14:61, DRB1*14:62, DRB1*14:63, DRB1*14:64, DRB1*14:65, DRB1*14:67, DRB1*14:68:01, DRB1*14:68:02, DRB1*14:69, DRB1*14:70, DRB1*14:71, DRB1*14:72, DRB1*14:73, DRB1*14:74, DRB1*14:75, DRB1*14:76, DRB1*14:77, DRB1*14:78, DRB1*14:79, DRB1*14:80, DRB1*14:81, DRB1*14:82, DRB1*14:83, DRB1*14:84, DRB1*14:85, DRB1*14:86, DRB1*14:87, DRB1*14:88, DRB1*14:89, DRB1*14:90, DRB1*14:91, DRB1*14:92N, DRB1*14:93, DRB1*14:94, DRB1*14:95, DRB1*14:96, DRB1*14:97, DRB1*14:98, DRB1*14:99, DRB1*15:01:01:01, DRB1*15:01:01:02, DRB1*15:01:01:03, DRB1*15:01:01:04, DRB1*15:01:01:05, DRB1*15:01:02, DRB1*15:01:03, DRB1*15:01:04, DRB1*15:01:05, DRB1*15:01:06, DRB1*15:01:07, DRB1*15:01:08, DRB1*15:01:09, DRB1*15:01:10, DRB1*15:01:11, DRB1*15:01:12, DRB1*15:01:13, DRB1*15:01:14, DRB1*15:01:15, DRB1*15:01:16, DRB1*15:01:17, DRB1*15:01:18, DRB1*15:01:19, DRB1*15:01:20, DRB1*15:01:21, DRB1*15:01:22, DRB1*15:01:23, DRB1*15:01:24, DRB1*15:01:25, DRB1*15:01:26, DRB1*15:01:27, DRB1*15:01:28, DRB1*15:01:29, DRB1*15:01:30, DRB1*15:01:31, DRB1*15:01:32, DRB1*15:01:33, DRB1*15:01:34, DRB1*15:01:35, DRB1*15:01:36, DRB1*15:01:37, DRB1*15:01:38, DRB1*15:01:39, DRB1*15:01:40, DRB1*15:01:41, DRB1*15:02:01:01, DRB1*15:02:01:02, DRB1*15:02:01:03, DRB1*15:02:02, DRB1*15:02:03, DRB1*15:02:04, DRB1*15:02:05, DRB1*15:02:06, DRB1*15:02:07, DRB1*15:02:08, DRB1*15:02:09, DRB1*15:02:10, DRB1*15:02:11, DRB1*15:02:12, DRB1*15:02:13, DRB1*15:02:14, DRB1*15:02:15, DRB1*15:02:16, DRB1*15:02:17, DRB1*15:02:18, DRB1*15:02:19, DRB1*15:03:01:01, DRB1*15:03:01:02, DRB1*15:03:01:03, DRB1*15:03:02, DRB1*15:03:03, DRB1*15:03:04, DRB1*15:04, DRB1*15:05, DRB1*15:06:01, DRB1*15:06:02, DRB1*15:06:03, DRB1*15:06:04, DRB1*15:07:01, DRB1*15:07:02, DRB1*15:07:03, DRB1*15:08, DRB1*15:09, DRB1*15:10, DRB1*15:100, DRB1*15:101, DRB1*15:102, DRB1*15:103, DRB1*15:104:01, DRB1*15:104:02, DRB1*15:104:03, DRB1*15:105:01, DRB1*15:105:02, DRB1*15:106, DRB1*15:107, DRB1*15:108, DRB1*15:109, DRB1*15:110, DRB1*15:111, DRB1*15:112, DRB1*15:113N, DRB1*15:114, DRB1*15:115N, DRB1*15:116, DRB1*15:117, DRB1*15:118, DRB1*15:119, DRB1*15:11:01, DRB1*15:11:02, DRB1*15:12, DRB1*15:120, DRB1*15:121, DRB1*15:122, DRB1*15:123, DRB1*15:124, DRB1*15:125, DRB1*15:126, DRB1*15:127, DRB1*15:128, DRB1*15:129N, DRB1*15:13, DRB1*15:130, DRB1*15:131, DRB1*15:132, DRB1*15:133, DRB1*15:134N, DRB1*15:135, DRB1*15:136, DRB1*15:137N, DRB1*15:138N, DRB1*15:139, DRB1*15:14, DRB1*15:140, DRB1*15:141, DRB1*15:142, DRB1*15:143, DRB1*15:144, DRB1*15:145, DRB1*15:146, DRB1*15:147, DRB1*15:148N, DRB1*15:149, DRB1*15:150, DRB1*15:151, DRB1*15:152, DRB1*15:153, DRB1*15:154N, DRB1*15:155, DRB1*15:156, DRB1*15:157, DRB1*15:158, DRB1*15:159N, DRB1*15:15:01, DRB1*15:15:02, DRB1*15:15:03, DRB1*15:16, DRB1*15:160, DRB1*15:161, DRB1*15:162, DRB1*15:163N, DRB1*15:164Q, DRB1*15:165, DRB1*15:166, DRB1*15:167, DRB1*15:168, DRB1*15:169, DRB1*15:170, DRB1*15:17N, DRB1*15:18, DRB1*15:19, DRB1*15:20, DRB1*15:21, DRB1*15:22, DRB1*15:23, DRB1*15:24, DRB1*15:25, DRB1*15:26, DRB1*15:27, DRB1*15:28, DRB1*15:29, DRB1*15:30, DRB1*15:31:01, DRB1*15:31:02, DRB1*15:32, DRB1*15:33, DRB1*15:34, DRB1*15:35, DRB1*15:36, DRB1*15:37:01, DRB1*15:37:02, DRB1*15:38, DRB1*15:39, DRB1*15:40, DRB1*15:41, DRB1*15:42, DRB1*15:43, DRB1*15:44, DRB1*15:45, DRB1*15:46, DRB1*15:47, DRB1*15:48, DRB1*15:49, DRB1*15:50N, DRB1*15:51, DRB1*15:52, DRB1*15:53, DRB1*15:54, DRB1*15:55, DRB1*15:56, DRB1*15:57, DRB1*15:58, DRB1*15:59, DRB1*15:60, DRB1*15:61, DRB1*15:62, DRB1*15:63, DRB1*15:64, DRB1*15:65, DRB1*15:66:01, DRB1*15:66:02, DRB1*15:67, DRB1*15:68, DRB1*15:69, DRB1*15:70, DRB1*15:71, DRB1*15:72, DRB1*15:73, DRB1*15:74, DRB1*15:75, DRB1*15:76, DRB1*15:77, DRB1*15:78, DRB1*15:79, DRB1*15:80N, DRB1*15:81, DRB1*15:82, DRB1*15:83, DRB1*15:84, DRB1*15:85, DRB1*15:86, DRB1*15:87, DRB1*15:88, DRB1*15:89, DRB1*15:90, DRB1*15:91, DRB1*15:92, DRB1*15:93, DRB1*15:94, DRB1*15:95, DRB1*15:96, DRB1*15:97, DRB1*15:98, DRB1*15:99, DRB1*16:01:01, DRB1*16:01:02, DRB1*16:01:03, DRB1*16:01:04, DRB1*16:01:05, DRB1*16:01:06, DRB1*16:01:07, DRB1*16:01:08, DRB1*16:01:09, DRB1*16:01:10, DRB1*16:01:11, DRB1*16:01:12, DRB1*16:01:13, DRB1*16:01:14, DRB1*16:01:15, DRB1*16:01:16, DRB1*16:02:01:01, DRB1*16:02:01:02, DRB1*16:02:01:03, DRB1*16:02:02, DRB1*16:02:03, DRB1*16:02:04, DRB1*16:02:05, DRB1*16:02:06, DRB1*16:02:07, DRB1*16:02:08, DRB1*16:03, DRB1*16:04:01, DRB1*16:04:02, DRB1*16:05:01, DRB1*16:05:02, DRB1*16:07, DRB1*16:08, DRB1*16:09:01, DRB1*16:09:02, DRB1*16:10:01, DRB1*16:10:02, DRB1*16:11, DRB1*16:12, DRB1*16:13N, DRB1*16:14, DRB1*16:15, DRB1*16:16, DRB1*16:17, DRB1*16:18, DRB1*16:19, DRB1*16:20, DRB1*16:21N, DRB1*16:22, DRB1*16:23, DRB1*16:24, DRB1*16:25, DRB1*16:26, DRB1*16:27, DRB1*16:28, DRB1*16:29, DRB1*16:30, DRB1*16:31, DRB1*16:32, DRB1*16:33, DRB1*16:34, DRB1*16:35, DRB1*16:36, DRB1*16:37, DRB1*16:38:01, DRB1*16:38:02, DRB1*16:39, DRB1*16:40, DRB1*16:41N, DRB1*16:42, DRB1*16:43, DRB1*16:44, DRB1*16:45, DRB1*16:46, DRB1*16:47, DRB1*16:48, DRB1*16:49, DRB1*16:50, DRB1*16:51, DRB1*16:52, DRB1*16:53, DRB1*16:54, DRB1*16:55N, DRB1*16:56, and any combination thereof.

In certain aspects, the MHC class II molecule comprises a DR beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 21, wherein the DR beta chain comprises (i) a tryptophan at a position corresponding to amino acid residue 114 of SEQ ID NO: 19, (ii) a methionine at a position corresponding to amino acid residue 143 of SEQ ID NO: 19, (iii) a histidine at a position corresponding to amino acid residue 118 of SEQ ID NO: 19; and (iv) an isoleucine at a position corresponding to amino acid residue 157 of SEQ ID NO: 19. In certain aspects, the MHC class II molecule comprises a DR beta chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 21, wherein the DR beta chain comprises (i) a tryptophan at a position corresponding to amino acid residue 114 of SEQ ID NO: 19, (ii) a methionine at a position corresponding to amino acid residue 143 of SEQ ID NO: 19, (iii) a histidine at a position corresponding to amino acid residue 118 of SEQ ID NO: 19; (iv) an isoleucine at a position corresponding to amino acid residue 157 of SEQ ID NO: 19; (v) a threonine at a position corresponding to amino acid residue 139 of SEQ ID NO: 19; (vi) a glutamine at a position corresponding to amino acid residue 146 of SEQ ID NO: 19; (vii) a methionine at a position corresponding to amino acid residue 163 of SEQ ID NO: 19; and (viii) a threonine at a position corresponding to amino acid residue 164 of SEQ ID NO: 19. In certain aspects, the MHC class II molecule comprises a DR beta chain comprising an amino acid sequence set forth in SEQ ID NO: 21.

II.A.3.b. HLA-DR Alpha Chain

In some aspects of the present disclosure, the MHC class II molecule further comprises an alpha chain. In some aspects, the alpha chain is a wild-type alpha chain. In some aspects, the alpha chain is a DR alpha chain. Any DR alpha chain can be used in the compositions and methods of the present disclosure. In some aspects, the DR alpha chain comprises an HLA-DRA1*01 allele.

In certain aspects, the DR alpha chain is selected from DRA*01:01:01:01, DRA*01:01:01:02, DRA*01:01:01:03, DRA*01:01:02, DRA*01:02:01, DRA*01:02:02, DRA*01:02:03, and any combination thereof.

In certain aspects, the MHC class II molecule comprises a DR alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 24. In certain aspects, the MHC class II molecule comprises a DR alpha chain comprising an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 26. In certain aspects, the MHC class II molecule comprises a DR alpha chain comprising an amino acid sequence set forth in SEQ ID NO: 24. In certain aspects, the MHC class II molecule comprises a DR alpha chain comprising an amino acid sequence set forth in SEQ ID NO: 26.

II.A.4. Signal Peptide

In some aspects, the beta chain and/or the alpha chain further comprises a signal peptide. Any signal peptide known in the art can be used in the compositions and methods disclosed herein. In some aspects the beta chain signal peptide is the same as the alpha signal peptide. In some aspects the beta chain signal peptide is different from the alpha signal peptide.

In some aspects, the signal peptide is derived from a native signal peptide. In some aspects, the signal peptide is derived from a naturally occurring DP beta chain signal peptide. In some aspects, the signal peptide comprises a naturally occurring DP beta chain signal peptide. In some aspects, the signal peptide is derived from a naturally occurring DP alpha chain signal peptide. In some aspects, the signal peptide comprises a naturally occurring DP alpha chain signal peptide.

In some aspects, the signal peptide is derived from a naturally occurring DQ beta chain signal peptide. In some aspects, the signal peptide comprises a naturally occurring DQ beta chain signal peptide. In some aspects, the signal peptide is derived from a naturally occurring DQ alpha chain signal peptide. In some aspects, the signal peptide comprises a naturally occurring DQ alpha chain signal peptide.

In some aspects, the signal peptide is derived from a naturally occurring DR beta chain signal peptide. In some aspects, the signal peptide comprises a naturally occurring DR beta chain signal peptide. In some aspects, the signal peptide is derived from a naturally occurring DR alpha chain signal peptide. In some aspects, the signal peptide comprises a naturally occurring DR alpha chain signal peptide.

In some aspects, the signal peptide is derived from a fibroin light chain (FibL) signal peptide. In some aspects, the signal peptide comprises SEQ ID NO: 9. In some aspects, the signal peptide is synthetic.

II.A.5. Transmembrane Domain

In some aspects, the beta chain and/or the alpha chain further comprises a transmembrane domain. The transmembrane domain can be any length and of any origin. In some aspects, the transmembrane domain is at least about 1 to at least about 50 amino acid in length. In some aspects, the transmembrane domain is derived from a naturally occurring transmembrane domain. In some aspects, the transmembrane domain comprises a naturally occurring transmembrane domain. In some aspects, the transmembrane domain is derived from a naturally occurring HLA transmembrane domain. In some aspects, the transmembrane domain comprises a naturally occurring HLA transmembrane domain.

In some aspects, the transmembrane domain is derived from a naturally occurring DP beta chain transmembrane domain. In some aspects, the transmembrane domain comprises a naturally occurring DP beta chain transmembrane domain. In some aspects, the transmembrane domain is derived from a naturally occurring DP alpha chain transmembrane domain. In some aspects, the transmembrane domain comprises a naturally occurring DP alpha chain transmembrane domain.

In some aspects, the transmembrane domain is derived from a naturally occurring DQ beta chain transmembrane domain. In some aspects, the transmembrane domain comprises a naturally occurring DQ beta chain transmembrane domain. In some aspects, the transmembrane domain is derived from a naturally occurring DQ alpha chain transmembrane domain. In some aspects, the transmembrane domain comprises a naturally occurring DQ alpha chain transmembrane domain.

In some aspects, the transmembrane domain is derived from a naturally occurring DR beta chain transmembrane domain. In some aspects, the transmembrane domain comprises a naturally occurring DR beta chain transmembrane domain. In some aspects, the transmembrane domain is derived from a naturally occurring DR alpha chain transmembrane domain. In some aspects, the transmembrane domain comprises a naturally occurring DR alpha chain transmembrane domain.

II.A.6. Leucine Zipper

In some aspects, the beta chain and/or the alpha chain further comprises one or more leucine zipper (LZip) sequences. Any LZip sequence known in the art can be used in the compositions and methods disclosed herein. In some aspects, the beta chain and/or the alpha chain comprises an acidic LZip (αLZip), a basic LZip (βLZip), or both. In some aspects, the one or more LZip sequences are derived from a naturally occurring LZip sequence. In some aspects, the one or more LZip sequences comprise a naturally occurring LZip sequence. In some aspects, the one or more LZip sequences are synthetic. In certain aspects, the one or more LZip sequences comprise the LZip sequences set forth in SEQ ID NO: 4, 7, 14, 17, 22,or 25.

II.A.7. Linker

In some aspects, the beta chain and/or the alpha chain useful for the disclosure further comprises a linker. Any linker known in the art can be used in the compositions and methods disclosed herein. In certain aspects, the linker comprises a Gly/Ser linker. In some aspects, the linker comprises an amino acid sequence selected from GlySer, Gly₂Ser, Gly₃Ser, and Gly₄Ser. In some aspects, the linker is positioned at the N-terminus of the extracellular domain of the alpha chain or the beta chain. In some aspects, the linker is positioned at the C-terminus of the extracellular domain of the alpha chain or the beta chain. In some aspects, the linker is positioned between the extracellular domain of the alpha chain or the beta chain and the transmembrane domain. In some aspects, the linker is positioned between the extracellular domain of the alpha chain or the beta chain and the one or more LZip sequences. In some aspects, the linker is positioned between the extracellular domain of the alpha chain or the beta chain and the signal peptide.

A linker of any length can be used in the compositions and methods disclosed herein. In some aspects, the linker is at least one amino acid in length. In some aspects, the linker is at least about 1 to at least about 100, at least about 1 to at least about 90, at least about 1 to at least about 80, at least about 1 to at least about 70, at least about 1 to at least about 60, at least about 1 to at least about 50, at least about 1 to at least about 40, at least about 1 to at least about 30, at least about 1 to at least about 20, at least about 1 to at least about 15, at least about 1 to at least about 14, at least about 1 to at least about 13, at least about 1 to at least about 12, at least about 1 to at least about 11, at least about 1 to at least about 10, at least about 1 to at least about 9, at least about 1 to at least about 8, at least about 1 to at least about 7, at least about 1 to at least about 6, at least about 1 to at least about 5, at least about 1 to at least about 4, at least about 1 to at least about 3 amino acids in length.

In some aspects, the linker is at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, at least about 50, at least about 60, at least about 70, at least about 80, at least about 90, at least about 100 amino acids in length. In certain aspects, the linker is about 3 amino acids in length. In certain aspects, the linker is about 4 amino acids in length. In certain aspects, the linker is about 5 amino acids in length.

II.B. Cells

In certain aspects of the present disclosure, the MHC class II molecule used in the methods of the present disclosure is linked to or associated with a membrane of a cell. Accordingly, some aspects of the present disclosure are directed to a method of identifying an MHC class II-specific TCR comprising contacting a T cell with a cell, wherein the cell comprises a complex comprising an MHC class II molecule disclosed herein and a peptide, e.g., an epitope. In certain aspects, the beta chain of the MHC class II molecule is linked or associated with a membrane of a cell. In certain aspects, the alpha chain of the MHC class II molecule is linked or associated with a membrane of a cell. In certain aspects, the alpha chain and the beta chain of the MHC class II molecule are linked or associated with a membrane of a cell.

Any cell can be used in the methods described herein. In certain aspects the cell is a mammalian cell. In some aspects, the cell is an insect cell. In some aspects, the cell is derived from a healthy cell, e.g., a health fibroblast cell. In some aspects the cell is derived from a tumor cell. Non-limiting examples of cells that are useful in the present disclosure include K562 cells, T2 cells, HEK293 cells, HEK293T cells, A375 cells, SK-MEL-28 cells, Me275 cells, COS cells, fibroblast cells, tumor cells, or any combination thereof. In certain aspects, the cell is any cell disclosed in Hasan et al., Adv. Genet. Eng. 4(3):130 (2015), which is incorporated by reference herein in its entirety.

In certain aspects, the cell is a professional APC. In certain aspects, the cell is a macrophage, a B cell, a dendritic cell, or any combination thereof.

In certain aspects, the cell lacks endogenous expression of one or more MHC class II allele. In some aspects the cell lacks endogenous expression of an HLA-DP allele. In some aspects the cell lacks endogenous expression of an HLA-DP alpha chain allele. In some aspects the cell lacks endogenous expression of an HLA-DP beta chain allele.

II.C. Soluble MHC Class II Molecules

In certain aspects, the MHC class II molecule used in the methods disclosed herein is not associated with a membrane of a cell, e.g., the MHC class II molecule is in a soluble form. As used herein, a soluble MHC class II molecule includes any MHC class II molecule or a portion thereof, described herein, that is not associated with a cell membrane. In certain aspects, the MHC class II molecule or portion thereof is unbound to any membrane. In some aspects, the MHC class II molecule or portion thereof is bound to an inert particle. In some aspects, the MHC class II molecule or portion thereof is bound to the membrane of an extracellular vesicle. In some aspects, the MHC class II molecule is bound to an artificial membrane or an artificial surface, e.g., the surface of an array plate.

Any inert particle known in the art can be used in the compositions and methods of the present disclosure. In some aspects, the inert particle is a bead. In some aspects, the bead is a glass bead, a latex bead, a metal bead, or any combination thereof. In some aspects, the inert particle is a nanoparticle (NP). Any NP known in the art can be used in the compositions and methods of the present disclosure. In certain aspects, the nanoparticle is selected from a pegylated iron oxide, chitosan, dextrane, gelatin, alginate, liposome, starch, branched polymer, carbon-based carrier, polylactic acid, poly(cyano)acrylate, polyethyleinemine, block copolymer, polycaprolactone, SPIONS, USPIONS, Cd/Zn-selenide, or silica nanoparticle. In particular aspects, the nanoparticle is a pegylated iron oxide nanoparticle. Nonlimiting examples of nanoparticles useful in the compositions and methods disclosed herein include those set forth in De Jong and Borm, Int. J. Nanomedicine 3(2):133-49 (2008) and Umeshappa et al., Nat. Commun. 10(1):2150 (May 14, 2019), each of which is incorporated by reference herein in its entirety.

In some aspects, the MHC class II molecule comprises a fragment of a full length MHC class II molecule, wherein one or more amino acids of the transmembrane domain of the alpha chain and/or the transmembrane domain of the beta chain are deleted. In some aspects, the MHC class II molecule comprises the extracellular domain of the alpha chain (e.g., as set forth in SEQ ID NO: 6) and/or the extracellular domain of the beta chain (e.g., as set forth in SEQ ID NO: 1 or 3). In some aspects, the MHC class II molecule comprises the extracellular domain of the alpha chain (e.g., as set forth in SEQ ID NO: 16) and/or the extracellular domain of the beta chain (e.g., as set forth in SEQ ID NO: 11 or 13). In some aspects, the MHC class II molecule comprises the extracellular domain of the alpha chain (e.g., as set forth in SEQ ID NO: 24) and/or the extracellular domain of the beta chain (e.g., as set forth in SEQ ID NO: 19 or 21).

In certain aspects, the MHC class II molecule comprises a DP alpha chain comprising an amino acid sequence having at least about 80%, at least about 85%, at least about 90%, at least about 95%, at least about 96%, at least about 97%, at least about 98%, or at least about 99% sequence identity to SEQ ID NO: 6. In some aspects, the MHC class II molecule comprises a DP alpha chain comprising an amino acid sequence set forth in SEQ ID NO: 6.

In certain aspects, the MHC class II molecule comprises a DQ alpha chain comprising an amino acid sequence having at least about 80%, at least about 85%, at least about 90%, at least about 95%. at least about 96%, at least about 97%, at least about 98%, or at least about 99% sequence identity to SEQ ID NO: 16. In some aspects, the MHC class II molecule comprises a DQ alpha chain comprising an amino acid sequence set forth in SEQ ID NO: 16.

In certain aspects, the MHC class II molecule comprises a DR alpha chain comprising an amino acid sequence having at least about 80%, at least about 85%, at least about 90%, at least about 95%, at least about 96%, at least about 97%, at least about 98%, or at least about 99% sequence identity to SEQ ID NO: 24. In some aspects, the MHC class II molecule comprises a DR alpha chain comprising an amino acid sequence set forth in SEQ ID NO: 24.

In certain aspects, the MHC class II molecule comprises a DP beta chain comprising an amino acid sequence having at least about 80%, at least about 85%, at least about 90%, at least about 95%, at least about 96%, at least about 97%, at least about 98%, or at least about 99% sequence identity to SEQ ID NO: 1. In some aspects, the MHC class II molecule comprises a DP beta chain comprising an amino acid sequence set forth in SEQ ID NO: 1. In certain aspects, the MHC class II molecule comprises a DP beta chain comprising an amino acid sequence having at least about 80%, at least about 85%, at least about 90%, at least about 95%, at least about 96%, at least about 97%, at least about 98%, or at least about 99% sequence identity to SEQ ID NO: 3. In some aspects, the MHC class II molecule comprises a DP beta chain comprising an amino acid sequence set forth in SEQ ID NO: 3. In certain aspects, the MHC class II molecule comprises a DP beta chain comprising an amino acid sequence having at least about 80%, at least about 85%, at least about 90%, at least about 95%, at least about 96%, at least about 97%, at least about 98%, or at least about 99% sequence identity to SEQ ID NO: 4. In some aspects, the MHC class II molecule comprises a DP beta chain comprising an amino acid sequence set forth in SEQ ID NO: 4. In certain aspects, the MHC class II molecule comprises a DP beta chain comprising an amino acid sequence having at least about 80%, at least about 85%, at least about 90%, at least about 95%, at least about 96%, at least about 97%, at least about 98%, or at least about 99% sequence identity to SEQ ID NO: 5. In some aspects, the MHC class II molecule comprises a DP beta chain comprising an amino acid sequence set forth in SEQ ID NO: 5.

In certain aspects, the MHC class II molecule comprises a DQ beta chain comprising an amino acid sequence having at least about 80%, at least about 85%, at least about 90%, at least about 95%, at least about 96%, at least about 97%, at least about 98%, or at least about 99% sequence identity to SEQ ID NO: 11. In some aspects, the MHC class II molecule comprises a DQ beta chain comprising an amino acid sequence set forth in SEQ ID NO: 11. In certain aspects, the MHC class II molecule comprises a DQ beta chain comprising an amino acid sequence having at least about 80%, at least about 85%, at least about 90%, at least about 95%, at least about 96%, at least about 97%, at least about 98%, or at least about 99% sequence identity to SEQ ID NO: 13. In some aspects, the MHC class II molecule comprises a DQ beta chain comprising an amino acid sequence set forth in SEQ ID NO: 13. In certain aspects, the MHC class II molecule comprises a DQ beta chain comprising an amino acid sequence having at least about 80%, at least about 85%, at least about 90%, at least about 95%, at least about 96%, at least about 97%, at least about 98%, or at least about 99% sequence identity to SEQ ID NO: 14. In some aspects, the MHC class II molecule comprises a DQ beta chain comprising an amino acid sequence set forth in SEQ ID NO: 14. In certain aspects, the MHC class II molecule comprises a DQ beta chain comprising an amino acid sequence having at least about 80%, at least about 85%, at least about 90%, at least about 95%, at least about 96%, at least about 97%, at least about 98%, or at least about 99% sequence identity to SEQ ID NO: 15. In some aspects, the MHC class II molecule comprises a DQ beta chain comprising an amino acid sequence set forth in SEQ ID NO: 15.

In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 19. In some aspects, the MHC class II molecule comprises a DR beta chain comprising an amino acid sequence set forth in SEQ ID NO: 19. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 21. In some aspects, the MHC class II molecule comprises a DR beta chain comprising an amino acid sequence set forth in SEQ ID NO: 21. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 22. In some aspects, the MHC class II molecule comprises a DR beta chain comprising an amino acid sequence set forth in SEQ ID NO: 22. In certain aspects, the MHC class II molecule comprises a DR beta chain comprising an amino acid sequence having at least about 80%, at least about 85%, at least about 90%, at least about 95%, at least about 96%, at least about 97%, at least about 98%, or at least about 99% sequence identity to SEQ ID NO: 23. In some aspects, the MHC class II molecule comprises a DR beta chain comprising an amino acid sequence set forth in SEQ ID NO: 23.

II.D. Nucleic Acid Molecules and Vectors

Certain aspects of the present disclosure are directed to a nucleic acid molecule encoding an MHC class II molecule disclosed herein. In some aspects the nucleic acid molecule encodes an MHC class II beta chain disclosed herein. In certain aspects, the nucleic acid molecule encoding the MHC class II beta chain comprises a nucleotide sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with a sequence set forth in SEQ ID NO: 2, 12, or 20.

In some aspects the nucleic acid molecule encodes an MHC class II alpha chain disclosed herein. In certain aspects, the nucleic acid molecule encoding the MHC class II alpha chain comprises a nucleotide sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with a sequence set forth in SEQ ID NO: 7, 17, or 25.

In some aspects, the nucleic acid molecule encodes both an MHC class II alpha chain disclosed herein and an MHC class II beta chain disclosed herein. In some aspects, the sequence encoding the MHC class II alpha chain is under the control of the same promoter as the sequence encoding the MHC class II beta chain. In some aspects, the sequence encoding the MHC class II alpha chain is under the control of a first promoter, and the sequence encoding the MHC class II beta chain is under the control of a second promoter.

In some aspects, the present disclosure is directed to a first nucleic acid molecule encoding an MHC class II beta chain disclosed herein and a second nucleic acid molecule encoding an MHC class II alpha chain disclosed herein.

Certain aspects of the present disclosure are directed to a vector or a set of vectors comprising a nucleic acid molecule disclosed herein. In some aspects, the vector is a viral vector. In some aspects, the vector is a viral particle or a virus. In some aspects, the vector is a mammalian vector. In some aspects, the vector is a bacterial vector.

In certain aspects, the vector is a retroviral vector. In some aspects, the vector is an adenoviral vector, a lentivirus, a Sendai virus, a baculoviral vector, an Epstein Barr viral vector, a papovaviral vector, a vaccinia viral vector, a herpes simplex viral vector, or an adeno associated virus (AAV) vector. In particular aspects, the vector is an AAV vector. In some aspects, the vector is a lentivirus. In particular aspects, the vector is an adenoviral vector. In some aspects, the vector is a Sendai virus. In some aspects, the vector is a hybrid vector. Examples of hybrid vectors that can be used in the present disclosure can be found in Huang and Kamihira, Biotechnol. Adv. 31(2):208-23 (2103), which is incorporated by reference herein in its entirety.

II.E. Methods of Treating a Tumor

In certain aspects, the methods disclosed herein further comprise treating a cancer in a subject in need thereof. In some aspects, the method further comprises administering a TCR identified using the methods disclosed herein to a subject in need thereof, wherein the subject has a cancer. In some aspects, the method comprises administering a cell to the subject, wherein the cell comprises a TCR identified using the methods disclosed herein. In some aspects, the cell is a T cell.

In some aspects, the cancer is selected from melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of the cancers. In some aspects, the cancer is melanoma.

In some aspects, the cancer is relapsed. In some aspects, the cancer is refractory. In some aspects, the cancer is advanced. In some aspects, the cancer is metastatic.

In some aspects, the methods disclosed herein treat a cancer in a subject. In some aspects, the methods disclosed herein reduce the severity of one or more symptom of the cancer. In some aspects, the methods disclosed herein reduce the size or number of a tumor derived from the cancer. In some aspects, the methods disclosed herein increase the overall survival of the subject, relative to a subject not provided the methods disclosed herein. In some aspects, the methods disclosed herein increase the progressive-free survival of the subject, relative to a subject not provided the methods disclosed herein. In some aspects, the methods disclosed herein lead to a partial response in the subject. In some aspects, the methods disclosed herein lead to a complete response in the subject.

Certain aspects of the present disclosure are directed to methods of treating an infection in a subject in need thereof, comprising administering to the subject an HLA class II molecule disclosed herein, a nucleic acid molecule disclosed herein, a vector disclosed herein, or a cell disclosed herein. Non-limiting examples of infections that can be treated using the compositions and methods disclosed herein include infection by a virus (including viroids and prions), a bacterium, a fungus, a parasite, or any combination thereof. In some aspects, the virus is herpesvirus, HIV, papvavirus, measles virus, rubella virus, human papillomavirus (HPV), human T-lymphotropic virus 1, Epstein-Barr virus, hepatitis A virus, hepatitis B virus, hepatitis C virus, influenza virus, norovirus, and any combination thereof. In some aspects, the bacterium is selected from Streptococcus, Staphylococcus, and E. coli. In some aspects, the bacterial infection is selected from Brucellosis, Campylobacter infections, Cat-scratch disease, Cholera, Escherichia coli, Gonorrhea, Klebsiella, Enterobacter, Serratia, Legionella infections, Meningococcal infection, Pertussis, Plague, Pseudomonas infection, Salmonella infection, Shigellosis, Typhoid fever, Tularemia, Anthrax, Diphtheria, Enterococcal infection, Erysipelothricosis, Listeriosis, Nocardiosis, Pneumococcal infection, Staphylococcal infection, Streptococcal infection, and any combination thereof. In some embodiments, the parasite infection is selected from pinworm, trichomononiasis, toxoplasmosis, giardiasis, cryptosporidiosis, malaria, hookwork, ringworm, tapeworm, fluke, and any combination thereof. In some aspects, the fungal infection is selected from Candida, Malassezia furfur, dermatophytes (e.g., Epidermophyton, Microsporum, and Trichophyton), or any combination thereof.

In some aspects, the methods disclosed herein comprise treating a cancer or an infection in a subject in need thereof, comprising administering to the subject a cell described herein, wherein the cell comprises an MHC class II molecule disclosed herein, a nucleic acid molecule disclosed herein, a vector disclosed herein, or any combination thereof.

In some aspects, the cell is obtained from the subject. In some aspects, the cell is obtained from a donor other than the subject.

All of the various aspects, aspects, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1—Generation of Affinity Matured HLA-DP Molecules

Cells

Peripheral mononuclear cells were obtained via density gradient centrifugation (Ficoll-Paque PLUS, GE Healthcare Life Sciences, Marlborough, Mass.). The K562 cell line is an erythroleukemic cell line with defective HLA class I/II expression. K562-based artificial APCs (aAPCs) individually expressing various HLA class II genes as a single HLA allele in conjunction with CD80 and CD83 have been reported previously (Butler et al., PloS One 7, e30229 (2012). The Jurkat 76 cell line is a T cell leukemic cell line lacking endogenous TCR, CD4, and CD8 expression. Jurkat 76/CD4 cells were generated by retrovirally transducing the human CD4 gene. A375, SK-MEL-21, SK-MEL-28, SK-MEL-37 and Me275 are melanoma cell lines. HEK293T cells and melanoma cell lines were grown in DMEM supplemented with 10% FBS and 50 μg/ml gentamicin (Thermo Fisher Scientific, Waltham, Mass.). The K562 and Jurkat 76 cell lines were cultured in RPMI 1640 supplemented with 10% FBS and 50 μg/ml gentamicin.

Peptides

Synthetic peptides were purchased from Genscript (Piscataway, N.J.) and dissolved at 50 μg/ml in DM50. The peptide sequences are shown in Table 6.

TABLE 6
Synthetic Peptide Sequences

| | | SEQ |
|---|---|---|
| | | ID |
| Name | Sequence | NO: |
| ABCC6₁₀₈₁₋₁₁₀₀ | EVLAPVILM | 50 |
| | LLNSFFNAI | |
| | ST | |
| ABCC6₁₆₂₋₁₈₁ | EGEISDPFR | 51 |
| | FTTFYIHFA | |
| | LV | |
| ABCC6₃₀₁₋₃₂₀ | KALLATFGS | 52 |
| | SFLISACFK | |
| | LI | |
| ABCC6₃₁₇₋₃₃₆ | FKLIQDLLS | 53 |
| | FINPQLLSI | |
| | LI | |
| ACRBP₄₅₀₋₄₆₉ | GCEDVRVSG | 54 |
| | WLQTEFLSF | |
| | QD | |
| AFP₂₃₉₋₂₅₈ | TVTKLSQKF | 55 |
| | TKVNFTEIQ | |
| | KL | |
| AIM2₁₈₋₃₇ | TDEELDRFK | 56 |
| | FFLSDEFNI | |
| | AT | |
| AIM2₂₀₅₋₂₂₄ | RIIIIARYY | 57 |
| | RHSGFLEVN | |
| | SA | |
| ALDH1A₁₄₃₋₁₆₂ | RTIPIDGNF | 58 |
| | FTYTRHEPI | |
| | GV | |
| ALK₁₁₆₀₋₁₁₇₉ | DELDFLMEA | 59 |
| | LIISKFNHQ | |
| | NI | |
| ANKRD30A₁₃₇₋₁₅₆ | VYGNTALHY | 60 |
| | AVYSEILSV | |
| | VA | |
| ANXA2₂₄₁₋₂₆₀ | LESIRKEVK | 61 |
| | GDLENAFLN | |
| | LV | |
| ARF4₇₃₋₉₂ | RIRPLWKHY | 62 |
| | FQNTQGLIF | |
| | VV | |
| BAGE1₅₋₂₄ | AVFLALSAQ | 63 |
| | LLQARLMKE | |
| | ES | |
| BAX₁₃₄₋₁₄₃ | RTIMGWTLD | 64 |
| | FLRERLLGW | |
| | IQ | |
| BCL2L1₁₃₋₃₂ | LSYKLSQKG | 65 |
| | YSWSQFSDV | |
| | EE | |
| BIRC5₈₇₋₁₀₆ | LSVKKQFEE | 66 |
| | LTLGEFLKL | |
| | DR | |
| BIRC7₁₃₉₋₁₅₈ | DPWTEHAKW | 67 |
| | FPSCQFLLR | |
| | SK | |
| BST2₁₅₄₋₁₇₃ | YPSSQDSSS | 68 |
| | AAAPQLLIV | |
| | LL | |
| CA9₃₃₁₋₃₅₀ | LTTPPCAQG | 69 |
| | VIWTVFNQT | |
| | VM | |
| CALCA₁₋₂₀ | MGFQKFSPF | 70 |
| | LALSILVLL | |
| | QA | |
| CCDC110₅₈₋₇₇ | VLQQQLESF | 71 |
| | QALRMQTLQ | |
| | NV | |
| CCNA1₃₆₆₋₃₈₅ | KYVAELSLL | 72 |
| | EADPFLKYL | |
| | PS | |
| CCNA1₄₃₈₋₄₅₇ | QQAIREKYK | 73 |
| | ASKYLCVSL | |
| | ME | |
| CCND1₂₁₉₋₂₃₈ | SPNNFLSYY | 74 |
| | RLTRFLSRV | |
| | IK | |
| CD274₁₋₂₀ | MRIFAVFIF | 75 |
| | MTYWHLLNA | |
| | FT | |
| CD45₁₀₁₂₋₁₀₃₁ | PSKYINASF | 76 |
| | IMSYWKPEV | |
| | MI | |
| CD45₁₀₃₆₋₁₀₅₅ | PLKETIGDF | 77 |
| | WQMIFQRKV | |
| | KV | |
| CD45₁₂₀₄₋₁₂₂₃ | KARPGMVST | 78 |
| | FEQYQFLYD | |
| | VI | |
| CDH3₂₁₀₋₂₂₉ | DHKPKFTQD | 79 |
| | TFRGSVLEG | |
| | VL | |
| CDKN1A₅₂₋₇₁ | DFVTETPLE | 80 |
| | GDFAWERVR | |
| | GL | |
| CEA₂₆₆₋₂₈₅ | PAQYSWFVN | 81 |
| | GTFQQSTQE | |
| | LF | |
| CEL₅₃₂₋₅₅₁ | RSLRTNFLR | 82 |
| | YWTLTYLAL | |
| | PT | |
| CLCA2₁₃₂₋₁₄₁ | CGKEGKYIH | 83 |
| | FTPNFLLND | |
| | NL | |
| CNTN2₄₇₈₋₄₉₇ | ISRSDEGKY | 84 |
| | TCFAENFMG | |
| | KA | |
| COTL1₅₀₋₆₉ | QQCTDDVRL | 85 |
| | FAFVRFTTG | |
| | DA | |
| CPSF1₂₀₂₋₂₂₁ | NIIDLQFLH | 86 |
| | GYYEPTLLI | |
| | LF | |
| CPSF1₄₇₆₋₄₉₅ | ANAAVGEPA | 87 |
| | FLSEEFQNS | |
| | PE | |
| CSAG2₁₁₋₃₀ | GVKRKDQGF | 88 |
| | LEKEFYHKT | |
| | NI | |
| CSF1₁₃₀₁₄₉ | HDKACVRTF | 89 |
| | YETPLQLLE | |
| | KV | |
| CSPG4₁₇₄₁₋₁₇₆₀ | QRSEHDVLF | 90 |
| | QVTQFPSRG | |
| | QL | |
| CSPG4₂₀₀₃₋₂₀₂₂ | FQIDQGEVV | 91 |
| | FAFTNFSSS | |
| | HD | |
| CSPG4₂₀₀₅₋₂₀₂₄ | IDQGEVVFA | 92 |
| | FTNFSSSHD | |
| | HF | |
| CT83₉₋₂₈ | SSILCALIV | 93 |
| | FWKYRRFQR | |
| | NT | |
| CTSG₄₄₋₆₃ | AGQSRCGGF | 94 |
| | LVREDFVLT | |
| | AA | |
| CYP1B1₂₆₂₋₂₈₁ | EQLNRNFSN | 95 |
| | FILDKFLRH | |
| | CE | |
| CYP1B1₃₃₆₋₃₅₅ | STALQWLLL | 96 |
| | LFTRYPDVQ | |
| | TR | |
| CYP1B1₉₋₂₈ | DPWPLNPLS | 97 |
| | IQQTTLLLL | |
| | LS | |
| DCT₁₇₄₋₁₉₃ | PQFANCSVY | 98 |
| | DFFVWLHYY | |
| | SV | |
| DCT₁₇₇₋₁₉₆ | ANCSVYDFF | 99 |
| | VWLHYYSVR | |
| | DT | |
| DCT₃₃₂₋₃₅₁ | QKFDNPPFF | 100 |
| | QNSTFSFRN | |
| | AL | |
| DDX43₃₇₀₋₃₈₉ | IATPGRLND | 101 |
| | LQMSNFVNL | |
| | KN | |
| DKK1₁₉₅₋₂₁₄ | CASGLCCAR | 102 |
| | HFWSKICKP | |
| | VL | |
| EGLN3₈₅₋₁₀₄ | EEGCEAISF | 103 |
| | LLSLIDRLV | |
| | LY | |
| ENAH₉₂₋₁₁₁ | GSKEDANVF | 104 |
| | ASAMMHALE | |
| | VL | |
| EPCAM₁₇₂₋₁₉₁ | TRYQLDPKF | 105 |
| | ITSILYENN | |
| | VI | |
| EPHA2₁₂₅₋₁₄₄ | ESDLDYGTN | 106 |
| | FQKRLFTKI | |
| | DT | |
| EPHA3₁₂₆₋₁₄₅ | ESDDDHGVK | 107 |
| | FREHQFTKI | |
| | DT | |
| EPOR₄₁₆₋₄₃₅ | PEGASAASF | 108 |
| | EYTILDPSS | |
| | QL | |
| ERBB2₉₉₂₋₁₀₁₁ | EDLGPASPL | 109 |
| | DSTFYRSLL | |
| | ED | |
| ERBB2₉₉₃₋₁₀₁₂ | DLGPASPLD | 110 |
| | STFYRSLLE | |
| | DD | |
| EXOSC5₂₂₅₋₂₃₄ | AASQHVFRF | 111 |
| | YRESLQRRY | |
| | SK | |
| EZH2₂₈₂₋₃₀₁ | HTLFCRRCF | 112 |
| | KYDCFLHPF | |
| | HA | |
| FGF5₁₋₁₉ | MSLSFLLLL | 113 |
| | FFSHLILSA | |
| | WA | |
| FOLHL₅₅₅₋₅₇₄ | VYETYELVE | 114 |
| | KFYDPMFKY | |
| | HL | |
| GAGE1₁₁₇₋₁₃₆ | VAQTGILWL | 115 |
| | LMNNCFLNL | |
| | SP | |
| GAGE3₁₋₂₀ | MNLSRGKST | 116 |
| | YYWPRPRRY | |
| | VQ | |
| gp100₆₂₁₋₆₄₀ | MKQDFSVPQ | 117 |
| | LPHSSSHWL | |
| | RL | |
| GPC3₁₃₅₋₁₅₄ | PSLTPQAFE | 118 |
| | FVGEFFTDV | |
| | SL | |
| GPR143₁₂₀₋₁₃₉ | AMWIQLLYS | 119 |
| | ACFWWLFCY | |
| | AV | |
| HLA- | TGPIRNGDW | 120 |
| DOB₁₇₁₋₁₉₀ | TFQTVVMLE | |
| | MT | |
| HPN₃₈₇₋₄₀₆ | PGVYTKVSD | 121 |
| | FREWIFQAI | |
| | KT | |
| HSD17B12₁₇₋₃₆ | GTVAYLALR | 122 |
| | ISYSLFTAL | |
| | RV | |
| HSD17B12₂₂₅₋₂₄₄ | VFVQSVLPY | 123 |
| | FVATKLAKI | |
| | RK | |
| Hsp70₂₈₉₋₃₀₈ | EGIDFYTSI | 124 |
| | TRARFEELC | |
| | SD | |
| IDO1₄₀₃₋₄₂₂ | SFRDGDCSK | 125 |
| | GFFLVSLLV | |
| | EI | |
| IL13RA2₂₁₇₋₂₃₆ | SSENKPIRS | 126 |
| | SYFTFQLQN | |
| | IV | |
| KAAG1₃₋₂₂ | DDAAPRVEG | 127 |
| | VPVAVHKHA | |
| | LH | |
| KDM5B₃₈₁₋₄₀₀ | TFGEMADAF | 128 |
| | KSDYFNMPV | |
| | HM | |
| KDR₇₆₅₋₇₈₄ | IIILVGTAV | 129 |
| | IAMFFWLLL | |
| | VI | |
| KIF20A₂₉₈₋₃₁₇ | RFSIWISFF | 130 |
| | EIYNELLYD | |
| | LL | |
| KIF2C₃₈₆₋₄₀₅ | NQPCYRKLG | 131 |
| | LEVYVTFFE | |
| | IY | |
| KLK4₁₀₂₋₁₂₁ | SVRHPEYNR | 132 |
| | PLLANDLML | |
| | IK | |
| LGALS3BP₃₇₄₋₃₉₃ | QKKTLQALE | 133 |
| | FHTVPFQLL | |
| | AR | |
| LGALS9₁₁₂₋₁₃₁ | VMVNGILFV | 134 |
| | QYFHRVPFH | |
| | RV | |
| LGSN₁₈₇₋₂₀₆ | IAKRQLSHL | 135 |
| | QASGFSLLS | |
| | AF | |
| LGSN₂₈₇₋₃₀₈ | TGVKEVARK | 136 |
| | YNYIASFFI | |
| | ET | |
| LGSN₂₉₆₋₃₁₅ | YNYIASFFI | 137 |
| | ETGFCDSGI | |
| | LS | |
| LGSN₇₈₋₉₇ | QAMAKNRLQ | 138 |
| | FVRFEATDL | |
| | HG | |
| LIMS1₃₄₋₅₃ | QCFVCAQCF | 139 |
| | QQFPEGLFY | |
| | EF | |
| LY6K₉₉₋₁₁₈ | EKRFLLEEP | 140 |
| | MPFFYLKCC | |
| | KI | |
| MAGE- | DEKVTDLVQ | 141 |
| A10₁₃₅₋₁₅₄ | FLLFKYQMK | |
| | EP | |
| MAGE- | ALSRKMAEL | 142 |
| A12₁₀₅₋₁₂₇ | VHFLLLKYR | |
| | AR | |
| MAGE- | PRKLLTQDL | 143 |
| A1₂₃₅₋₂₅₄ | VQEKYLEYR | |
| | QV | |
| MAGE- | AISRKMVEL | 144 |
| A2₁₀₈₋₁₂₇ | VHFLLLKYR | |
| | AR | |
| MAGE- | RKMVELVHF | 145 |
| A2₁₁₁₋₁₃₀ | LLLKYRARE | |
| | PV | |
| MAGE- | YEFLWGPRA | 146 |
| A4₂₇₀₋₂₈₉ | LAETSYVKV | |
| | LE | |
| MAGE- | LGSVVGNWQ | 147 |
| A6₁₃₆₋₁₅₅ | YFFPVIFSK | |
| | AS | |
| MAGE- | PKKLLTQYF | 148 |
| A6₂₄₂₋₂₆₁ | VQENYLEYR | |
| | QV | |
| MAGE- | YFPVIFGKA | 149 |
| A9₁₄₅₋₁₆₄ | SEFMQVIFG | |
| | TD | |
| MAGE- | PRKFITQDL | 150 |
| B1₂₄₁₋₂₆₀ | VQEKYLKYE | |
| | QV | |
| MAGE- | PWKLITKDL | 151 |
| B2₂₄₄₋₂₆₃ | VQEKYLEYK | |
| | QV | |
| MAGE- | QSPLQNPAS | 152 |
| C1₁₂₅₋₁₄₄ | SFFSSALLS | |
| | IF | |
| MAGE- | QSPLQIPVS | 153 |
| C1₁₉₅₋₂₁₄ | RSFSSTLLS | |
| | IF | |
| MAGE- | SPLQIPGSP | 154 |
| C1₃₇₁₋₃₉₀ | SFSSTLLSL | |
| | FQ | |
| MAGE- | SPLQIPMTS | 155 |
| C1₄₀₆₋₄₂₅ | SFSSTLLSI | |
| | LQ | |
| MAGEC2₃₇₃₋₃₉₂ | PLSSCCSSF | 156 |
| | SWSSFSEES | |
| | SS | |
| MART1₃₂₋₅₁ | ILTVILGVL | 157 |
| | LLIGCWYCR | |
| | RR | |
| MC1R₁₃₉₋₁₅₈ | AVDRYISIF | 158 |
| | YALRYHSIV | |
| | TL | |
| MC1R₂₄₅₋₂₆₄ | ILLGIFFLC | 159 |
| | WGPFFLHLT | |
| | LI | |
| MDK₁₋₂₀ | MQHRGFLLL | 160 |
| | TLLALLALT | |
| | SA | |
| MDM2₄₇₋₆₆ | TYTMKEVLF | 161 |
| | YLGQYIMTK | |
| | RL | |
| MET₁₃₃₄₋₁₃₅₃ | VSRISAIFS | 162 |
| | TFIGEHYVH | |
| | VN | |
| MET₃₅₉₋₃₇₈ | RSAMCAFPI | 163 |
| | KYVNDFFNK | |
| | IV | |
| MGAT5₁₃₋₃₂ | KLGFFLVTF | 164 |
| | GFIWGMMLL | |
| | HF | |
| MMP2₄₇₉₋₄₉₈ | AQIRGEIFF | 165 |
| | FKDRFIWRT | |
| | VT | |
| MMP2₅₂₆₋₅₄₅ | APQEEKAVF | 166 |
| | FAGNEYWIY | |
| | SA | |
| MMP2₅₇₅₋₅₉₄ | SKNKKTYIF | 167 |
| | AGDKFWRYN | |
| | EV | |
| MMP2₆₂₃₋₆₄₂ | LQGGGHSYF | 168 |
| | FKGAYYLKL | |
| | EN | |
| MMP7₂₉₋₄₈ | ELQWEQAQD | 169 |
| | YLKRFYLYD | |
| | SE | |
| MOK₁₅₆₋₁₇₅ | QPYTEYIST | 170 |
| | RWYRAPECL | |
| | LT | |
| MPO₆₅₄₋₆₇₃ | KGRVGPLLA | 171 |
| | CIIGTQFRK | |
| | LR | |
| MSH3₁₀₄₂₋₁₀₆₁ | DPGAAEQVP | 172 |
| | DFVTFLYQI | |
| | TR | |
| MSLN₃₃₅₋₃₅₄ | QMDRVNAIP | 173 |
| | FTYEQLDVL | |
| | KH | |
| MUC1₁₀₃₅₋₁₀₅₄ | QLSTGVSFF | 174 |
| | FLSFHISNL | |
| | QF | |
| MUC16₁₀₁₄₇₋₁₀₁₆₆ | SMPANFETT | 175 |
| | GFEAEPFSH | |
| | LT | |
| MUC16₁₀₃₂₃₋₁₀₃₄₂ | SLPSSTPVP | 176 |
| | FSSSTFTTT | |
| | DS | |
| MUC16₁₁₉₈₈₋₁₂₀₀₇ | AKTTTTFNT | 177 |
| | LAGSLFTPL | |
| | TT | |
| MUC16₂₉₄₄₋₂₉₆₃ | STKAISASS | 178 |
| | FQSTGFTET | |
| | PE | |
| MUC2₉₄₋₁₁₃ | ILLTIKDDT | 179 |
| | IYLTRHLAV | |
| | LN | |
| MUC5AC₄₉₂₂₋₄₉₄₁ | SGWGDPHYI | 180 |
| | TFDGTYYTF | |
| | LD | |
| Nuf2₅₀₋₆₉ | MRALQIVYG | 181 |
| | IRLEHFYMM | |
| | PV | |
| OR51E2₂₀₄₋₂₂₃ | LVMGVDVMF | 182 |
| | ISLSYFLII | |
| | RT | |
| p53₇₋₂₆ | DPSVEPPLS | 183 |
| | QETFSDLWK | |
| | LL | |
| PAK2₃₄₄₋₃₆₃ | QIAAVCREC | 184 |
| | LQALEFLHA | |
| | NQ | |
| PAK2₄₈₅₋₅₀₄ | VEKRGSAKE | 185 |
| | LLQHPFLKL | |
| | AK | |
| PAPOLA₁₂₁₋₁₄₀ | PRHVDRSDF | 186 |
| | FTSFYDKLK | |
| | LQ | |
| PASD1₂₅₈₋₂₇₇ | MFVDSDSTY | 187 |
| | CSSTVFLDT | |
| | MP | |
| PAX3₂₁₇₋₂₃₆ | RKQRRSRTT | 188 |
| | FTAEQLEEL | |
| | ER | |
| PAX5₃₃₂₋₃₅₁ | LTGMVPGSE | 189 |
| | FSGSPYSHP | |
| | QY | |
| PGK1₃₃₅₋₃₅₄ | VWNGPVGVF | 190 |
| | EWEAFARGT | |
| | KA | |
| PLAC1₁₈₀₋₁₉₉ | QAGAQEAQP | 191 |
| | LQPSHFLDI | |
| | SE | |
| PLIN2₁₈₋₃₇ | NLPLVSSTY | 192 |
| | DLMSSAYLS | |
| | TK | |
| POTEE₉₅₂₋₉₇₁ | NERFRCPEA | 193 |
| | LFQPCFLGM | |
| | ES | |
| PPIB₇₉₋₉₈ | LATGEKGFG | 194 |
| | YKNSKFHRV | |
| | IK | |
| PRAME₂₉₄₋₃₁₃ | SLQCLQALY | 195 |
| | VDSLFFLRG | |
| | RL | |
| PRDM1₆₅₉₋₆₇₈ | YQCKVCPAK | 196 |
| | FTQFVHLKL | |
| | HK | |
| PSA₂₈₃₋₃₀₂ | EVAAKTLPF | 197 |
| | YKDYFNVPY | |
| | PL | |
| PSA₅₈₀₋₅₉₉ | KLNLGTVGF | 198 |
| | YRTQYSSAM | |
| | LE | |
| PSA₆₆₇₋₆₈₆ | SHTDFYEEI | 199 |
| | QEFVKDVFS | |
| | PI | |
| PSA₈₃₇₋₈₅₆ | ELYNRYQGG | 200 |
| | FLISRLIKL | |
| | SV | |
| PTTG1IP₈₁₋₁₀₀ | CKLSSARWG | 201 |
| | VCWVNFEAL | |
| | II | |
| PXDN₁₂₂₆₋₁₂₄₅ | SRLGPTLMC | 202 |
| | LLSTQFKRL | |
| | RD | |
| RAB38₁₃₁₋₁₅₀ | QGKDVLMNN | 203 |
| | GLKMDQFCK | |
| | EH | |
| RCVRN₂₀₋₃₉ | NTKFSEEEL | 204 |
| | CSWYQSFLK | |
| | DC | |
| RGS5₁₆₂₋₁₈₁ | MEKDSLPRF | 205 |
| | VRSEFYQEL | |
| | IK | |
| RGS5₇₃₋₉₂ | YGLASFKSF | 206 |
| | LKSEFSEEN | |
| | LE | |
| RhoC₁₆₋₃₅ | CGKTCLLIV | 207 |
| | FSKDQFPEV | |
| | YV | |
| RNF43₁₉₅₋₂₁₄ | PDYDVWILM | 208 |
| | TVVGTIFVI | |
| | IL | |
| RPS2₂₅₀₋₂₆₉ | YLTPDLWKE | 209 |
| | TVFTKSPYQ | |
| | EF | |
| SAGE1₈₅₇₋₈₇₆ | KVKRQFVEF | 210 |
| | TIKEAARFK | |
| | KV | |
| SART1₂₂₇₋₂₄₆ | MDQEFGVST | 211 |
| | LVEEEFGQR | |
| | RQ | |
| SART3₁₃₅₋₁₅₄ | RQKMSEIFP | 212 |
| | LTEELWLEW | |
| | LH | |
| SCGB2A2₇₄₋₉₃ | LSNVEVFMV | 213 |
| | ISFSSYKLF | |
| | KS | |
| SCRN1₂₅₇₋₂₅₆ | TLRDKASGV | 214 |
| | CIDSEFFLT | |
| | TA | |
| SDC1₂₆₂₋₂₈₁ | VGLIFAVCL | 215 |
| | VGFMLYRMK | |
| | KK | |
| SIM2₁₄₇₋₁₆₆ | HHHLLQEYE | 216 |
| | IERSFFLRM | |
| | KC | |
| SLAMF7₂₃₂₋₂₅₁ | LLVPLLLSL | 217 |
| | FVLGLFLWF | |
| | LK | |
| SLC45A3₂₋₂₁ | VQRLWVSRL | 218 |
| | LRHRKAQLL | |
| | LV | |
| SOX10₃₇₆₋₃₉₅ | SQIAYTSLS | 219 |
| | LPHYGSAFP | |
| | SI | |
| SOX4₄₁₃₋₄₃₂ | NFESMSLGS | 220 |
| | FSSSSALDR | |
| | DL | |
| SPA17₂₉₋₄₈ | REQPDNIPA | 221 |
| | FAAAYFESL | |
| | LE | |
| SSX2₂₃₋₄₂ | KAFDDIAKY | 222 |
| | FSKEEWEKM | |
| | KA | |
| SSX4₂₃₋₄₂ | KAFDDIAKY | 223 |
| | FSKKEWEKM | |
| | KS | |
| STEAP1₂₆₃₋₂₈₂ | LLGTIHALI | 224 |
| | FAWNKWIDI | |
| | KQ | |
| STEAP1₂₉₆₋₃₁₅ | FLPIVVLIF | 225 |
| | KSILFLPCL | |
| | RK | |
| STEAP1₇₄₋₉₃ | PIKIAAIIA | 226 |
| | SLTFLYTLL | |
| | RE | |
| STEAP1₇₆₋₉₅ | KIAAIIASL | 227 |
| | TFLYTLLRE | |
| | VI | |
| STEAP3₂₁₈₋₂₃₇ | LALGLFVCF | 228 |
| | YAYNFVRDV | |
| | LQ | |
| TCL1₁₀₋₂₉ | AVTDHPDRL | 229 |
| | WAWEKFVYL | |
| | DE | |
| TERT₅₅₇₋₅₇₆ | LRSFFYVTE | 230 |
| | TTFQKNRLF | |
| | FY | |
| TERT₅₅₈₋₅₇₇ | RSFFYVTET | 231 |
| | TFQKNRLFF | |
| | YR | |
| TM4SF1₁₂₂₋₁₄₁ | LDSLGQWNY | 232 |
| | TFASTEGQY | |
| | LL | |
| TPBG₂₄₁₋₂₆₀ | LSNNSLVSL | 233 |
| | TYVSFRNLT | |
| | HL | |
| TRPC1₃₇₁₋₃₉₀ | APKSQFGRI | 234 |
| | IHTPFMKFI | |
| | IH | |
| TRPC1₃₈₈₋₄₀₇ | IIHGASYFT | 235 |
| | FLLLLNLYS | |
| | LV | |
| TRPC1₄₅₆₋₄₇₅ | NQLSFVMNS | 236 |
| | LYLATFALK | |
| | VV | |
| TRPC1₅₇₈₋₅₉₇ | QQSNDTFHS | 237 |
| | FIGTCFALF | |
| | WY | |
| TYMS₁₂₂₋₁₄₁ | PLLTTKRVF | 238 |
| | WKGVLEELL | |
| | WF | |
| TYR₃₈₃₋₄₀₂ | DPIFLLHHA | 239 |
| | FVDSIFEQW | |
| | LR | |
| UBXN11₂₅₈₋₂₇₇ | QRCLRDILD | 240 |
| | GFFPSELQR | |
| | LY | |
| VENTXP1₁₄₋₃₃ | LAAASGQNR | 241 |
| | MTQGQHFLQ | |
| | KV | |
| WDR46₂₇₃₋₂₉₂ | RRCDRVTRL | 242 |
| | EFLPFHFLL | |
| | AT | |
| XAGE1A₃₃₋₅₂ | CATWKVICK | 243 |
| | SCISQTPGI | |
| | NL | |
| XBP1₁₉₆₋₂₁₅ | LQIQSLISC | 244 |
| | WAFWTTWTQ | |
| | SC | |
| ZBTB7A₉₉₋₁₁₈ | VSTANVGDI | 245 |
| | LSAARLLEI | |
| | PA | |
| CLIP | LPKPPKPVS | 246 |
| | KMRMATPLL | |
| | MQALPM | |
| MAGE- | KKLLTQHFV | 247 |
| A3₂₄₃₋₂₅₈ | QENYLEY | |
| WT1₃₂₈₋₃₄₈ | PGCNKRYFK | 248 |
| | LSHLQMHSR | |
| | KHT | |
| NY- | SLLMWITQC | 249 |
| ESO- | FLPVF | |
| 1₁₅₇₋₁₇ | | |
| NY- | YLAMPFATP | 250 |
| ESO- | MEAELARRS | |
| 1₉₁₋₁₁₀ | LA | |
| Influenza | PKYVKQNTL | 251 |
| HA₃₀₆₋₃₁₈ | KLAT | |
| HIV | FRDYVDRFY | 252 |
| Gag₂₉₃₋₃₁₂ | KTLRAEQAS | |
| | QE | |
| DDX3Y₁₇₁₋₁₉₀ | TGSNCPPHI | 253 |
| | ENFSDIDMG | |
| | EI | |
| Bet V | ETLLRAVES | 254 |
| 1₁₄₂₋₁₅₃ | YLL | |
| Influenza | RGYFKMRTG | 255 |
| HA₂₅₅₋₂₇₀ | KSSIMRS | |

Genes Novel TCR genes were cloned via 5′-rapid amplification of cDNA ends (RACE) PCR using SMARTer RACE 5′/3′ Kit (Takara Bio, Shiga, Japan) and sequenced as previously described. All genes were cloned into the pMX retroviral vector and transduced into cell lines using the 293GPG and PG13 cell-based retrovirus system.

Antibodies

The following antibodies were used for flow cytometry analysis: PE-conjugated anti-class 11 (9-49 (13)), APC-Cy7-conjugated anti-CD4 (RPA-T4, Biolegend, San Diego, Calif.)⁴⁴, FITC-conjugated anti-NGFR (ME20.4, Biolegend, San Diego, Calif.), PE-conjugated anti-His tag (AD1.1.10, Abcam, Cambridge, Mass.), and FITC-conjugated anti-Vβ22 (IMMU 546, Beckman Coulter, Brea, Calif.). Biotinylated DP4/NY-ESO1₁₅₇₋₁₇₀ and DP4/WT1₃₂₉₋₃₄₈ monomers were multimerized using PE-conjugated streptavidin (Thermo Fisher Scientific, Waltham, Mass.) according to the manufacturer's instructions. Dead cells were distinguished with the LIVE/DEAD Fixable Near-IR Dead Cell Stain Kit 465 (Thermo Fisher Scientific, Waltham, Mass.). Stained cells were analyzed with Canto II or LSRFortessa X-20 (BD Biosciences, Franklin Lakes, N.J.). Cell sorting was conducted using a FACS Aria II (BD Biosciences, Franklin Lakes, N.J.). Data analysis was performed using FlowJo software (Tree Star, Ashland, Oreg.).

The following antibodies were used for immunoblot analysis: anti-β-actin (C4, Santa Cruz Biotechnology, Santa Cruz, Calif.), rabbit polyclonal anti-MAGE-A2 (Abcam, Cambridge, Mass.), anti-CCND1 (EPR2241, Abcam, Cambridge, Mass.), HRP-conjugated goat anti-mouse IgG (H+L) secondary antibody (Promega, Fitchburg, Wis.), and HRP-conjugated anti-rabbit IgG (H+L) secondary antibody (Promega, Fitchburg, Wis.).

TCR Transduction into Primary T Cells

CD3⁺ and CD4⁺ T cells were purified using the Pan T Cell Isolation Kit (Miltenyi Biotec, Bergisch Gladbach, Germany) and CD4⁺ T Cell Isolation Kit (Miltenyi Biotec, Bergisch Gladbach, Germany), respectively. Purified T cells were stimulated with aAPC/mOKT3 irradiated with 200 Gy at an E:T ratio of 20:1. Starting the following day, activated T cells were retrovirally transduced with the cloned TCR genes via centrifugation for 1 hour at 1,000×g at 32° C. for 3 consecutive days or using a Retronectin-coated plate (Takara Bio, Shiga, Japan). On the following day, 100 IU/ml IL-2 and 10 ng/ml IL-15 were added to the TCR-transduced T cells. The culture medium was replenished every 2-3 days.

Staining with Soluble CD4

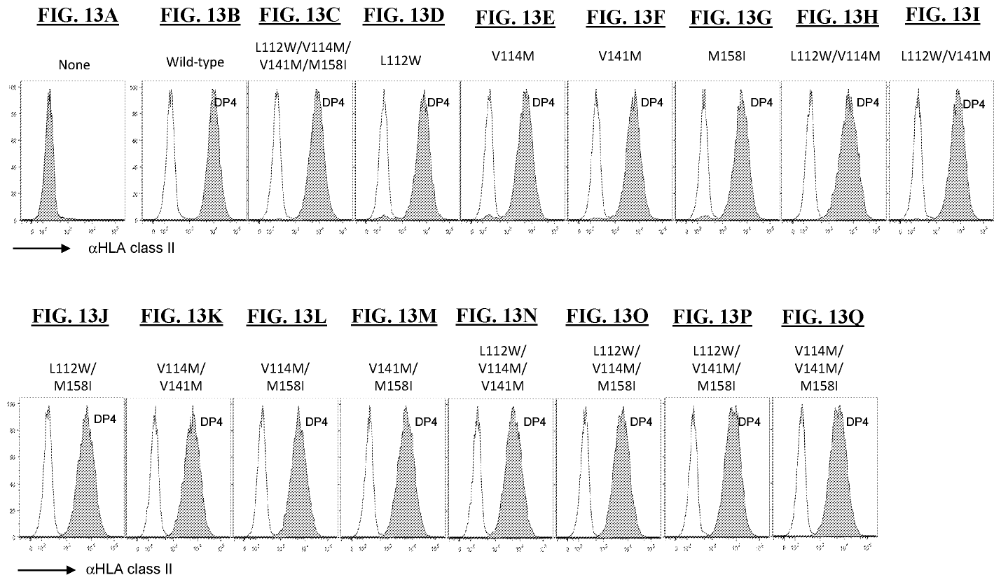

The soluble CD4 (sCD4) gene was generated by fusing the human CD4 extracellular domain with a 6×His tag via a GS linker. HEK293T cells were retrovirally transduced with the sCD4 gene, and the culture supernatant containing the sCD4 monomer was harvested. sCD4 was dimerized with a PE-labeled anti-6×His tag mAb (AD1.1.10, Abcam, Cambridge, Mass.) and used. HLA class II-expressing K562 cells were stained with dimerized sCD4 in the presence of goat serum for 30 min at room temperature. The surface HLA class II expression in K562-derived cells individually expressing various class II genes was as demonstrated in FIGS. 13A-13Q.

Construction and Screening of a Multisite-Directed DPB1*04:01 Mutant cDNA Library

Multisite-directed random mutations were inserted into the DPB1*04:01 cDNA by using PCR and the following primer sets: forward: 5′-CACCACAACNNNCTTNNNTGCCACGTG-3′ (SEQ ID NO: 30) and reverse: 5′-CACGTGGCANNNAAGNNNGTTGTGGTG-3′ (SEQ ID NO: 31) for L112 and V114; forward: 5′- ACAGCTGGGGTCNNNTCCACCAACCTG-3′ (SEQ ID NO: 32) and reverse: 5′-CAGGTTGGTGGANNNGACCCCAGCTGT-3′ (SEQ ID NO: 33) for V141; forward: 5′-CAGATCNNNGTGNNNCTGGAAATGACC-3′ (SEQ ID NO: 34) and reverse: 5′-GGTCATTTCCAGNNNCACNNNGATCTG-3′ (SEQ ID NO: 35) for L156 and M158. N stands for any nucleotide. The resultant PCR fragments were fused to each other to construct a mutant full-length DPB1*04:01 cDNA expression library carrying random mutations at the positions L112, V114, V141, L156, and M158. K562 cells stably expressing the DPA1*01:03 gene were infected with recombinant retroviruses produced using the packaging cell line 293GPG at a transduction efficiency of less than 30%. The infected K562 cells were stained with soluble CD4 dimer, and the dimer-positive cells were collected using a flow cytometry cell sorter. The mutant DPB1*04:01 gene was cloned from the collected cells and retrovirally transduced into K562 cells along with the wild-type DPA1*01:03 gene as described above.

Generation of the HLA Class H Monomer and Dimer

The extracellular domain of the wild-type class II α gene was fused with an acidic leucine zipper via a GGGS linker followed by a 6×His tag via a GS linker (see SEQ ID NO: 8). The ectodomain of the class II β gene carrying mutations (see SEQ ID NO: 3) was similarly linked with a basic leucine zipper via a GGGS linker (see SEQ ID NO: 4). HEK293T cells were transfected with the α and β genes using the 293GPG cell-based retrovirus system and cultured in DMEM supplemented with 10% FBS and 50 μg/ml gentamicin. For DP4 dimer staining, HEK293 T cells stably secreting soluble DP4^{L112W/V141M} protein were grown until confluent, and the medium was changed to serum-free 293 SFM II medium (Thermo Fisher Scientific, Waltham, Mass.). After forty-eight hours, the conditioned medium was harvested and concentrated using Amicon Ultra filters (molecular weight cut-off (MWCO) 10 kDa) (MilliporeSigma, Burlington, Mass.). The soluble HLA class II-containing supernatant was then mixed with 100 μg/ml peptide of interest for 20-24 hours at 37° C. for in vitro peptide exchange. Monomer that was not subjected to peptide exchange was used as a control. The concentration of the monomer was measured by specific ELISA using a nickel-coated plate (XPressBio, Frederick, Md.) and an anti-His tag biotinylated mAb (AD1.1.10, R&D Systems, Minneapolis, Minn.). Soluble HLA class II monomer was dimerized using PE-conjugated anti-His mAb (AD1.1.10, Abcam, Cambridge, Mass.) at a 2:1 molar ratio for 1.5 hours at 4° C. for staining.

Stimulation of DP4-Restricted Antigen-Specific CD4⁺ T Cells

CD4⁺ T cells were purified using a CD4⁺ T Cell Isolation Kit (Miltenyi Biotec, Bergisch Gladbach, Germany). Purified T cells were stimulated with DP4-expressing aAPCs pulsed with DP4-restricted peptides at 10 μg/ml and irradiated at 200 Gy at an E:T ratio of 20:1. After forty-eight hours, 10 IU/ml IL-2 and 10 ng/ml IL-15 were added to the CD4⁺ T cells. The culture medium supplemented with IL-2 (10 IU/ml) and IL-15 (10 ng/ml) was replenished every 2-3 days. After 2 weeks of stimulation, the T cells were subjected to DP4^{L112W/V141M} dimer staining.

HLA Class II Dimer and Tetramer Staining

Primary T cells and Jurkat 76/CD4 T cells transduced with exogenous TCR gene were pretreated with 50 nM dasatinib (LC Laboratories, Woburn, Mass.) for 30 min at 37° C. and stained with 5-15 μg/ml class II dimer for 4-5 hours at room temperature. After washing, cell surface molecules were counterstained with an APC-Cy7-conjugated anti-CD4 mAb, a FITC-conjugated anti-NGFR mAb, and a PE-conjugated anti-V022 mAb.

ELISPOT Assay

Cytokine ELISPOT assays were performed as previously reported (see, e.g., Yamashita et al., Nat. Commun. 8:15244 (2017); and Anczurowski et al., Sci. Rep. 8:4804 (2018)).

Immunoblotting

Immunoblot analysis was performed as previously reported (see, e.g., Yamashita et al., Nat. Commun. 8:15244 (2017); and Anczurowski et al., Sci. Rep. 8:4804 (2018)).

Protein Modeling

The HLA-DP4 and human CD4 complex model structures were predicted based on structures from PDB IDs 3S5L and 3TOE using Swiss-Model workspace for quaternary structure prediction.

Statistical Analysis

Statistical analysis was performed using GraphPad Prism 6.0 software (GraphPad Software, San Diego, Calif.). Unpaired two-tailed Student's t-tests were used for two-sample comparisons. No statistical method was used to predetermine sample size. The investigators were not blinded to allocation during the experiments or outcome assessment. The experiments were not randomized.

Biolayer Interferometry Sensorgram

The extracellular domain of human CD4 (residues 26-440 of NP_000607.1) followed by a GS linker and 10× histidine (His) tag was stably expressed in the human cell line A375 (SEQ ID NOs: 262-263; Table 7). Recombinant 10× His-tagged CD4 protein was purified from the supernatant with TALON metal affinity resin (Takara Bio, Shiga, Japan). The eluted protein was concentrated using an Amicon Ultra-15 spin column (MilliporeSigma, Burlington, Mass.) with a 10 kDa MWCO. Buffer was exchanged to HBS-EP (GE Healthcare Life Sciences, Marlborough, Mass.) using 10 kDa MWCO MINI Dialyzer (Thermo Fisher Scientific, Waltham, Mass.). The purity of the recombinant CD4 protein was consistently >90%, as confirmed by SDS-PAGE.

The recombinant DP4 protein consisted of extracellular domains of DPA1*01:03, and the wild-type DPB1*04:01 or L112W/V141M mutant. DPA1*01:03 was followed by an acid leucine zipper, a GS linker and a 10× histidine tag, while wild-type and mutant DPB1 was followed by a basic leucine zipper, a GS linker, and a biotinylation sequence (GLNDIFEAQKIEWHE; SEQ ID NO: 265). Both DPA and DPB genes were stably expressed in A375-BirA cells, which were transduced with the codon-optimized BirA gene encoding a leader sequence at the 5′ end and an ER retention KDEL motif at the 3′ end. Recombinant DP4 protein was purified from the supernatant with TALON metal affinity resin (Takara Bio, Shiga, Japan). Eluted protein was concentrated using Vivaspin 500 spin column (GE Healthcare Life Sciences, Marlborough, Mass.) with a 10 kDa MWCO, and reconstituted to working volume in PBS.

Binding for wild-type DP4 and DP4^{L112W/V141M} with CD4 was measured by the Octet Red system (ForteBio, Fremont, Calif.). Experiments were performed at 25° C. using a 96-well OptiPlate (Perkin Elmer, Waltham, Mass.), with a 200-1 sample volume and constant shaking at 1,000 rpm. The biotinylated recombinant DP4 was loaded onto streptavidin-coated biosensors (ForteBio, Fremont, Calif.) until saturation, followed by baseline measurement in the HBS-EP buffer. Association was measured by incubating the loaded sensors for 400 sec with titrated concentrations of recombinant CD4 (0.8125 to 26 μM) before 300 sec dissociation in HBS-EP buffer alone. The steady-state analysis was fitted using a one-site specific binding model in GraphPad Prism 7.0

TABLE 7
Soluble 10x His-tagged CD4 Nucleic Acid Sequence

Fibroin L Signal Peptide; CD4; Gly/Ser Linker and
His tag sequences (10X))(SEQ ID NO: 262)
ATGATGCGGCCCATCGTGCTGGTGCTGCTGTTTGCCACATCTGCCCTGGC
CAAGAAAGTGGTGCTGGGCAAAAAAGGGGATACAGTGGAACTGACCTGTA
CAGCTTCCCAGAAGAAGAGCATACAATTCCACTGGAAAAACTCCAACCAG
ATAAAGATTCTGGGAAATCAGGGCTCCTTCTTAACTAAAGGTCCATCCAA
GCTGAATGATCGCGCTGACTCAAGAAGAAGCCTTTGGGACCAAGGAAACT
TTCCCCTGATCATCAAGAATCTTAAGATAGAAGACTCAGATACTTACATC
TGTGAAGTGGAGGACCAGAAGGAGGAGGTGCAATTGCTAGTGTTCGGATT
GACTGCCAACTCTGACACCCACCTGCTTCAGGGGCAGAGCCTGACCCTGA
CCTTGGAGAGCCCCCCTGGTAGTAGCCCCTCAGTGCAATGTAGGAGTCCA
AGGGGTAAAAACATACAGGGGGGGAAGACCCTCTCCGTGTCTCAGCTGGA
GCTCCAGGATAGTGGCACCTGGACATGCACTGTCTTGCAGAACCAGAAGA
AGGTGGAGTTCAAAATAGACATCGTGGTGCTAGCTTTCCAGAAGGCCTCC
AGCATAGTCTATAAGAAAGAGGGGGAACAGGTGGAGTTCTCCTTCCCACT
CGCCTTTACAGTTGAAAAGCTGACGGGCAGTGGCGAGCTGTGGTGGCAGG
CGGAGAGGGCTTCCTCCTCCAAGTCTTGGATCACCTTTGACCTGAAGAAC
AAGGAAGTGTCTGTAAAACGGGTTACCCAGGACCCTAAGCTCCAGATGGG
CAAGAAGCTCCCGCTCCACCTCACCCTGCCCCAGGCCTTGCCTCAGTATG
CTGGCTCTGGAAACCTCACCCTGGCCCTTGAAGCGAAAACAGGAAAGTTG
CATCAGGAAGTGAACCTGGTGGTGATGAGAGCCACTCAGCTCCAGAAAAA
TTTGACCTGTGAGGTGTGGGGACCCACCTCCCCTAAGCTGATGCTGAGCT
TGAAACTGGAGAACAAGGAGGCAAAGGTCTCGAAGCGGGAGAAGGCGGTG
TGGGTGCTGAACCCTGAGGCGGGGATGTGGCAGTGTCTGCTGAGTGACTC
GGGACAGGTCCTGCTGGAATCCAACATCAAGGTTCTGCCCACATGGGGCA
GCCACCACCACCATCACCATCATCATCACCATTGA
Fibroin L Signal Peptide; CD4; Gly/Ser Linker and
His tag sequences (10X))(SEQ ID NO: 263)
MMRPIVLVLLFATSALAKKVVLGKKGDTVELTCTASQKKSIQFHWKNSNQ
IKILGNQGSFLTKGPSKLNDRADSRRSLWDQGNFPLIIKNLKIEDSDTYI
CEVEDQKEEVQLLVFGLTANSDTHLLQGQSLTLTLESPPGSSPSVQCRSP
RGKNIQGGKTLSVSQLELQDSGTWTCTVLQNQKKVEFKIDIVVLAFQKAS
SIVYKKEGEQVEFSFPLAFTVEKLTGSGELWWQAERASSSKSWITFDLKN
KEVSVKRVTQDPKLQMGKKLPLHLTLPQALPQYAGSGHLTLALEAKTGKL
HQEVNLVVMRATQLQKNLTCEVWGPTSPKLMLSLKLENKEAKVSKREKAV
WVLNPEAGMWQCLLSDSGQVLLESNIKVLPTWGSHHHHHHHHHH

### Example 2—L112W/V141M Substitutions of the DPβ Chain Enhance the Binding of DP to CD4

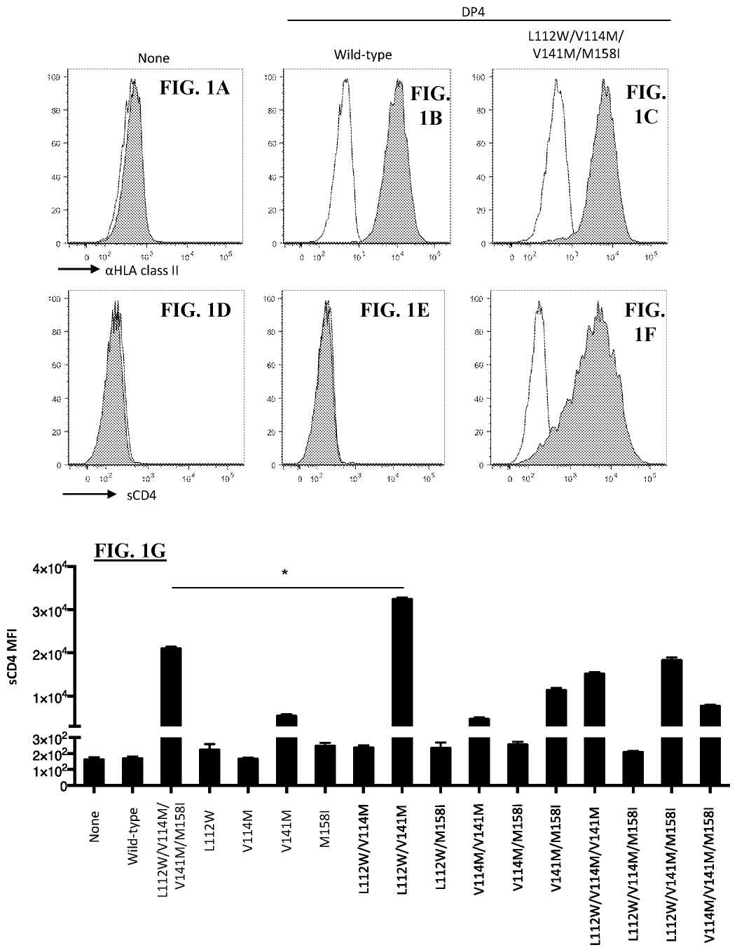

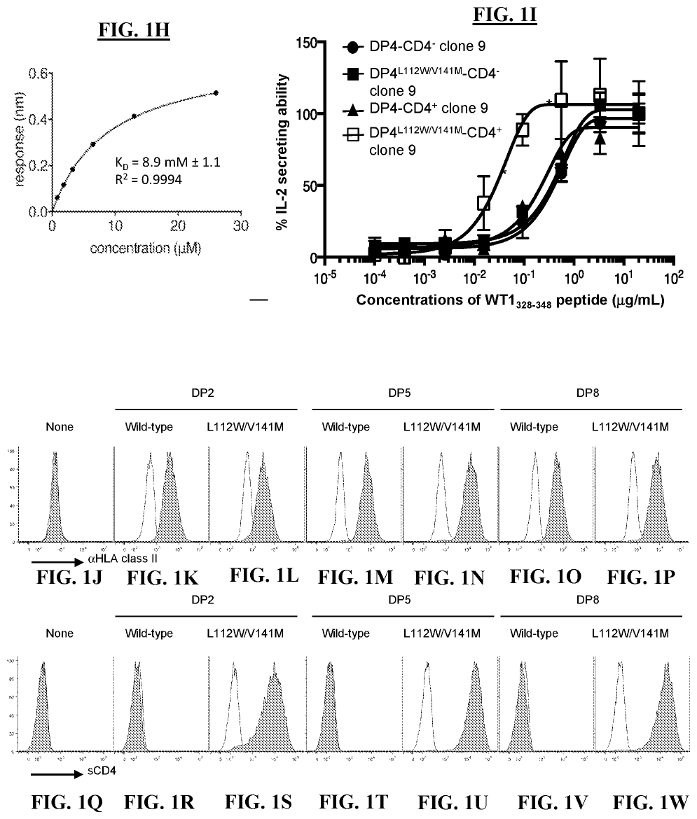

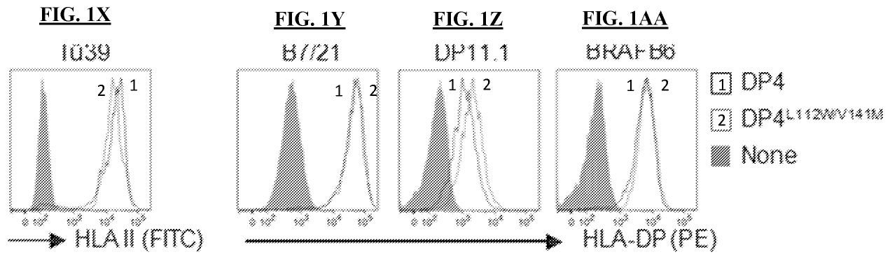

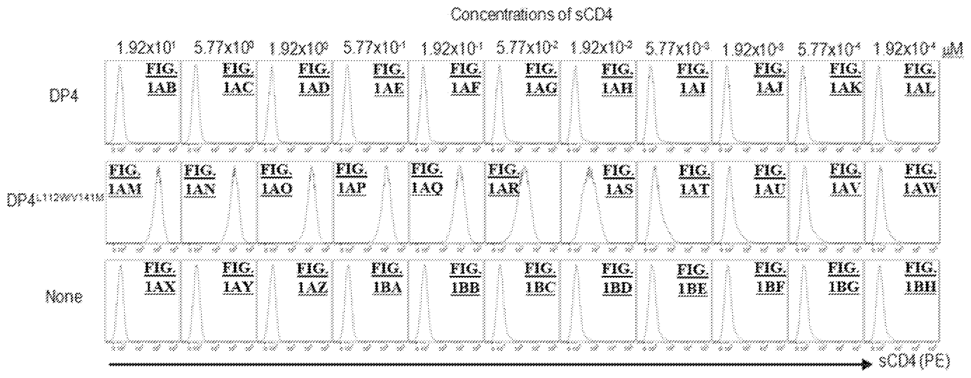

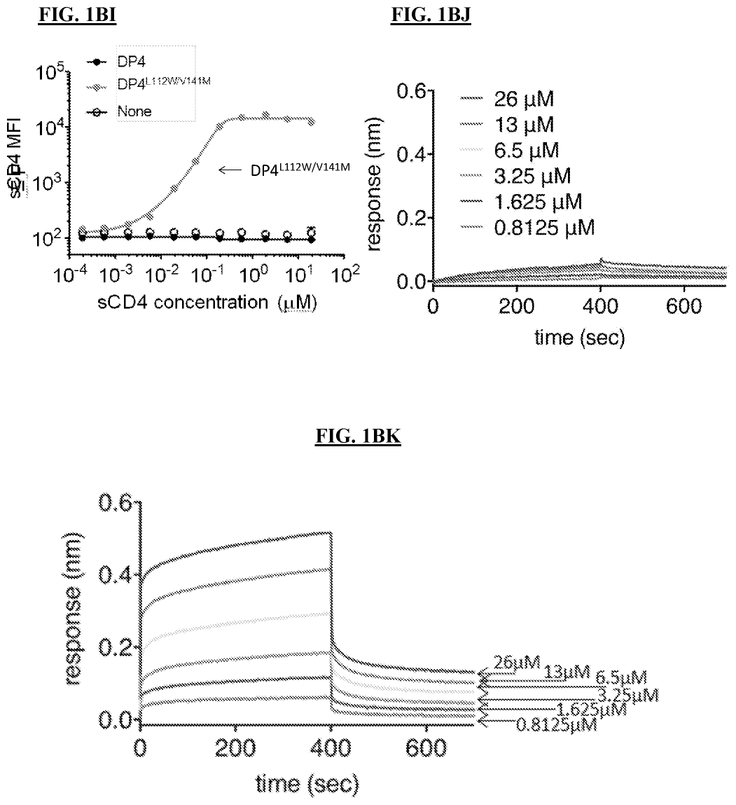

A cDNA expression library was generated of the DPB1*04:01 (DP40) gene carrying random mutations at L112, V114, V141, L156, and M158, which corresponds to L114, V116, V143, L158, and M160 of the DR1β chain, respectively, and coexpressed the library along with the wild-type DPA1*01:03 (DPα) gene in class II-deficient K562 cells. After two rounds of screening using soluble CD4 protein (sCD4), cell populations with enhanced CD4 binding were isolated, from which a mutant DP4β gene carrying L112W, V114M, V141M, and M158I substitutions was molecularly cloned. When ectopically expressed in the K562 cells, the mutant DP4 molecules consisting of the wild-type DPα chain and cloned mutant DP4β chain carrying L112W, V114M, V141M, and M158I substitutions (DP4^{L112W/V114M/V141M/M158I}) indeed showed enhanced binding to sCD4 compared with the wild-type DP4 molecules, excluding the possibility that enhanced CD4 binding was an artifact of screening processes (FIGS. 1A-1F).

To determine which of the four mutations is critical for enhanced CD4 binding, a reversion mutagenesis study was conducted. All the possible reversion DP4 mutants were reconstituted on class II-negative K562 cells and stained with sCD4. Both the L112W and V141M but not V114M or M158I single substitutions individually enhanced the binding of DP4 to sCD4 (FIG. 1G). Importantly, the L112W/V141M double mutations (DP4^{L112W/V141M}) synergistically enhanced the DP4/CD4 binding (FIG. 1G). Interestingly, both the V114M and M158I single replacements appeared to have a negative effect on the enhanced binding enabled by the DP4^{L112W/V141M} mutations (FIG. 1G). Previous studies have estimated that the K_{D} value between CD4 and HLA class II is >2 mM. Using biolayer interferometry (BLI) binding assay, the affinity of DPR^{L112/V141M} for CD4 was measured. While no binding was detected between wild-type DP4 and CD4, DP4^{L112W/V141M} bound to CD4 with a K_{D} of 8.9 μM±1.1 (FIG. 1H and FIGS. 1X-1BK). This value represents an at least 200-fold improvement in the binding affinity. Further, the observed affinity between CD4 and DP4^{L112W/V141M} is higher than that between human CD8 and HLA class I (˜200 μM) and is comparable to that between mouse CD8 and mouse MHC Class I (˜10 μM). To confirm that enhanced binding between DP4^{L112W/V141M} and CD4 leads to an enhanced CD4⁺ T cell response, a comparison was conducted of the immunostimulatory capacity of artificial APCs (aAPCs) expressing either wild-type DP4 or DP4^{L112W/V141M} as a single class II allele using DP4/WT1 TCR (clone 9)-transduced CD4⁻ and CD4⁺ Jurkat 76 T cells as responder cells. As expected, DP4^{L112W/V141M} carrying aAPCs demonstrated enhanced T cell stimulatory activity in a CD4-dependent manner (FIG. 1I).

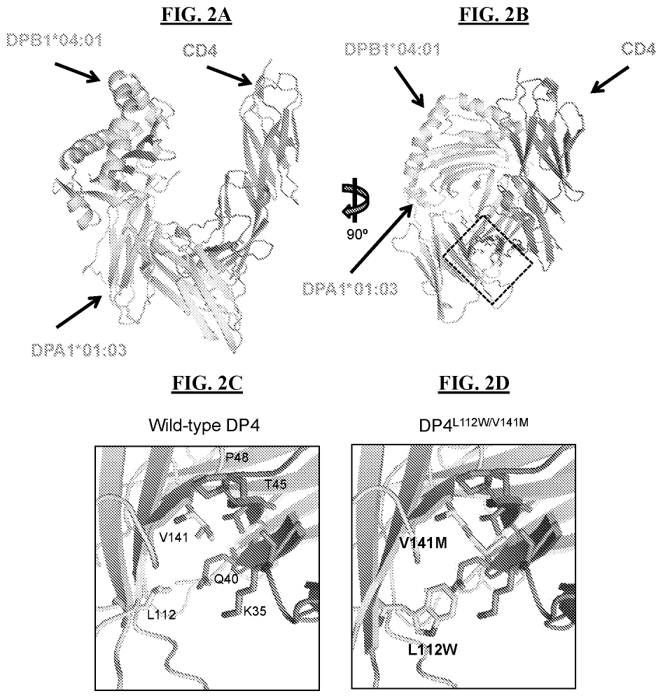

Next other DP alleles to CD4 were analyzed to determine whether the L112W/V141M mutations also enhance binding. Although none of the wild-type DP2, DP5, or DP8 bound to CD4, all three molecules bound to CD4 strongly when the L112W/V141M double mutations were introduced in the DPβ chains of these molecules (FIGS. 1I-1W). A structural model (FIGS. 2A-2D) constructed based on a previous report revealed that in the DP4^{L112W/V141M}-CD4 complex, the two L112W/V141M mutations apparently induced a hydrophobic effect at the positions, K35, Q40, and T45 of CD4. These results show that L112W/V141M mutations can enhance the CD4 binding of at least all 4 of the DP alleles tested.

### Example 3—Affinity-Matured DP4^{L112W/V141M} Multimers Specifically Stain Cognate TCRs

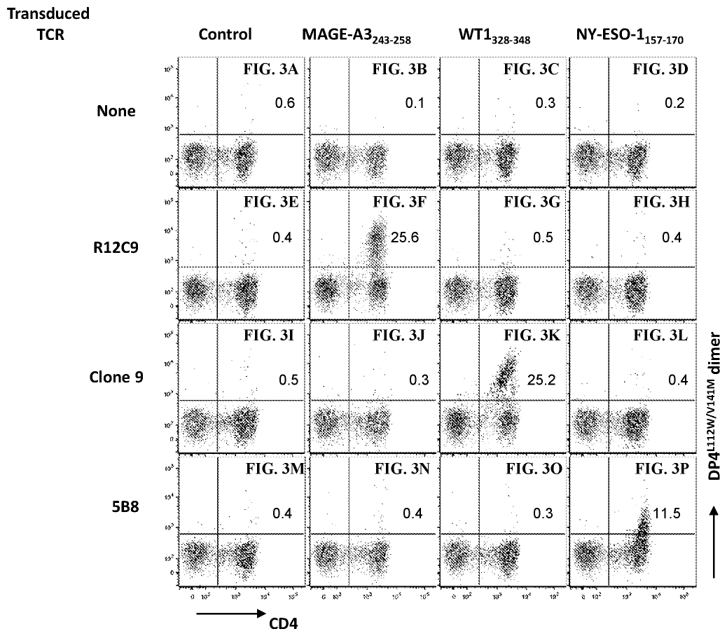

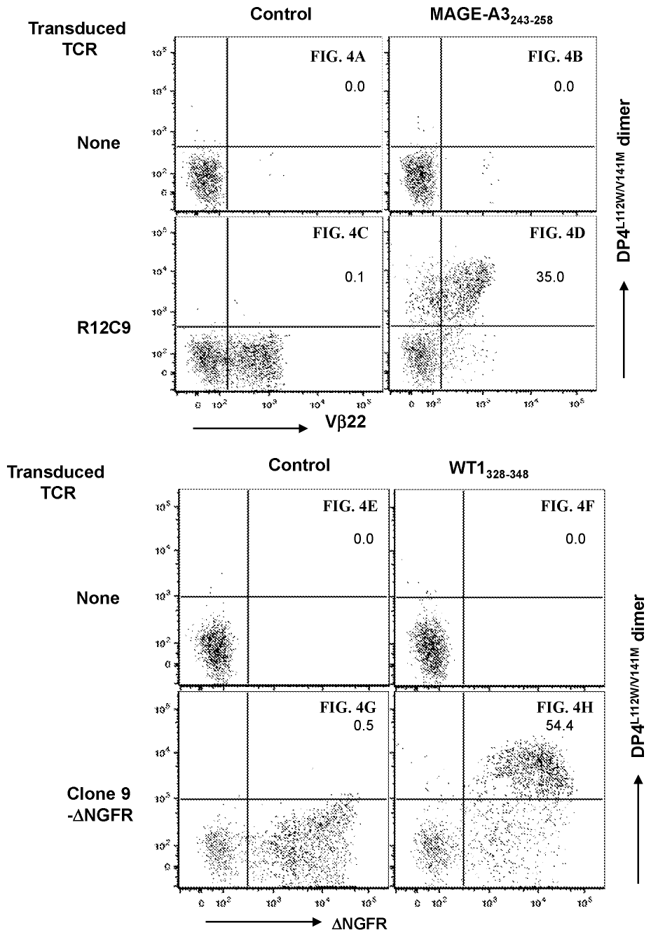

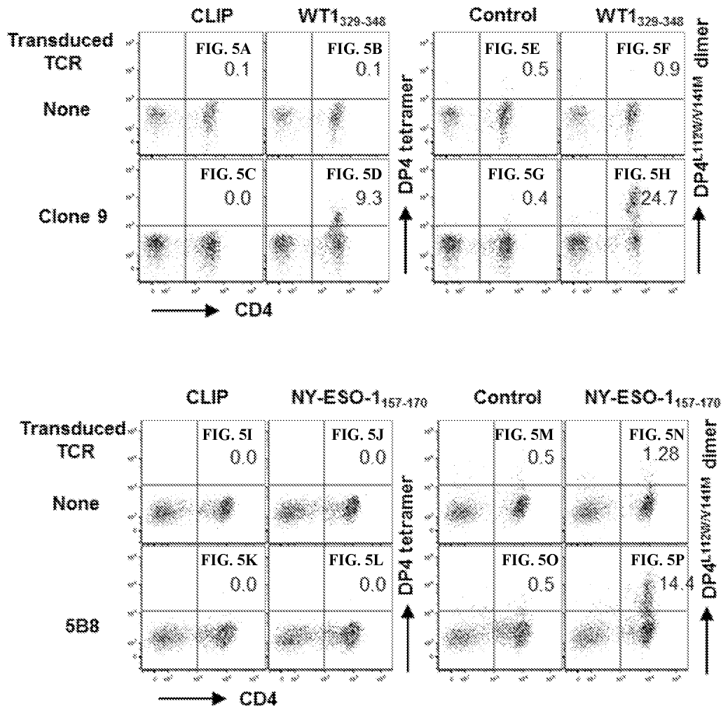

To determine the effect of the L112W/V141M double mutations of DP40 on DP4 multimer staining, a soluble DP4^{L112W/V141M} monomer was produced, which was then dimerized with an anti-His tag mAb. Primary T cells were individually transduced with three different DP4-restricted TCRs specific for MAGE-A3 (clone R12C9), WT1 (clone 9), and NY-ESO-1 (clone 5B8) and then stained with cognate DP4^{L112W/V141M} dimers. As shown in FIGS. 3A-3P, each DP4^{L112W/V141M} dimer specifically stained CD4⁺ T cells expressing the cognate TCR. Costaining of R12C9- and clone 9-transduced T cells with an anti-Vβ22 mAb and anti-NGFR mAb, respectively, along with the respective DP4^{L112W/V141M} dimer confirmed that virtually all TCR-transduced CD4⁺ T cells were successfully stained with the respective DP4^{L112W/V141M} dimers (FIGS. 4A-4H). Compared with conventional wild-type DP4 tetramers, our novel DP4^{L112W/V141M} dimers stained both DP4/WT1 and DP4/NY-ESO-1 T cells better than conventional wild-type DP4 tetramers (FIGS. 5A-5P). Notably, the conventional wild-type DP4/NY-ESO-1 tetramer was unable to stain cognate T cells even at the highest concentration available (data not shown).

### Example 4—DP4^{L112W/V141M} Dimer Technology is Robust and Versatile

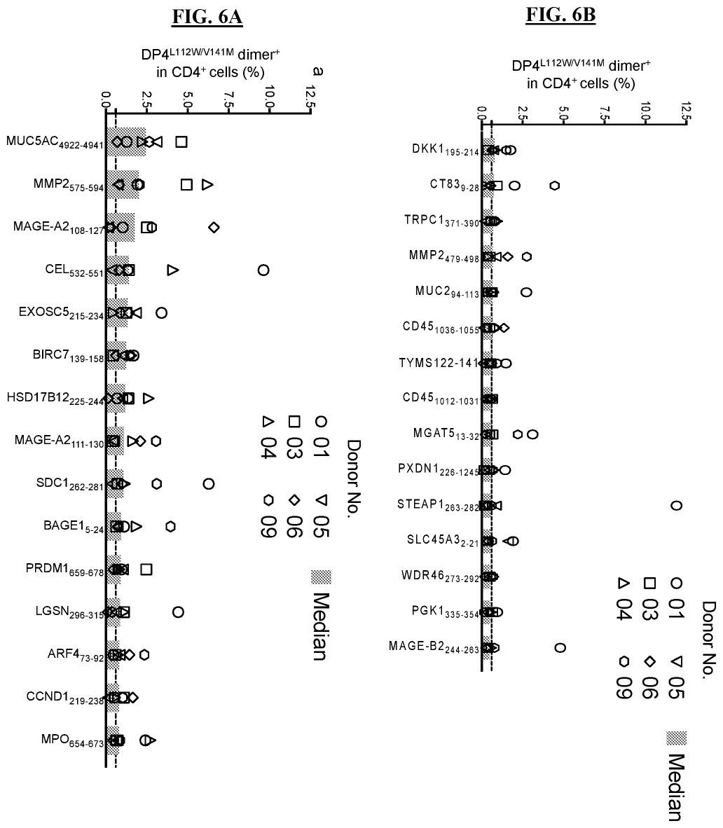

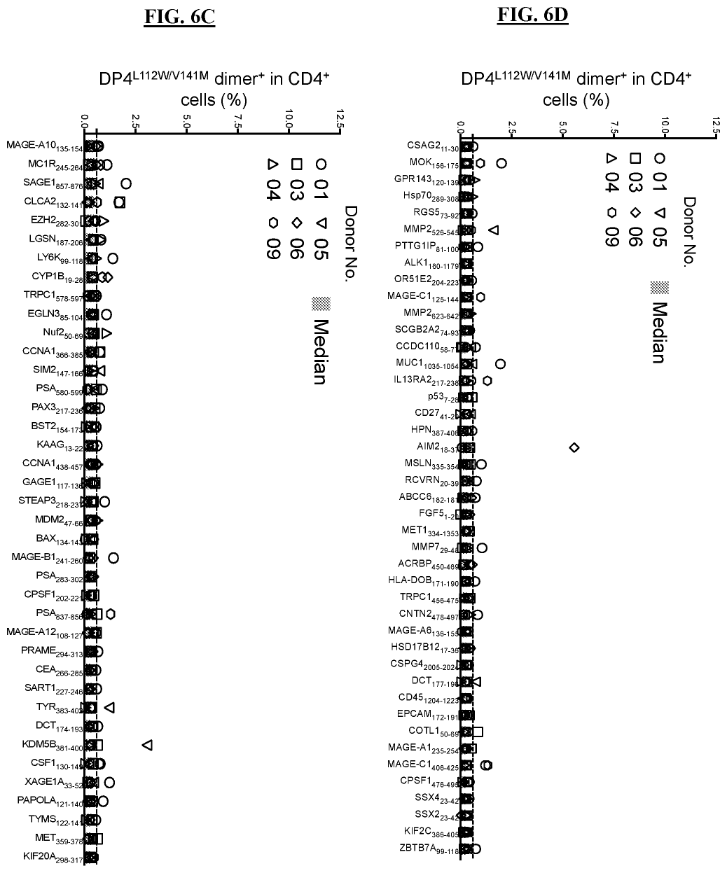

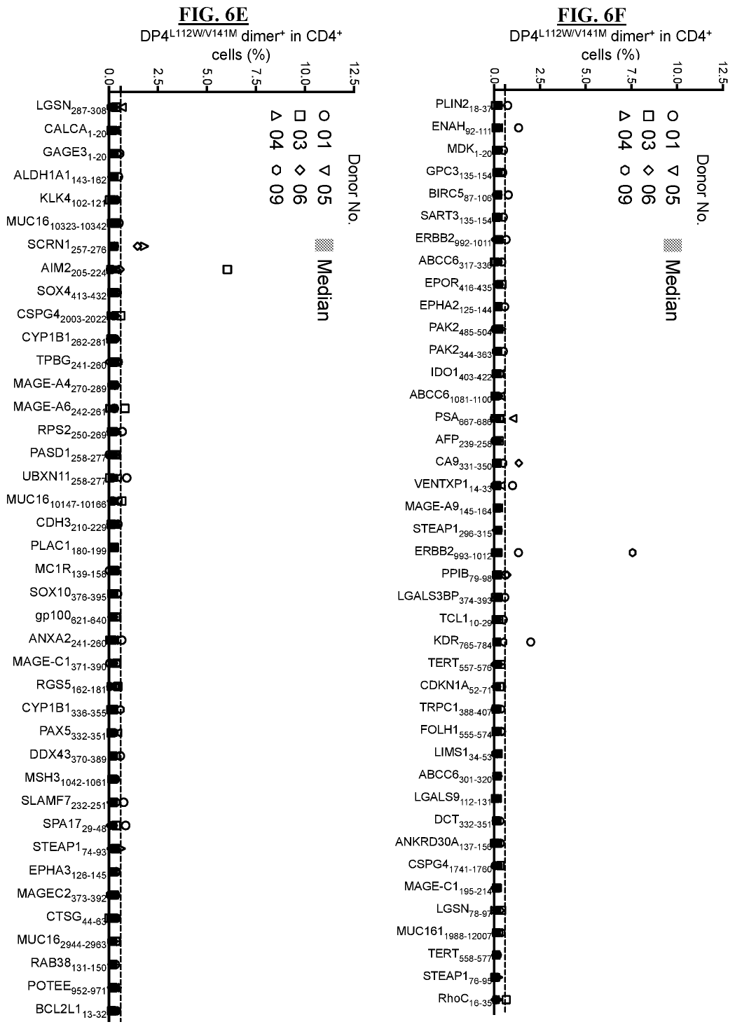

To demonstrate the robustness and versatility of the DP4^{L112W/V141M} multimer staining, a comprehensive screening was performed for the in vitro immunogenicity of potential DP4-restricted peptides derived from an array of tumor-associated antigens (Table 6). One hundred and ninety-six DP4-restricted and tumor-associated antigen-derived 20-mer peptides were predicted using a peptide prediction algorithm (NetMHC2 ver.2.2) and chemically synthesized (Table 6). The frequency of antigen-specific CD4⁺ T cells is generally very low in the periphery; therefore, primary CD4⁺ T cells isolated from six DP4⁺ melanoma patients were stimulated only once with DP4-aAPCs individually pulsed with the 196 peptides and stained with cognate DP4^{L112W/V141M} dimers. To avoid potential in vitro priming, weak stimulatory conditions were utilized. As shown in FIGS. 6A-6F, 103 predicted DP4 peptides were immunogenic, at least in vitro.

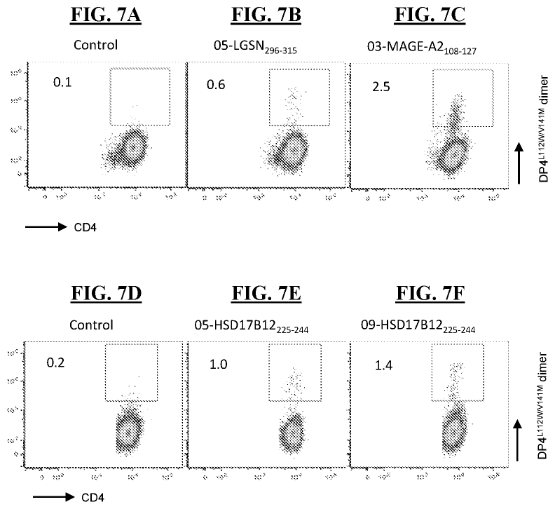

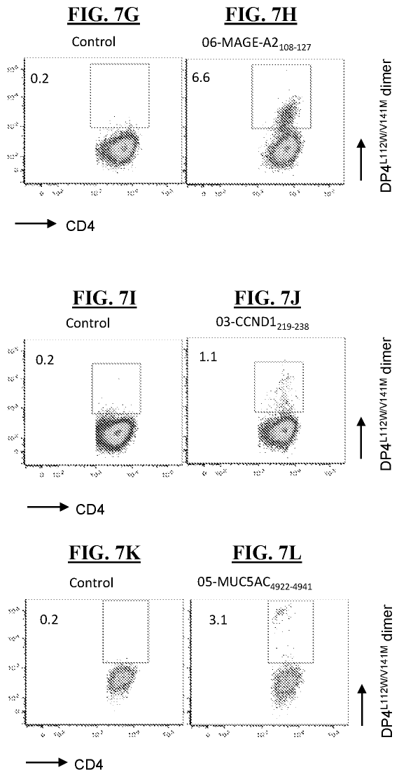

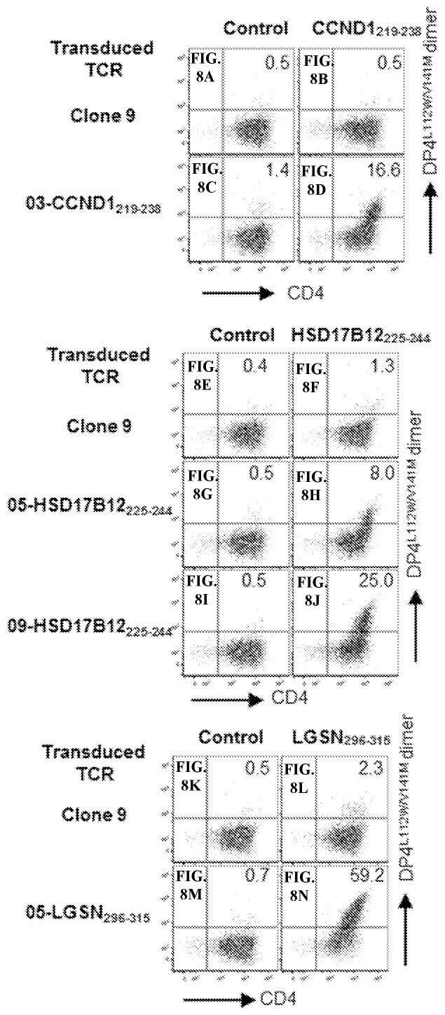

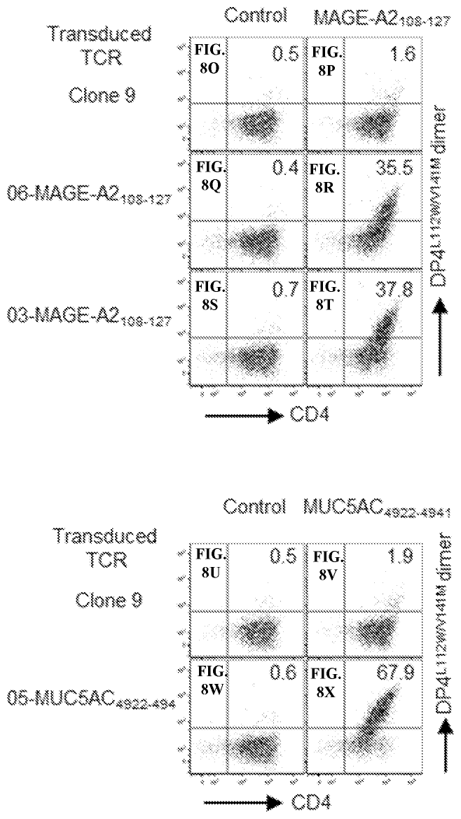

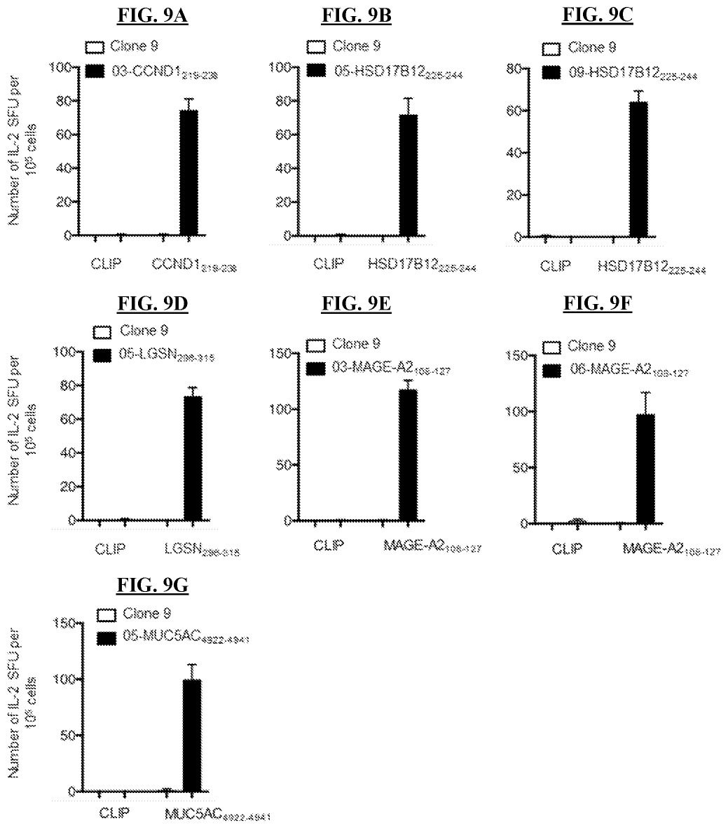

To validate the dimer staining results, we cloned seven DP4-restricted TCR genes specific for CCND1₂₁₉₋₂₃₈, HSD17B12₂₂₅₋₂₄₄, LGSN₂₉₆₋₃₁₅, MAGE-A2₁₀₈₋₁₂₇, and MUC5AC₄₉₂₂₋₄₉₄₁ (FIGS. 7A-7L and Table 8) from the dimer-positive T cells. When clonotypically reconstituted in human CD4⁺ TCR-deficient T cells, all these TCRs were successfully stained by the cognate DP4^{L112W/V141M} dimers (FIGS. 8A-8X) and were functional in a DP4-restricted and antigen-specific manner (FIGS. 9A-9G).

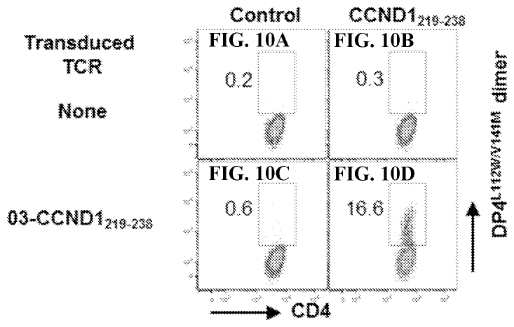

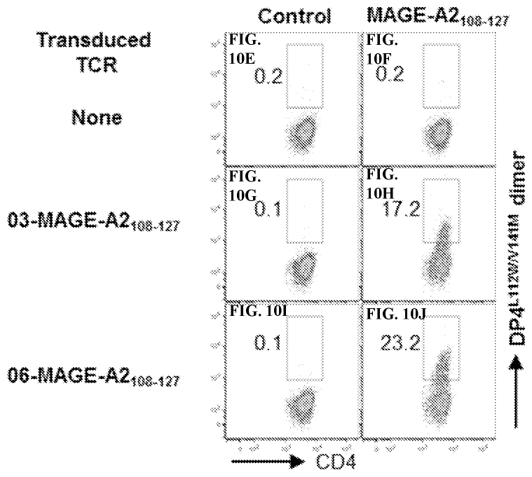

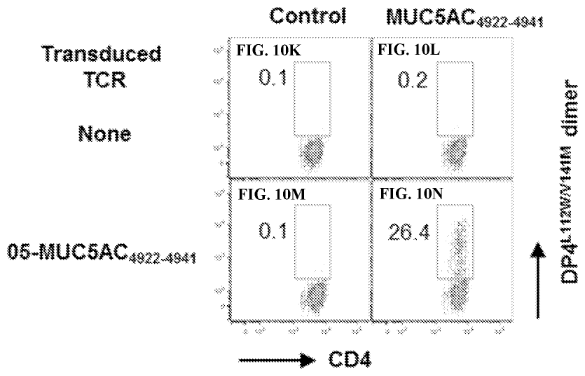

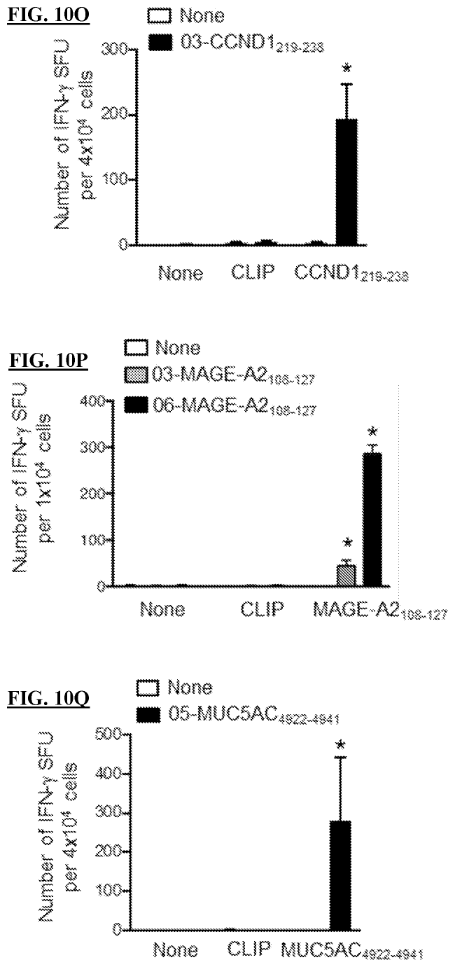

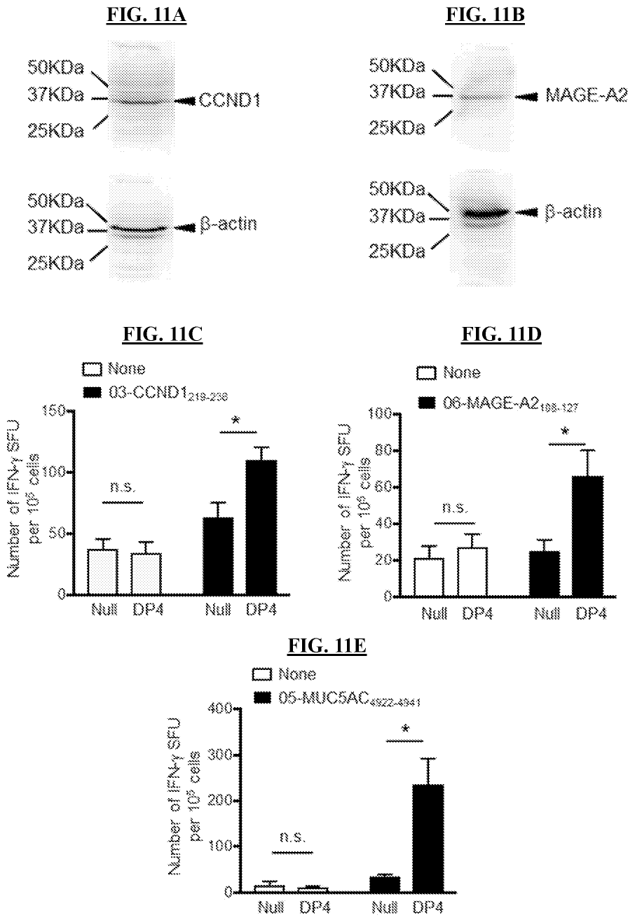

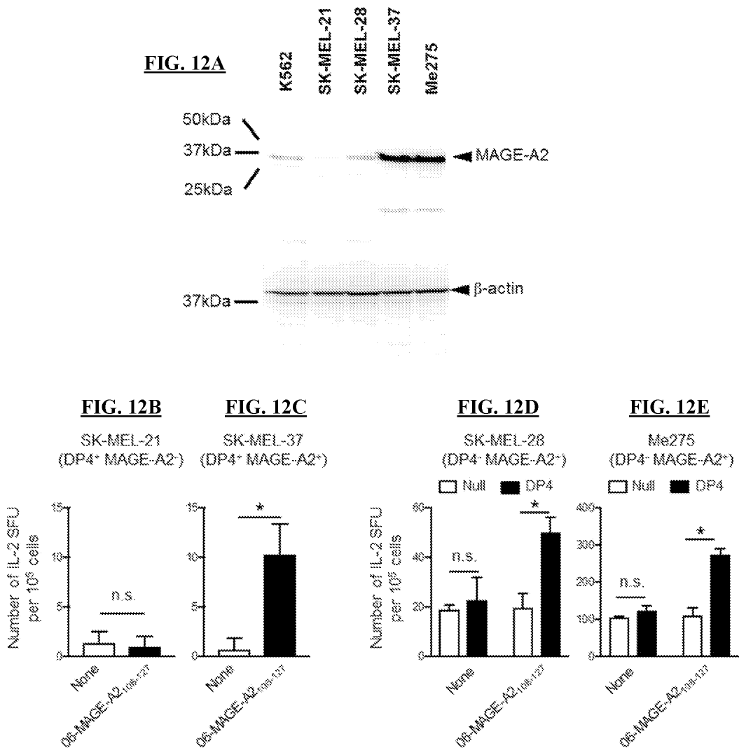

Among the four TCRs individually expressed in primary T cells, three TCRs, i.e., 03-CCND1₂₁₉₋₂₃₈, 06-MAGE-A2₁₀₈₋₁₂₇, and 05-MUC5AC₄₉₂₂₋₄₉₄₁, were able to recognize cognate peptides that were endogenously processed and presented by DP4 (FIGS. 10A-10Q and 11A-11E). Importantly, 06-MAGE-A2₁₀₈₋₁₂₇-transduced primary T cells were able to recognize melanoma cell lines in a DP4- and MAGE-A2-dependent manner (FIGS. 12A-12E).

TABLE 8
DP4-Restricted TCRs

| No. | Peptide | TRAV | TRAJ | TCR-alpha CDR 3 | TRBV | TRBJ | TCR-beta CDR 3 |
|---|---|---|---|---|---|---|---|
| 03 | CCND1₂₁₉₋₂₃₈ | 2*01 | 21*01 | CAVCTLYNFNKFYF | 6- | 2-1*01 | CASLTDNNEQFF |
| | | | | (SEQ ID NO: 36) | 5*01 | | (SEQ ID NO: 43) |
| 05 | HSD17B12₂₂₅₋₂₄₄ | 22*01 | 18*01 | CAVAPYDRGSTLGRLY | 19*01 | 2-5*01 | CASSTGQGLETQYF |
| | | | | F (SEQ ID NO: 37) | | | (SEQ ID NO: 44) |
| 09 | HSD17B12₂₂₅₋₂₄₄ | 27*01 | 33*01 | CAGVKDSNYQLIW | 30*01 | 2-1*01 | CAWSSYNEQFF (SEQ |
| | | | | (SEQ ID NO: 38) | | | ID NO: 45) |
| 05 | LGSN₂₉₆₋₃₁₅ | 9-2*03 | 32*01 | CALSDLSYGGATNKLI | 27*01 | 1-5*01 | CASSKGQGLGNQPQHF |
| | | | | F (SEQ ID NO: 39) | | | (SEQ ID NO: 46) |
| 03 | MAGE-A2₁₀₈₋₁₂₇ | 36/DV7* | 40*01 | CAVEVNSGTYKYIF | 2*01 | 1-1*01 | CASRRDLAAFF (SEQ |
| | | 04 | | (SEQ ID NO: 40) | | | ID NO: 47) |
| 06 | MAGE-A2₁₀₈₋₁₂₇ | 19*01 | 40*01 | CALSVGTYKYIF(SEQ | 7- | 2-5*01 | CASSPGTGGRETQYF |
| | | | | ID NO: 41) | 9*01 | | (SEQ ID NO: 48) |
| 05 | MUC5AC₄₉₂₂₋₄₉₄₁ | 38-1*03 | 58*01 | CAFMKRAETSGSRLTF | 6- | 2-5*01 | CASSYWPTRETQYF |
| | | | | (SEQ ID NO: 42) | 2*01 | | (SEQ ID NO: 49) |

In contrast to CD8, the role and function of CD4 as a coreceptor has yet to be fully elucidated. This lack of information exists mainly because the binding between CD4 and class II is exceptionally weak, which significantly limits research on the role of the association between CD4 and class II. In this study, an affinity-matured form of HLA-DP4, i.e., DP4^{L112W/V141M}, was isolated with enhanced CD4 binding, and a novel DP4^{L112W/V141M} dimer technology was developed, which introduces robustness and rigorousness in the detection of DP4-restricted antigen-specific CD4⁺ T cells.

Using this DP4^{L112W/V141M} dimer technology, DP4-restricted antitumor T cell responses were comprehensively studied in vitro and multiple DP4-restricted immunogenic peptides and cognate TCR genes were identified. HLA-DP4 is the most prevalent HLA allele in many ethnic groups and belongs to the DP^{84Gly} group. Unlike other class II molecules, DP^{84Gly} molecules such as DP4 constitutively present peptides derived from endogenous sources regardless of the invariant chain and HLA-DM expression. The improved presentation of endogenous peptides via class II is correlated with improved survival of cancer patients. Notably, a first-in-human class II-restricted TCR gene therapy indeed targeted a DP4-restricted MAGE-A3 peptide (see, e.g., Yao et al., J. Immunother. 39:191-201 (2016)). The DP^{84Gly} genotype, such as in DP2 and DP4, acts as a risk allele for anti-neutrophil cytoplasmic autoantibody-associated vasculitis. DP4 molecules, which can constitutively present peptides derived from endogenous tumor-associated antigens, may induce more clinically relevant antitumor responses than other class II molecules, serving as a protective class II allele.

To identify affinity-matured class II molecules, the present examples detail multiple mutations in the β-chain but not the α-chain because the β-chain has a more direct interaction with CD4 than the α chain. It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

In conclusion, CD4⁺ T cells play a critical role in the development of autoimmune diseases and protection against pathogenic infections and cancers. The novel HLA class II multimer technology described herein may better facilitate the study of HLA class II-restricted CD4⁺ T cell responses across HLA-DP alleles.

### Example 5—Generation of Affinity Matured HLA-DQ Molecules

Cells

Peripheral mononuclear cells were obtained via density gradient centrifugation (Ficoll-Paque PLUS, GE Healthcare Life Sciences, Marlborough, Mass.). The K562 cell line is an erythroleukemic cell line with defective HLA class I/II expression. A375 is melanoma cell lines. HEK293T cells and A375 cells were grown in DMEM supplemented with 10% FBS and 50 μg/ml gentamicin (Thermo Fisher Scientific, Waltham, Mass.). The K562 and Jurkat 76 cell lines were cultured in RPMI 1640 supplemented with 10% FBS and 50 μg/ml gentamicin.

Peptides

Synthetic peptides were purchased from Genscript (Piscataway, N.J.) and dissolved at 50 μg/ml in DMSO.

Antibodies

The following antibodies were used for flow cytometry analysis: PE-conjugated anti-class II (9-49 (I3), Beckman Coulter, Brea, Calif.; T039), APC-Cy7-conjugated anti-CD4 (RPA-T4, Biolegend, San Diego, Calif.) and PE-conjugated anti-His tag (AD1.1.10, Abcam, Cambridge, Mass.). Dead cells were distinguished with the LIVE/DEAD Fixable Near-IR Dead Cell Stain Kit 465 (Thermo Fisher Scientific, Waltham, Mass.). Stained cells were analyzed with Canto II or LSRFortessa X-20 (BD Biosciences, Franklin Lakes, N.J.). Cell sorting was conducted using a FACS Aria II (BD Biosciences, Franklin Lakes, N.J.). Data analysis was performed using FlowJo software (Tree Star, Ashland, Oreg.).

TCR Transduction into Primary T Cells

CD3⁺ and CD4⁺ T cells were purified using the Pan T Cell Isolation Kit (Miltenyi Biotec, Bergisch Gladbach, Germany) and CD4⁺ T Cell Isolation Kit (Miltenyi Biotec, Bergisch Gladbach, Germany), respectively. Purified T cells were stimulated with aAPC/mOKT3 irradiated with 200 Gy at an E:T ratio of 20:1. Starting the following day, activated T cells were retrovirally transduced with the cloned TCR genes via centrifugation for 1 hour at 1,000×g at 32° C. for 3 consecutive days or using a Retronectin-coated plate (Takara Bio, Shiga, Japan). On the following day, 100 IU/ml IL-2 and 10 ng/ml IL-15 were added to the TCR-transduced T cells. The culture medium was replenished every 2-3 days.

Staining with Soluble CD4

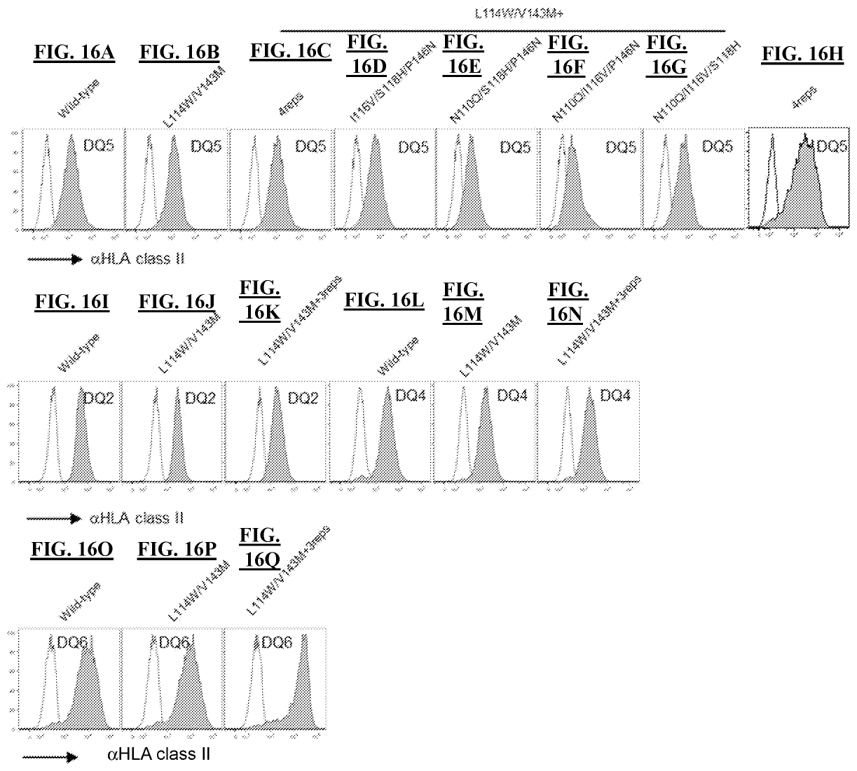

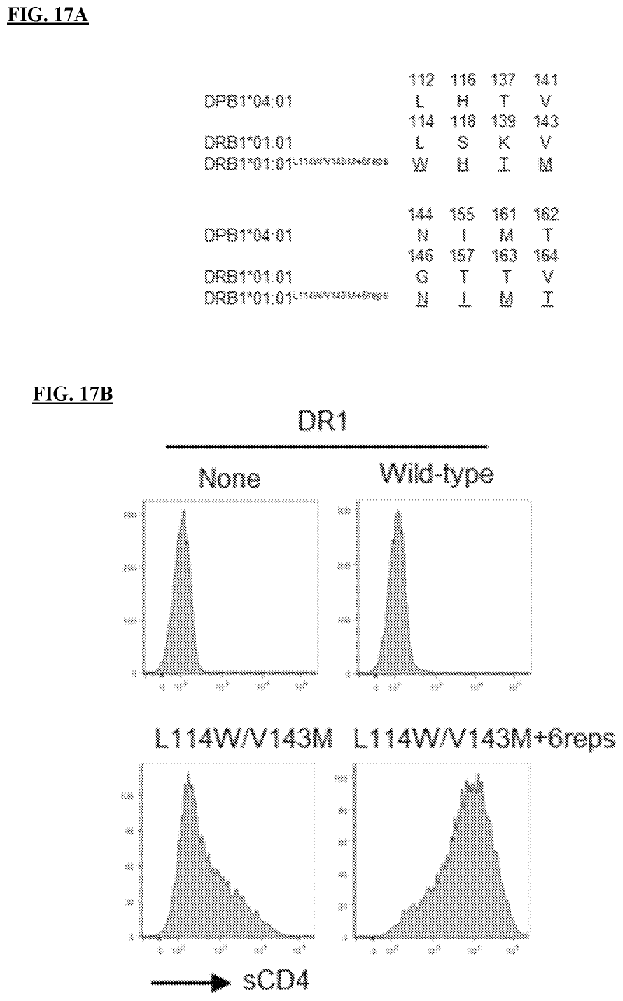

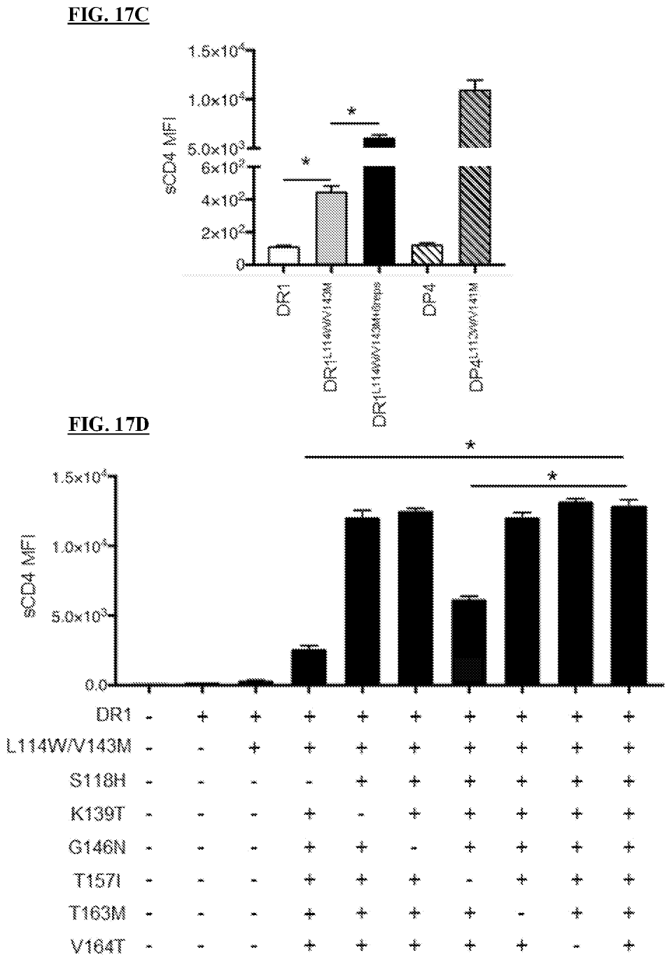

The soluble CD4 (sCD4) gene was generated by fusing the human CD4 extracellular domain with a 6×His tag via a GS linker. HEK293T cells were retrovirally transduced with the sCD4 gene, and the culture supernatant containing the sCD4 monomer was harvested. sCD4 was dimerized with a PE-labeled anti-6×His tag mAb (AD1.1.10, Abcam, Cambridge, Mass.) and used. HLA class II-expressing K562 cells were stained with dimerized sCD4 in the presence of goat serum for 30 min at room temperature. The surface HLA class II expression in K562-derived cells individually expressing various class II genes was as demonstrated in FIGS. 16A-16Q.

Generation of the HLA Class II Monomer and Dimer

The extracellular domain of the wild-type class II a gene was fused with an acidic leucine zipper via a GGGS linker followed by a 6×His tag via a GS linker (see SEQ ID NO: 18). The ectodomain of the class II β gene carrying mutations (see SEQ ID NO: 13) was similarly linked with a basic leucine zipper via a GGGS linker (see SEQ ID NO: 14). HEK293T cells and A375 cells were transfected with the α and β genes using the 293GPG cell-based retrovirus system and cultured in DMEM supplemented with 10% FBS and 50 μg/ml gentamicin. For dimer staining, A375 cells stably secreting soluble DQ5^{L114W/V143M+4reps} (which possesses the N110Q/I116V/S118H/P146N replacements (4reps) in addition to L114W/V143M) and DQ6^{L114W/V143M+3reps} (which possesses the N110Q/S118H/P146N replacements (3reps) in addition to L114W/V143M) protein were grown until confluent, and after forty-eight hours, the medium was harvested. The soluble HLA class II-containing supernatant was then mixed with 100 μg/ml peptide of interest for 20-24 hours at 37° C. for in vitro peptide exchange. Monomer that was not subjected to peptide exchange was used as a control. The concentration of the monomer was measured by specific ELISA using a nickel-coated plate (XPressBio, Frederick, Md.) and an anti-His tag biotinylated mAb (AD1.1.10, R&D Systems, Minneapolis, Minn.). Soluble HLA class II monomer was dimerized using PE-conjugated anti-His mAb (AD1.1.10, Abcam, Cambridge, Mass.) at a 2:1 molar ratio for 1.5 hours at 4° C. for staining.

HLA Class II Dimer Staining

Primary T cells transduced with exogenous TCR gene were pretreated with 50 nM dasatinib (LC Laboratories, Woburn, Mass.) for 30 min at 37° C. and stained with 5-15 μg/ml class II dimer for 4-5 hours at room temperature. After washing, cell surface molecules were counterstained with an APC-Cy7-conjugated anti-CD4 mAb.

Statistical Analysis

Statistical analysis was performed using GraphPad Prism 6.0 software (GraphPad Software, San Diego, Calif.). Unpaired two-tailed Student's t-tests were used for two-sample comparisons. No statistical method was used to predetermine sample size. The investigators were not blinded to allocation during the experiments or outcome assessment. The experiments were not randomized.

### Example 6—DQ Molecules with Enhanced CD4 Binding Capacities

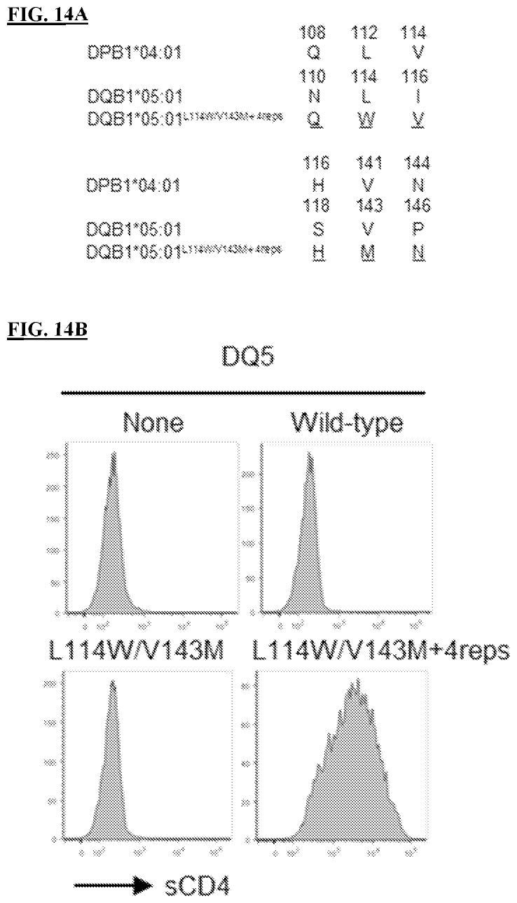

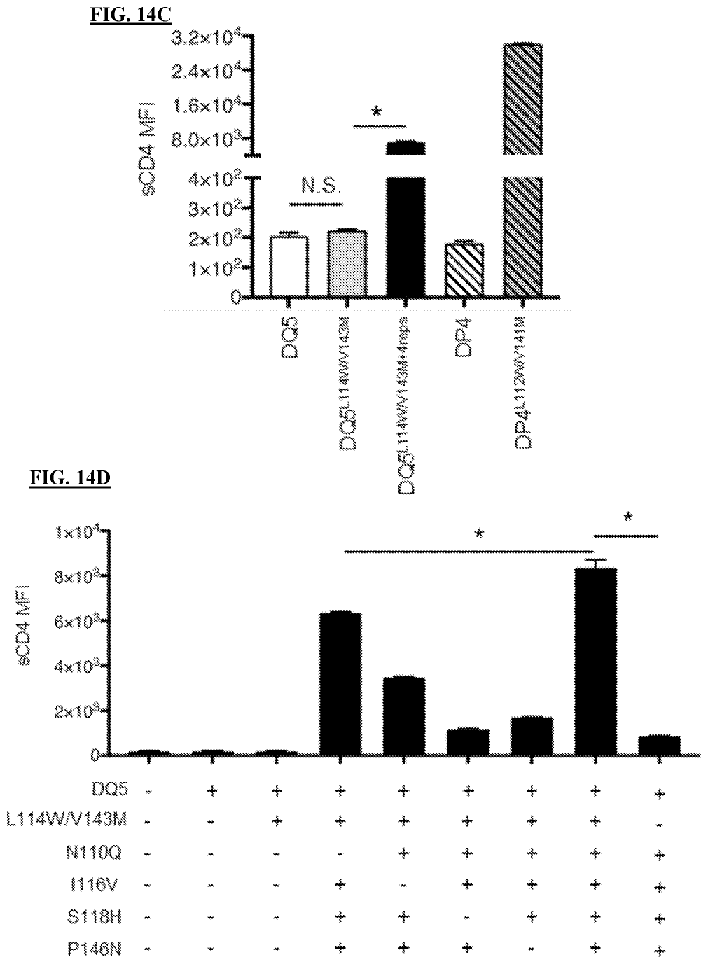

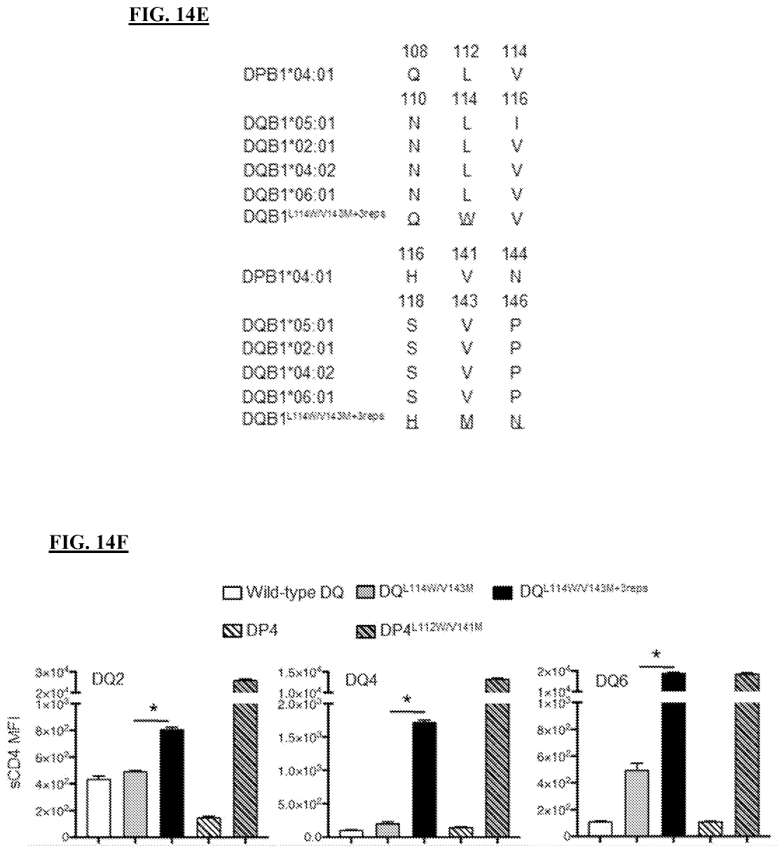

Affinity enhanced DQ molecules were generated by introducing L114W/V143M mutations, to determine if these substitutions could improve the binding of HLA-DQ molecules such as DQ5 (DQA1*01:01-DQB1*05:01) to CD4. DQB1*05:01 encodes four different amino acids at positions 110, 116, 118, and 146 in addition to 114 and 143. We therefore generated K562 cells expressing DQ5^{L114W/V143M+4reps}, which possesses the N110Q/I116V/S118H/P146N replacements (4reps) in addition to L114W/V143M (FIG. 14A), and stained the cells with sCD4. K562 cells expressing DQ5^{L114W/V143M+4reps} but not DQ5^{L114W/V143M}, DQ5^{4reps}, or wild-type DQ5 demonstrated enhanced CD4 binding (FIGS. 14B-14C). Importantly, a series of K562 cells individually expressing various DQ5^{L114W/V143M+4reps} mutants with a single amino acid reversal at one of the four positions lacked the enhanced CD4 binding capability (FIG. 14D). These results suggest that the four additional replacements at N110Q, I116V, S118H, and P146N are critical for the effectiveness of the L114W/V143M mutations in the observed enhanced DQ5:CD4 binding.

DQβ chains such as DQB1*02:01, 04:02, and 06:01 encode distinct amino acids at positions 110, 118, and 146 but not at 116 (FIG. 14E). Unlike DQB1*05:01, DQB1*02:01, 04:02, and 06:01 encode Val at position 116, similar to DPB1*04:01, which codes for Val at position 114. All the DQ2^{L114W/V143M+3reps}, DQ4^{L114W/V143M+3reps}, and DQ6^{L114W/V143M+3reps} mutants, the β chains of which carry the N110Q, S118H, and P146N replacements (3reps) along with L114W/V143M, showed enhanced CD4 binding activity (FIG. 14F).

### Example 7—Affinity-Matured DQ Dimers Specifically and Robustly Stained Cognate TCRs

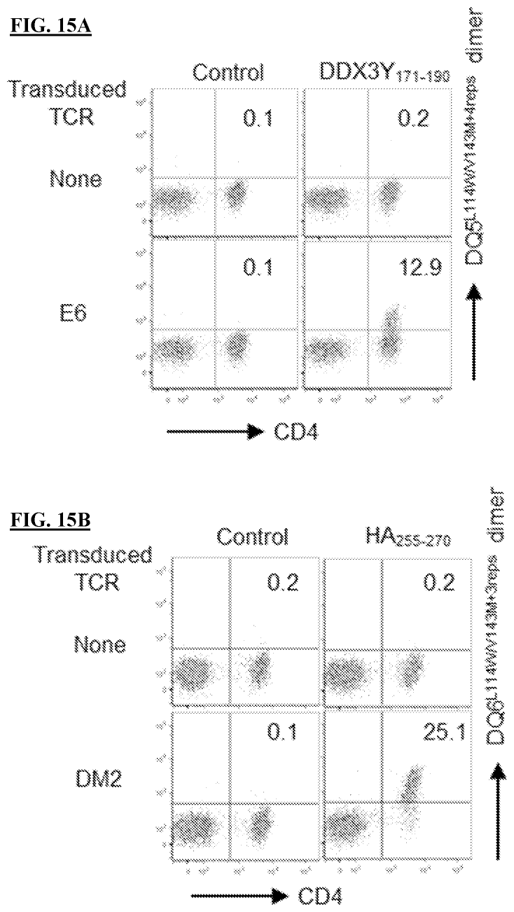

The ability of the affinity-matured DQ dimers carrying the mutations described in example 2 were evaluated for the ability to identify antigen-specific CD4⁺ T cells. The DQ5^{L114W/V143M+4reps} and DQ6^{L114W/V143M+3reps} dimers successfully stained the DQ5-restricted DDX3Y-specific TCR (E6) and DQ6-restricted influenza virus-specific TCR (DM2), respectively (FIGS. 15A-15B).

To identify affinity-matured class II molecules, the present examples detail multiple mutations in the β-chain but not the α-chain because the β-chain has a more direct interaction with CD4 than the α chain. It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

In conclusion, CD4⁺ T cells play a critical role in the development of autoimmune diseases and protection against pathogenic infections and cancers. The novel HLA class II multimer technology described herein may better facilitate the study of HLA class II-restricted CD4⁺ T cell responses across HLA-DQ alleles.

### Example 8—Generation of Affinity Matured HLA-DR Molecules

Cells

Peripheral mononuclear cells were obtained via density gradient centrifugation (Ficoll-Paque PLUS, GE Healthcare Life Sciences, Marlborough, Mass.). The K562 cell line is an erythroleukemic cell line with defective HLA class I/II expression. K562-based artificial APCs (aAPCs) individually expressing various HLA class II genes as a single HLA allele in conjunction with CD80 and CD83 have been reported previously (Butler et al., PloS One 7, e30229 (2012). HEK293T cells were grown in DMEM supplemented with 10% FBS and 50 μg/ml gentamicin (Thermo Fisher Scientific, Waltham, Mass.). The K562 cells were cultured in RPMI 1640 supplemented with 10% FBS and 50 μg/ml gentamicin.

Peptides

Synthetic peptides were purchased from Genscript (Piscataway, N.J.) and dissolved at 50 μg/ml in DMSO.

Antibodies

The following antibodies were used for flow cytometry analysis: PE-conjugated anti-class II (9-49 (I3)), APC-Cy7-conjugated anti-CD4 (RPA-T4, Biolegend, San Diego, Calif.)⁴⁴, PE-conjugated anti-His tag (AD1.1.10, Abcam, Cambridge, Mass.), and FITC-conjugated anti-V022 (IMMU 546, Beckman Coulter, Brea, Calif.). Dead cells were distinguished with the LIVE/DEAD Fixable Near-IR Dead Cell Stain Kit 465 (Thermo Fisher Scientific, Waltham, Mass.). Stained cells were analyzed with Canto II or LSRFortessa X-20 (BD Biosciences, Franklin Lakes, N.J.). Cell sorting was conducted using a FACS Aria II (BD Biosciences, Franklin Lakes, N.J.). Data analysis was performed using FlowJo software (Tree Star, Ashland, Oreg.).

TCR Transduction into Primary T Cells

CD4⁺ T cells were purified using the CD4⁺ T Cell Isolation Kit (Miltenyi Biotec, Bergisch Gladbach, Germany). Purified T cells were stimulated with aAPC/mOKT3 irradiated with 200 Gy at an E:T ratio of 20:1. Starting the following day, activated T cells were retrovirally transduced with the cloned TCR genes via centrifugation for 1 hour at 1,000×g at 32° C. for 3 consecutive days or using a Retronectin-coated plate (Takara Bio, Shiga, Japan). On the following day, 100 IU/ml IL-2 and 10 ng/ml IL-15 were added to the TCR-transduced T cells. The culture medium was replenished every 2-3 days.

Staining with Soluble CD4

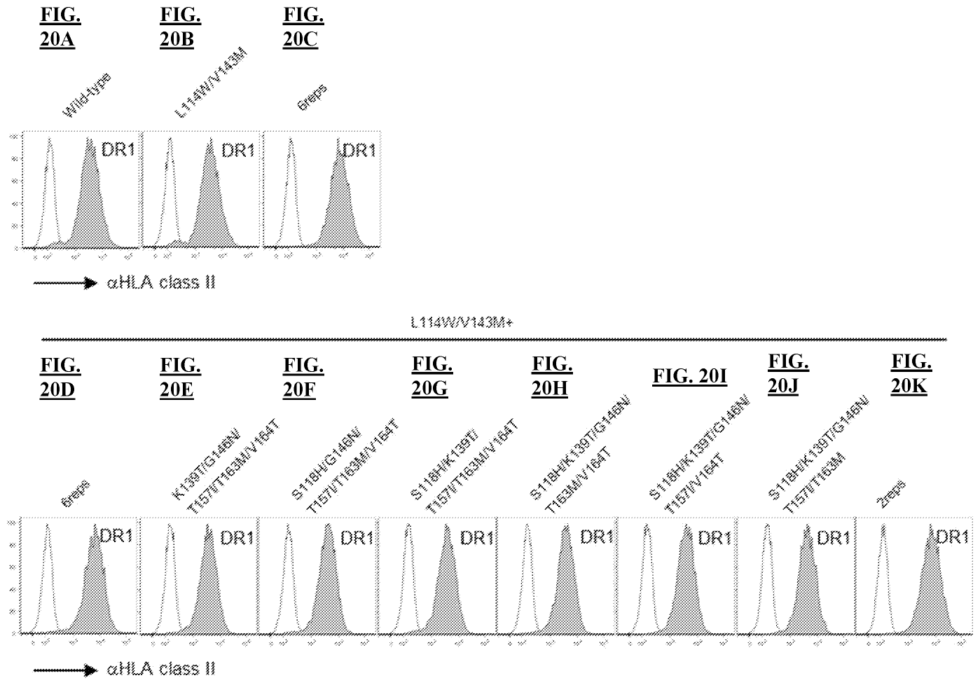

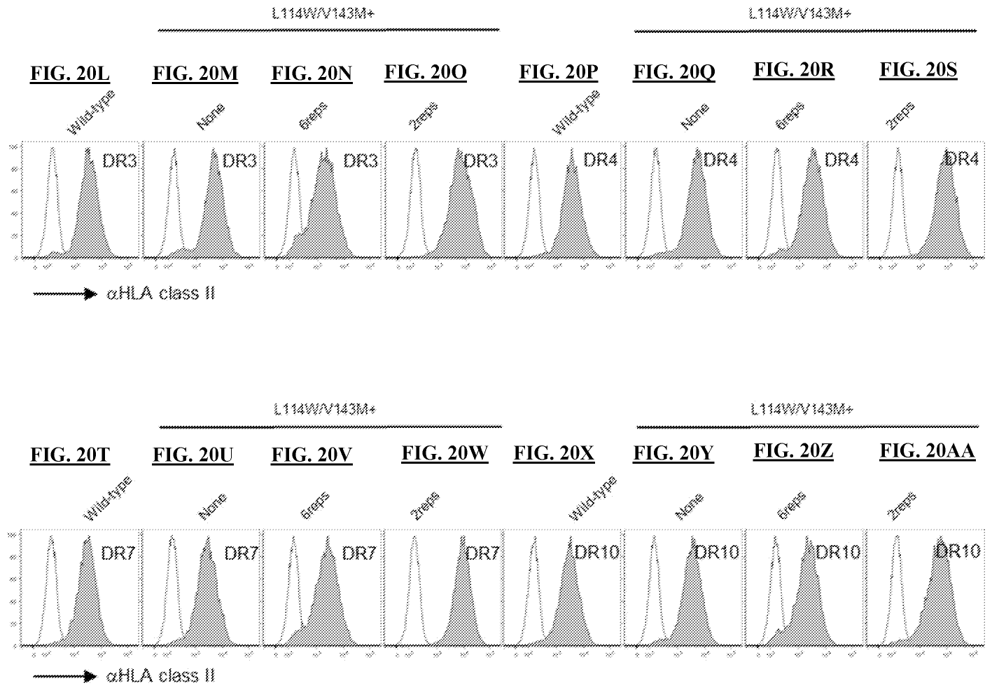

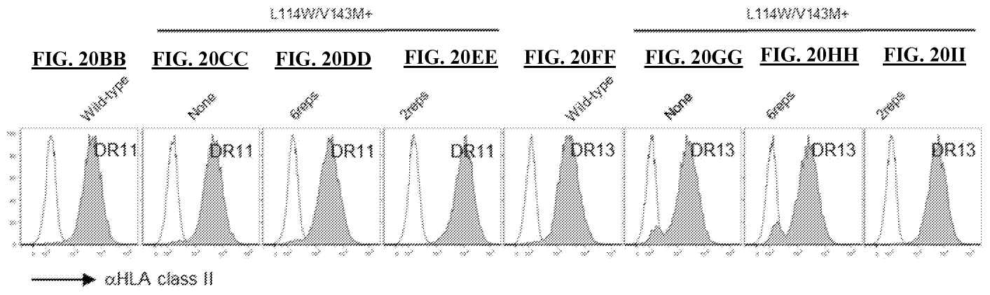

The soluble CD4 (sCD4) gene was generated by fusing the human CD4 extracellular domain with a 6×His tag via a GS linker. HEK293T cells were retrovirally transduced with the sCD4 gene, and the culture supernatant containing the sCD4 monomer was harvested. sCD4 was dimerized with a PE-labeled anti-6×His tag mAb (AD1.1.10, Abcam, Cambridge, Mass.) and used. HLA class II-expressing K562 cells were stained with dimerized sCD4 in the presence of goat serum for 30 min at room temperature. The surface HLA class II expression in K562-derived cells individually expressing various class II genes was as demonstrated in FIGS. 20A-20II.

Generation of the HLA Class H Monomer and Dimer

The extracellular domain of the wild-type class II α gene was fused with an acidic leucine zipper via a GGGS linker followed by a 6×His tag via a GS linker (see SEQ ID NO: 26). The ectodomain of the class II β gene carrying mutations (see SEQ ID NO: 21) was similarly linked with a basic leucine zipper via a GGGS linker (see SEQ ID NO: 22). HEK293T cells were transfected with the α and β genes using the 293GPG cell-based retrovirus system and cultured in DMEM supplemented with 10% FBS and 50 μg/ml gentamicin. For DR1 dimer staining, HEK293T cells stably secreting soluble DR1^{L114W/V143M+2reps}, DR7^{L114W/V143M+2reps} and DR11^{L114W/V143M+2reps} (which possesses the S118H/T157I replacements (2reps) in addition to L114W/V143M) protein were grown until confluent, and the after forty-eight hours, the medium was harvested. The soluble HLA class II-containing supernatant was then mixed with 100 μg/ml peptide of interest for 20-24 hours at 37° C. for in vitro peptide exchange. Monomer that was not subjected to peptide exchange was used as a control. The concentration of the monomer was measured by specific ELISA using a nickel-coated plate (XPressBio, Frederick, Md.) and an anti-His tag biotinylated mAb (AD1.1.10, R&D Systems, Minneapolis, Minn.). Soluble HLA class II monomer was dimerized using PE-conjugated anti-His mAb (AD1.1.10, Abcam, Cambridge, Mass.) at a 2:1 molar ratio for 1.5 hours at 4° C. for staining.

HLA Class H Dimer Staining

Primary T cells transduced with exogenous TCR gene were pretreated with 50 nM dasatinib (LC Laboratories, Woburn, Mass.) for 30 min at 37° C.⁴⁶ and stained with 5-15 μg/ml class II dimer for 4-5 hours at room temperature. After washing, cell surface molecules were counterstained with an APC-Cy7-conjugated anti-CD4 mAb and a PE-conjugated anti-V022 mAb.

Protein Modeling

The HLA-DR1 and human CD4 complex model structures were predicted based on structures from PDB IDs 3S5L and 3TOE using Swiss-Model workspace for quaternary structure prediction.

Statistical Analysis

Statistical analysis was performed using GraphPad Prism 6.0 software (GraphPad Software, San Diego, Calif.). Unpaired two-tailed Student's t-tests were used for two-sample comparisons. No statistical method was used to predetermine sample size. The investigators were not blinded to allocation during the experiments or outcome assessment. The experiments were not randomized.

Biolayer Interferometry and Steady-State Analysis

The extracellular domain of human CD4 (residues 26-440 of NP_000607.1) followed by a GS linker and 10× histidine (His) tag was stably expressed in the human cell line A375 (SEQ ID NOs: 262-263; Table 7). Recombinant 10× His-tagged CD4 protein was purified from the supernatant with TALON metal affinity resin (Takara Bio, Shiga, Japan). The eluted protein was concentrated using an Amicon Ultra-15 spin column (MilliporeSigma, Burlington, Mass.) with a 10 kDa MWCO. Buffer was exchanged to HBS-EP (GE Healthcare Life Sciences, Marlborough, Mass.) using 10 kDa MWCO MINI Dialyzer (Thermo Fisher Scientific, Waltham, Mass.). The purity of the recombinant CD4 protein was consistently >90%, as confirmed by SDS-PAGE.

The recombinant DR1 protein consisted of extracellular domains of DRA1*01:01, and the wild-type DRB1*01:01 or L114W/V143M+2reps mutant. DRA1*01:01 was followed by an acid leucine zipper, a GS linker and a 10×histidine tag, while wild-type and mutant DRB1 was followed by a basic leucine zipper, a GS linker, and a biotinylation sequence (GLNDIFEAQKIEWHE; SEQ ID NO: 264). Both DRA and DRB genes were stably expressed in A375-BirA cells, which were transduced with the codon-optimized BirA gene encoding a leader sequence at the 5′ end and an ER retention KDEL motif at the 3′ end. Recombinant DR1 protein was purified from the supernatant with TALON metal affinity resin (Takara Bio, Shiga, Japan). Eluted protein was concentrated using Vivaspin 500 spin column (GE Healthcare Life Sciences, Marlborough, Mass.) with a 10 kDa MWCO, and reconstituted to working volume in PBS.

Binding for wild-type DR1 and DR1^{L114W/V143M+2reps} with CD4 was measured by the Octet Red system (ForteBio, Fremont, Calif.). Experiments were performed at 25° C. using a 96-well OptiPlate (Perkin Elmer, Waltham, Mass.), with a 200-μl sample volume and constant shaking at 1,000 rpm. The biotinylated recombinant DR1 was loaded onto streptavidin-coated biosensors (ForteBio, Fremont, Calif.) until saturation, followed by baseline measurement in the HBS-EP buffer. Association was measured by incubating the loaded sensors for 400 sec with titrated concentrations of recombinant CD4 (0.8125 to 26 μM) before 300 sec dissociation in HBS-EP buffer alone. The steady-state analysis was fitted using a one-site specific binding model in GraphPad Prism 7.0.

### Example 9—DR Molecules with Enhanced CD4 Binding Capacities

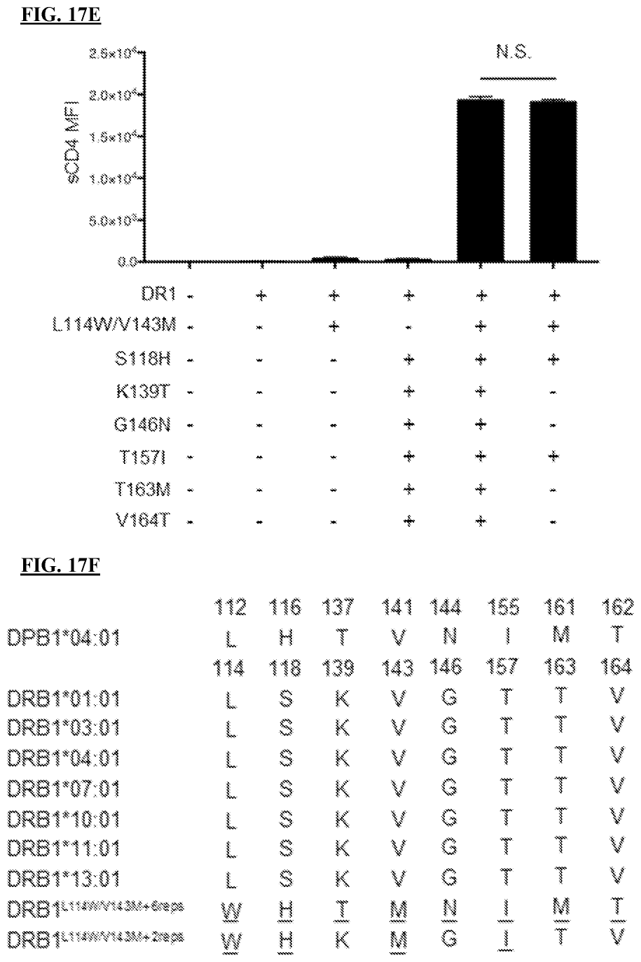

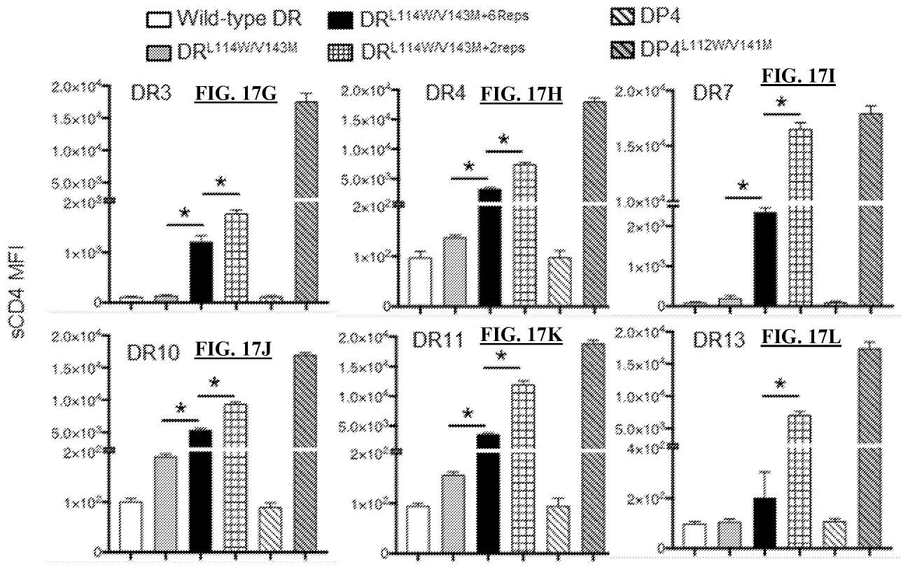

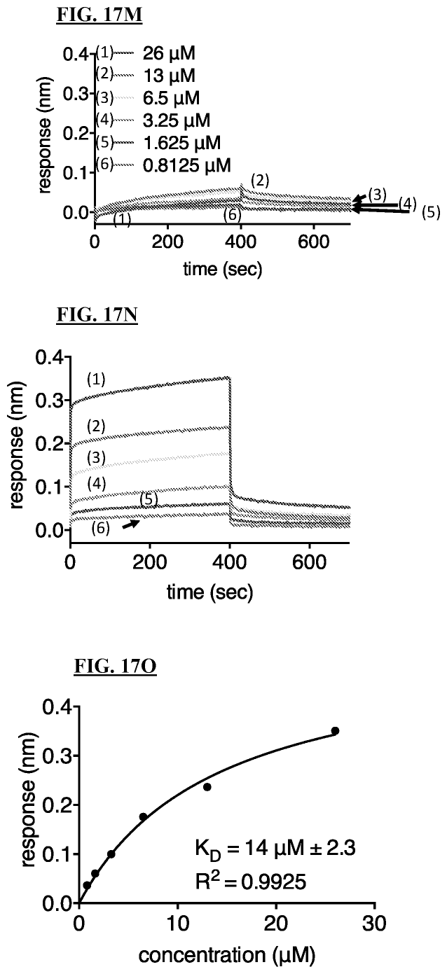

Affinity enhanced DR molecules were generated by introducing L114W/V143M mutations, to determine if these substitutions could improve the binding of HLA-DR molecules such as DR1 allele (DRA1*01:01-DRB1*01:01) to CD4. DRB1*01:01 encodes six different amino acids at positions 118, 139, 146, 157, 163 and 164 in addition to 114 and 143 (FIG. 17A). DR1^{L114W/V143M+6reps}, showed enhanced CD4 binding compared with DR1^{L114W/V143M} and wild-type DR1 (FIGS. 17B and 17C). A library of DR1^{L114W/V143M+6reps}-derived mutants with a single amino acid reversal at either S118H or T157I but not at the 4 other positions showed decreased CD4 binding capability, suggesting that both the S118H and T157I mutations are critical (FIG. 17D).

Indeed, the CD4 binding capacity of DR1^{L114W/V143M+2reps}, which possesses the L114W/V143M+S118H/T157I replacements (2reps) in the β chain, was comparable to that of DR1^{L114W/V143M+6reps} (FIG. 17E). These results suggest that the two additional replacements at S118H and T157I are pivotal for the function of the L114W/V143M mutations in the improvement of the binding of DR1 to CD4.

DRβ chains such as DRB1*03:01, 04:01, 07:01, 10:01, 11:01, and 13:01 encode different amino acids at positions 118, 139, 146, 157, 163, and 164 in addition to 114 and 143 (FIG. 17F). Interestingly, comparison of CD4 binding activity between the DR1^{L114W/V143M+2reps} and DR1^{L114W/V143M+6reps} mutants showed that, unlike for DR1, the L114W/V143M+2reps mutations enabled improved CD4 binding compared to the L114W/V143M+6reps mutations for DR3, DR4, DR7, DR10, DR11, and DR13 (FIGS. 17G-17L).

Using a biolayer interferometry (BLI) binding assay, the affinities of wildtype DR1 and DR1^{L114W/V143M+2reps} for CD4 were measured. While no binding was detected between wildtype DR1 and CD4 (FIG. 17M), DR1^{L114W/V143M+2reps} bound to CD4 with a KD of 14 μM+2.3 (FIGS. 17N-17O).

### Example 10—Affinity-Matured DR Dimers Specifically and Robustly Stained Cognate TCRs

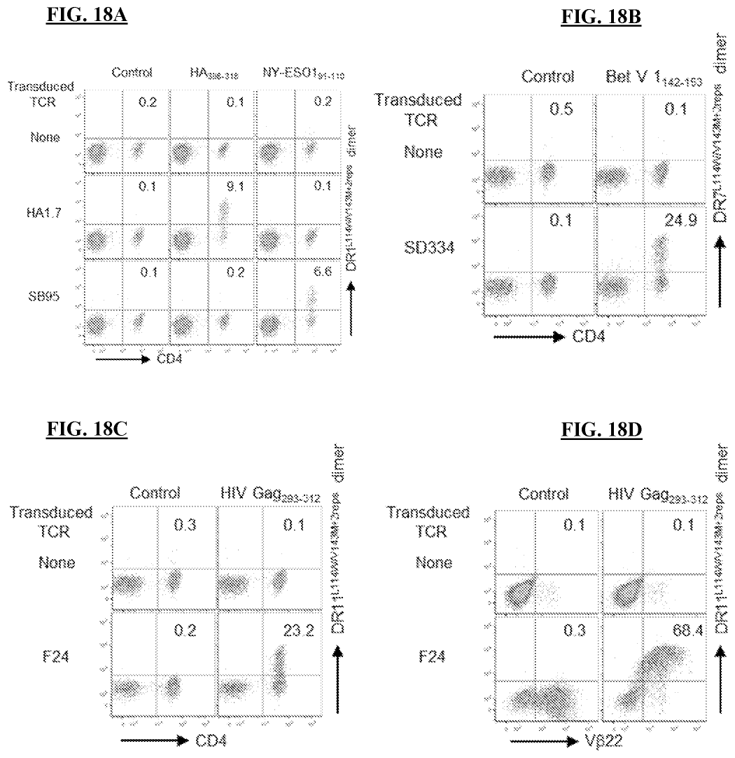

The ability of the affinity-matured DR dimers carrying the mutations described in example 2 were evaluated for the ability to identify antigen-specific CD4⁺ T cells. The DR1^{L114W/V143M+2reps}, DR7^{L114W/V143M+2reps}, and DR11^{L114W/V143M+2reps} dimers specifically stained the DR1-restricted TCRs HA1.7 and SB95, DR7-restricted TCR SD334, and DR11-restricted TCR F24, respectively (FIGS. 18A-18C). Costaining of F24-transduced CD4⁺ T cells with an anti-Vβ22 mAb, along with the respective DR11^{L114W/V143M+2reps} dimers, confirmed that virtually all the TCR-transduced CD4⁺ T cells were successfully stained with the respective DR11^{L114W/V143M+2reps} dimers (FIG. 18D).

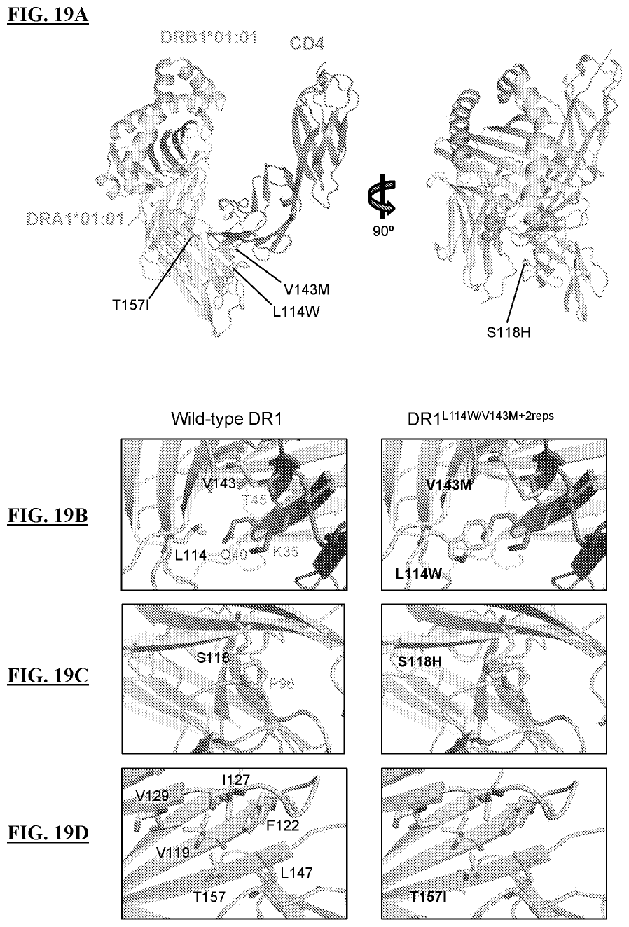

A structural model of the complex consisting of CD4 and DR1^{L114W/V143M+2reps} also showed a potential hydrophobic effect of the L114W/V143M replacements (FIGS. 19A-19B). Furthermore, hydrophobic stacking was observed between P96 of the α-chain and S118H of the 3-chain (FIG. 19C), and the T157I replacement was found to localize in the β-sheet surrounding Vi 19, F112, I127, V129, L147 and T157 (FIG. 19D). It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

To identify affinity-matured class II molecules, the present examples detail multiple mutations in the β-chain but not the α-chain because the β-chain has a more direct interaction with CD4 than the α chain. It is possible that additional mutations of the α- and/or β-chains can further enhance the binding between class II and CD4. However, the use of such soluble class II molecules with excessive CD4 binding capabilities may cause nonspecific staining of CD4⁺ T cells, thereby having a detrimental effect.

In conclusion, CD4⁺ T cells play a critical role in the development of autoimmune diseases and protection against pathogenic infections and cancers. The novel HLA class II multimer technology described herein may better facilitate the study of HLA class II-restricted CD4⁺ T cell responses across HLA-DR alleles.

### Example 11

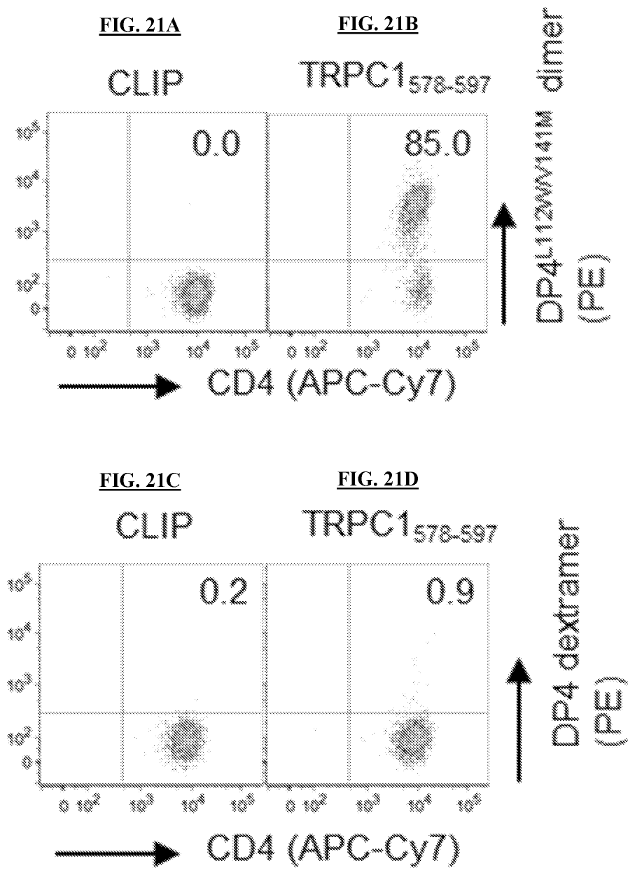

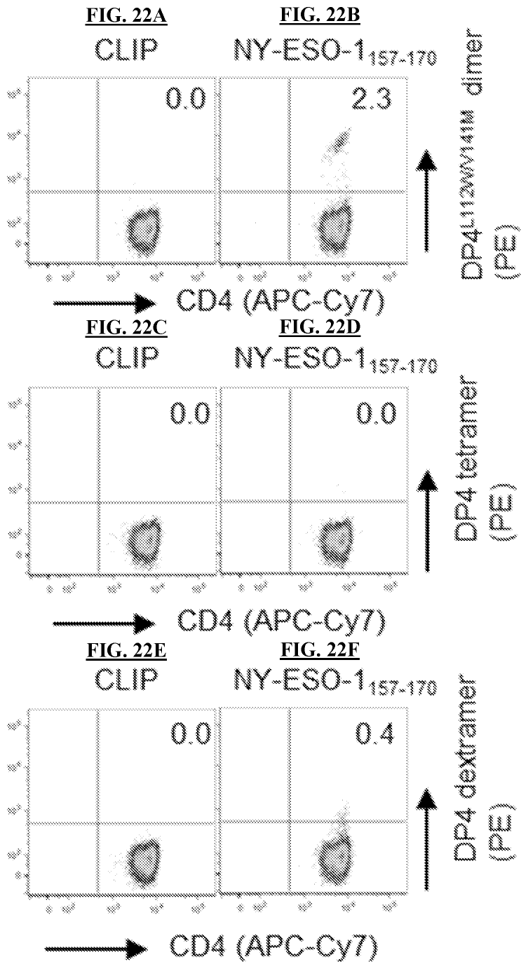

DP4 multimer staining of endogenous (untransduced) antigen specific CD4⁺ T cells was analyzed. The novel DP4^{L112W/V141M} dimers positively stained endogenous TRPC1₅₇₈₋₅₉₇-specific CD4⁺ T cells (FIGS. 21A-21B) more strongly than the conventional DP4 dextramer (FIGS. 21C-21D). The DP4^{L112W/V141M} dimers showed markedly improved staining of endogenous (untransduced) NY-ESO-1₁₅₇₋₁₇₀ specific CD4⁺ T cells (FIGS. 22A-22B; Table 9) compared with conventional tetramers (FIGS. 22C-22D) or dextramers (FIGS. 22E-22F).

TABLE 9
DP4-Restricted TCRs

| Donor | | | | | | |
|---|---|---|---|---|---|---|
| No. | TRAV | TRAJ | TCR-alpha CDR3 | TRBV | TRBJ | TCR-beta CDR3 |
| HD04 | 8-2*01 | 32*02 | CVVSGGVNGGATNKLIF | 7-9*01 | 2-7*01 | CASSLTGGVSYEQYF |
| | | | SEQ ID NO: 278 | | | SEQ ID NO: 279 |
| c6 | 13-1*01 | 18*01 | CAASVRGSTLGRLYF | 7-8*01 | 2-2*01 | CASSLGTGGTGELFF |
| | | | SEQ ID NO: 280 | | | SEQ ID NO: 281 |
| c12 | 8-4*01 | 18*01 | CAVSGGRGSTLGRLYF | 29*01 | 1-2*01 | CSVQGGLDSNYGYTF |
| | | | SEQ ID NO: 282 | | | SEQ ID NO: 283 |
| c17 | 13-1*01 | 18*01 | CAASVRGSTLGRLYF | 7-8*01 | 2-2*01 | CASSLGTGGTGELFF |
| | | | SEQ ID NO: 284 | | | SEQ ID NO: 285 |
| c23 | 13-1*01 | 18*01 | CAASVRGSTLGRLYF | 7-8*01 | 2-2*01 | CASSLGTGGTGELFF |
| | | | SEQ ID NO: 286 | | | SEQ ID NO: 287 |
| c26 | 38-2/ | 21*01 | CAYRSNNFNKFYF | 5-1*01 | 1-2*01 | CASSLNTGAGYGYTF |
| | DV8*01 | | SEQ ID NO: 288 | | | SEQ ID NO: 289 |
| c31 | 13-1*01 | 18*01 | CAASVRGSTLGRLYF | 7-8*01 | 2-2*01 | CASSLGTGGTGELFF |
| | | | SEQ ID NO: 290 | | | SEQ ID NO: 291 |
| c37 | 13-1*01 | 18*01 | CAASVRGSTLGRLYF | 7-8*01 | 2-2*01 | CASSLGTGGTGELFF |
| | | | SEQ ID NO: 292 | | | SEQ ID NO: 293 |
| c39 | 2*01 | 9*01 | CAVEERTGGFKTIF | 2*01 | 2-2*01 | CASSLPSGGAPGTGELFF |
| | | | SEQ ID NO: 294 | | | SEQ ID NO: 295 |
| c52 | 8-4*01 | 18*01 | CAVSGGRGSTLGRLYF | 29*01 | 1-2*01 | CSVQGGLDSNYGYTF |
| | | | SEQ ID NO: 296 | | | SEQ ID NO: 297 |
| c87 | 13-1*01 | 18*01 | CAASVRGSTLGRLYF | 7-8*01 | 2-2*01 | CASSLGTGGTGELFF |
| | | | SEQ ID NO: 298 | | | SEQ ID NO: 299 |
| c2 | 4*01 | 39*01 | CLVGDLGANAGNMLTF | 19*01 | 2-2*01 | CASSIATTNTGELFF |
| | | | SEQ ID NO: 300 | | | SEQ ID NO: 301 |
| c4 | 4*01 | 39*01 | CLVGDLGANAGNMLTF | 11-3*01 | 2-7*01 | CASSLETGTNYEQYF |
| | | | SEQ ID NO: 302 | | | SEQ ID NO: 303 |
| c6 | 8-3*01 | 32*02 | CAVALYGGATNKLIF | 7-9*3 | 2-1*01 | CASSLDIGNNEQFF |
| | | | SEQ ID NO: 304 | | | SEQ ID NO: 305 |
| c9 | 25*01 | 53*01 | CAGRSGGSNYKLTF | 19*01 | 2-2*01 | CASSIATTNTGELFF |
| | | | SEQ ID NO: 306 | | | SEQ ID NO: 307 |
| c29 | 25*01 | 53*01 | CAGRSGGSNYKLTF | 19*01 | 2-2*01 | CASSIATTNTGELFF |
| | | | SEQ ID NO: 308 | | | SEQ ID NO: 309 |
| c30 | 25*01 | 53*01 | CAGRSGGSNYKLTF | 19*01 | 2-2*01 | CASSIATTNTGELFF |
| | | | SEQ ID NO: 310 | | | SEQ ID NO: 311 |
| c32 | 25*01 | 53*01 | CAGRSGGSNYKLTF | 19*01 | 2-2*01 | CASSIATTNTGELFF |
| | | | SEQ ID NO: 312 | | | SEQ ID NO: 313 |
| c33 | 25*01 | 53*01 | CAGRSGGSNYKLTF | 19*01 | 2-2*01 | CASSIATTNTGELFF |
| | | | SEQ ID NO: 314 | | | SEQ ID NO: 315 |

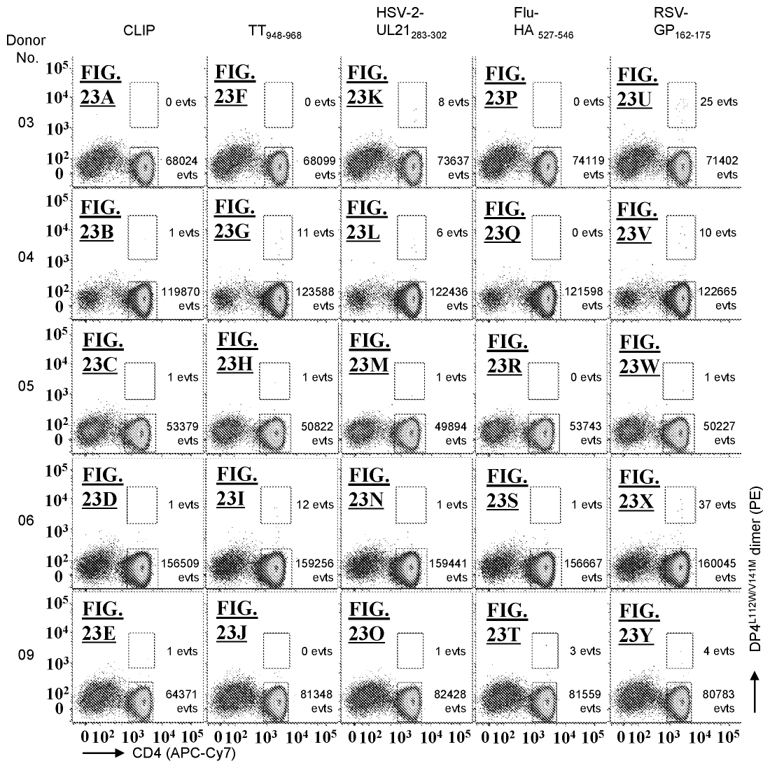

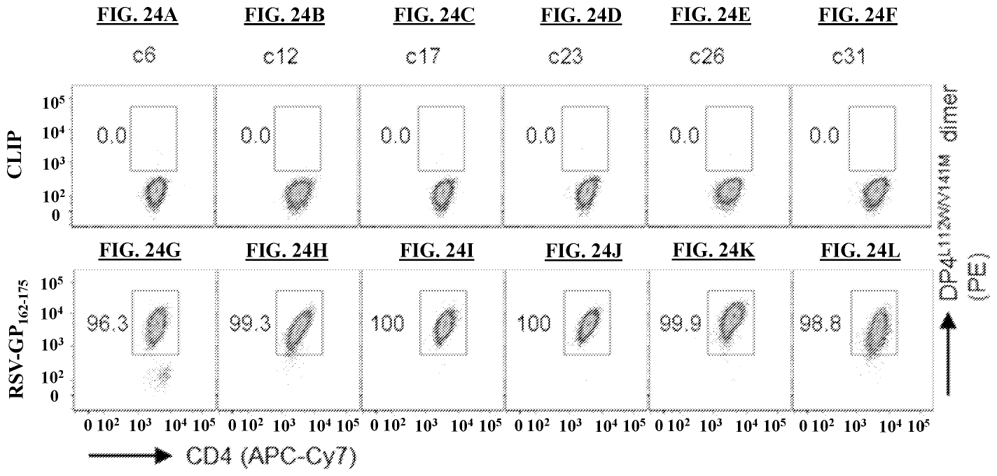

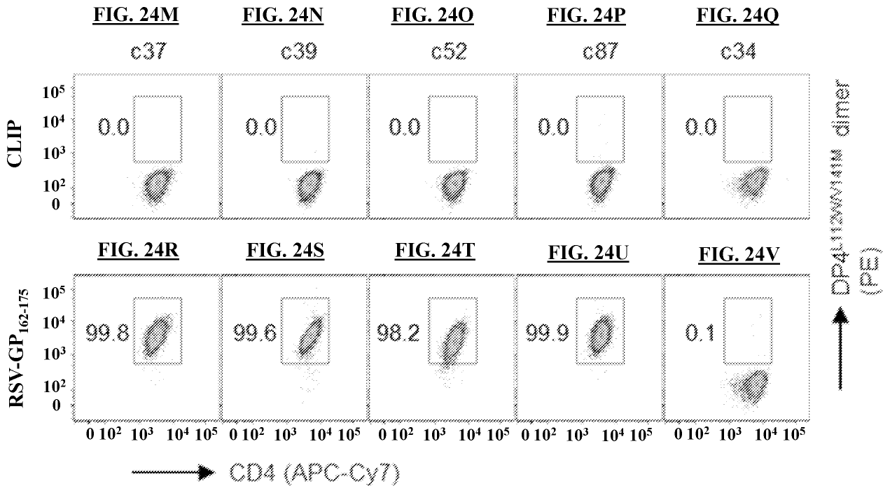

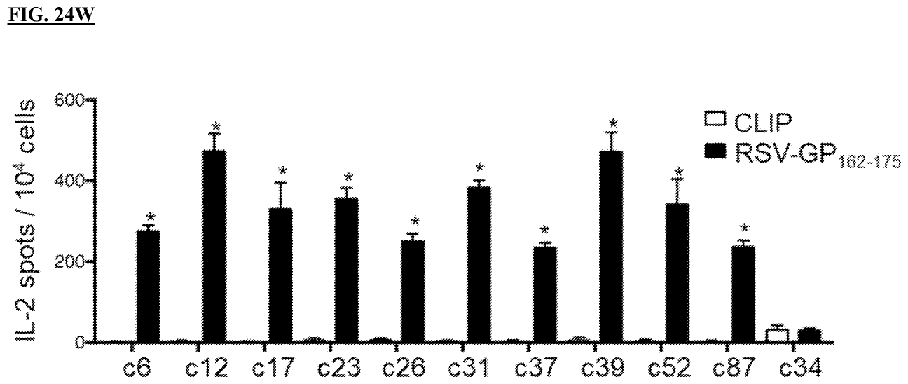

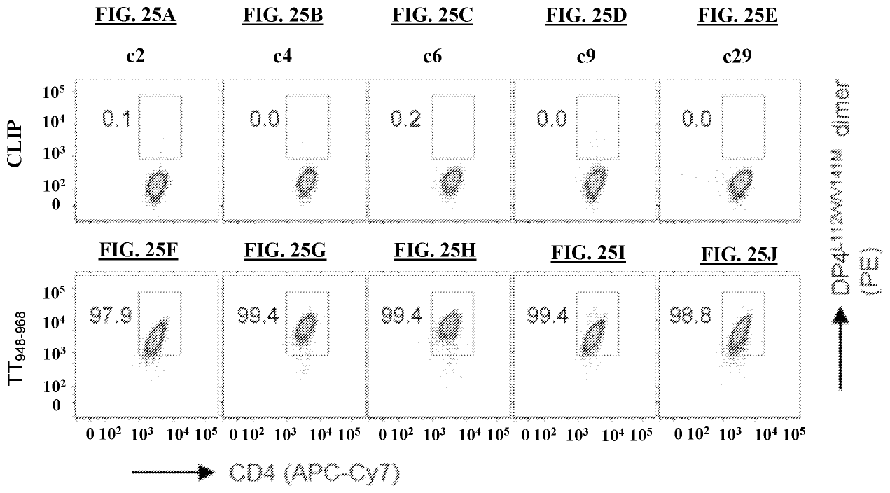

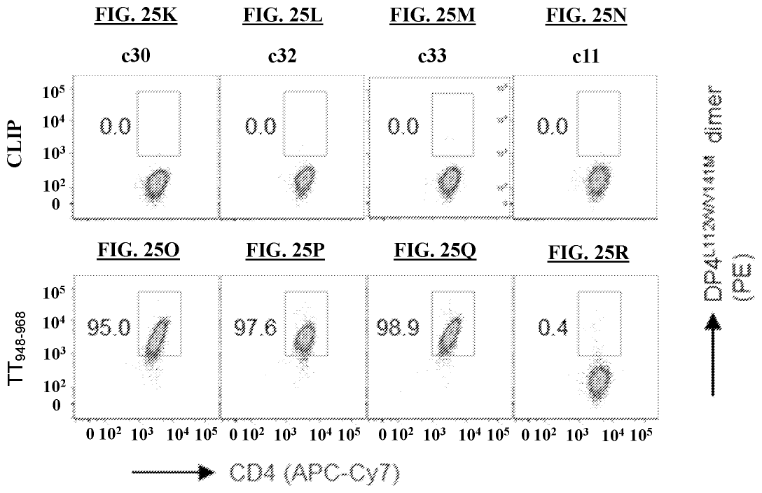

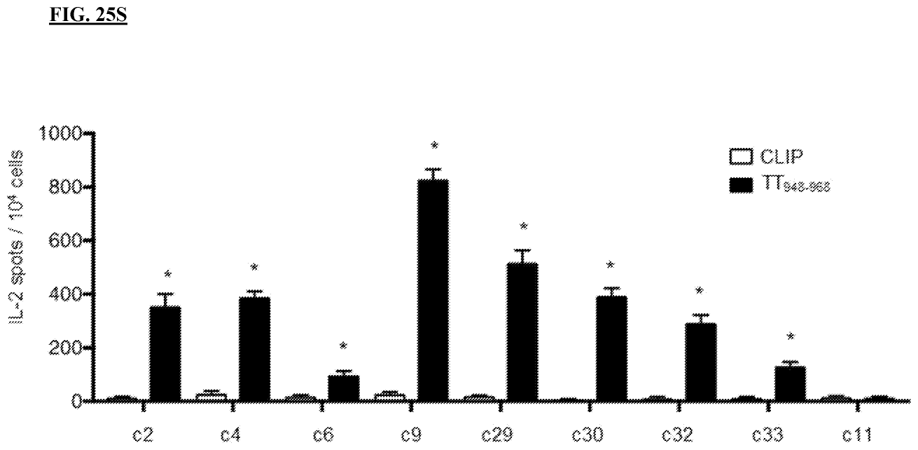

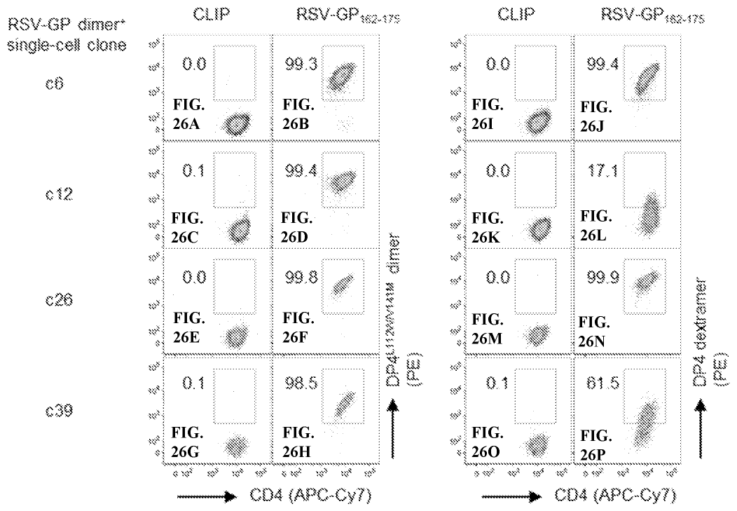

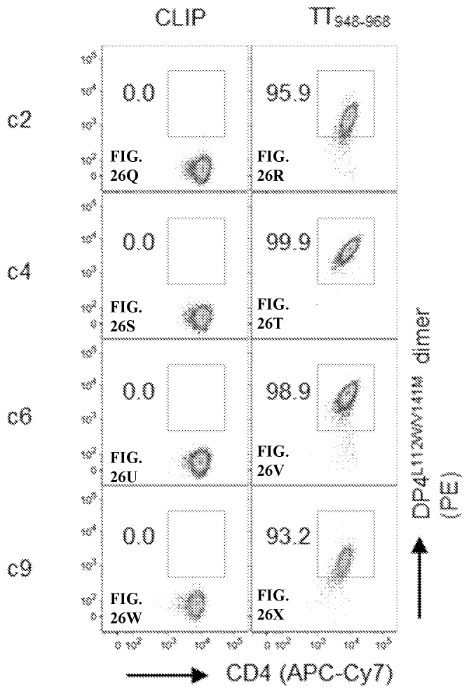

Next, ex vivo staining was performed of memory CD4⁺ T cells with DP4^{L112W/V141M} dimers specific to a series of pathogen-associated peptides without in vitro stimulation. A small subset of the CD4⁺ T cells were positively stained with DP4^{L112W/V141M} dimers for tetanus toxin₉₄₈₋₉₆₈ (TT₉₄₈₋₉₆₈), herpes simplex virus type-2-UL21₂₈₃₋₃₀₂ (HSV-2-UL21₂₈₃₋₃₀₂), and respiratory syncytial virus glycoprotein₁₆₂₋₁₇₅ (RSV-GP₁₆₂₋₁₇₅) (FIGS. 23A-23Y). Next, we established endogenous (untransduced) single-cell clones by limiting dilution from RSV-GP₁₆₂₋₁₇₅ (FIGS. 24A-24V) and TT₉₄₈₋₉₆₈ dimer⁺ CD4⁺ T cells (FIGS. 25A-25R). These T cell clones showed IL-2 production in an antigen-specific manner (FIGS. 24W and 24S). Multiple TCRαβ pairs, including one dominant pair, were isolated from both DP4^{L112W/V141M} RSV-GP and TT dimer⁺ single-cell clones (Table 9). In FIGS. 24A-24W and 25A-25S, single-cell clones were established by limiting dilution from RSV-GP₁₆₂₋₁₇₅ and TT₉₄₈₋₉₆₈ dimer⁺ cells. When these RSV-GP and TT dimer⁺ single-cell clones were individually stained with three different DP4 multimers (DP4^{L112W/V141M} dimers, wild-type DP4 tetramers, or wild-type DP4 dextramers), the DP4^{L112W/V141M} dimers showed better staining of RSV-GP- (c12 and c39) and TT-specific clones (c2 and c9) than the conventional wild-type DP4 RSV-GP dextramers and wild-type DP4 TT tetramers and dextramers (FIGS. 26A-26NN).

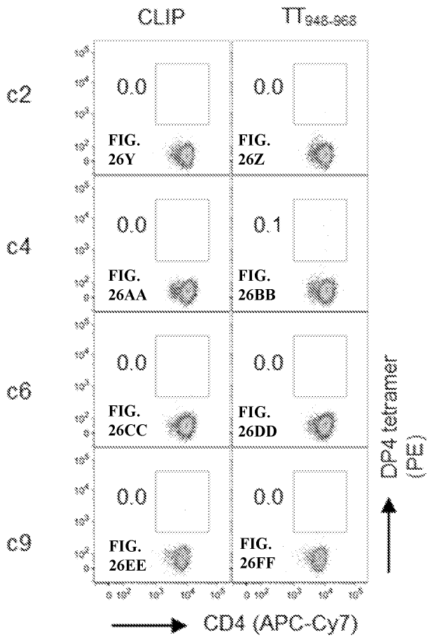

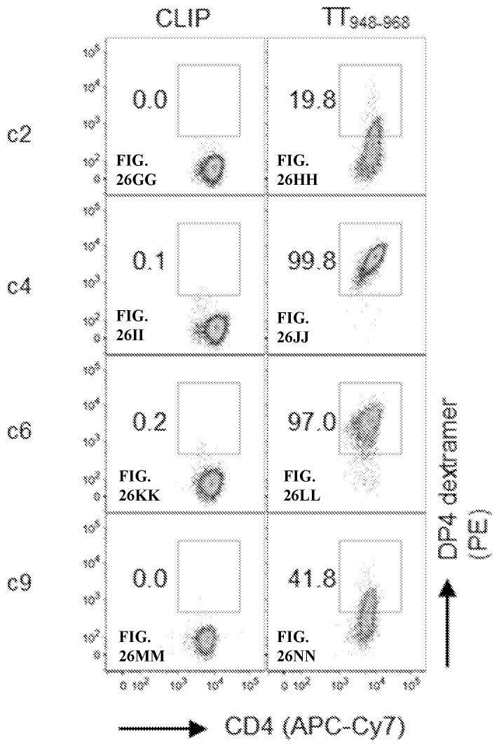

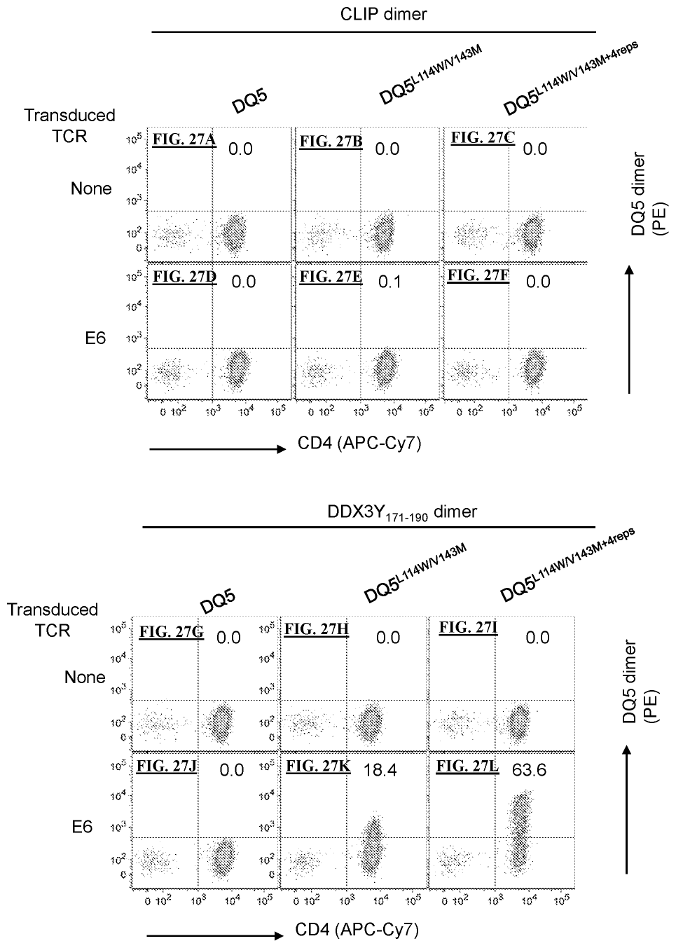

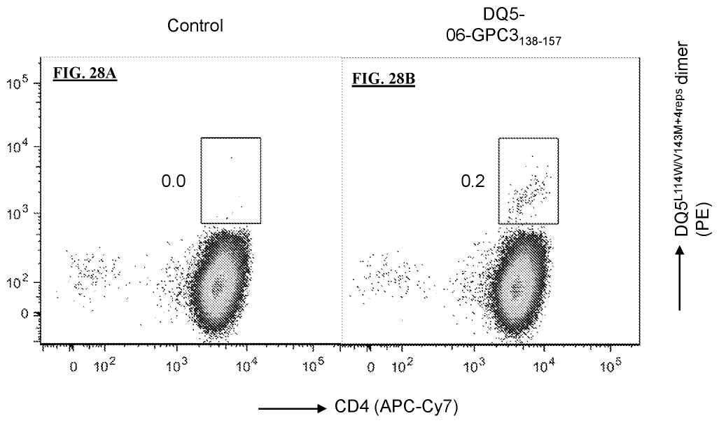

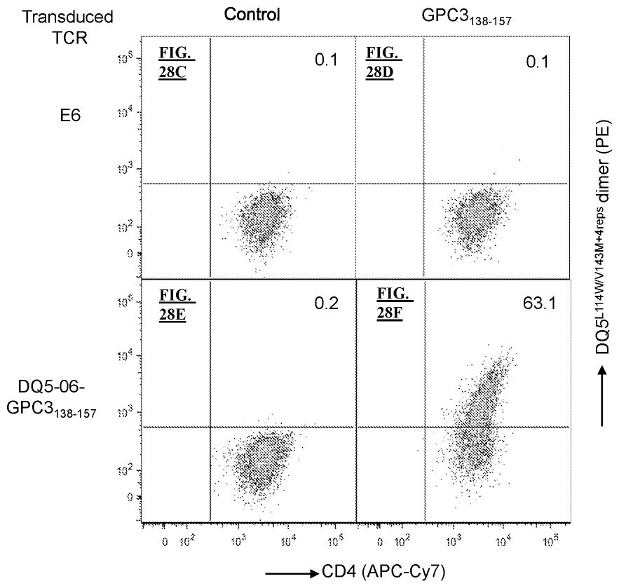

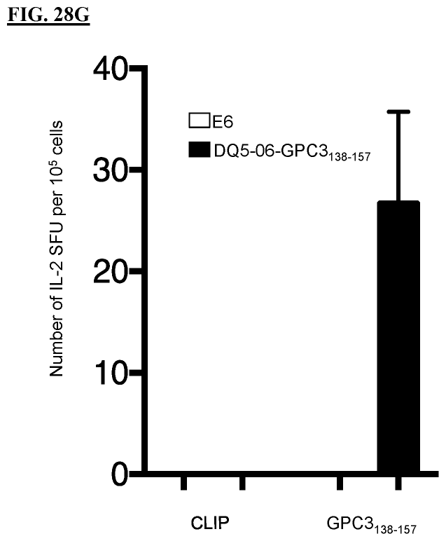

Wild-type DQ5 and DQ5^{L114W/V143M} dimers (Table 10) and DQ5^{L114W/V143M+4reps} dimers were produced and their staining of TCR-transduced CD4⁺ T cells was compared. The wild-type DQ5 dimers could not detect E6-transduced CD4⁺ T cells. The DQ5^{L114W/V143M} dimers showed only weak staining of the E6-transduced CD4⁺ T cells compared to the DQ5^{L114W/V143M+4reps} dimers, which instead showed robust staining (FIGS. 27A-27L). To validate DQ5^{L114W/V143M+4reps} dimer staining, we cloned a DQ5-restricted TCR gene specific to GPC3₁₃₈₋₁₅₇ from dimer⁺ CD4⁺ T cells in vitro expanded in a peptide-specific manner. When clonotypically reconstituted in human CD4⁺ TCR-deficient T cells, the TCR was successfully stained by the cognate DQ5^{L114W/V143M+4reps} dimer and were functional in a DQ5-restricted and antigen-specific manner (FIGS. 28A-28G).

TABLE 10
TCR Sequences

| No. | Peptide | TRAV | TRAJ | TCRα CDR3 | TRBV | TRBJ | TCRβ CDR3 |
|---|---|---|---|---|---|---|---|
| 06 | GPC3₁₃₈₋₁₅₇ | 9-2*02 | 27*01 | CALYTNAGKSTF | 15*02 | 2-3*01 | CATSRDVSSTDTQYF |
| | | | | (SEQ ID NO: 316) | | | (SEQ ID NO: 317) |

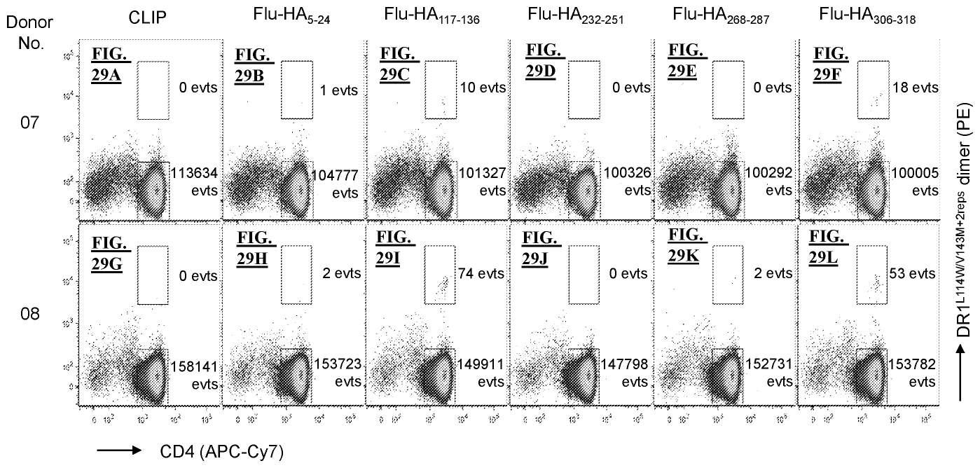

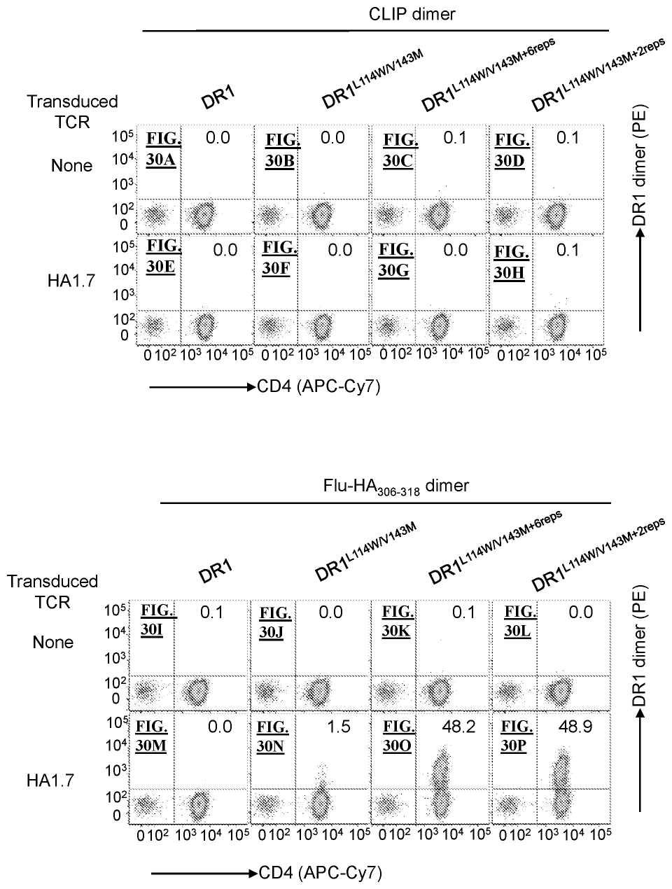

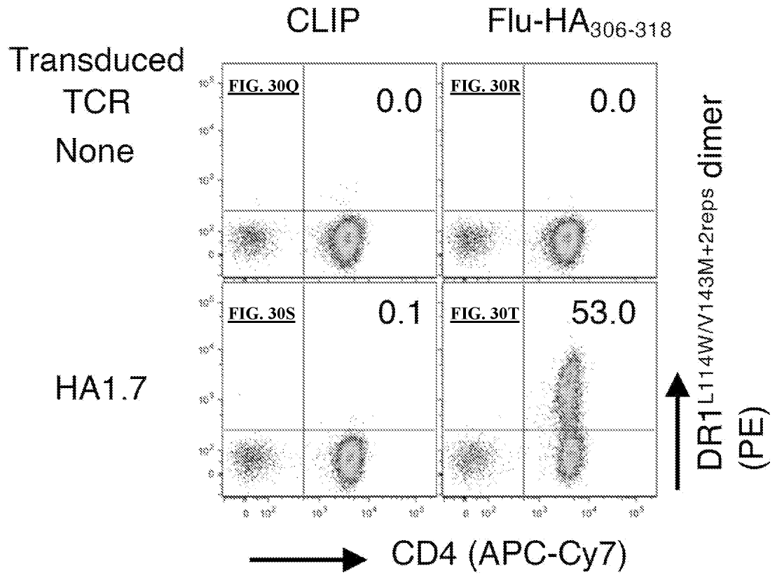

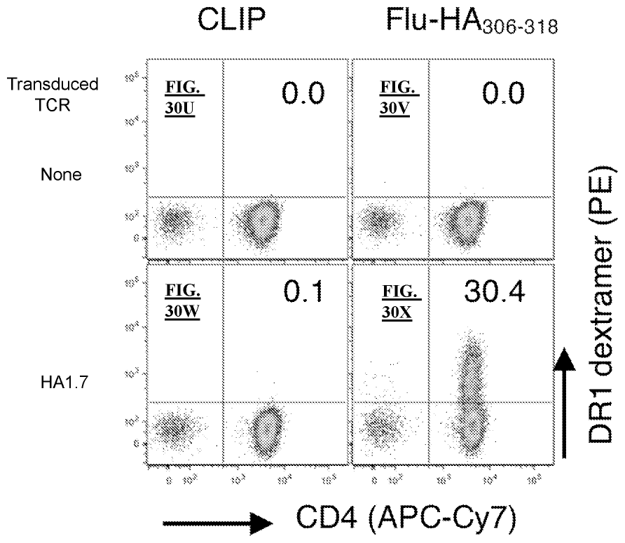

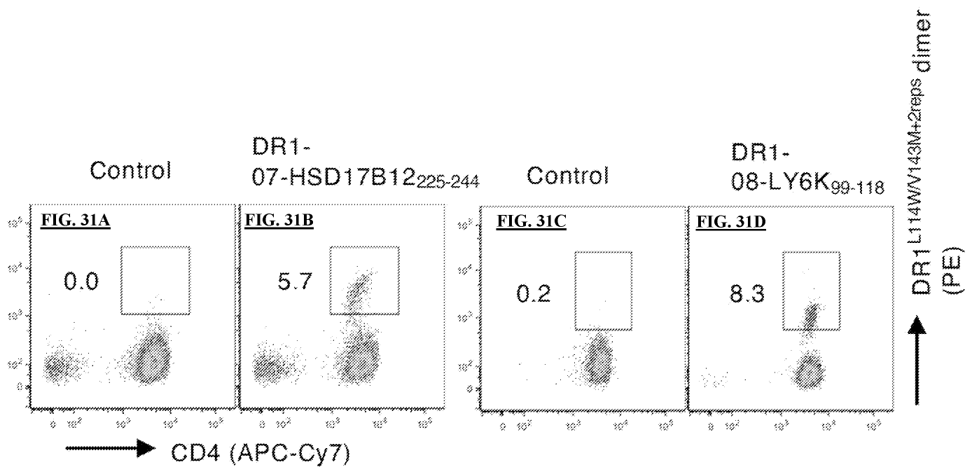

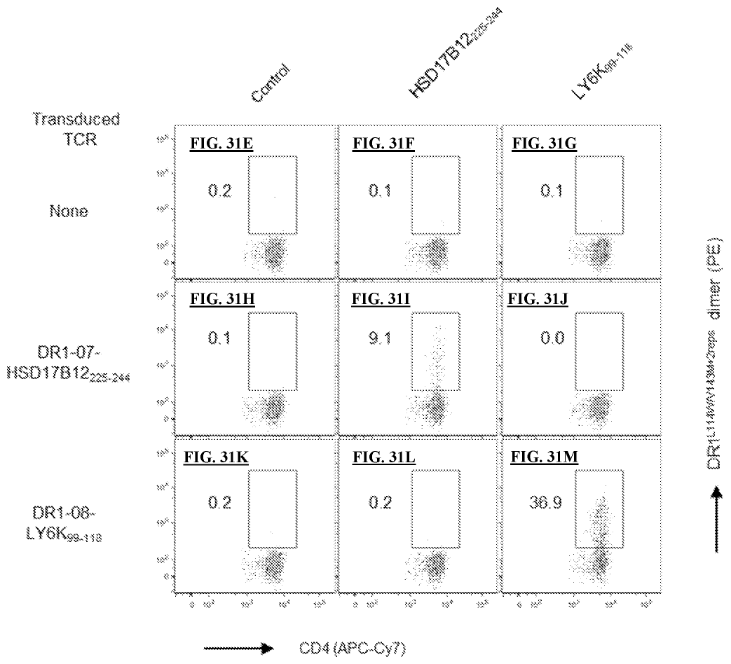

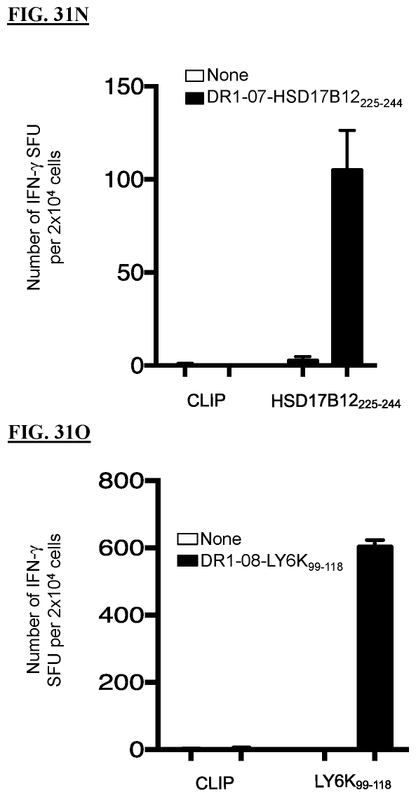

Ex vivo staining was performed of memory CD4⁺ T cells with DR1^{L114W/V143M+2reps} dimers specific to influenza virus hemagglutinin (Flu-HA) peptides without in vitro stimulation. A small subset of the CD4⁺ T cells were positively stained with DR1^{L114W/V143M+2reps} dimers for Flu-HA₁₁₇₋₁₃₆- and Flu-HA₃₀₆₋₃₁₈ (FIGS. 29A-29L). Wild-type DR1, DR1^{L114W/V143M} and DR1^{L114W/V143M+6reps} dimers and DR1^{L114W/V143M+2reps} dimers were produced and their staining of TCR-transduced CD4⁺ T cells was compared. Both wild-type DR1 and DR1^{L114W/V143M} dimers detected very little of the cognate TCR (HA1.7) on CD4⁺ T cells, while DR1^{L114W/V143M+2reps} and DR1^{L114W/V143M+6reps} dimers showed similar robust staining. Importantly, DR1^{L114W/V143M+2reps} dimers stained HA1.7-transduced CD4⁺ T cells more robustly and with better separation than the wild-type DR1 dextramer (FIGS. 30A-30X). To validate DR1^{L114W/V143M+2reps} dimer staining, DR1-restricted TCR genes specific to HSD17B12₂₂₅₋₂₄₄ and LY6K₉₉₋₁₁₈ were cloned from dimer⁺ CD4⁺ T cells in vitro expanded in a peptide-specific manner. When clonotypically reconstituted in primary CD4⁺ T cells, the two TCRs (Table 11) were successfully stained by the cognate DR1^{L114W/V143M+2reps} dimers and were functional in a DR1-restricted and antigen-specific manner (FIGS. 31A-310).

TABLE 11
TCR Sequences

| No. | Peptide | TRAV | TRAJ | TCRα CDR3 | TRBV | TRBJ | TCRβ CDR3 |
|---|---|---|---|---|---|---|---|
| 07 | HSD17B12₂₂₅₋₂₄₄ | 5*01 | 4*01 | CADLSGGYNKLIF | 11*01 | 2-3*01 | CASSPTLGTDTQYF |
| | | | | (SEQ ID | | | (SEQ ID |
| | | | | NO: 318) | | | NO: 319) |
| 08 | LY6K₉₉₋₁₁₈ | 38- | 52*01 | CAYRSFLNAG | 20- | 1-6*01 | CAASRESKWS |
| | | 2/DV8*01 | | GTSYGKLTF | 1*01 | | SYNSPLHF |
| | | | | (SEQ ID | | | (SEQ ID |
| | | | | NO: 320) | | | NO: 321) |

Methods

Cells

Peripheral mononuclear cells were obtained via density gradient centrifugation. K562-based artificial antigen presenting cells (aAPCs) individually expressing various HLA class II genes as a single HLA allele in conjunction with CD80 and CD83 have been reported previously (see Butler, M. O. et al., PLoS One 7, e30229 (2012)). The Jurkat 76 cell line is a T cell leukemic cell line lacking endogenous TCR, CD4, and CD8 expression (see. Heemskerk, M. H. et al., Blood 102, 3530-3540 (2003)). Jurkat 76/CD4 cells were generated by retrovirally transducing the human CD4 gene. A375 cells are a melanoma cell line. HEK293T cells and A375 cells were grown in DMEM supplemented with 10% FBS and 50 μg/ml gentamicin. The Jurkat 76 cell line was cultured in RPMI 1640 supplemented with 10% FBS and 50 μg/ml gentamicin.

Peptides/Antibodies

Synthetic peptides were dissolved at 50 mg/ml in DMSO. The following antibodies were used for flow cytometry analysis: APC-Cy7-conjugated anti-CD4 (RPA-T4, BIOLEGEND, San Diego, Calif.; see Wooldridge, L. et al., Eur J Immunol 36, 1847-1855 (2006)) and PE-conjugated anti-His tag (AD1.1.10, ABCAM, Cambridge, Mass.). Dead cells were distinguished with the LIVE/DEAD Fixable Aqua Dead Cell Stain Kit. Stained cells were analyzed with FACSCanto II or LSRFortessa X-20. Cell sorting was conducted using a FACSAria II. Data analysis was performed using FlowJo software (version 9.9.6).

Genes

Novel TCR genes were cloned via 5′-rapid amplification of cDNA ends (RACE) PCR and sequenced as previously described (see, e.g., Nakatsugawa, M. et al., Sci Rep 6, 23821 (2016); Nakatsugawa, M. et al., J Immunol 194, 3487-3500 (2015); Ochi, T. et al., Cancer Immunol Res 3, 1070-1081 (2015); each of which is incorporated by reference herein in its entirety). All genes were cloned into the pMX retroviral vector and transduced into cell lines using the 293GPG and PG13 cell-based retrovirus system (see, e.g., Hirano, N. et al., Blood 107, 1528-1536 (2006); Butler, M. O. et al., Clin Cancer Res 13, 1857-1867 (2007); Hirano, N. et al., Clin Cancer Res 12, 2967-2975 (2006); each of which is incorporated by reference herein in its entirety).

Generation of the HLA Class II Monomer and Dimer

HEK293T cells were transfected with the α and β genes using the 293GPG cell-based retrovirus system (see Hirano, N. et al., Blood 107, 1528-1536 (2006); Butler, M. O. et al., Clin Cancer Res 13, 1857-1867 (2007); Hirano, N. et al., Blood 108, 2662-2668 (2006)) and cultured in DMEM supplemented with 10% FBS and 50 μg/ml gentamicin. For DP4 dimer staining, HEK293T cells stably secreting soluble DP4^{L112W/V141M} protein were grown until confluent, and the medium was changed to serum-free 293 SFM II medium (Thermo Fisher Scientific, Waltham, Mass.).

A375 cells were transfected with the α and β genes using the 293GPG cell-based retrovirus system (see, e.g., Hirano, N. et al., Blood 107, 1528-1536 (2006); Butler, M. O. et al. Clin Cancer Res 13, 1857-1867 (2007); and Hirano, N. et al., Blood 108, 2662-2668 (2006); each of which is incorporated by reference herein in its entirety) and cultured in DMEM supplemented with 10% FBS and 50 μg/ml gentamicin.

After forty-eight hours, the conditioned medium was harvested and concentrated using Amicon Ultra filters (molecular weight cut-off (MWCO) 10 kDa) (MilliporeSigma, Burlington, Mass.). The soluble HLA class II-containing supernatant was then mixed with 100 μg/ml peptide of interest for 20-24 hours at 37° C. for in vitro peptide exchange. The concentration of the monomer was measured by specific ELISA using a nickel-coated plate and an anti-His tag biotinylated mAb. Soluble HLA class II monomer was dimerized using a PE-conjugated anti-His mAb at a 2:1 molar ratio for 1.5 hours at 4° C. for staining.

Stimulation of DP4-Restricted Antigen-Specific CD4⁺ T Cells

CD4⁺ T cells were purified and stimulated with DP4-expressing aAPCs pulsed with DP4-restricted peptides at 10 μg/ml and irradiated at 200 Gy at an E:T ratio of 20:1. After forty-eight hours, 10 IU/ml IL-2 and 10 ng/ml IL-15 were added to the CD4⁺ T cells. The culture medium supplemented with IL-2 (10 IU/ml) and IL-15 (10 ng/ml) was replenished every 2-3 days. After 2 weeks of stimulation, the T cells were subjected to DP4^{L112W/V141M} dimer staining.

Stimulation of DQ5-Restricted Antigen-Specific CD4⁺ T Cells

CD4⁺ T cells were purified and then stimulated with DQ5-expressing aAPCs pulsed with GPC3₁₃₈₋₁₅₇ at 10 μg/ml and irradiated at 200 Gy at an E:T ratio of 20:1. After forty-eight hours, 10 IU/ml IL-2 and 10 ng/ml IL-15 were added to the CD4⁺ T cells. The culture medium supplemented with IL-2 (10 IU/ml) and IL-15 (10 ng/ml) was replenished every 2-3 days. Two weeks later, the T cells were subjected to DQ5^{L114W/V143M+4reps} dimer staining.

Stimulation of DR1-Restricted Antigen-Specific CD4⁺ T Cells

CD4⁺ T cells were purified and then stimulated with DR1-expressing aAPCs pulsed with DR1-restricted peptides at 10 μg/ml and irradiated at 200 Gy at an E:T ratio of 20:1. After forty-eight hours, 10 IU/ml IL-2 and 10 ng/ml IL-15 were added to the CD4⁺ T cells. The culture medium supplemented with IL-2 (10 IU/ml) and IL-15 (10 ng/ml) was replenished every 2-3 days. Two weeks later, the T cells were subjected to DR1^{L114W/V143M+2reps} dimer staining.

HLA Class H Dimer, Tetramer, and Dextramer Staining

DP4 tetramers and dextramers, and DR1 dextramers were compared in multimer staining analysis.

Primary CD4⁺ T cells and Jurkat 76/CD4 T cells transduced with antigen-specific TCR genes were pretreated with 50 nM dasatinib for 30 min at 37° C. and stained with 5-15 μg/ml class II dimers for 4-5 hours at room temperature. After washing, cell surface molecules were counterstained with an APC-Cy7-conjugated anti-CD4 mAb.

Dimer Staining of Unstimulated CD4⁺ T Cells from PBMCs from Melanoma Patients

One million CD4⁺ T cells were purified and pretreated with 50 nM dasatinib for 30 min at 37° C. The cells were stained with 5-15 μg/ml class II dimers for 4-5 hours at room temperature. After washing, cell surface molecules were counterstained with an APC-Cy7-conjugated anti-CD4 mAb. The absolute counts of the dimer⁺ cells were determined by flow cytometry.

Expansion of DP4 Dimer⁺ T Cells and Establishment of Single T Cell Clones.

To expand DP4^{L112W/V141M} dimer⁺ T cells, CD4⁺ T cells were stimulated and stained with DP4^{L112W/V141M} dimers as described above. The dimer⁺ cells were sorted by using anti-PE magnetic beads and expanded by using artificial APC/mOKT3 irradiated at 200 Gy at an E:T ratio of 5-20:1 (see Butler, M. O. et al., PLoS One 7, e30229 (2012)). The culture medium supplemented with IL-2 (10 IU/ml) and IL-15 (10 ng/ml) was replenished every 2-3 days. Two to three weeks later, the T cells were subjected to DP4^{L112W/V141M} dimer staining. DP4^{L112W/V141M} dimer⁺ single-cell clones were generated by limiting dilution as previously described (see Su, L. F. et al., Immunity 38, 373-383 (2013)). Briefly, memory CD4⁺ T cells were purified and stained with DP4^{L112W/V141M} dimers without dasatinib pretreatment. The dimer⁺ cells were sorted and then stimulated with 5 μg/ml PHA-P and PBMCs from multiple allogeneic donors irradiated at 20 Gy in a 96-well plate. The culture medium was supplemented and replenished after 1 week of stimulation with IL-2 (100 IU/ml) and IL-15 (10 ng/ml). Two weeks later, single-cell clones were stained with DP4^{L112W/V141M} dimers.

ELISPOT Assay

Cytokine ELISPOT assays were performed as previously reported (see, e.g., Yamashita, Y. et al., Nat Commun 8, 15244 (2017); and Anczurowski, M. et al., Sci Rep 8, 4804 (2018)); each of which is incorporated by reference herein in its entirety).

1. A method of identifying an MHC class II-specific T cell receptor (TCR) comprising contacting a T cell with a complex comprising an MHC class II molecule and a peptide;
wherein the T cell expresses CD4 and one or more TCRs;
wherein the MHC class II molecule comprises an alpha chain and a beta chain, wherein the MHC class II molecule has a higher affinity for CD4 than a naturally occurring MHC class II molecule has for the CD4; and
wherein the MHC class II-specific TCR specifically binds the complex comprising the MHC class II molecule and the peptide.

2. The method of claim 1, wherein the beta chain of the MHC class II molecule comprises an amino acid sequence having one or more mutations relative to a wild-type beta chain of a MHC class II molecule.

3. The method of claim 1 or 2, wherein the alpha chain of the MHC class II molecule comprises an amino acid sequence having one or more mutations relative to a wild-type alpha chain of a MHC class II molecule.

4. The method of claim 2 or 3, wherein the one or more mutations comprise a substitution mutation.

5. The method of any one of claims 1 to 4, wherein the MHC class II molecule is an HLA-DP, HLA-DQ, or HLA-DR allele, or any combination thereof.

6. The method of any one of claims 1 to 5, wherein (i) the beta chain of the HLA class II molecule is an HLA-DP allele, (ii) the alpha chain of the HLA class II molecule is an HLA-DP allele, or (iii) both (i) and (ii).

7. The method of any one of claims 1 to 6, wherein the beta chain of the HLA class II molecule is a DP1, DP2, DP3, DP4, DP5, DP6, DP8, or DP9 allele.

8. The method of any one of claims 1 to 7, wherein the beta chain of the MHC class II molecule comprises an HLA allele selected from the group consisting of DPB1*01, DPB1*02, DPB1*03, DPB1*04, DPB1*05, DPB1*06, DPB1*08, DPB1*09, DPB1*10, DPB1*100, DPB1*101, DPB1*102, DPB1*103, DPB1*104, DPB1*105, DPB1*106, DPB1*107, DPB1*108, DPB1*109, DPB1*110, DPB1*111, DPB1*112, DPB1*113, DPB1*114, DPB1*115, DPB1*116, DPB1*117, DPB1*118, DPB1*119, DPB1*11, DPB1*120, DPB1*121, DPB1*122, DPB1*123, DPB1*124, DPB1*125, DPB1*126, DPB1*127, DPB1*128, DPB1*129, DPB1*130, DPB1*131, DPB1*132, DPB1*133, DPB1*134, DPB1*135, DPB1*136, DPB1*137, DPB1*138, DPB1*139, DPB1*13, DPB1*140, DPB1*141, DPB1*142, DPB1*143, DPB1*144, DPB1*145, DPB1*146, DPB1*147, DPB1*148, DPB1*149, DPB1*14, DPB1*150, DPB1*151, DPB1*152, DPB1*153, DPB1*154, DPB1*155, DPB1*156, DPB1*157, DPB1*158, DPB1*159, DPB1*15, DPB1*160, DPB1*161, DPB1*162, DPB1*163, DPB1*164, DPB1*165, DPB1*166, DPB1*167, DPB1*168, DPB1*169, DPB1*16, DPB1*170, DPB1*171, DPB1*172, DPB1*173, DPB1*174, DPB1*175, DPB1*176, DPB1*177, DPB1*178, DPB1*179, DPB1*17, DPB1*180, DPB1*181, DPB1*182, DPB1*183, DPB1*184, DPB1*185, DPB1*186, DPB1*187, DPB1*188, DPB1*189, DPB1*18, DPB1*190, DPB1*191, DPB1*192, DPB1*193, DPB1*194, DPB1*195, DPB1*196, DPB1*197, DPB1*198, DPB1*199, DPB1*19, DPB1*200, DPB1*201, DPB1*202, DPB1*203, DPB1*204, DPB1*205, DPB1*206, DPB1*207, DPB1*208, DPB1*209, DPB1*20, DPB1*210, DPB1*211, DPB1*212, DPB1*213, DPB1*214, DPB1*215, DPB1*216, DPB1*217, DPB1*218, DPB1*219, DPB1*21, DPB1*220, DPB1*221, DPB1*222, DPB1*223, DPB1*224, DPB1*225, DPB1*226, DPB1*227, DPB1*228, DPB1*229, DPB1*22, DPB1*230, DPB1*231, DPB1*232, DPB1*233, DPB1*234, DPB1*235, DPB1*236, DPB1*237, DPB1*238, DPB1*239, DPB1*23, DPB1*240, DPB1*241, DPB1*242, DPB1*243, DPB1*244, DPB1*245, DPB1*246, DPB1*247, DPB1*248, DPB1*249, DPB1*24, DPB1*250, DPB1*251, DPB1*252, DPB1*253, DPB1*254, DPB1*255, DPB1*256, DPB1*257, DPB1*258, DPB1*259, DPB1*25, DPB1*260, DPB1*261, DPB1*262, DPB1*263, DPB1*264, DPB1*265, DPB1*266, DPB1*267, DPB1*268, DPB1*269, DPB1*26, DPB1*270, DPB1*271, DPB1*272, DPB1*273, DPB1*274, DPB1*275, DPB1*276, DPB1*277, DPB1*278, DPB1*279, DPB1*27, DPB1*280, DPB1*281, DPB1*282, DPB1*283, DPB1*284, DPB1*285, DPB1*286, DPB1*287, DPB1*288, DPB1*289, DPB1*28, DPB1*290, DPB1*291, DPB1*292, DPB1*293, DPB1*294, DPB1*295, DPB1*296, DPB1*297, DPB1*298, DPB1*299, DPB1*29, DPB1*300, DPB1*301, DPB1*302, DPB1*303, DPB1*304, DPB1*305, DPB1*306, DPB1*307, DPB1*308, DPB1*309, DPB1*30, DPB1*310, DPB1*311, DPB1*312, DPB1*313, DPB1*314, DPB1*315, DPB1*316, DPB1*317, DPB1*318, DPB1*319, DPB1*31, DPB1*320, DPB1*321, DPB1*322, DPB1*323, DPB1*324, DPB1*325, DPB1*326, DPB1*327, DPB1*328, DPB1*329, DPB1*32, DPB1*330, DPB1*331, DPB1*332, DPB1*333, DPB1*334, DPB1*335, DPB1*336, DPB1*337, DPB1*338, DPB1*339, DPB1*33, DPB1*340, DPB1*341, DPB1*342, DPB1*343, DPB1*344, DPB1*345, DPB1*346, DPB1*347, DPB1*348, DPB1*349, DPB1*34, DPB1*350, DPB1*351, DPB1*352, DPB1*353, DPB1*354, DPB1*355, DPB1*356, DPB1*357, DPB1*358, DPB1*359, DPB1*35, DPB1*360, DPB1*361, DPB1*362, DPB1*363, DPB1*364, DPB1*365, DPB1*366, DPB1*367, DPB1*368, DPB1*369, DPB1*36, DPB1*370, DPB1*371, DPB1*372, DPB1*373, DPB1*374, DPB1*375, DPB1*376, DPB1*377, DPB1*378, DPB1*379, DPB1*37, DPB1*380, DPB1*381, DPB1*382, DPB1*383, DPB1*384, DPB1*385, DPB1*386, DPB1*387, DPB1*388, DPB1*389, DPB1*38, DPB1*390, DPB1*391, DPB1*392, DPB1*393, DPB1*394, DPB1*395, DPB1*396, DPB1*397, DPB1*398, DPB1*399, DPB1*39, DPB1*400, DPB1*401, DPB1*402, DPB1*403, DPB1*404, DPB1*405, DPB1*406, DPB1*407, DPB1*408, DPB1*409, DPB1*40, DPB1*410, DPB1*411, DPB1*412, DPB1*413, DPB1*414, DPB1*415, DPB1*416, DPB1*417, DPB1*418, DPB1*419, DPB1*41, DPB1*420, DPB1*421, DPB1*422, DPB1*423, DPB1*424, DPB1*425, DPB1*426, DPB1*427, DPB1*428, DPB1*429, DPB1*430, DPB1*431, DPB1*432, DPB1*433, DPB1*434, DPB1*435, DPB1*436, DPB1*437, DPB1*438, DPB1*439, DPB1*440, DPB1*441, DPB1*442, DPB1*443, DPB1*444, DPB1*445, DPB1*446, DPB1*447, DPB1*448, DPB1*449, DPB1*44, DPB1*450, DPB1*451, DPB1*452, DPB1*453, DPB1*454, DPB1*455, DPB1*456, DPB1*457, DPB1*458, DPB1*459, DPB1*45, DPB1*460, DPB1*461, DPB1*462, DPB1*463, DPB1*464, DPB1*465, DPB1*466, DPB1*467, DPB1*468, DPB1*469, DPB1*46, DPB1*470, DPB1*471, DPB1*472, DPB1*473, DPB1*474, DPB1*475, DPB1*476, DPB1*477, DPB1*478, DPB1*479, DPB1*47, DPB1*480, DPB1*481, DPB1*482, DPB1*483, DPB1*484, DPB1*485, DPB1*486, DPB1*487, DPB1*488, DPB1*489, DPB1*48, DPB1*490, DPB1*491, DPB1*492, DPB1*493, DPB1*494, DPB1*495, DPB1*496, DPB1*497, DPB1*498, DPB1*499, DPB1*49, DPB1*500, DPB1*501, DPB1*502, DPB1*503, DPB1*504, DPB1*505, DPB1*506, DPB1*507, DPB1*508, DPB1*509, DPB1*50, DPB1*510, DPB1*511, DPB1*512, DPB1*513, DPB1*514, DPB1*515, DPB1*516, DPB1*517, DPB1*518, DPB1*519, DPB1*51, DPB1*520, DPB1*521, DPB1*522, DPB1*523, DPB1*524, DPB1*525, DPB1*526, DPB1*527, DPB1*528, DPB1*529, DPB1*52, DPB1*530, DPB1*531, DPB1*532, DPB1*533, DPB1*534, DPB1*535, DPB1*536, DPB1*537, DPB1*538, DPB1*539, DPB1*53, DPB1*540, DPB1*541, DPB1*542, DPB1*543, DPB1*544, DPB1*545, DPB1*546, DPB1*547, DPB1*548, DPB1*549, DPB1*54, DPB1*550, DPB1*551, DPB1*552, DPB1*553, DPB1*554, DPB1*555, DPB1*556, DPB1*557, DPB1*558, DPB1*559, DPB1*55, DPB1*560, DPB1*561, DPB1*562, DPB1*563, DPB1*564, DPB1*565, DPB1*566, DPB1*567, DPB1*568, DPB1*569, DPB1*56, DPB1*570, DPB1*571, DPB1*572, DPB1*573, DPB1*574, DPB1*575, DPB1*576, DPB1*577, DPB1*578, DPB1*579, DPB1*57, DPB1*580, DPB1*581, DPB1*582, DPB1*583, DPB1*584, DPB1*585, DPB1*586, DPB1*587, DPB1*588, DPB1*589, DPB1*58, DPB1*590, DPB1*591, DPB1*592, DPB1*593, DPB1*594, DPB1*595, DPB1*596, DPB1*597, DPB1*598, DPB1*599, DPB1*59, DPB1*600, DPB1*601, DPB1*602, DPB1*603, DPB1*604, DPB1*605, DPB1*606, DPB1*607, DPB1*608, DPB1*609, DPB1*60, DPB1*610, DPB1*611, DPB1*612, DPB1*613, DPB1*614, DPB1*615, DPB1*616, DPB1*617, DPB1*618, DPB1*619, DPB1*61, DPB1*620, DPB1*621, DPB1*622, DPB1*623, DPB1*624, DPB1*625, DPB1*626, DPB1*627, DPB1*628, DPB1*629, DPB1*62, DPB1*630, DPB1*631, DPB1*632, DPB1*633, DPB1*634, DPB1*635, DPB1*636, DPB1*637, DPB1*638, DPB1*639, DPB1*63, DPB1*640, DPB1*641, DPB1*642, DPB1*643, DPB1*644, DPB1*645, DPB1*646, DPB1*647, DPB1*648, DPB1*649, DPB1*64, DPB1*650, DPB1*651, DPB1*652, DPB1*653, DPB1*654, DPB1*655, DPB1*656, DPB1*657, DPB1*658, DPB1*659, DPB1*65, DPB1*660, DPB1*661, DPB1*662, DPB1*663, DPB1*664, DPB1*665, DPB1*666, DPB1*667, DPB1*668, DPB1*669, DPB1*66, DPB1*670, DPB1*671, DPB1*672, DPB1*673, DPB1*674, DPB1*675, DPB1*676, DPB1*677, DPB1*678, DPB1*679, DPB1*67, DPB1*680, DPB1*681, DPB1*682, DPB1*683, DPB1*684, DPB1*685, DPB1*686, DPB1*687, DPB1*688, DPB1*689, DPB1*68, DPB1*690, DPB1*691, DPB1*692, DPB1*693, DPB1*694, DPB1*695, DPB1*696, DPB1*697, DPB1*698, DPB1*699, DPB1*69, DPB1*700, DPB1*701, DPB1*702, DPB1*703, DPB1*704, DPB1*705, DPB1*706, DPB1*707, DPB1*708, DPB1*709, DPB1*70, DPB1*710, DPB1*711, DPB1*712, DPB1*713, DPB1*714, DPB1*715, DPB1*716, DPB1*717, DPB1*718, DPB1*719, DPB1*71, DPB1*720, DPB1*721, DPB1*722, DPB1*723, DPB1*724, DPB1*725, DPB1*726, DPB1*727, DPB1*728, DPB1*729, DPB1*72, DPB1*730, DPB1*731, DPB1*732, DPB1*733, DPB1*734, DPB1*735, DPB1*736, DPB1*737, DPB1*738, DPB1*739, DPB1*73, DPB1*740, DPB1*741, DPB1*742, DPB1*743, DPB1*744, DPB1*745, DPB1*746, DPB1*747, DPB1*748, DPB1*749, DPB1*74, DPB1*750, DPB1*751, DPB1*752, DPB1*753, DPB1*754, DPB1*755, DPB1*756, DPB1*757, DPB1*758, DPB1*759, DPB1*75, DPB1*760, DPB1*761, DPB1*762, DPB1*763, DPB1*764, DPB1*765, DPB1*766, DPB1*767, DPB1*768, DPB1*769, DPB1*76, DPB1*770, DPB1*771, DPB1*772, DPB1*773, DPB1*774, DPB1*775, DPB1*776, DPB1*777, DPB1*778, DPB1*779, DPB1*77, DPB1*780, DPB1*781, DPB1*782, DPB1*783, DPB1*784, DPB1*785, DPB1*786, DPB1*787, DPB1*788, DPB1*789, DPB1*78, DPB1*790, DPB1*791, DPB1*792, DPB1*794, DPB1*795, DPB1*796, DPB1*797, DPB1*798, DPB1*799, DPB1*79, DPB1*800, DPB1*801, DPB1*802, DPB1*803, DPB1*804, DPB1*805, DPB1*806, DPB1*807, DPB1*808, DPB1*809, DPB1*80, DPB1*810, DPB1*811, DPB1*812, DPB1*813, DPB1*814, DPB1*815, DPB1*816, DPB1*817, DPB1*818, DPB1*819, DPB1*81, DPB1*820, DPB1*821, DPB1*822, DPB1*823, DPB1*824, DPB1*825, DPB1*826, DPB1*827, DPB1*828, DPB1*829, DPB1*82, DPB1*830, DPB1*831, DPB1*832, DPB1*833, DPB1*834, DPB1*835, DPB1*836, DPB1*837, DPB1*838, DPB1*839, DPB1*83, DPB1*840, DPB1*841, DPB1*842, DPB1*843, DPB1*844, DPB1*845, DPB1*846, DPB1*847, DPB1*848, DPB1*849, DPB1*84, DPB1*850, DPB1*851, DPB1*852, DPB1*853, DPB1*854, DPB1*855, DPB1*856, DPB1*857, DPB1*858, DPB1*859, DPB1*85, DPB1*860, DPB1*861, DPB1*862, DPB1*863, DPB1*864, DPB1*865, DPB1*866, DPB1*867, DPB1*868, DPB1*869, DPB1*86, DPB1*870, DPB1*871, DPB1*872, DPB1*873, DPB1*874, DPB1*875, DPB1*876, DPB1*877, DPB1*878, DPB1*879, DPB1*87, DPB1*880, DPB1*881, DPB1*882, DPB1*883, DPB1*884, DPB1*885, DPB1*886, DPB1*887, DPB1*888, DPB1*889, DPB1*88, DPB1*890, DPB1*891, DPB1*892, DPB1*893, DPB1*894, DPB1*895, DPB1*896, DPB1*897, DPB1*898, DPB1*899, DPB1*89, DPB1*900, DPB1*901, DPB1*902, DPB1*903, DPB1*904, DPB1*905, DPB1*906, DPB1*907, DPB1*908, DPB1*909, DPB1*90, DPB1*910, DPB1*911, DPB1*912, DPB1*913, DPB1*914, DPB1*915, DPB1*916, DPB1*917, DPB1*918, DPB1*919, DPB1*91, DPB1*920, DPB1*921, DPB1*922, DPB1*923, DPB1*924, DPB1*925, DPB1*926, DPB1*927, DPB1*928, DPB1*929, DPB1*92, DPB1*930, DPB1*931, DPB1*932, DPB1*933, DPB1*934, DPB1*935, DPB1*936, DPB1*937, DPB1*938, DPB1*939, DPB1*93, DPB1*940, DPB1*941, DPB1*942, DPB1*943, DPB1*944, DPB1*945, DPB1*946, DPB1*947, DPB1*948, DPB1*949, DPB1*94, DPB1*950, DPB1*951, DPB1*952, DPB1*953, DPB1*954, DPB1*955, DPB1*956, DPB1*957, DPB1*958, DPB1*959, DPB1*95, DPB1*960, DPB1*961, DPB1*962, DPB1*963, DPB1*964, DPB1*965, DPB1*96, DPB1*97, DPB1*98, and DPB1*99 allele.

9. The method of any one of claims 1 to 8, wherein the alpha chain of the MHC class II molecule comprises an HLA-DPA1*01, HLA-DPA1*02, HLA-DPA1*03, or HLA-DPA1*04 allele.

10. The method of any one of claims 6 to 9, wherein the beta chain of the MHC class II molecule comprises an amino acid other than leucine at a position corresponding to amino acid residue 112 of SEQ ID NO: 1.

11. The method of any one of claims 6 to 10, wherein the beta chain of the MHC class II molecule comprises an amino acid other than valine at a position corresponding to amino acid residue 141 of SEQ ID NO: 1.

12. A method of identifying a MHC class II-specific T cell receptor (TCR) comprising contacting a T cell with a complex comprising an MHC class II molecule and a peptide;
wherein the T cell expresses CD4 and one or more TCRs;
wherein the MHC class II molecule comprises an alpha chain and a beta chain, wherein the beta chain of the MHC class II molecule comprises (i) an amino acid other than leucine at a position corresponding to amino acid residue 112 of SEQ ID NO: 1, (ii) an amino acid other than valine at a position corresponding to amino acid residue 141 of SEQ ID NO: 1, or (iii) both (i) and (ii); and
wherein the MHC class II-specific TCR specifically binds the complex comprising the MHC class II molecule and the peptide.

13. The method of claim 12, wherein the MHC class II molecule has a higher affinity for CD4 than a naturally occurring MHC class II molecule has for CD4.

14. The method of any one of claims 10 to 13, wherein the amino acid other than leucine at a position corresponding to amino acid residue 112 of SEQ ID NO: 1 comprises a hydrophobic side chain.

15. The method of any one of claims 10 to 14, wherein the amino acid other than leucine at a position corresponding to amino acid residue 112 of SEQ ID NO: 1 is selected from the group consisting of an alanine, a valine, an isoleucine, a methionine, a phenylalanine, a tyrosine, and a tryptophan.

16. The method of any one of claims 10 to 15, wherein the amino acid other than leucine at a position corresponding to amino acid residue 112 of SEQ ID NO: 1 is a tryptophan.

17. The method of any one of claims 10 to 16, wherein the amino acid other than valine at a position corresponding to amino acid residue 141 of SEQ ID NO: 1 comprises a hydrophobic side chain.

18. The method of any one of claims 10 to 17, wherein the amino acid other than valine at a position corresponding to amino acid residue 141 of SEQ ID NO: 1 is selected from an alanine, an isoleucine, a leucine, a methionine, a phenylalanine, a tyrosine, and a tryptophan.

19. The method of any one of claims 10 to 18, wherein the amino acid other than valine at a position corresponding to amino acid residue 141 of SEQ ID NO: 1 is a methionine.

20. The method of any one of claims 1 to 8, wherein (i) the beta chain of the HLA class II molecule is an HLA-DQ allele, (ii) the alpha chain of the HLA class II molecule is an HLA-DQ allele, or (iii) both (i) and (ii).

21. The method of claim 20, wherein the beta chain of the HLA class II molecule comprises a DQ2, DQ3, DQ4, DQ5, or DQ6 allele.

22. The method of claim 20 or 21, wherein the beta chain of the MHC class II molecule comprises an HLA-DQB1*02, HLA-DQB1*03, HLA-DQB1*04, HLA-DQB1*05, or HLA-DQB1*06 allele.

23. The method of any one of claims 20 to 22, wherein the alpha chain of the MHC class II molecule comprises an HLA-DQA1*01, HLA-DQA1*02, HLA-DQA1*03, HLA-DQA1*04, HLA-DQA1*05, or HLA-DQA1*06 allele.

24. The method of any one of claims 20 to 23, wherein the beta chain of the MHC class II molecule comprises
(a) an amino acid other than leucine at a position corresponding to amino acid residue 114 of SEQ ID NO: 11;
(b) an amino acid other than valine at a position corresponding to amino acid residue 143 of SEQ ID NO: 11; and
(c) at least three of:
(i) an amino acid other than asparagine at a position corresponding to amino acid residue 110 of SEQ ID NO: 11,
(ii) an amino acid other than isoleucine at a position corresponding to amino acid residue 116 of SEQ ID NO: 11,
(iii) an amino acid other than serine at a position corresponding to amino acid residue 118 of SEQ ID NO: 11, and
(iv) an amino acid other than proline at a position corresponding to amino acid residue 146 of SEQ ID NO: 11.

25. The method of any one of claims 20 to 24, wherein the beta chain of the MHC class II molecule comprises
(a) an amino acid other than leucine at a position corresponding to amino acid residue 114 of SEQ ID NO: 11;
(b) an amino acid other than valine at a position corresponding to amino acid residue 143 of SEQ ID NO: 11;
(c) an amino acid other than asparagine at a position corresponding to amino acid residue 110 of SEQ ID NO: 11;
(d) an amino acid other than isoleucine at a position corresponding to amino acid residue 116 of SEQ ID NO: 11;
(e) an amino acid other than serine at a position corresponding to amino acid residue 118 of SEQ ID NO: 11; and
(f) an amino acid other than proline at a position corresponding to amino acid residue 146 of SEQ ID NO: 11.

26. The method of claim 24 or 25, wherein the amino acid other than leucine at a position corresponding to amino acid residue 114 of SEQ ID NO: 11 comprises a hydrophobic side chain.

27. The method of any one of claims 24 to 26, wherein the amino acid other than leucine at a position corresponding to amino acid residue 114 of SEQ ID NO: 11 is selected from the group consisting of an alanine, a valine, an isoleucine, a methionine, a phenylalanine, a tyrosine, and a tryptophan.

28. The method of any one of claims 24 to 27, wherein the amino acid other than leucine at a position corresponding to amino acid residue 114 of SEQ ID NO: 11 is a tryptophan.

29. The method of any one of claims 24 to 28, wherein the amino acid other than valine at a position corresponding to amino acid residue 143 of SEQ ID NO: 11 comprises a hydrophobic side chain.

30. The method of any one of claims 24 to 29, wherein the amino acid other than valine at a position corresponding to amino acid residue 143 of SEQ ID NO: 11 is selected from an alanine, an isoleucine, a leucine, a methionine, a phenylalanine, a tyrosine, and a tryptophan.

31. The method of any one of claims 24 to 30, wherein the amino acid other than valine at a position corresponding to amino acid residue 143 of SEQ ID NO: 11 is a methionine.

32. The method of any one of claims 20 to 31, wherein the beta chain of the MHC class II molecule comprises an amino acid other than asparagine at a position corresponding to amino acid residue 110 of SEQ ID NO: 11.

33. The method of any one of claims 24 to 32, wherein the amino acid other than asparagine at a position corresponding to amino acid residue 110 of SEQ ID NO: 11 is selected from a serine, a threonine, and a glutamine.

34. The method of any one of claims 24 to 33, wherein the amino acid other than asparagine at a position corresponding to amino acid residue 110 of SEQ ID NO: 11 is a glutamine.

35. The method of any one of claims 20 to 33, wherein the beta chain of the MHC class II molecule comprises an amino acid other than isoleucine at a position corresponding to amino acid residue 116 of SEQ ID NO: 11.

36. The method of any one of claims 24 to 35, wherein the amino acid other than isoleucine at a position corresponding to amino acid residue 116 of SEQ ID NO: 11 is selected from an alanine, a valine, a leucine, a methionine, a phenylalanine, a tyrosine, and a tryptophan.

37. The method of any one of claims 24 to 36, wherein the amino acid other than isoleucine at a position corresponding to amino acid residue 116 of SEQ ID NO: 11 is valine.

38. The method of any one of claims 20 to 37, wherein the beta chain of the MHC class II molecule comprises an amino acid other than serine at a position corresponding to amino acid residue 118 of SEQ ID NO: 11.

39. The method of any one of claims 24 to 38, wherein the amino acid other than serine at a position corresponding to amino acid residue 118 of SEQ ID NO: 11 is selected from an arginine, a histidine, and a lysine.

40. The method of any one of claims 24 to 39, wherein the amino acid other than serine at a position corresponding to amino acid residue 118 of SEQ ID NO: 11 is a histidine.

41. The method of any one of claims 20 to 40, wherein the beta chain of the MHC class II molecule comprises an amino acid other than proline at a position corresponding to amino acid residue 146 of SEQ ID NO: 11.

42. The method of any one of claims 24 to 41, wherein the amino acid other than proline at a position corresponding to amino acid residue 146 of SEQ ID NO: 11 is selected from a serine, a threonine, an asparagine, and a glutamine.

43. The method of any one of claims 24 to 42, wherein the amino acid other than proline at a position corresponding to amino acid residue 146 of SEQ ID NO: 11 is a glutamine.

44. The method of any one of claims 1 to 8, wherein (i) the beta chain of the HLA class II molecule is an HLA-DR allele, (ii) the alpha chain of the HLA class II molecule is an HLA-DR allele, of (iii) both (i) and (ii).

45. The method of claim 44, wherein the beta chain of the HLA class II molecule comprises a DR2, DR3, DR4, DR5, DR6, DR7, DR8, DR9, DR10, DR11, DR12, DR13, DR14, DR15, or DR16 allele.

46. The method of claim 44 or 45, wherein the beta chain of the MHC class II molecule comprises an HLA allele selected from the group consisting of DRB1*01, DRB1*03, DRB1*04, DRB1*07, DRB1*08, DRB1*09, DRB1*10, DRB1*11, DRB1*12, DRB1*13, DRB1*14, DRB1*15, and DRB1*16.

47. The method of any one of claims 44 to 46, wherein the alpha chain of the MHC class II molecule comprises an HLA-DRA1*01 allele.

48. The method of any one of claims 44 to 47, wherein the beta chain comprises:
(a) an amino acid other than leucine at a position corresponding to amino acid residue 114 of SEQ ID NO: 19;
(b) an amino acid other than valine at a position corresponding to amino acid residue 143 of SEQ ID NO: 19; and
(c) at least two of:
(i) an amino acid other than serine at a position corresponding to amino acid residue 118 of SEQ ID NO: 19,
(ii) an amino acid other than lysine at a position corresponding to amino acid residue 139 of SEQ ID NO: 19,
(iii) an amino acid other than glycine at a position corresponding to amino acid residue 146 of SEQ ID NO: 19,
(iv) an amino acid other than threonine at a position corresponding to amino acid residue 157 of SEQ ID NO: 19,
(v) an amino acid other than threonine at a position corresponding to amino acid residue 163 of SEQ ID NO: 19, and
(vi) an amino acid other than valine at a position corresponding to amino acid residue 164 of SEQ ID NO: 19.

49. The method of claim 48, wherein the beta chain comprises:
(c) at least three of:
(i) an amino acid other than serine at a position corresponding to amino acid residue 118 of SEQ ID NO: 19,
(ii) an amino acid other than lysine at a position corresponding to amino acid residue 139 of SEQ ID NO: 19,
(iii) an amino acid other than glycine at a position corresponding to amino acid residue 146 of SEQ ID NO: 19,
(iv) an amino acid other than threonine at a position corresponding to amino acid residue 157 of SEQ ID NO: 19,
(v) an amino acid other than threonine at a position corresponding to amino acid residue 163 of SEQ ID NO: 19, and
(vi) an amino acid other than valine at a position corresponding to amino acid residue 164 of SEQ ID NO: 19.

50. The method of claim 48 or 49, wherein the beta chain comprises:
(c) at least four of:
(i) an amino acid other than serine at a position corresponding to amino acid residue 118 of SEQ ID NO: 19,
(ii) an amino acid other than lysine at a position corresponding to amino acid residue 139 of SEQ ID NO: 19,
(iii) an amino acid other than glycine at a position corresponding to amino acid residue 146 of SEQ ID NO: 19,
(iv) an amino acid other than threonine at a position corresponding to amino acid residue 157 of SEQ ID NO: 19,
(v) an amino acid other than threonine at a position corresponding to amino acid residue 163 of SEQ ID NO: 19, and
(vi) an amino acid other than valine at a position corresponding to amino acid residue 164 of SEQ ID NO: 19.

51. The method of any one of claims 44 to 50, wherein the beta chain comprises:
(a) an amino acid other than leucine at a position corresponding to amino acid residue 114 of SEQ ID NO: 19,
(b) an amino acid other than valine at a position corresponding to amino acid residue 143 of SEQ ID NO: 19,
(c) an amino acid other than serine at a position corresponding to amino acid residue 118 of SEQ ID NO: 19, and
(d) an amino acid other than threonine at a position corresponding to amino acid residue 157 of SEQ ID NO: 19.

52. The method of any one of claims 44 to 51, wherein the beta chain comprises:
(a) an amino acid other than leucine at a position corresponding to amino acid residue 114 of SEQ ID NO: 19,
(b) an amino acid other than valine at a position corresponding to amino acid residue 143 of SEQ ID NO: 19,
(c) an amino acid other than serine at a position corresponding to amino acid residue 118 of SEQ ID NO: 19,
(d) an amino acid other than lysine at a position corresponding to amino acid residue 139 of SEQ ID NO: 19,
(e) an amino acid other than glycine at a position corresponding to amino acid residue 146 of SEQ ID NO: 19,
(f) an amino acid other than threonine at a position corresponding to amino acid residue 157 of SEQ ID NO: 19,
(g) an amino acid other than threonine at a position corresponding to amino acid residue 163 of SEQ ID NO: 19, and
(h) an amino acid other than valine at a position corresponding to amino acid residue 164 of SEQ ID NO: 19.

53. The method of any one of claims 48 to 52, wherein the amino acid other than leucine at a position corresponding to amino acid residue 114 of SEQ ID NO: 19 comprises a hydrophobic side chain.

54. The method of any one of claims 48 to 53, wherein the amino acid other than leucine at a position corresponding to amino acid residue 114 of SEQ ID NO: 19 is selected from the group consisting of an alanine, a valine, an isoleucine, a methionine, a phenylalanine, a tyrosine, and a tryptophan.

55. The method of any one of claims 48 to 54, wherein the amino acid other than leucine at a position corresponding to amino acid residue 114 of SEQ ID NO: 19 is a tryptophan.

56. The method of any one of claims 48 to 55, wherein the amino acid other than valine at a position corresponding to amino acid residue 143 of SEQ ID NO: 19 comprises a hydrophobic side chain.

57. The method of any one of claims 48 to 56, wherein the amino acid other than valine at a position corresponding to amino acid residue 143 of SEQ ID NO: 19 is selected from an alanine, an isoleucine, a leucine, a methionine, a phenylalanine, a tyrosine, and a tryptophan.

58. The method of any one of claims 48 to 57, wherein the amino acid other than valine at a position corresponding to amino acid residue 143 of SEQ ID NO: 19 is a methionine.

59. The method of any one of claims 44 to 58, wherein the beta chain of the MHC class II molecule comprises an amino acid other than serine at a position corresponding to amino acid residue 118 of SEQ ID NO: 19.

60. The method of any one of claims 48 to 59, wherein the amino acid other than serine at a position corresponding to amino acid residue 118 of SEQ ID NO: 19 is selected from an arginine, a histidine, and a lysine.

61. The method of any one of claims 48 to 60, wherein the amino acid other than serine at a position corresponding to amino acid residue 118 of SEQ ID NO: 19 is a histidine.

62. The method of any one of claims 44 to 61, wherein the beta chain of the MHC class II molecule comprises an amino acid other than lysine at a position corresponding to amino acid residue 139 of SEQ ID NO: 19.

63. The method of any one of claims 48 to 62, wherein the amino acid other than lysine at a position corresponding to amino acid residue 139 of SEQ ID NO: 19 is selected from a serine, a threonine, and a glutamine.

64. The method of any one of claims 48 to 63, wherein the amino acid other than lysine at a position corresponding to amino acid residue 139 of SEQ ID NO: 19 is a threonine.

65. The method of any one of claims 44 to 64, wherein the beta chain of the MHC class II molecule comprises an amino acid other than glycine at a position corresponding to amino acid residue 146 of SEQ ID NO: 19.

66. The method of any one of claims 48 to 65, wherein the amino acid other than glycine at a position corresponding to amino acid residue 146 of SEQ ID NO: 19 is selected from a serine, an asparagine, a threonine, and a glutamine.

67. The method of any one of claims 48 to 66, wherein the amino acid other than glycine at a position corresponding to amino acid residue 146 of SEQ ID NO: 19 is a glutamine.

68. The method of any one of claims 44 to 67, wherein the beta chain of the MHC class II molecule comprises an amino acid other than threonine at a position corresponding to amino acid residue 157 of SEQ ID NO: 19.

69. The method of any one of claims 48 to 68, wherein the amino acid other than threonine at a position corresponding to amino acid residue 157 of SEQ ID NO: 19 is selected from an alanine, a valine, an isoleucine, a leucine, a methionine, a phenylalanine, a tyrosine, and a tryptophan.

70. The method of any one of claims 48 to 69, wherein the amino acid other than threonine at a position corresponding to amino acid residue 157 of SEQ ID NO: 19 is an isoleucine.

71. The method of any one of claims 44 to 70, wherein the beta chain of the MHC class II molecule comprises an amino acid other than threonine at a position corresponding to amino acid residue 163 of SEQ ID NO: 19.

72. The method of any one of claims 48 to 71, wherein the amino acid other than threonine at a position corresponding to amino acid residue 163 of SEQ ID NO: 19 is selected from an alanine, a valine, an isoleucine, a leucine, a methionine, a phenylalanine, a tyrosine, and a tryptophan.

73. The method of any one of claims 48 to 72, wherein the amino acid other than threonine at a position corresponding to amino acid residue 163 of SEQ ID NO: 19 is a methionine.

74. The method of any one of claims 44 to 73, wherein the beta chain of the MHC class II molecule comprises an amino acid other than valine at a position corresponding to amino acid residue 164 of SEQ ID NO: 19.

75. The method of any one of claims 48 to 74, wherein the amino acid other than valine at a position corresponding to amino acid residue 164 of SEQ ID NO: 19 is selected from a serine, an asparagine, a threonine, and a glutamine.

76. The method of any one of claims 48 to 75, wherein the amino acid other than valine at a position corresponding to amino acid residue 164 of SEQ ID NO: 19 is a threonine.

77. The method of any one of claims 44 to 76, wherein the beta chain comprises:
(a) a tryptophan at a position corresponding to amino acid residue 114 of SEQ ID NO: 19,
(b) a methionine at a position corresponding to amino acid residue 143 of SEQ ID NO: 19,
(c) a histidine at a position corresponding to amino acid residue 118 of SEQ ID NO: 19, and
(d) an isoleucine at a position corresponding to amino acid residue 157 of SEQ ID NO: 19.

78. The method of any one of claims 1 to 11 and 13 to 77, wherein the naturally occurring MHC class II molecule comprises:
(a) a leucine at a position corresponding to amino acid residue 112 of SEQ ID NO: 1 or amino acid residue 114 of SEQ ID NO: 11 or 19,
(b) a valine at a position corresponding to amino acid residue 141 of SEQ ID NO: 1 or amino acid residue 143 of SEQ ID NO: 11 or 19, or
(c) both (a) and (b).

79. The method of any one of claims 1 to 11 and 13 to 78, wherein the naturally occurring MHC class II molecule comprises:
(a) a leucine at a position corresponding to amino acid residue 112 of SEQ ID NO: 1 or amino acid residue 114 of SEQ ID NO: 11 or 19,
(b) a valine at a position corresponding to amino acid residue 141 of SEQ ID NO: 1 or amino acid residue 143 of SEQ ID NO: 11 or 19,
(c) an asparagine at a position corresponding to amino acid residue 110 of SEQ ID NO: 11;
(d) an isoleucine at a position corresponding to amino acid residue 116 of SEQ ID NO: 11;
(e) a serine at a position corresponding to amino acid residue 118 of SEQ ID NO: 11 or 19; and
(f) a proline at a position corresponding to amino acid residue 146 of SEQ ID NO: 11
(g) a lysine at a position corresponding to amino acid residue 139 of SEQ ID NO: 19,
(h) a glycine at a position corresponding to amino acid residue 146 of SEQ ID NO: 19,
(i) a threonine at a position corresponding to amino acid residue 157 of SEQ ID NO: 19,
(j) a threonine at a position corresponding to amino acid residue 163 of SEQ ID NO: 19,
(k) a valine at a position corresponding to amino acid residue 164 of SEQ ID NO: 19, or
(l) any combination of (a) to (k).

80. The method of any one of claims 1 to 79, wherein the MHC class II molecule is a dimer.

81. The method of any one of claims 1 to 80, wherein the MHC class II molecule is a trimer.

82. The method of any one of claims 1 to 81, wherein the MHC class II molecule is a tetramer.

83. The method of any one of claims 1 to 82, wherein the peptide comprises a fragment of a protein.

84. The method of claim 83, wherein the protein is expressed by a diseased cell

85. The method of claim 83 or 84, wherein the protein is expressed by a tumor cell.

86. The method of any one of claims 1 to 85, wherein the peptide comprises at least about 10 amino acids.

87. The method of claim 86, wherein the peptide comprises about 10 to about 100 amino acids, about 10 to about 90 amino acids, about 10 to about 80 amino acids, about 10 to about 70 amino acids, about 10 to about 60 amino acids, about 10 to about 50 amino acids, about 10 to about 40 amino acids, about 10 to about 30 amino acids, about 10 to about 25 amino acids, about 10 to about 20 amino acids, about 10 to about 15 amino acids, about 15 to about 100 amino acids, 20 to about 100 amino acids, 25 to about 100 amino acids, 30 to about 100 amino acids, 35 to about 100 amino acids, 40 to about 100 amino acids, 50 to about 100 amino acids, 60 to about 100 amino acids, 70 to about 100 amino acids, 80 to about 100 amino acids, or 90 to about 100 amino acids.

88. The method of claim 86 or 87, wherein the peptide comprises about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, or about 100 amino acids.

89. The method of any one of claims 1 to 88, wherein the MHC class II molecule is expressed on the surface of an antigen presenting cell.

90. The method of any one of claims 1 to 89, wherein the T cell is obtained from a human subject.

91. The method of any one of claims 1 to 90, wherein the T cell is a tumor infiltrating lymphocyte (TIL).

92. The method of any one of claims 1 to 91, wherein the MHC class II molecule has an affinity for CD4 that is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, or at least about 100-fold higher than the binding affinity of a naturally occurring MHC class II molecule to CD4.

93. The method of any one of claims 1 to 92, further comprising selecting the T cell that is bound by the MHC class II molecule.

94. The method of any one of claims 1 to 93, further comprising isolating the TCR that is bound to the MHC class II molecule.

95. The method of claim 94, further comprising sequencing the TCR.

96. The method of claims 94 or 95, further comprising cloning the TCR.

97. The method of any one of claims 94 to 96, further comprising recombinantly expressing the TCR in a host cell.

98. The method of any one of claims 1 to 97, wherein the MHC class II molecule binds CD4 with a K_{D} of less than about 100 μM, less than about 50 μM, less than about 20 μM, or less than about 10 μM.

99. The method of any one of claims 1 to 97, wherein the MHC class II molecule binds CD4 with a K_{D} of about 14 μM or less.

100. The method of any one of claims 1 to 97, wherein the MHC class II molecule binds CD4 with a K_{D} of about 8.9 μM or less.
